(19) United States
(12) Patent Application Publication
(10) Pub. No.: US 2011/0206638 A1
(43) Pub. Date: Aug. 25, 2011

(54) COMPOSITIONS AND METHODS FOR REDUCING THE MUTATION RATE OF VIRUSES

(75) Inventor: Jinah Choi, Merced, CA (US)

(73) Assignee: The Regents of the University of California

(21) Appl. No.: 13/009,737

(22) Filed: Jan. 19, 2011

Related U.S. Application Data
(60) Provisional application No. 61/296,769, filed on Jan. 20, 2010.

(51) Int. Cl. C12Q 1/70; C12N 5/10; A61K 38/21; A61K 31/33; A61K 31/7056; A61K 31/195; A61K 31/444; A61K 31/44; A61K 31/215; A61K 31/41; A61K 31/34; A61K 31/355; A61K 31/015

(52) U.S. Cl. 424/85.7; 435/375; 424/85.4; 514/183; 514/43; 514/566; 514/332; 514/354; 514/507; 514/348; 514/383; 514/562; 514/474; 514/458; 514/763; 435/5

(57) ABSTRACT

The disclosure provides compositions and methods for reducing the mutation rate of a virus, such as an RNA virus or a DNA virus, in a cell infected with the virus by, in one aspect, contacting the cell with an effective amount of an iron chelator or an antioxidant. Also provided are compositions and methods for enhancing the efficacy of an agent or a therapy directed at a virus.

### CROSS-REFERENCE TO RELATED APPLICATION

This application claims the benefit under 35 U.S.C. §119(e) of U.S. Provisional Ser. No. 61/296,769, filed Jan., 20 2010, the content of which is incorporated by reference in its entirety into the present disclosure.

### BACKGROUND

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

RNA viruses are ubiquitous intracellular parasites with compacted genome. RNA viruses are characterized by rapid replication and a particularly high level of genetic variability and exist as a population of closely related but distinct genetic variants, referred to as “quasispecies”. Such high rate of mutation allows these viruses to operate near the error threshold, for maximal adaptability to changing environment while conserving essential genetic information. The genetic plasticity of RNA viruses can help increase their rates of evolution by millions-fold over those of the eukaryotic hosts. Consequently, the quasispecies nature of viruses has been a subject of active investigation in the study of drug resistance and immune escape. The pronounced genetic variability of RNA viruses has traditionally been attributed to increased rate of mutations due to the lack of proofreading activity of viral replicases as well as gene duplication, redundancy, and recombination. In addition, viral evolution is significantly affected by the environment, and host/environmental factors may also play a role in the generation of quasispecies through previously unknown mechanism(s).

Hepatitis C virus (HCV) is a single, positive-sense stranded RNA virus of Flaviviridae Family. The HCV genome is about 9.6 kb in length and consists of the 5′ untranslated region (UTR), the structural protein coding regions (C, E1, E2) the nonstructural (p7, NS2, NS3, NS4A/B, NS5A/B) protein-coding regions, and the 3′ UTR (FIG. 1A). HCV is estimated to infect about 170 million people worldwide, including four million people in the U.S. alone. Current anti-HCV therapy, that combines the use of interferon alpha and a nucleoside analog, ribavirin, results in sustained virological response (SVR) in only about 50-60% of patients undergoing treatment, the remainder showing resistance to therapy.

Both the efficacy of antiviral therapy and the success of re-treating resistant population with antiviral compounds are strongly affected by HCV genotype, indicating an importance of the viral sequence in the antiviral resistance. Specific sequences within the HCV RNA genome have also been identified that may determine the sensitivity of the virus to interferon. In addition, alcohol consumption has been associated with interferon resistance while antioxidants can improve the outcome of anti-HCV therapy. Furthermore, like other RNA viruses, HCV circulates as a population of different but highly homologous genomes, and the heterogeneous nature of HCV genomes complicate anti-HCV therapy and the development of vaccines. Recent reports also indicate a correlation between alcohol drinking behavior and increased heterogeneity of HCV RNA in patients, although no mechanism was presented.

Ethanol metabolism generates acetaldehyde which can directly bind DNA to induce DNA damage. Ethanol also increases the generation of ROS, decreases glutathione (GSH) content, causes lipid peroxidation, and can lead to selenium deficiency through multiple mechanisms. ROS and lipid peroxidation products can attack the bases and the deoxyribose backbone of DNA, causing single- and double strand breaks and other genotoxic/mutagenic effects. Reactive nitrogen species (RNS) can also induce DNA damage such as by nitrosative deamination and other modifications of bases and sugar. HCV infection itself is also associated with increased oxidative/nitrosative stress as well as decreased GSH and selenium concentrations. Increased amount of 8-hydroxydeoxyguanosine (8-OH-dG) has also been documented in hepatitis C patients and various in vitro models of HCV; 8-OH-dG is the most well-characterized product of oxidative/nitrosative modification of DNA which is mutagenic.

The presence of 8-OH-dG in hepatitis C patients suggests similar damages occurring on the cellular and viral RNAs. In fact, although chemical modification of nucleic acids is most extensively studied in the context of DNA damage in chemical carcinogenesis, reactive species can chemically modify RNAs as well as the DNA. RNA damage that ensues is less likely to be repaired than the corresponding changes, for example, to the host DNA, which would be particularly important for viruses that rely on RNA genomes. RNA damage will amplify the error rate of already error-prone viral replicases and increase the mutability of the viral RNA genomes, which in turn would provide the virus with a greater genetic repertoire to escape the host immune response and to develop resistance to antivirals and vaccines. Therefore, ethanol may synergize with the virally induced stress to promote the heterogeneity of HCV RNA by its genotoxic effects. In fact, alcohol, genotype 1, iron overload, age, co-infection with HIV, steatosis, fibrosis, and male gender, all of which are associated with increased reactive oxygen/nitrogen species (ROS/RNS) and/or decreased antioxidant capacity, have each been linked with interferon resistance.

### SUMMARY

This disclosure provides methods for reducing the mutation rate of a RNA or DNA virus which decreases the ability of the virus to persist in a cell infected with the virus, comprising, or alternatively consisting essentially of, or alternatively consisting of, contacting the cell with an effective amount of an iron chelator or an antioxidant, thereby reducing the mutation rate of the RNA or the DNA virus in the cell. Further, the iron chelator or the antioxidant may assist the immune system's ability to clear or limit infection by reducing the ability of the virus to escape the immune system by mutation. Thus, this disclosure provides this therapy as well. The contacting can be conducted in vitro or in vivo.

Another aspect of the disclosure provides methods for enhancing the efficacy of an agent or a therapy to reduce or prevent infection of a cell by an RNA or a DNA virus, comprising or alternatively consisting essentially of, or alternatively consisting of, contacting the cell with (i) an effective amount of an iron chelator or an antioxidant and (ii) the agent or the therapy, thereby enhancing the efficacy of the agent or the therapy to reduce or prevent infection of the cell by the RNA or the DNA virus. The contacting of (i) the iron chelator or antioxidant and (ii) the agent or the therapy to the cell can be sequential or concurrent. The contacting can be conducted in vitro or in vivo.

Suitable examples of agents or therapies directed at an RNA or a DNA virus include, but are not limited to administering an effective amount of one or more of interferon, plerixafor, ribavirin, pegylated interferon-alpha-2a, pegylated interferon-alpha-2b, immunoglobulins, or vaccines. The agents or therapies, alone or in combination, can be formulated into pharmaceutical compositions or they can be directly contacted with the cell.

Suitable iron chelators include, for example, diethylene triamine pentaacetic acid (DETAPAC), dipyridyl, pyridoxal isonicotinoyl hydrazone (PIH), desferrioxamine (DFO), deferiprone (DFP) or deferasirox (DFS). The iron chelators, alone or in combination, can be formulated into pharmaceutical compositions or they can be in direct contact with the cell.

Suitable antioxidants include, for example, glutathione, N-acetylcysteine, ascorbic acid, vitamin E, beta-carotene, a polyphenol, flavonoid and an agent that decreases the generation of free radical and non-radical reactive species, including, for example, a CYP2E1 inhibitor, an NAD(P)H oxidase inhibitor or a nitric oxide synthase inhibitor. The antioxidants, alone or in combination, can be formulated into pharmaceutical compositions or they can be directly contacted with the cell.

Suitable examples of RNA or DNA viruses that are inhibited by these methods described herein include, but are not limited to, human immunodeficiency viruses (HIV), influenza viruses, rous sarcoma viruses (RSV), hepatitis B viruses (HBV), cucumber mosaic virus (CMV), Herpes simplex virus-1 (HSV-1), encephalomyocarditis viruses (EMCV), Japanese encephalitis viruses, Epstein-Barr viruses (EBV), Sendai viruses, Feline immunodeficiency virus (FIV) or vesicular stomatitis virus (VSV). Suitable examples of RNA viruses can also be positive sense single strand RNA viruses, such as, but not limited to, Flaviviridae family viruses (e.g., HCV), Dengue viruses, yellow fever viruses and West Nile Viruses.

This disclosure provides a method for treating or preventing infection by an RNA or DNA virus in a subject, comprising or alternatively consisting essentially of, or alternatively consisting of, administering an effective amount of an iron chelator or an antioxidant to the subject which decreases the ability of the virus to persist, thereby treating or preventing infection by the RNA or DNA virus in the subject. In one aspect, the method further comprises, or alternatively consisting essentially of, or yet further consisting of, administering to the subject an effective amount of an agent or a therapy directed at an RNA or DNA virus. In some embodiments, the administering of the iron chelator or antioxidant and the administering of the agent or therapy is concurrent or sequential. These methods can be combined by art recognized therapies for a synergistic effect.

A subject suitably treated by one or more methods of this disclosure include, for examples, any animal that can be infected by an RNA or DNA virus, e.g., murines, rats, rabbit, simians, bovines, ovine, porcine, canines, feline, farm animals, sport animals, pets, equine, and primate, particularly human.

### DESCRIPTION OF THE FIGURES

FIG. 1A-B illustrate the genomic organization of HCV (A) and subgenomic HCV replicon (Con1) (B).

FIG. 2A-D show NS5A mutations by codon. (A-B) Cumulative synonymous and non-synonymous mutations were plotted against codons via SNAP. (C) Location of amino acid substitutions involving serines. Putative basal phosphorylation regions are shaded. (D) HCV NS5A protein. Numbers=NS5A amino acids numbering; Numbers in parenthesis=HCV polypeptide numbering, based on Con1.

FIG. 3A-C show the effect of ethanol on RNA damage. Huh7 cells were exposed to ethanol or GO+glucose for 24 hr (A). Con1 replicon cells were incubated with 0.5% ethanol, 20 μM BSO, or NiCl daily for 48 hr (B) or with 0.1% ethanol, BSO, or NiCl daily for 2 weeks (C). Then, samples were analyzed for 8-OHG by Northwestern (A) or ELISA (B-C). Untreated Huh7 and in vitro oxidized RNA's were used as the controls in (A). The bottom panel of (A) shows RNA loading by ethidium bromide stain.

FIG. 4 shows decreased RNA damage after certain treatments. SgPC2 Con1 replicon cells were incubated with 0.5% ethanol or 20 μM BSO daily for 48 hr with and without PIH, dipyridyl, GSH ester, NMA, DPI, or DADS. Then, the samples were analyzed for 8-OHG by ELISA.

FIG. 5A-B show the effects of ethanol and reactive species on oxidative RNA damage. (A) Con1 replicon cells were incubated with 0.5% (v/v) ethanol, 20 μM BSO, or 250 μM NiCl₂ daily for 48 hr. Then, samples were analyzed for 8-OHG by ELISA using 8-OHG as standard. Data were expressed as fold increase from the control (0.134±0.013 ng/ml). (B) Con1 replicon cells were incubated with 0.1% (v/v) ethanol, 20 μM BSO, or 50 μM NiCl₂ daily for 2 weeks and analyzed for 8-OHG by ELISA. Data were expressed as fold increase from the control (0.418±0.109 ng/ml). * Indicates statistically significant difference from the control (P<0.05; n=4 in panel A, n=6 in panel B).

FIG. 6A-C show effects of dipyridal and GSH ester on the oxidative RNA damage and HCV RNA sequence. Con1 replicon cells were incubated with 0.5 ethanol or 20 μM BSO daily for 48 hr with and without 10 μM dipyridyl or 2 mM GSH ester. Then, the samples were analyzed for 8-OHG by ELISA using 8-OHG as standard (A) or sequenced and analyzed for nt. (B) and amino acid substitution rates (C). Data were expressed as fold increase from the control where the control value in (A) was 0.122±0.027 ng/ml (n=6 per group). Control values in (B) and (C) were 2.88±0.77×10⁻⁴ nt. changes per site and 2.80±0.61×10⁻⁴ amino acid changes per site, respectively. Letter a indicates statistically significant difference from control (no inhibitor, P<0.05). Letters b and c represent statistically significant change from ethanol (no inhibitor) and BSO (no inhibitor), respectively (P<0.05).

FIG. 7A-B show oxidative RNA damage by HCV. Huh7 cells were transfected with JFH1 RNA and incubated with (A) 0.5% (v/v) ethanol or 20 μM BSO with and without 5 μM dipyridyl or 1 mM GSH ester daily for 48 hrs, or (B) 0.1% (v/v) ethanol or 20 μM BSO, with and without 5 μM dipyridyl or 1 mM GSH ester daily for 2 weeks. Then, the samples were analyzed for 8-OHG by ELISA using 8-OHG as standard. Data were expressed as fold increase from the control where the control value was 0.106±0.012 ng/ml and 0.291±0.054 ng/ml for the 48 hr and 2 week time points, respectively. Letters a and b indicate statistically significant difference from the −HCV control and +HCV control, respectively, and letter c indicates statistically significant difference from the respective no inhibitor controls (control no inhibitor, ethanol no inhibitor, and BSO no inhibitor) (P<0.05; n=4).

FIG. 8A-D show synonymous and nonsynonymous amino acid substitutions in the NS5A coding region. (A) Percentage of sites undergoing positive selection (dn/ds ratio>1) in control, ethanol, and BSO groups. ^{a} Indicates statistically significant difference from control (no inhibitor, P<0.05). ^{b} Indicates statistically significant difference from respective no inhibitor controls (control no inhibitor, ethanol no inhibitor, and BSO no inhibitor) (P<0.05). (B-D) Cumulative synonymous and non-synonymous mutations in (B) control, (C) ethanol, and (D) BSO groups were plotted against codons via SNAP. Numbers represent NS5A codon number.

FIG. 9 shows sites undergoing positive selection. Location of sites undergoing positive selection in the HCV database. Sites also found to undergo positive selection in this study are indicated by color (control, medium gray; ethanol, dark gray; and BSO, light gray).

FIG. 10A-C show mutations involving serine, threonine, and tyrosine by position. (A) Structural and functional domains of HCV NS5A. Numbers represent NS5A condon number; numbers in parenthesis depict Con1 polypeptide codon number. The figure is adapted from Huang Y. et al. (2007) Virology 364:1-9. (B) Location of amino acid substitutions involving serines. (C) Location of amino acid substitutions involving threonines and tyrosines. Putative basal phosphorylation regions are shaded.

FIG. 11 shows Y321C and cyclosporine resistance. Con1(wt) and Con1 (Y321 C) replicon cells were treated with 1 mM cyclosporine (CsA) for 48 hrs. Total RNA was extracted and HCV RNA was determined by real time qRT-PCR. Letter a indicates a statistically significant difference between control and cyclosporine treatment. Letter b Indicates a statistically significant difference between Con1(wt) and Con(Y321C) treated with cyclosporine.

FIG. 12 shows inhibition of the RNA damage by certain agents. Con1 replicon cells were incubated with 0.5% ethanol or 20 μM BSO daily for 48 hr with and without 0.1 mM DETAPAC, 5 μM PIH, 1 mM L-NMA, 40 μM DPI, or 25 μM DADS. Then, the samples were analyzed for 8-OHG by ELISA, as described. Data were expressed as fold increase from the control where the control value was 0.076±0.026 ng/ml. * Denotes statistically significant difference from the control (P<0.05; n=4).

FIG. 13 shows the sites undergoing positive selection, by group. Location of sites undergoing positive selection in control, ethanol, and BSO groups with and without dipyridyl or GSH ester, and the HCV database. Grey lines indicate sites undergoing positive selection that match between this study and the database.

FIG. 14 shows the position of amino acid variations in the genotype 1b NS5A sequences from database involving serines, threonines, and tyrosines.

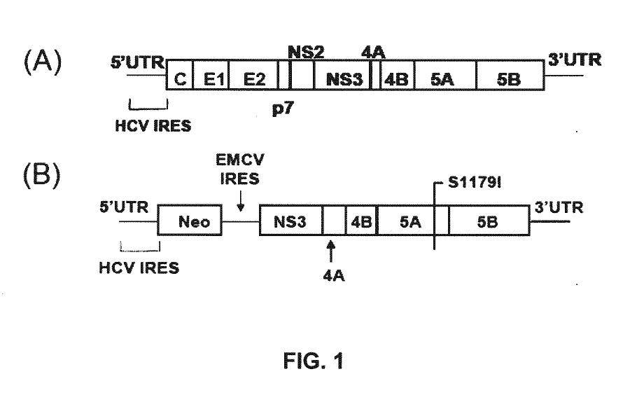

### DETAILED DESCRIPTION

### Definitions

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Techique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

An “RNA virus” is a virus that has RNA (ribonucleic acid) as its genetic material. This nucleic acid is usually single-stranded RNA (ssRNA) but may be double-stranded RNA (dsRNA). RNA viruses can be further classified according to the sense or polarity of their RNA into negative-sense and positive-sense (“positive sense single strand RNA virus”), or ambisense RNA viruses. Positive-sense viral RNA is similar to mRNA and thus can be immediately translated by the host cell. Negative-sense viral RNA is complementary to mRNA and thus must be converted to positive-sense RNA by an RNA polymerase before translation. As such, purified RNA of a positive-sense virus can directly cause infection though it may be less infectious than the whole virus particle. Purified RNA of a negative-sense virus is not infectious by itself as it needs to be transcribed into positive-sense RNA, however each virion can be transcribed to several positive-sense RNAs. Ambisense RNA viruses resemble negative-sense RNA viruses, except they also translate genes from the positive strand.

A “DNA virus” is a virus that has DNA as its genetic material and replicates using a DNA dependent DNA polymerase. DNA virus includes double-stranded DNA (dsDNA) and single-stranded DNA (ssDNA). Non-limiting examples of DNA virus include Herpes Simplex 1 Virus (HSV-1), Epstein Barr Virus (EBV), and Hepatitis B Virus (HBV).

The term “Flaviviridae” intends a family of viruses that are primarily spread through arthropod vectors (mainly ticks and mosquitoes). The family gets its name from Yellow Fever virus, a type virus of Flaviviridae; flavus means yellow in Latin. The Flaviviridae family includes the following genera: Genus Flavivirus, Genus Hepacivirus, Genus Pestivirus. Non-limiting examples of Flaviviridae family viruses include Yellow fever viruses, GB virus B, Japanese Encephaloviruses (JEV), West Nile viruses, Dengue Fever viruses, Hepatitis C viruses, Bovine virus diarrhea, swine fever or hog cholera viruses.

“Hepatitis C virus” or “HCV” intends a small (55-65 nm in size), enveloped, positive sense single strand RNA virus in the family Flaviviridae. The HCV particle consists of a core of genetic material (RNA), surrounded by an icosahedral protective shell of protein, and further encased in a lipid (fatty) envelope of cellular origin. Two viral envelope glycoproteins, E1 and E2, are embedded in the lipid envelope. Hepatitis C virus has a positive sense RNA genome that consists of a single open reading frame of 9600 nucleoside bases. At the 5′ and 3′ ends of the RNA are the UTR regions, that are not translated into proteins but are important to translation and replication of the viral RNA. The 5′ UTR has a ribosome binding site (IRES—Internal ribosome entry site) that starts the translation of a 3000 amino acid containing protein that is later cut by cellular and viral proteases into 10 active structural and non-structural smaller proteins.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

As used herein, a “mutation” is a randomly derived change to the nucleotide sequence of the genetic material of an organism. Mutations can be caused by copying errors in the genetic material during cell division, or by exposure to mutagens (ultraviolet or ionizing radiation, mutagenic chemicals, or viruses), or can be induced by the organism itself, by cellular processes such as hypermutation. In one aspect, a mutation can be caused by DNA or RNA damage or modification. In some embodiments, a RNA damage is caused by oxidative stress. A non-limiting example of RNA damage is modification of guanine to 8-Hydroxyguanosine (8-OHG).

The “infectivity” of a virus intends the ability of the virus to infect the host. Viral infection is affected by the infectivity, replicative fitness, and the ability of the virus to evade the host's immune response and develop resistance to antivirals.

“Chelation” intends the formation or presence of two or more separate bindings between a polydentate ligand and a single central atom. A “chelant” or “chelator” refers to a chemical that form a soluble and complex molecule with certain metal ions, inactivating the ions so that they cannot normally react with other elements or ions to produce precipitates or scale.

An “iron chelator” refers to a chelator that chelates with an iron ions, e.g., Fe³⁺. Non-limiting examples of iron chelators include diethylene triamine pentaacetic acid (DETAPAC), dipyridyl, pyridoxal isonicotinoyl hydrazone (PIH), desferrioxamine (DFO), deferiprone (DFP) or deferasirox (DFS) which chelates iron and inhibits metal-catalyzed reactions that produce free radical and non-radical reactive species, such as hydroxyl radical (HO^{.}).

“Diethylene triamine pentaacetic acid”, “DETAPAC” or “DTPA” intends a polyamino carboxylic acid consisting of a diethylenetriamine backbone modified with five carboxymethyl groups. The molecule can be viewed as an expanded version of ethylenediaminetetraacetic acid (EDTA). DETAPAC is used as its conjugate base, often undefined, which has a high affinity for metal cations. Upon complexation to lanthanide and actinide ions, DETAPAC exists as the pentaanionic form, i.e. all five carboxylic acid groups are deprotonated. In contrast to EDTA the compound possesses 7 centers to form coordination bonds with metals.

A “dipyridyl” or “bypyridine” refers to a compound formed by linking two pyridine rings by a single C—C bond, with the formula (C₅H₄N)₂. One of the isoforms of dipyridyl is 2,2′-Bipyridine which is a chelating ligand that forms complexes with most transition metal ions.

“Pyridoxal isonicotinoyl hydrazone” or “PIH” refers to an aroylhydrazone iron chelator disclosed in Ponka et al., (1979) FEBS Lett 97:317-321 and Baker et al., (1992) Hepatology 15:492-501.

“Desferrioxamine” or “DFO” (also known as desferrioxamine B, desferoxamine B, DFO-B, DFOA, DFB or desferal) is a bacterial siderophore produced by the actinobacter Streptomyces pilosus. It has medical applications as a chelating agent used to remove excess iron from the body. The mesylate salt of DFO-B is commercially available such as Desferal® marketed by Novartis AG.

“Deferiprone” or “DFP” refers to an oral drug that chelates iron and is used to treat thalassaemia major. DFP is commercially available as Ferriprox® marketed by ApoPharma Inc.

“Deferasirox” or “DFS” is an oral iron chelator. Its main use is to reduce chronic iron overload in patients who are receiving long-term blood transfusions for conditions such as beta-thalassemia and other chronic anemias. It is the first oral medication approved in the USA for this purpose. DFS is marketed as Exjade® by Novartis AG.

An “antioxidant” is a molecule capable of slowing or preventing the oxidation of other molecules. Oxidation is a chemical reaction that transfers electrons from a substance to an oxidizing agent. Oxidation reactions can produce free radicals, which cause oxidative stress and start chain reactions that damage cells. “Oxidative stress” is caused by an imbalance between the production of reactive oxygen and a biological system's ability to readily detoxify the reactive intermediates or easily repair the resulting damage. All forms of life maintain a reducing environment within their cells. This reducing environment is preserved by enzymes that maintain the reduced state through a constant input of metabolic energy. Disturbances in this normal redox state can cause toxic effects through the production of peroxides and free radicals that damage all components of the cell, including proteins, lipids, and DNA. Antioxidants terminate these chain reactions by removing free radical intermediates, and inhibit other oxidation reactions by being oxidized themselves. Examples of antioxidants include, but are not limited to, glutathione, N-acetylcysteine, ascorbic acid, vitamin E, beta-carotene, a polyphenol, flavonoid and an agent that decreases the generation of free radical and non-radical reactive species, including, for example, a CYP2E1 inhibitor, an NAD(P)H oxidase inhibitor or a nitric oxide synthase inhibitor.

“Ascorbic acid” or “vitamin C” refers a monosaccharide antioxidant found in both animals and plants. As one of the enzymes needed to make ascorbic acid has been lost by mutation during human evolution, it must be obtained from the diet and is a vitamin. Most other animals are able to produce this compound in their bodies and do not require it in their diets. In cells, it is maintained in its reduced form by reaction with glutathione, which can be catalysed by protein disulfide isomerase and glutaredoxins. Ascorbic acid is a reducing agent and can reduce, and thereby neutralize, reactive oxygen species such as hydrogen peroxide. In addition to its direct antioxidant effects, ascorbic acid is also a substrate for the antioxidant enzyme ascorbate peroxidase, a function that is particularly important in stress resistance in plants. Ascorbic acid is present at high levels in all parts of plants and can reach concentrations of 20 millimolar in chloroplasts. Ascorbic acid can be used in combination with iron chelator because it can act as a pro-oxidant in the presence of iron by reducing iron to Fe2+, which would increase the generation of potent oxidants that would damage the nucleic acids.

“Glutathione” intends a cysteine-containing peptide found in most forms of aerobic life. It is not required in the diet and is instead synthesized in cells from its constituent amino acids. Glutathione has antioxidant properties since the thiol group in its cysteine moiety is a reducing agent and can be reversibly oxidized and reduced. In cells, glutathione is maintained in the reduced form by the enzyme glutathione reductase and in turn reduces other metabolites and enzyme systems, such as ascorbate in the glutathione-ascorbate cycle, glutathione peroxidases and glutaredoxins, as well as reacting directly with oxidants. In some organisms glutathione is replaced by other thiols, such as by mycothiol in the Actinomycetes, or by trypanothione in the Kinetoplastids. Plasma and liver glutathione concentrations can be raised by oral administration of S-adenosylmethionine (SAMe). Glutathione precursors rich in cysteine include N-acetylcysteine (NAC) and undenatured whey protein, and these supplements have been shown to increase glutathione content within the cell. N-Acetylcysteine, is available both as a drug and as a generic supplement. Alpha Lipoic Acid has also been shown to restore intracellular glutathione. Melatonin has been shown to stimulate a related enzyme, glutathione peroxidase, and silymarin or milk thistle has also demonstrated an ability to replenish glutathione levels. Of all of these methods, the two methods that are the most thoroughly researched for efficacy in raising intracellular glutathione are variants of cysteine. N-acetyl-cysteine, which is a pharmaceutical over the counter drug, and bonded cysteine as is found in the undenatured whey protein nutraceutical Immunocal, are both proven to be efficacious in raising glutathione values. Also, glutathione can be supplied in the form of glutathione esters.

“Melatonin”, known chemically as N-acetyl-5-methoxytryptamine, refers to a naturally occurring hormone found in animals and in some other living organisms, including algae. Circulating levels vary in a daily cycle, and melatonin is important in the regulation of the circadian rhythms of several biological functions. Many biological effects of melatonin are due to its role as a pervasive and powerful antioxidant with a particular role in the protection of nuclear and mitochondrial DNA. Melatonin is an antioxidant that can easily cross cell membranes and the blood-brain barrier. Unlike other antioxidants, melatonin does not undergo redox cycling, which is the ability of a molecule to undergo repeated reduction and oxidation. Melatonin, once oxidized, forms several stable end-products upon reacting with free radicals.

“Vitamin E” is the collective name for a set of eight related tocopherols and tocotrienols, which are fat-soluble vitamins with antioxidant properties. A non-limiting example, α-tocopherol has been most studied as it has the highest bioavailability, with the body preferentially absorbing and metabolising this form. α-tocopherol protects membranes from oxidation by reacting with lipid radicals produced in the lipid peroxidation chain reaction. This removes the free radical intermediates and prevents the propagation reaction from continuing. This reaction produces oxidised α-tocopheroxyl radicals that can be recycled back to the active reduced form through reduction by other antioxidants, such as ascorbate, retinol or ubiquinol. This is in line with findings showing that α-tocopherol, but not water-soluble antioxidants, efficiently protects glutathione peroxidase 4 (GPX4)-deficient cells from cell death. Vitamin E is available from dietary sources such as asparagus, avocado, egg, milk, nuts, seeds, spinach, unheated vegetable oil, wheat germ or wholegrain foods.

“Beta-carotene” is a terpenoid, a red-orange pigment abundant in plants and fruits. As a carotene with beta-rings at both ends, it is the most common form of carotene. It is a precursor (inactive form) of vitamin A. Being highly conjugated, it is deeply colored, and as a hydrocarbon lacking functional groups, it is lipophilic. Beta-carotene is commercially available as Solatene® marketed by Roche Labs.

A “flavonoid” or “bioflavonoid”, also collectively known as Vitamin P and citrin, refers to a class of plant secondary metabolites. They can be classified into: flavonoids, derived from 2-phenylchromen-4-one (2-phenyl-1,4-benzopyrone) structure (examples: quercetin, rutin); isoflavonoids, derived from 3-phenylchromen-4-one (3-phenyl-1,4-benzopyrone) structure; neoflavonoids, derived from 4-phenylcoumarine (4-phenyl-1,2-benzopyrone) structure. The terms flavonoid and bioflavonoid have also been more loosely used to describe non-ketone polyhydroxy polyphenol compounds which are more specifically termed flavanoids, flavan-3-ols, or catechins (although catechins are actually a subgroup of flavanoids). Flavonoid is available from dietary sources such as citrus, ginkgo, tea, win and dark chocolate.

“NAD(P)H oxidase inhibitors” are known in the art and can include, but not limited to, apocynin and Diphenylene iodonium (DPI). “NAD(P)H oxidase” is intended to mean nicotinamide adenine dinucleotide phosphate-oxidase. Apocynin, also known as acetovanillone, is a natural organic compound structurally related to vanillin. It has been isolated from a variety of plant sources and is being studied for its variety of pharmacological properties. New inhibitors of NAD(P)H oxidase are further disclosed in, e.g., United States Patent Publication No: 20070082910.

“Nitric oxide synthase inhibitors” are known in the art and can include, but no limited to, N^{G}-methyl-L-arginine acetate (L-NMA), N^{ω}-Nitro-L-Arginine Methyl Ester (L-NAME), NO-Arg, L-N(G) methyl arginine hydrochloride and KD7332 (Bonnefous et al. (2009) J. Med. Chem. 52(9):3047-62).

“CYP2E1 inhibitors” refers to agents that inhibit the activity of Cytochrome P450 2E1 (CYP2E1), a member of the cytochrome P450 mixed-function oxidase system. Examples of CYP2E1 inhibitors includes, but are not limited to, diethyldithiocarbamate, cimetidine, disulfuram and diallyldisulfide.

Agents or therapies are known by the skilled artisan for treating and/or preventing infections of RNA viruses. Interferon, plerixafor, ribavirin, pegylated interferon-alpha-2a or pegylated interferon-alpha-2b have generally used for treating RNA viral infection.

“Interferons” (IFNs) are natural cell-signaling proteins produced by the cells of the immune system of most vertebrates in response to challenges such as viruses, parasites and tumor cells. They belong to the large class of glycoproteins known as cytokines and are produced by a wide variety of cells in response to the presence of double-stranded RNA, a key indicator of viral infection. Interferons assist the immune response by inhibiting viral replication within host cells, activating natural killer cells and macrophages, increasing antigen presentation to T lymphocytes, and increasing the resistance of host cells to viral infection. There are 3 known classes of interferons; type I, type II and type III. Interferons are commercially available, with brand names such as Roferon-A, Intron-A, Rebetron, Alferon-N, Peg-Intron, Avonex, Betaseron, Infergen, Actimmune and Pegasys.

“Plerixafor”, “MOZOBIL”, “JM 3100” or “AMD3100” refers to a macrocyclic compound and potential fusion inhibitor. It is an antagonist of the alpha-chemokine receptor CXCR4 & an allosteric agonist of CXCR7 (Kalatskaya et al. (2009) Molecular Pharmocology, 75:1240-1247).

“Ribavirin” intends an anti-viral drug indicated for severe RNA viral infections. Ribavirin is a prodrug, which when metabolized resembles purine RNA nucleotides. In this form it interferes with RNA metabolism required for viral replication. Multiple mechanisms may be responsible for its actions. Ribavirin is commercially available with brand names such as Copegus, Rebetol, Ribasphere, Vilona and Virazole.

“Pegylated interferon-alpha-2a” is an antiviral drug which has a dual mode of action—both antiviral and on the immune system. The addition of polyethylene glycol to the interferon, through a process known as pegylation, enhances the half-life of the interferon when compared to its native form. Pegylated interferon-alpha-2a is commercially available, e.g., Pegasys® from F. Hoffmann-La Roche.

“Pegylated interferon-alpha-2b” refers to a pegylated interferon composed of 165 amino acids. The PEG (polyethylene glycol) protects the molecule from proteolytic breakdown and increases the biological half-life of the interferon protein. While PEG-interferon alpha-2b is commonly used as a treatment for hepatitis C, it has also been shown to have optimistic effects in the treatment of malignant melanoma. It is also used in patients with neurofibromatosis to shrink neurofibromas. Pegylated interferon-alpha-2b is commercially available as Peg Intron® marketed by Schering-Plough.

Agents or therapies are known by the skilled artisan for treating and/or preventing infections of DNA viruses. Nucleoside analogs such as acyclovir (Zovirax®), famciclovir (Famvir®), valaciclovir (Valtrex®), lamivudine (Epivir®), adefovir (Hepsera®), tenofovir (Viread®), telbivudine (Tyzeka®), entecavir (Baraclude®), interferon-alpha 2 and pegylated interferon alpha 2a (Pegasys®) have generally been used for treating DNA viral infection.

A “composition” is intended to mean a combination of active polypeptide, polynucleotide or antibody and another compound or composition, inert (e.g. a detectable label) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present technology is also useful for veterinary treatment of companion mammals, exotic animals and domesticated animals, including mammals, rodents, and the like which is susceptible to RNA viral infection. In one embodiment, the mammals include horses, dogs, and cats. In another embodiment of the present disclosure, the human is an adolescent or infant under the age of eighteen years of age.

The terms “disease,” “disorder,” and “condition” are used inclusively and refer to any condition mediated at least in part by infection by an RNA virus such as HCV.

Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to reduce RNA virus mutation or damage in vitro or in vivo.

As used herein, the term “reduced” intends a lower level as compared to a control or a prior measurement or value. In one aspect, a reduced mutation rate of an RNA virus in a cell treated with an iron chelator or an antioxidant refers to a level of mutation rate that is lower than the level of mutation rate of the RNA virus in a cell not treated with the iron chelator or the antioxidant or alternatively, prior to such treatment. In another aspect, it is a lower mutation rate as compared to treatment with another, different agent, alone or in combination with the iron chelator or the antioxidant. Reduced intends a reduction by at least about 5%, or alternatively about 10%, or alternatively about 15%, or alternatively about 20%, or alternatively about 25%, or alternatively about 30%, or alternatively about 35%, or alternatively about 40%, or alternatively about 45%, or alternatively about 50%, or alternatively about 55%, or alternatively about 60%, or alternatively about 65%, or alternatively about 70%, or alternatively about 75%, or alternatively about 80%, or alternatively about 85%, or alternatively about 90%, or alternatively about 95%, or alternatively or about 100% as compared to a control or prior measurement or value.

As used herein, the term “enhanced” intends a higher level as compared to a control or a prior measurement or value. In one aspect, an enhanced efficacy of an agent or a therapy to reduce or prevent infection of a cell by an RNA virus, which cell is treated with an iron chelator or an antioxidant, is a higher efficacy as compared to the agent or therapy to reduce or prevent infection of the cell by the RNA virus, which cell is not treated with the iron chelator or the antioxidant. In another aspect, it is a higher efficacy as compared to treatment with another, different agent, alone or in combination with the iron chelator or the antioxidant. Enhanced intends an increase by at least about 5%, or alternatively about 10%, or alternatively about 15%, or alternatively about 20%, or alternatively about 25%, or alternatively about 30%, or alternatively about 35%, or alternatively about 40%, or alternatively about 45%, or alternatively about 50%, or alternatively about 55%, or alternatively about 60%, or alternatively about 65%, or alternatively about 70%, or alternatively about 75%, or alternatively about 80%, or alternatively about 85%, or alternatively about 90%, or alternatively about 95%, or alternatively or about 100%, as compared to a control or prior measurement or value.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. When the contacting is in vitro, the contacting can occur by adding the composition containing the active agent to the cell culture or alternatively, the active ingredients or agents can be directly added to the cell culture medium as needed. The disclosure is not limited by the route of administration, the formulation or dosing schedule.

### Methods to Reduce Viral Mutation

This disclosure provides a method for reducing the mutation rate of an RNA or DNA virus in a cell infected with the virus by reducing the ability of the virus to persist in the cell or host, comprising or alternatively consisting essentially of, or alternatively consisting of, contacting the cell with an effective amount of an iron chelator or an antioxidant, thereby reducing the mutation rate of the RNA or DNA virus in the cell. Further, the iron chelator or the antioxidant may assist the immune system's ability to clear or limit infection by reducing the ability of the virus to escape the immune system by mutation. Accordingly, also provided is a method for reducing infection by an RNA or a DNA virus, comprising or alternatively consisting essentially of, or alternatively consisting of, contacting the cell with an effective amount of an iron chelator or an antioxidant by reducing the mutation rate (e.g., selection of the RNA or DNA virus sequences that display increased replicative fitness and/or infectivity).

Another aspect of the disclosure provides a method for enhancing the efficacy of an agent or a therapy to reduce or prevent infection of a cell by an RNA or a DNA virus or to reduce the ability of the virus to persist in the cell or host, comprising or alternatively consisting essentially of, or alternatively consisting of, contacting the cell with (i) an effective amount of an iron chelator or an antioxidant and (ii) the agent or the therapy, thereby enhancing the efficacy of the agent or the therapy to reduce or prevent infection by RNA or DNA virus. The contacting of (i) the iron chelator or antioxidant and (ii) the agent or the therapy to the cell can be sequential or concurrent. As used herein, to enhance the efficacy intends to increase the efficacy of the agent or the therapy in the cell contacted with the iron chelator or the antioxidant as compared to in a cell not contacted or a prior measurement with the iron chelator or the antioxidant. Methods to determine efficacy are known in the art and include, without limitation, plaque assay.

Reduction of the mutation rate of an RNA or a DNA virus, in one aspect, refers to reduced damage of the RNA or DNA genome or provirus in an infected cell. In another aspect, mutations of RNA or DNA virus can be measured by noting RNA or DNA damages. For example, 8-hydroxyguanosine (8-OHG) and 8-hydroxydeoxyguanosine (8-OHdG) are abundant forms of oxidatively modified RNA and DNA species, respectively. To determine the level or rate of RNA damage, cells infected with the RNA or DNA virus (or, transfected with replicating viral RNAs) can be treated with a test agent for 24 hrs and analyzed for the level of 8-OHG in the cell (Shan et al. (2003) J. Neurosci. 23:4913-21). Alternatively or in addition, the genome is sequenced and analyzed and compared to the genome sequence in a cell not treated with an iron chelator or an antioxidant. In addition to sequencing, specific sequence changes can be probed, such as by the use of molecular beacons.

Furthermore, RNA mutation rate can be monitored by examining the rate of escape mutation (mutation that either restores a function or destroys a function that can be assayed for), which are known in the art. For example, cells can be transfected with replication-defective viral RNA harboring a critical point mutation, and the rate in which the replication-defective viral genome gains the ability to replicate can be monitored as an evidence of back-mutation. Alternatively, cells can be transfected with a reporter RNA encoding, for example, a mutated protein. Then, the re-gain of its function (by back-mutation) can be monitored. Then, one can monitor the test agent's ability to decrease these escape mutations.

All of these measurements (RNA damage, mutation rates) can be done in the presence of a genotoxin, to examine the agent's ability to slow down the RNA damage and genome mutation rate, when challenged with a genotoxin. For example, cells infected with RNA or DNA virus (or cells replicating an RNA or DNA viral genome) can be treated with a test agent and exposed to nickel, ethanol/acetaldehyde and other compounds that will increase the chemical modification of the RNAs in the cell. Then, the effects on the RNA or DNA damage and mutation rates can be compared to cells not treated with the agent, as described above.

Also, to measure the efficacy of a test agent in slowing down the mutation rate, cells infected with an RNA or DNA virus (or cells replicating an RNA viral genome) can be treated with a test agent in combination with interferon or other treatments of interest, and then the agent's ability to decrease the emergence of resistant viral sequences (persistence of infection) can be monitored by: 1. quantifying viral titer by conventional approaches known in the art; 2. sequencing the viral genome; and 3. monitoring the reversion mutation rates (see above for different methods), and comparing these to cells not treated with the agent.

Reduction of infection of a cell by an RNA or DNA virus intends a lower infection as compared to a cell not treated or prior to the treatment by an iron chelator or an antioxidant which can be measured by the viral load or number of copies of viral RNA in the cell, or alternatively by numbers of cells in a cell population infected by the virus. Methods of measure virus copies and the number of cells infected with viruses are known in the art and include, for example viral plaque assay.

Suitable examples of agents or therapies directed at an RNA or DNA virus include, but are not limited to interferon, plerixafor, ribavirin, pegylated interferon-alpha-2a or pegylated interferon-alpha-2b, or vaccines/immunoglobulins.

As used herein, any suitable cell that supports RNA or DNA viral reproduction and genomic replication is suitable for these methods. Examples of such include, eukaryotic cells such as animals, e.g., murines, rats, rabbit, simians, bovines, ovine, porcine, canines, feline, farm animals, sport animals, pets, equine, and primate, particularly human. The cells can be cultured cells or they can be primary cells. Cultured cell lines can be purchased from vendors such as the American Type Culture Collection (ATCC), U.S.A. In one particular embodiment, the cells are liver cells, e.g., cultured liver cells or primary liver cells. The cells are infected with RNA or DNA virus and may further exhibit pathology such as a liver carcinoma.

Suitable iron chelators include, for example, diethylene triamine pentaacetic acid (DETAPAC), dipyridyl, pyridoxal isonicotinoyl hydrazone (PIH), desferrioxamine (DFO), deferiprone (DFP) or deferasirox (DFS). The iron chelators, alone or in combination, can be formulated into pharmaceutical compositions or they can be directly contacted with the cell.

Suitable antioxidants include, for example, glutathione, N-acetylcysteine, ascorbic acid, vitamin E, beta-carotene, a polyphenol, flavonoid, and an agent that decreases the generation of free radical and non-radical reactive species, including, for example, a CYP2E1 inhibitor, an NAD(P)H oxidase inhibitor or a nitric oxide synthase inhibitor. The antioxidants, alone or in combination, can be formulated into pharmaceutical compositions or they can be directly contacted with the cell.

In one aspect, one or more iron chelators and one or more antioxidants can be formulated into pharmaceutical compositions or they can be directly contacted with the cell.

Suitable examples of RNA or DNA viruses that are inhibited by these methods include, but are not limited to, human immunodeficiency viruses (HIV), influenza viruses, rous sarcoma viruses (RSV), hepatitis B viruses (HBV), cucumber mosaic virus (CMV), Herpes simplex virus-1 (HSV-1), encephalomyocarditis viruses (EMCV), Japanese encephalitis viruses, Epstein-Barr viruses (EBV), Sendai viruses, Feline immunodeficiency virus (FIV) or vesicular stomatitis virus (VSV). Suitable examples of RNA viruses can also be positive sense single strand RNA viruses, such as, but not limited to Flaviviridae family viruses (e.g., HCV, Dengue viruses, yellow fever viruses and West Nile Viruses).

Further provided is a method for screening an agent useful for reducing the mutation rate of an RNA or DNA virus in a cell, comprising contacting the cell with an agent, the cell being infected with an RNA or DNA virus or in contact with an RNA or DNA virus, wherein a reduction of the mutation rate of the RNA or DNA virus in the cell as compared to a cell not being contacted with the agent indicates that the agent is useful for reducing the mutation rate of the RNA or DNA virus. In some embodiments, the cell is further in contact with an agent or a therapy directed at the RNA or DNA virus as described herein. One of skill in the art can determine if mutation rate has been reduced by measuring the damages on certain nucleotides such as the formation of 8-Hydroxyguanosine. Reduced intends a reduction by at least about 5%, or alternatively about 10%, or alternatively about 15%, or alternatively about 20%, or alternatively about 25%, or alternatively about 30%, or alternatively about 35%, or alternatively about 40%, or alternatively about 45%, or alternatively about 50%, or alternatively about 55%, or alternatively about 60%, or alternatively about 65%, or alternatively about 70%, or alternatively about 75%, or alternatively about 80%, or alternatively about 85%, or alternatively about 90%, or alternatively about 95%, or alternatively or about 100%.

Suitable examples of RNA or DNA viruses that are inhibited by these methods include, but are not limited to, human immunodeficiency viruses (HIV), influenza viruses, rous sarcoma viruses (RSV), hepatitis B viruses (HBV), cucumber mosaic virus (CMV), Herpes simplex virus-1 (HSV-1), encephalomyocarditis viruses (EMCV), Japanese encephalitis viruses, Epstein-Barr viruses (EBV), Sendai viruses, Feline immunodeficiency virus (FIV) or vesicular stomatitis virus (VSV). Suitable examples of RNA viruses can also be positive sense single strand RNA viruses, such as, but not limited to Flaviviridae family viruses (e.g., HCV, Dengue viruses, yellow fever viruses and West Nile Viruses). Suitable examples of DNA viruses include HBV, HSV-1, and EBV.

The methods can be practiced in vitro or in vivo. When practiced in vitro, they are effective means to identify and test therapeutic agents and regimens before advancement into the clinic. By having two cell systems, one can test or screen a potential therapeutic and compare its activity to those agents and combinations described herein. The methods can be modified for high-throughput testing of agents and potential therapeutics.

In vivo practice of the disclosure in an animal such as a rat or mouse provides a convenient animal model system that can be used prior to clinical testing of the agent. In this system, a potential agent, compound or composition will be successful if the mutation rate of the virus is reduced or the symptoms of the infection are ameliorated as compared to an untreated, infected animal. It also can be useful to have a separate negative control group of cells or animals which has not been infected, which provides a basis for comparison.

When practiced in vivo, the candidate is administered or delivered to the animal in effective amounts. As used herein, the term “administering” for in vivo and ex vivo purposes means providing the subject with an effective amount of the agent as described herein effective to inhibit retroviral replication as described herein. In these instances, the agent, compound or composition may be administered with a pharmaceutically acceptable carrier. These agents and combinations also can be used in the manufacture of medicaments and for the treatment of humans and other animals by administration in accordance with conventional procedures, such as an active ingredient in pharmaceutical compositions.

This disclosure provides a method for treating or preventing infection by an RNA or DNA virus in a subject, comprising administering an effective amount of an iron chelator or an antioxidant to the subject that reduces the ability of the virus to persist in the cell or host, thereby treating or preventing infection by the RNA or DNA virus in the subject. In one aspect, the method further comprises administering to the subject an effective amount of an agent or a therapy directed at an RNA or DNA virus. In some embodiments, the administering of the iron chelator or antioxidant and the administering of the agent or therapy is concurrent or sequential. “To treat the infection” intends (i) reducing replication of RNA or DNA virus in the subject, (ii) reducing or preventing infection of the RNA or DNA virus in uninfected cells in the subject, (iii) reducing the amount of RNA or DNA virus or (iv) eliminating the active, infective RNA or DNA virus in the subject. Treatment efficacy and viral numbers are determined by methods know in the art such as plaque assay or evaluated by the symptoms of the subject being treated. As used herein and when appropriate, treatment also intends a reduction or amelioration of clinical symptoms of the RNA or DNA viral infection.

A subject suitably treated by one or more methods of this disclosure include, for examples, any animal that can be infected by an RNA or DNA virus, e.g., murines, rats, rabbit, simians, bovines, ovine, porcine, canines, feline, farm animals, sport animals, pets, equine, and primate, particularly human.

Suitable examples of agents or therapies directed at an RNA or DNA virus include, but are not limited to interferon, plerixafor, ribavirin, pegylated interferon-alpha-2a or pegylated interferon-alpha-2b, or vaccines/immunoglobulins.

Suitable iron chelators include, for example, diethylene triamine pentaacetic acid (DETAPAC), dipyridyl, pyridoxal isonicotinoyl hydrazone (PIH), desferrioxamine (DFO), deferiprone (DFP) or deferasirox (DFS). The iron chelators, alone or in combination, can be formulated into pharmaceutical compositions or they can be directly contacted with the cell.

Suitable antioxidants include, for example, glutathione, N-acetylcysteine, ascorbic acid, vitamin E, beta-carotene, flavonoid, a CYP2E1 inhibitor, an NAD(P)H oxidase inhibitor or a nitric oxide synthase inhibitor. The antioxidants, alone or in combination, can be formulated into pharmaceutical compositions or they can be directly contacted with the cell.

In one aspect, one or more iron chelators and one or more antioxidants can be formulated into pharmaceutical compositions or they can be directly contacted with the cell.

Suitable examples of RNA or DNA viruses that are inhibited by these methods include, but are not limited to, human immunodeficiency viruses (HIV), influenza viruses, rous sarcoma viruses (RSV), hepatitis B viruses (HBV), cucumber mosaic virus (CMV), Herpes simplex virus-1 (HSV-1), encephalomyocarditis viruses (EMCV), Japanese encephalitis viruses, Epstein-Barr viruses (EBV), Sendai viruses, Feline immunodeficiency virus (FIV) or vesicular stomatitis virus (VSV). Suitable examples of RNA viruses can also be positive sense single strand RNA viruses, such as, but not limited to, Flaviviridae family viruses (e.g., HCV, Dengue viruses, yellow fever viruses and West Nile Viruses).

The methods of this disclosure present unexpected advantage by reducing the mutation rate of an RNA or DNA virus. Methods to determine the mutation rate or the rate of the damages leading to mutation are known in the art and briefly described herein.

Also provided herein is a method for treating diseases incident to DNA or RNA viral infection, e.g., liver disease incident to Hepatitis C Viral infection, in a subject by administering to the subject an effective amount of an iron chelator or antioxidant that reduces the mutation rate of an RNA or DNA virus in the subject. Suitable iron chelators and antioxidants are described herein. In one aspect, the method further comprising administering to the subject an effective amount of an agent or a therapy directed at an RNA or DNA virus. In some embodiments, the administering of the iron chelator or antioxidant and the administering of the agent or therapy is concurrent or sequential.

A subject suitably treated by one or more methods of this disclosure include, for examples, any animal that can be infected by an RNA or DNA virus, e.g., murines, rats, rabbit, simians, bovines, ovine, porcine, canines, feline, farm animals, sport animals, pets, equine, and primate, particularly human.

Examples of liver disease incident to HCV include, but are not limited to cirrhosis, steatosis or hepatocellular carcinoma.

Subjects that are suitably treated are described above and include any animal, vertebrate or mammal that is susceptible to RNA viral, and for example, HCV infection. Persons at risk for HCV infection include injecting drug users, recipients of clotting factors made before 1987, hemodialysis patients, recipients of blood and/or solid organs before 1992, people with undiagnosed liver problems, infants born to infected mothers and healthcare/public safety workers after known exposure.

Whether the treatment is successful can be determined upon whether it renders beneficial or desired results. In one aspect, the desired result is relief of RNA viral infection symptoms. In another aspect, the desired result is reduced RNA mutation or cellular damage in an infected cell which in turn increases the likelihood that the therapeutic agent will be effective in inhibiting viral replication and/or infection or reducing the symptoms of infection. In a further aspect, the desired result is reduced infection by the RNA or DNA virus.

### Compositions

This disclosure also provides compositions containing an effective amount of an iron chelator or an antioxidant and an effective amount of an agent or a therapy directed at an RNA or DNA virus as described herein to reduce or inhibit RNA or DNA viral mutation, the amounts being effective to inhibit or reduce RNA or DNA viral mutation. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this disclosure, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

This disclosure also provides a pharmaceutical combination comprising, or alternatively consisting essentially of, or yet further, consisting of, an effective amount of an iron chelator or an antioxidant and an effective amount of an agent or therapy directed at an RNA or DNA virus, wherein the effective amount is selected to effect one or more of the methods as described herein. As used herein, a pharmaceutical combination intends a pharmaceutical dosage of two or more agents that are to utilized in combination. The agents may be formulated separately but administered either concurrently or sequentially, as is deemed most effective. The combinations can in one aspect, allow for a reduced dosage of one of the agents as compared to the dosage when used as a monotherapy.

In one aspect for the composition or the combination, the iron chelator is one or more selected from the group diethylene triamine pentaacetic acid (DETAPAC), dipyridyl, pyridoxal isonicotinoyl hydrazone (PIH), desferrioxamine (DFO), deferiprone (DFP) or deferasirox (DFS).

In another aspect for the composition or the combination, the antioxidant is one or more selected from the group glutathione, N-acetylcysteine, ascorbic acid, vitamin E, beta-carotene, a polyphenol, flavonoid, and an agent that decreases the generation of free radical and non-radical reactive species, including, for example, a CYP2E1 inhibitor, an NAD(P)H oxidase inhibitor or a nitric oxide synthase inhibitor.

In a further aspect for the composition or the combination, the RNA virus is a positive sense single strand RNA virus. In another aspect, the positive sense single strand RNA virus is a Flaviviridae family virus. In a yet further aspect, the Flaviviridae family virus is selected from the group of a Yellow fever virus, a West Nile virus, a Dengue Fever virus or a hepatitis C virus (HCV). In an alternative aspect, the RNA virus is HCV.

In a further aspect, the virus is a DNA virus, examples of which are provided herein.

The compositions and combinations can include a carrier which includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provision that they be acceptable for use in vivo. For examples of carriers, stabilizers and adjuvants, see Martin REMINGTON′S PHARM. SCI., 15th Ed. (Mack Publ. Co., Easton (1975) and Williams & Williams, (1995), and in the “PHYSICIAN′S DESK REFERENCE”, 52^{nd} ed., Medical Economics, Montvale, N.J. (1998).

An “effective amount” of the agent or composition is contacted with the cell, in vitro or can be administered to the subject such as a human patient, in vivo. An effective amount is an amount sufficient to effect beneficial or desired results. An effective amount can be administered in one or more administrations, applications or dosages.

The disclosure provides an article of manufacture, comprising packaging material and at least one vial comprising a solution of at least one agent or composition with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The disclosure further comprises an article of manufacture, comprising packaging material, a first vial comprising at least one agent or composition and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

In some aspects, the agent or composition or combination is prepared to a concentration or administered to an in vitro system to includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

The compositions or combination of the present disclosure can be prepared by a process which comprises mixing at least one agent or composition and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions or combinations can be provided to patients as clear solutions or as dual vials comprising a vial of agent or composition that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include pen-injector devices for delivery of a solution such as BD Pens, BD Autojectore, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a therapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the disclosure locally to the area in need of treatment; this may be achieved by, for example, and not by way of limitation, local infusion during surgery, by injection or by means of a catheter.

Solutions containing the agent(s) can be prepared in suitable diluents such as water, ethanol, glycerol, liquid polyethylene glycol(s), various oils, and/or mixtures thereof, and others known to those skilled in the art.

The pharmaceutical forms of the agent(s) suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

Further provided is a kit for use in treating or preventing infection by an RNA or DNA virus comprising a pharmaceutical composition comprising, or alternatively consisting essentially of, or yet further consisting of, an iron chelator or an antioxidant and an agent or therapy directed to an RNA or DNA virus and instructions to use. The iron chelator or antioxidant and the agent or therapy directed to the RNA or DNA virus can be combined into a composition or provided as a combination.

In one aspect, the iron chelator is one or more selected from the group diethylene triamine pentaacetic acid (DETAPAC), dipyridyl, pyridoxal isonicotinoyl hydrazone (PIH), desferrioxamine (DFO), deferiprone (DFP) or deferasirox (DFS).

In another aspect, the antioxidant is one or more selected from the group glutathione, N-acetylcysteine, ascorbic acid, vitamin E, beta-carotene, a polyphenol, flavonoid, and an agent that decreases the generation of free radical and non-radical reactive species, including, for example, a CYP2E1 inhibitor, an NAD(P)H oxidase inhibitor or a nitric oxide synthase inhibitor.

In one embodiment, the components of the kit are effective against a positive sense single strand RNA virus, or alternatively a Flaviviridae family virus. In a yet further aspect, the Flaviviridae family virus is selected from the group of a Yellow fever virus, a West Nile virus, a Dengue Fever virus or a hepatitis C virus (HCV). In a yet further aspect of the kit, the components are effective against HCV. In a separate embodiment, the components of the kit are effective against a DNA virus, examples of which are provided herein.

Another aspect of the disclosure provides use of any of the above-described pharmaceutical compositions in the manufacture of a medicament for treating or preventing RNA viral infection by decreasing the ability of the virus to mutate and persist in the cell or host. In a further aspect of the disclosure provides use of any of the above-described pharmaceutical compositions in the manufacture of a medicament for treating or preventing DNA viral infection by decreasing the ability of the virus to mutate and persist in the cell or host.

The following examples are intended to illustrate but not limit the disclosure.

### Example 1

This example demonstrates that alcohol and oxidative stress can increase oxidative RNA damage and the mutability of HCV RNA genome and the RNA damage can be decreased by iron chelators, antioxidants, and inhibitors of reactive oxygen species (ROS)/reactive nitrogen species (RNS) generation. Thus, chemical agents that decrease ROS/RNS-induced RNA damage may be useful at slowing down the mutation rate of HCV and therefore provide means for treating and preventing HCV infection.

Although the example provided herein utilizes RNA virus, the results can be extended to DNA virus.

### Methods

### A. HCV Constructs and Cell Culture:

Huh7 cell clones (SgPC2 cells) supporting continuous replication of a subgenomic HCV replicon of genotype 1b (Con1 sequence) were used (Choi J. et al. (2004) Hepatology 39:81-89; Blight K J et al. (2000) Science 290:1972). The subgenomic replicons support HCV RNA replication but no virus is formed. The in vitro transcription, transfection of HCV RNA, and Huh7 human hepatoma cell culture were performed by methods known in the art and as previously described (Choi J. et al. (2004) Hepatology 39:81-89; Blight K J et al. (2000) Science 290:1972).

### B. Sequencing and Sequences Analysis

After various treatments, total RNAs were collected using Trizol (Invitrogen). Then, the NS5A region (nt. 4612 to 6013 of Con1 replicon) was reverse transcribed/amplified with AccuScript High Fidelity RT-PCR System (Stratagene). The cDNAs were cloned into pUC19 and sequenced at the Joint Genome Institute (Livermore, Calif.). Sequences were aligned against Con1(S11791) sequence, using BioEdit/MEGA 4.0 Molecular Evolutionary Genetics Analysis software.

### C. Determination of 8-OH-G Content

Total RNA was isolated using Trizol (Invitrogen) and analyzed for 8-OH-guanosine (8-OH-G) by northwestern, as described (Shan X et al. (2003) J. Neurosci. 23:4913-4921). Briefly, RNA was size-fractionated by agarose gel electrophoresis, transferred to a nylon membrane, blocked with 1% BSA, incubated sequentially with monoclonal anti-8-OH-G/dG antibodies (QED Bioscience, Inc.) and then with goat anti-mouse HRP-conjugated secondary antibody (Santa Cruz Biotechnology), and signals were detected, using an ECL kit (Amersham). In addition, RNA was digested with nuclease P1 (Sigma, US Biological) and alkaline phosphatase (Promega) and analyzed for 8-OH-G content using RNA Damage Kit (Cell Biolabs, Inc.), following manufacturer's protocols.

### Results

### A. Oxidative Stress and Ethanol Increases the Mutation Frequency of HCV RNA

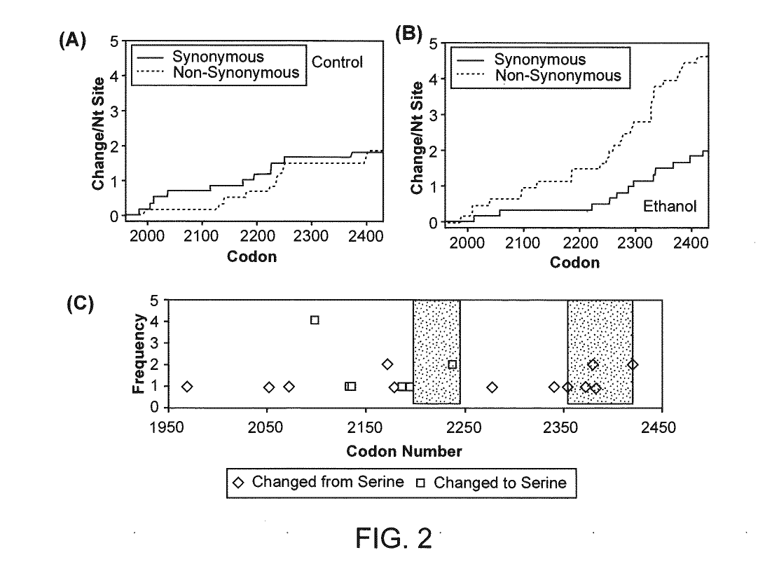

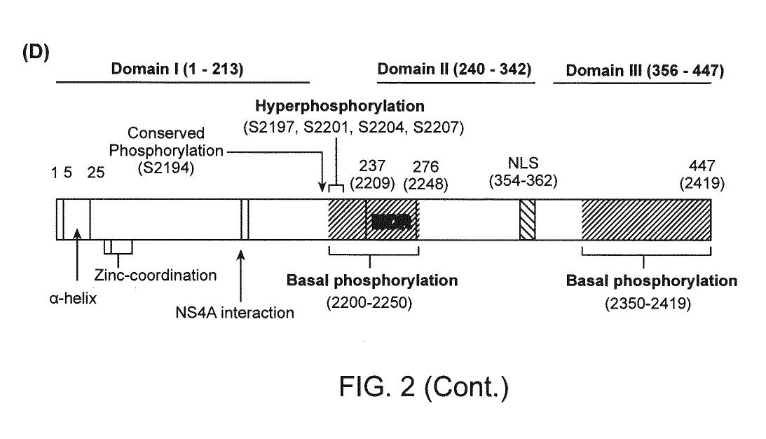

To test whether ethanol can increase the mutation rate of HCV, SgPC2 Con1 replicon cells that support genotype 1b subgenomic HCV RNA replication (FIG. 2B) were incubated with ethanol (0.5%), BSO (amplifies the effects of endogenous ROS/RNS by decreasing the level of glutathione in the cell), H₂O₂ (100 μM), or acetaldehyde (10 μM) once daily for 48 hrs. Then, the RNAs were collected, and the NS5A region (nt. 4612 to 6013 of Con1 replicon) was reverse transcribed/amplified with AccuScript High Fidelity RT-PCR System (Stratagene). Then, the cDNAs were cloned into pUC19 and sequenced at the Joint Genome Institute (Livermore, Calif.). T7 HCV RNA transcript, initially used to transfect the Con1 cells, was also sequenced to assess the basal level of RNA heterogeneity, such as due to any heterogeneity of the DNA template prior to in vitro transcription as well as any errors generated during transcription and RT-PCR cloning. The experiments were focused on NS5A because of its moderate level of diversity and its role in viral replication as well as interferon resistance. Sequences were aligned against Con1(S1179I) sequence, using BioEdit/MEGA 4.0 Molecular Evolutionary Genetics Analysis software.

The control T7 HCV RNA transcript showed 1.27×10⁻³ nucleotide changes per site and 2.02×10⁻³ amino acids change per codon. After normalizing the mutation frequencies by the T7 baseline values, the mutation frequency of HCV RNA in the control untreated cells was determined to be 1.59×10⁻⁴ nucleotide changes per site. Considering the cell passage number, this represents about 5.8×10⁻⁴ nucleotide substitutions per genome site per year, which is less than that observed in patients. This difference may be attributed to higher rate of viral mutation in vivo as well as the basal error rate of RT-PCR cloning, which was not accounted for in the patient study; also, the replicon cells used already contained a cell-culture generated “adaptive” mutation, S1179I, and already represented a positively selected sequence. Ethanol group showed an increased mutation frequency, showing 1.33×10⁻³ nucleotide changes per site, with 3.05×10⁻³ amino acid changes per codon, which are ˜8 and 24 fold higher than the control group, respectively (P<0.001). BSO group also showed a significant increase in the mutation rate, at 6.74×10⁻⁴ nucleotide changes per site with 1.67×10⁻³ amino acid substitutions per codon, which is about 4 and 12 fold higher than the control (P<0.05). The increases in H₂O₂ and acetaldehyde groups were not statistically significant. Therefore, ethanol and HCV-induced oxidative stress may together accelerate the mutation rate of HCV in the alcoholic liver. The observation that BSO increased the mutation rate of HCV more than exogenous H₂O₂ or acetaldehyde suggests that there is a substantial level of oxidative/nitrosative stress already present in these cells, most likely induced by HCV. The data also suggest that ROS/RNS may have a greater effect on HCV sequence than physiological levels of acetaldehyde. Importantly, like in the BSO-treated cells, GSH is severely decreased during HCV infection, which would potentially increase the mutation rate of HCV during natural infection.

HCV tends to show more transition (Tₛ) than transversion mutations (Tᵥ), and the Tₛ/Tᵥ ratio was approximately 2.7 in the control group. Ethanol mostly increased the transition mutations so that the Tₛ/Tᵥ ratio rose to 5.7. Analysis of all nucleotide substitutions in 91 unique genotype 1b sequences in the NIAID HCV sequences database (http://hcv.lanl.gov/content/hcv-db/index) revealed that A [symbol] G and C [symbol] U transitions account for over 80% of all nt. substitutions, the most common being C [symbol] U transitions. Similarly, transitions comprised the majority of the nt. substitutions in the control and treatment groups. Thus, the types of nt. substitutions were similar in control and treatment groups, but the overall frequency of mutations increased with treatments. Whether these represent the most common errors produced by NS5B or that plus errors introduced by ROS/RNS/ethanol is unclear but free radicals are known to induce transitions.

To examine whether HCV sequences were subject to positive selection, the dn/ds ratio was computed, using Mega 4.0 and K-Estimator. Average comparisons of 91 genotype 1b sequences in the database revealed that about 16% of the NS5A-coding region experiences positive selection while 82% shows purifying selection, with dn/ds ratio of 0.104, as reported by others. The dn/ds ratio of the control group was 0.33 but it increased to 0.76 and 0.59 in the ethanol and BSO groups, respectively. Percentage of sites showing positive selection in the study increased from 2.36 control group to 5.14 and 6.42% in ethanol and BSO groups, respectively. These results suggest that ethanol and BSO increased positive selection within 48 hrs. To examine whether these mutations clustered at any specific loci, cumulative synonymous and non-synonymous amino acid mutations were plotted against codons using SNAP (www.hiv.lanl.gov) (FIG. 2). Vertical steps represent either synonymous or nonsynonymous mutations occurring at that codon. Mutations in the control group tended to occur throughout the NS5A sequence. In contrast, synonymous and nonsynonymous mutations clustered in the C-terminal half of NS5A in the ethanol group. BSO, H₂O₂, and acetaldehyde groups showed similar clustering as the ethanol group.

The C-terminal half of NS5A contains basal phosphorylation sites (FIG. 2D). Interestingly, it was found that 50.5% of all nonsynonymous mutations either added or removed serine, threonine, or tyrosine residues in the treatment groups, versus only 9.1% in the control group (P<0.05). Mutations that either generated or removed serines accounted for 25.7% of total nonsynomous mutations in the treatment groups, compared to 0% in the control group. Furthermore, all of the mutations involving serine in the C-terminal basal phosphorylation region removed rather than generate serine (FIG. 2C). Mutations that involve theonines did not show obvious clustering. The most frequent changes from serine were to proline and phenylalanine, suggesting a possible change in protein structure. Other mutations could be found in the nuclear localization signal (NLS), α-helix, double stranded RNA-activated interferon induced protein kinase (PKR) region, and the putative interferon sensitivity determining region (ISDR) of NS5A (FIG. 2D). Of particular interest were F2099S and N2041T which occurred frequently in the study; these substitutions do occur naturally in patients (per HCV database). In contrast, S434 (equivalent to S457 that regulates JFH1 virion production), hyperphosphorylation sites, and zinc coordination motif were not changed.

### B. Ethanol, Oxidative Stress, and RNA Damage

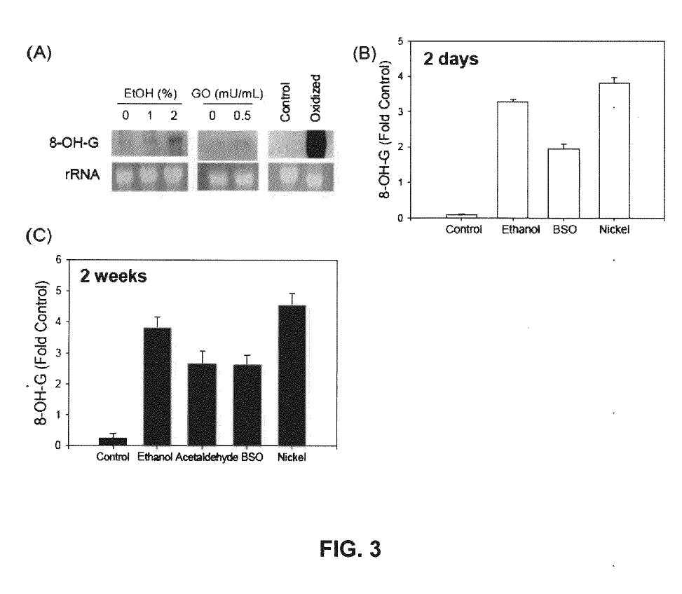

8-Hydroxyguanosine (8-OHG) is the best-characterized and abundant form of oxidatively modified RNA species. To determine whether ethanol and H₂O₂ induce oxidative RNA damage, Huh7 cells were treated with ethanol or GO plus glucose for 24 hrs and analyzed for 8-OHG. GO continuously generates H₂O₂ extracellularly, in the presence of glucose and can be used to mimic inflammation and continued oxidative stress seen during chronic hepatitis C. GO and ethanol (1-2%, v/v) elevated 8-OHG in these cells (FIG. 3). An in vitro-oxidized control Huh7 RNA (incubated with H₂O₂+cytochrome C) was used as a positive control (FIG. 3A). Also, 0.5% ethanol and BSO increased 8-OH-G in SgPC2 cells within 2 days (FIG. 3B). Daily treatment of SgPC2 cells with 0.1% ethanol, 5 μM acetaldehyde, or 20 μM BSO also led to significant increases in the detection of 8-OH-G (FIG. 3C). NiCl₂ was used as a positive control genotoxin in FIGS. 3B and 3C. These data show that ethanol and ROS induce chemical modification of the RNA.

### C. Iron Chelators and Antioxidants can Decrease Oxidative RNA Damage

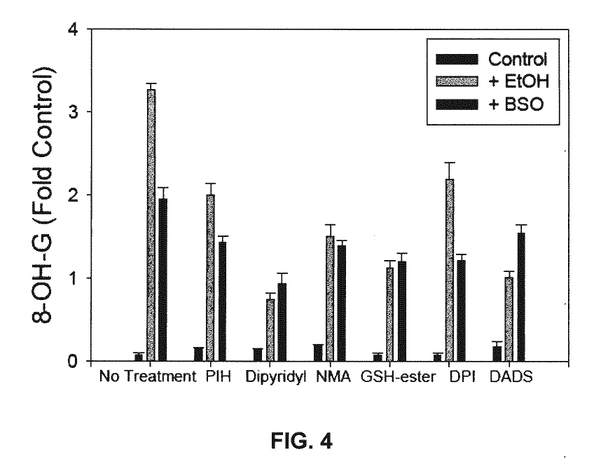

Then, it was examined whether these increases in 8-OH-G could be decreased by antioxidant and chemical agents that inhibit the generation of ROS/RNS. It was found that low concentration of DETAPAC (0.1 mM), an iron-chelator, decreased the 8-OHG levels by 28.0±0.3, 13.8±0.3, and 19.7±0.2% in control, ethanol, as well as BSO-treated SgPC2 cells, respectively (P<0.05), supporting a role of the Fenton chemistry in the RNA damage. Furthermore, αα′-dipyridyl and pyridoxal isonicotinoyl hydrazone (cell-permeable iron chelators; organic synthesized in the PI's laboratory according to published protocols) significantly decreased 8-OHG in ethanol and BSO-treated samples (FIG. 4). As shown in FIG. 4, 2 mM GSH ester, 40 mM diphenylene iodonium (DPI; an inhibitor of flavoproteins such as Nox enzymes), N^{G}-methyl-L-arginine acetate (L-NMA; an inhibitor of nitric oxide synthase), and 25 μM diallyldisulfide (DADS; an inhibitor of CYP2E1) could also reduce the level of 8-OH-G in these cells.

Many virus infections are associated with the perturbation of host redox status and increased generation of ROS/RNS, and there is increasing evidence that reactive species are associated with increased mutability and/or virulence of RNA viruses. For example, selenium deficiency has been associated with an increased mutability and virulence of influenza virus and coxsackievirus, and nitric oxide can accelerate the mutation of Sendai virus in vivo. Mechanisms are thought to include modulation of host immune system as well as modification of the viral sequences. Using HCV, a hepatotropic human pathogen, plus alcohol, which is consumed in relatively high quantities and metabolized by the liver, here presented is evidence that chemical agents can directly increase the mutation frequency of an RNA virus in the context of hepatocytes alone, in the absence of the host immune cells. Increased mutation frequency was associated with oxidative RNA damage. Iron chelator, antioxidant glutathione, and inhibitors of ROS/RNS generating enzymes in the cell decreased RNA damage, suggesting a role of reactive species in the RNA damage triggered by ethanol, BSO, and/or the virus itself. These findings raise an important possibility that endogenous and exogenous agents might affect the evolution of viruses by direct chemical modification of the RNA genomes and suggests a role of human behavior in the evolution of viruses. Increased heterogeneity of virus genomes would facilitate drug resistance and immune escape. To the applicants' knowledge, this is the first study pointing to a range of chemical approach to decrease the mutation rate of a positive sense RNA virus.

In sequence analysis, mutations within the ethanol and BSO treatment groups could be mapped to specific regions within NS5A region with increased do/ds ratios, suggesting that there might also be some selection. The data also show that ethanol and endogenous ROS may change the ability of NS5A to be basal phosphorylated. Increased detection of NS5A sequences that remove the putative basal phosphorylation sites further show that the basal phosphorylation of NS5A has a significant effect on HCV so that the removal of these sites allows those sequences to persist in the cell. Indeed, NS5A phosphorylation can alter the replication fitness of HCV and act as a molecular switch that regulates viral RNA genome replication, translation, and secretion of the virion.

Therefore, ethanol and oxidants can increase oxidative RNA damage and increase the mutation frequency of HCV in hepatocytes in the absence of other components of the host immune system. Oxidative RNA damage can also be decreased by chemical interventions.

### Example 2

This example investigated whether ethanol and reactive species could increase the sequence heterogeneity of HCV RNA, affecting its mutation rate in hepatocytes. Human hepatoma cells supporting a continuous replication of genotype 1b HCV RNA (Con1, AJ242652) were exposed to ethanol, acetaldehyde, hydrogen peroxide, or L-buthionine-S,R-sulfoximine (BSO), which decreases glutathione, to mimic glutathione depletion seen in patients. Then, HCV NS5A region was sequenced and compared with genotype 1b HCV sequences found in the database. Ethanol and BSO elevated both nucleotide and amino acid substitution rates of HCV RNA by 4-18 folds within 48 hrs, which were accompanied by oxidative RNA damage. A cell-permeable iron chelator and glutathione ethyl ester decreased the RNA damage as well as sequence heterogeneity of the HCV RNA, suggesting a role of hydroxyl radical. HCV (JFH1, AB047639) itself also induced oxidative RNA damage which could be exacerbated by ethanol and BSO. In addition, the do/ds ratio and percentage of sites undergoing positive selection increased with ethanol and reactive species, leading to increased detection of sequence variants previously implicated in antiviral resistance to interferon alpha, cyclosporine, and ribavirin, immune tolerance, and modulation of viral replication. Therefore, this example shows that alcohol can synergize with virus-induced oxidative/nitrosative stress to increase the mutation rate of HCV, contributing to antiviral resistance. Conversely, agents that decrease oxidative/nitrosative stress can help attenuate the mutation rate of HCV.

### Materials & Methods

### HCV Constructs, Cells, and Reagents

Huh7 cell clones (SgPC2 cells) supporting continuous replication of a subgenomic HCV replicon of genotype 1b (Con1S1179I sequence) were used (Choi J. et al. (2004) Hepatology 39:81-89; Blight K J et al. (2000) Science 290:1972). Subgenomic replicons support the HCV RNA genome replication by the viral replicase but no virus is formed. The in vitro transcription, transfection of the viral RNA, and Huh7 human hepatoma cell culture were performed as described (Choi J. et al. (2004) Hepatology 39:81-89; Blight K J et al. (2000) Science 290:1972). All biochemicals including ethanol (molecular biology grade), acetaldehyde, BSO, H₂O₂, dipyridal, and GSH ethyl ester were obtained from SigmaAldrich except PIH, which was synthesized according to published methods (Kicic A et al., (2001) Cancer 92: 3093-3110; Ponka P. et al., (1979) FEBS Lett 97: 317-321).

### Sequencing and Sequences Analysis

Total RNAs were collected using Trizol (Invitrogen). Then, the NS5A region (nt. 4612 to 6013 of Con1 replicon) was reverse transcribed/amplified with AccuScript High Fidelity RT-PCR System (Agilent). The reverse primer used for reverse transcription and PCR amplification was 5′-ATCAATCGATTGTCTAGAGCTGAAGAGGCTTCACCAG-3′ (SEQ ID NO: 1), and the forward primer was 5′-GCAATCTTGTACAAGCTTCGCAGCGCATGGCGTGAT-3′ (SEQ ID NO: 2). The XbaI and HindIII restriction enzyme sites are underlined. The cDNA products were cloned into pUC19 via HindIII and XbaI sites and sequenced at the Joint Genome Institute (Livermore, Calif.) and University of California Berkeley Sequencing (Berkeley, Calif.). Sequences were aligned against Con1(S1179I) sequence, using BioEdit/MEGA 4.0 Molecular Evolutionary Genetics Analysis software. Cumulative synonymous and nonsynonymous mutations were computed using Synonymous Nonsynonomous Analysis Program (SNAP; www.hcv.lanl.gov). The do/ds ratio was calculated using SNAP. Amino acid substitution trends were analyzed by position with MutationCounter, a mutation counting program, developed for this project. The sequences were compared with 1,212 genotype 1b NS5A sequences pooled from the Lawrence Livermore HCV sequence database (www.hcv.lanl.gov) which included sequences from the European, Asian, and Russian databases. Sequences harboring gaps, undetermined nucleotides, or incomplete NS5A sequences were removed prior to the analysis. The experiment was repeated twice and the first trial (sequenced by the Joint Genome Institute) and the second trial (sequenced by UC Berkeley Sequencing) yielded the same conclusions, and the compiled data from the two studies are reported in Table I.

### Determination of 8-OH-G Content

Total RNA was isolated using Trizol (Invitrogen) and analyzed for 8-OHG by ELISA. Briefly, RNA was incubated with 10 U DNase I (GE Healthcare), 10 U nuclease P1 (Sigma or US Biological), and 10 U alkaline phosphatase (Promega) and analyzed for 8-OHG content with RNA Damage Kit (Cell Biolabs, Inc.) and 8-OHG standards, following manufacturer's protocols.

### Site-Directed Mutagenesis

An HCV replicon bearing Y321C was generated by site-directed mutagenesis using QuikChange II XL Site-Directed Mutagenesis Kit (Agilent). The sense primer used was 5′-GCACGCCCGGATTGCAACCCTCCACTG-3′ (SEQ ID NO: 3) and the antisense primer was 5′-CAGTGGAGGGTTGCAATCCGGGCGTGC-3′ (SEQ ID NO: 4).

### HCV RNA Quantitation

HCV RNA was quantified by quantitative reverse transcriptase polymerase chain reaction (qRT-PCR) as described (Seronello S. et al., (2010) J Biol Chem 285: 845-854). Intracellular HCV RNA levels were normalized by glyceraldehydes 3-phosphate dehydrogenase (GAPDH) mRNA content. Standard curves were generated using in vitro-transcribed HCV RNA's.

### Statistics

Data were analyzed using Student's t test, one-way analysis of variance, and z-test or Chi-square analysis of proportions using SigmaStat 3.1 (Jandel Scientific). A p value 0.05 was considered significant.

### Results

RNA viruses are ubiquitous intracellular parasites with compacted genomes. RNA viruses are characterized by rapid replication and a particularly high level of genetic variability, existing as a population of closely related but distinct genetic variants, referred to as quasispecies. High rate of mutation allows these viruses to operate near the error threshold, for maximal adaptability to the changing environment while conserving essential genetic information. These features of RNA viruses pose significant challenge to antiviral therapy and development of vaccines and have been attributed to the lack of proofreading activity of viral replicases among other factors although additional, previously unidentified mechanisms might also play a role.

Hepatitis C virus (HCV) is a positive-sense RNA virus of Flaviviridae family. The HCV genome is about 9.6 kb in length and consists of the 5′ untranslated region (UTR), the structural protein coding regions (C, E1, E2), the nonstructural (p7, NS2, NS3, NS4A/B, NS5A/B) protein regions, and the 3′ UTR. HCV is estimated to infect about 170 million people worldwide, including at least four million people in the U.S. alone. Current anti-HCV therapy, which combines the use of pegylated interferon alpha and a nucleoside analog, ribavirin, results in a sustained virological response in only about 50-60% of patients undergoing treatment. Both the efficacy of antiviral therapy and success of re-treating the resistant population with antiviral compounds are strongly affected by HCV sequence. Specific regions in the HCV genome have also been identified that can affect the sensitivity of the virus to interferon, such as the NS5A region. HCV, like other RNA viruses, also exists as a quasispecies. Recent reports indicate a correlation between drinking alcohol and the heterogeneity of HCV RNA in patients, although underlying mechanism remains unclear.

Ethanol metabolism generates acetaldehyde and increases the levels of reactive oxygen species (ROS), reactive nitrogen species (RNS), and lipid peroxidation products while decreasing glutathione (GSH) and selenium. In addition, HCV itself increases oxidative/nitrosative stress that can be exacerbated by ethanol, and GSH is depleted in patients infected with HCV. ROS, RNS, and lipid peroxidation products can alter cell metabolism and signaling, altering the intracellular milieu for viral replication, which may pose selection pressure to HCV. Furthermore, acetaldehyde, ROS, RNS, and lipid peroxidation products can attack the bases and deoxyribose backbone of DNA as well as RNA and potentially act as mutagen to the viral RNA. It is also interesting to note that alcohol, genotype 1 HCV infection, iron overload, age, co-infection with HIV, steatosis, fibrosis, and male gender, all of which are associated with increased ROS/RNS and/or decreased antioxidant capacity, have each been linked with interferon resistance. However, whether ethanol and virus-induced oxidative/nitrosative stress can affect the mutation rate of HCV and antiviral resistance, via selection and/or RNA genotoxicity, is unknown.

### Ethanol and Reactive Species Increase the Mutation Frequency of HCV RNA

To test whether ethanol can affect the HCV RNA sequence, Con1 replicon cells that support genotype 1b subgenomic HCV RNA replication were incubated with ethanol (0.5% v/v), BSO (20 μM), H₂O₂ (100 μM), or acetaldehyde (10 μM) once daily for 48 hrs with a change of the cell culture medium at the time of each treatment. Acetaldehyde and H₂O₂ are generated during hepatic metabolism of ethanol. BSO, by inhibiting GSH biosynthesis, mimics GSH depletion seen in hepatitis C patients. GSH participates in detoxification reactions that remove reactive species such as H₂O₂ and acetaldehyde. Thus, by decreasing GSH, BSO also amplifies the effects of endogenous ROS and RNS, allowing one to test the effects of endogenous ROS/RNS. Then, the RNA was collected, and the NS5A region corresponding to nt. 4612 to 6013 of Con1 replicon was reverse transcribed using AccuScript reverse transcriptase and amplified with PfuUltra (Agilent). PfuUltra is a high fidelity DNA polymerase with an error rate of 4.3×10⁻⁹; AccuScript reverse transcriptase has an error rate of 1.61×10⁻⁷. Then, the cDNA products were cloned into pUC19 and sequenced by the Sanger method. The T7 HCV RNA transcript, initially used to transfect Huh7 human hepatoma cells to generate the replicon cells, was also sequenced to assess the basal level of RNA heterogeneity potentially rising from the heterogeneity of HCV DNA used in the in vitro transcription reaction as well as errors generated during transcription and/or RT-PCR cloning. The focus was on NS5A because of its moderate level of diversity and its role in viral replication cycle and interferon resistance. Sequencing data were aligned against Con1(511791) sequence, using BioEdit and MEGA 4.0.

After subtracting the T7 baseline values, the nucleotide and amino acid substitution rates of the control untreated cells were determined to be 2.89×10⁻⁴ nucleotide changes per site and 2.80×10⁻⁴ amino acid changes per codon (Table I). The ethanol group showed an increased nucleotide substitution rate, with 1.71×10⁻³ nucleotide changes per site and 4.97×10⁻³ amino acid changes per codon, which represented about 6 and 18 fold increases over the control, respectively (P<0.001). BSO group also displayed a significant increase in the sequence heterogeneity of HCV RNA, showing 1.20×10⁻³ nucleotide changes per site and 3.24×10⁻³ amino acid substitutions per codon, which were about 4 and 12 fold higher than the control, respectively (P<0.05). On the other hand, H₂O₂ and acetaldehyde treatments did not increase the nucleotide substitution rates significantly over the control (5.28×10⁻⁴ and 4.25×10⁻⁴ nt. change per site, respectively, P>0.05). The amino acid substitution rate did not increase with acetaldehyde but it increased by about 10 fold with H₂O₂. Therefore, ethanol and reactive species could significantly increase the nucleotide and amino acid substitution rates of HCV RNA over 48 hr.

TABLE I
Nucleotide and amino acid substitution rates in control and treatment
groups^{a}

| Treat- | | | | Amino acid | | | |
|---|---|---|---|---|---|---|---|
| ment | | Nt. change | Fold | change | Fold | Ts/ | |
| Group | n^{b} | per site^{c} | increase | per site^{d} | increase | Tv | dn/ds |
| Control | 21 | 2.89 × 10⁻⁴ | — | 2.80 × 10⁻⁴ | — | 3.3 | 0.31 |
| Ethanol | 21 | 1.71 × 10⁻³ | 5.9 | 4.97 × 10⁻³ | 17.8 | 6.8 | 1.05 |
| | | (P < 0.001)^{e} | | (P < 0.001)^{e} | | | |
| BSO | 28 | 1.20 × 10⁻³ | 4.2 | 3.24 × 10⁻³ | 11.6 | 3.3 | 0.72 |
| | | (P < 0.001)^{e} | | (P < 0.001)^{e} | | | |

^{a}Nucleotide and amino acid sequences used in the study were compared against Con1 sequence.
^{b}Number of sequences analyzed in each group.
^{c,d}Data were normalized by the T7 baseline values which were 1.27 × 10⁻³ nt. change per site and 1.96 × 10⁻³ amino acid change per site.
^{e}Statistically significant difference from control.

HCV tends to show more transitions (Tₛ) than transversions (Tᵥ). Analysis of 1,212 genotype 1b sequences in the HCV sequence database (http://hcv.lanl.gov/content/hcv-db/index) revealed that transitions account for over 83.5% of all nt. substitutions (Table II). The Ts/Tv ratio of genotype 1b HCV sequences in the database was determined to be 5.05. Likewise, transitions comprised the majority of the nt. substitutions in both control and treatment groups (Tables I and II). Ethanol increased transitions more than transversions such that the Tₛ/Tᵥ ratio rose from 3.3 to 6.8 whereas it remained at 3.3 in the BSO group. The most common nt. substitutions were U→C and A→G transitions in ethanol and BSO groups similar to the database (Table II).

TABLE II
Nucleotide substitution trends in control and treatment groups ^{a}

| Nt. substitution | Control | Ethanol | BSO | Database ^{b} |
|---|---|---|---|---|
| U→C | 18.18 | 29.87 | 29.49 | 23.57 |
| A→G | 18.18 | 25.97 | 24.36 | 19.62 |
| C→U | 27.37 | 18.18 | 8.97 | 17.58 |
| G→A | 15.15 | 12.99 | 16.67 | 22.7 |
| A→C | 6.06 | 1.30 | 1.28 | 1.54 |
| A→U | 3.03 | 2.60 | 2.56 | 1.97 |
| G→C | 0 | 6.49 | 3.85 | 3.95 |
| G→U | 0 | 2.60 | 3.85 | 2.08 |
| U→A | 0 | 0 | 0 | 3.01 |
| U→G | 3.03 | 0 | 0 | 0.46 |
| C→A | 6.06 | 0 | 3.85 | 1.73 |
| C→G | 3.03 | 0 | 5.13 | 1.77 |

^{a} Data represent percentage of each type of nucleotide substitutions in each group. Transition mutations are shown in the first four rows.
^{b} Percentage of each type of nucleotide substitutions in the genotype 1b HCV sequences in the database compared to Con1 sequence used in the study.

### Ethanol, Reactive Species, and Oxidative RNA Damage

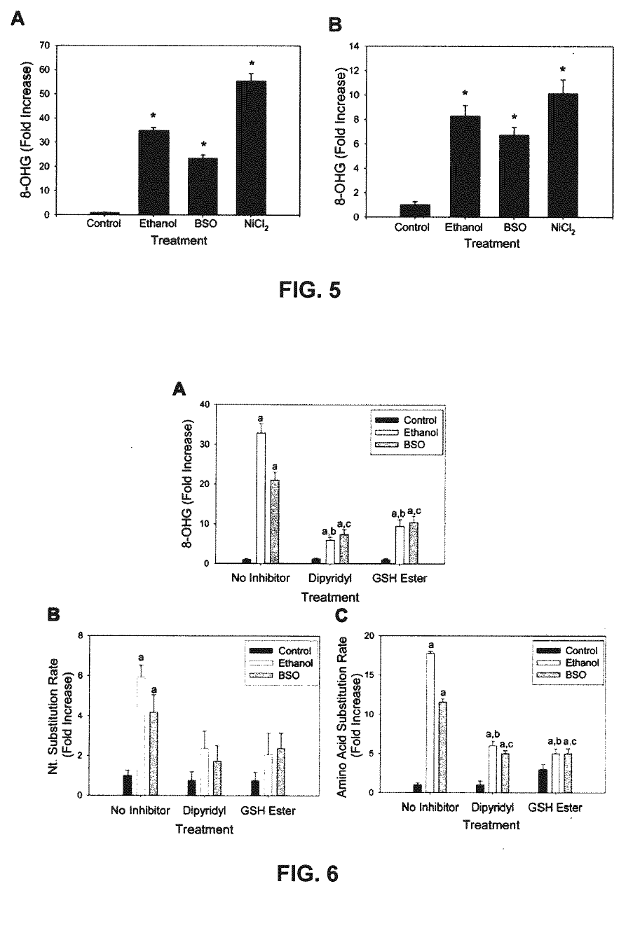

The observation that GSH depletion with BSO had a greater effect on the HCV RNA sequence than exogenous H₂O₂ or acetaldehyde suggested that there was a substantial level of endogenous oxidative/nitrosative stress in these cells and that endogenous ROS/RNS might have a greater effect on the HCV RNA than acetaldehyde. Thus, to substantiate the occurrence of oxidative/nitrosative stress and to test the potential role of oxidative RNA damage in the above increases in the sequence variability of HCV RNA in the treatment groups (Table I), this example next measured oxidative RNA damage. Huh7 cells were treated with ethanol or BSO for 48 hrs and analyzed for 8-hydroxyguanosine (8-OHG); 8-OHG is the best-characterized and abundant form of ROS/RNS-generated RNA species. NiCl₂ was included in these assays as a positive control. NiCl₂ induced a 55.4±3.1 fold increase in 8-OHG content over the control (P<0.05) (FIG. 5A). In addition, ethanol increased 8-OHG content by 34.9±1.2 fold (P<0.05). 8-OHG also increased significantly, by 23.4±1.5 fold in the BSO group (P<0.05) (FIG. 5A). Therefore, ethanol and reactive species could damage the RNA under the same conditions that elevated the sequence heterogeneity of HCV RNA. Daily treatment of Con1 replicon cells with 0.1% ethanol (v/v) or 20 μM BSO for 2 weeks likewise led to significant increases in 8-OHG in these cells (FIG. 5B).

### Effect of Decreasing Reactive Species

Then, agents that decrease ROS/RNS were used to test how these agents would affect the sequence heterogeneity of HCV RNA increased by ethanol and BSO. HCV infection is associated with iron overload which in turn has been associated with oxidative DNA damage. Therefore, control, ethanol, and BSO-treated cells were co-incubated with or without an iron chelator, then monitored 8-OHG content and sequenced the viral RNA. α,α′-Dipyridyl, a cell permeable iron chelator, significantly decreased the level of 8-OHG in both ethanol and BSO groups (FIG. 6A). GSH ester was used to supply GSH exogenously, and GSH ester effectively decreased the level of 8-OHG in ethanol and BSO groups (FIG. 6A). Similarly, the RNA damage could be decreased by pyridoxal isonicotinoyl hydrazone (PIH, another cell-permeable iron chelator), 1,1,4,7,7-diethylenetriaminepentaacetic acid (DETAPAC, an iron chelator), diphenylene iodonium (DPI, an inhibitor of flavoproteins such as NAD(P)H oxidases), N^{G}-methyl-L-arginine acetate (L-NMA, an inhibitor of nitric oxide synthase), and diallyl disulfide (DADS; an inhibitor of CYP2E1) (FIG. 12).

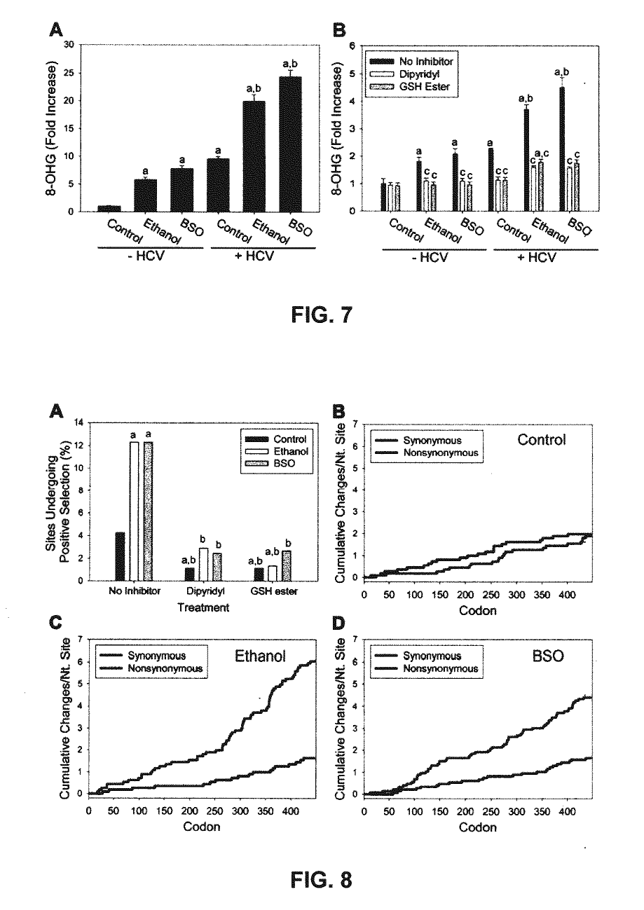

Dipyridyl decreased the nt. substitution rates of both ethanol and BSO groups so that there was no longer any significant increase over the control (FIG. 6B). In addition, dipyridyl effectively abolished any increase in the amino acid substitution rate caused by BSO and significantly attenuated the amino acid substitution rate in the ethanol group (P<0.05) (FIG. 6C). Similarly, GSH ester decreased nt. and amino acid substitution rates in both ethanol and BSO groups to levels not significantly above the control (FIGS. 6B and 7C). These data indicate a strong association between reactive species/induction of RNA damage and the mutation rate of HCV. In addition, the data indicate that the mutation rate of HCV, which increased under conditions mimicking GSH depletion seen in patients, can be attenuated by restoring GSH.

### HCV Induces RNA Damage

HCV induces severe oxidative/nitrosative stress in the host by increasing the generation of reactive species. Thus, JFH1 strain of genotype 2a that supports complete replication cycle of HCV and produces infectious HCV in cell culture was used next to determine whether HCV was sufficient to induce oxidative RNA damage even in the absence of ethanol and BSO. Huh7 cells were transfected with JFH1 RNA and either exposed to 0.5% ethanol (v/v) or 20 μM BSO daily for 48 hrs, or to 0.1% ethanol (v/v) or 20 μM BSO with and without 5 μM dipyridyl or 1 mM GSH ester daily with a change of medium prior to each treatment for 2 weeks. As shown in FIG. 7, HCV itself significantly increased 8-OHG at 48 hr and 2 weeks, both of which were exacerbated by ethanol as well as BSO (FIG. 7). These data, together with the literature describing oxidative DNA damage in various cell/animal models of HCV and in patients, suggest that there is indeed a significant level of oxidative/nitrosative stress in the HCV-infected cells and that, in addition to DNA damage, RNA damage occurs as a direct consequence of HCV infection even in the absence of ethanol, which is exacerbated under glutathione-deficient conditions.

### Sequence Analysis Indicates Increased Positive Selection

To further examine whether HCV sequences might be undergoing positive selection, the dn/ds ratio was computed using SNAP. The dn/ds ratio of the control group was 0.31, and it increased to 1.05 and 0.72 in the ethanol and BSO groups, respectively (Table I). Percentage of sites showing positive selection in the study increased from 4.25% in control group to 12.3% in ethanol and 12.3% in BSO groups (FIG. 8A). These results indicated that ethanol and BSO, in addition to inducing RNA damage, also increased positive selection within 48 hrs. Average comparisons of 1,212 genotype 1b sequences in the database revealed that 19.5% of the NS5A-coding region experiences positive selection while about 79% undergoes purifying selection. Dipyridyl and GSH ester co-treatments reduced the dn/ds ratio in the ethanol group from 1.05 to 0.46 and 0.43, respectively. Dipyridyl and GSH ester also decreased the dn/ds ratio in the BSO group from 0.72 to 0.46 and 0.21, respectively. Dipyridyl did not affect the dn/ds ratio of the control group whereas GSH ester decreased it slightly from 0.31 to 0.24. The percentage of sites undergoing positive selection decreased with dipyridyl and GSH ester co-treatments (FIG. 8A).

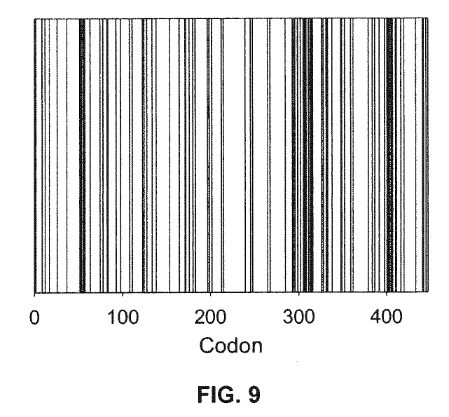

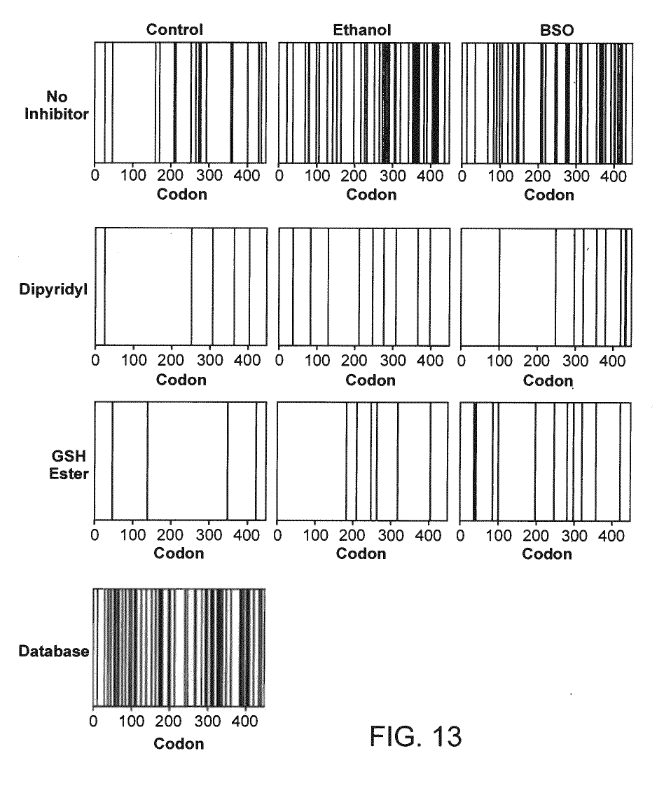

To determine whether mutations clustered at any specific loci, cumulative synonymous and non-synonymous amino acid mutations were plotted against codons using SNAP (FIG. 8B). Vertical steps represent either synonymous or nonsynonymous mutations occurring at that codon. Mutations in the control group tended to occur throughout the NS5A sequence (FIG. 8B). In contrast, mutations clustered in the C-terminal half of NS5A in ethanol and BSO groups (FIGS. 8C and 8D). H₂O₂ and acetaldehyde groups showed similar clustering of mutations as ethanol and BSO groups (data not shown). Sites undergoing positive selection in the control, ethanol, and BSO groups, with and without dipyridyl and GSH ester co-treatments, as well as HCV sequence database are mapped in FIGS. 9 and 13, which show a greater similarity between the database and the treatment groups than the control group. More specifically, 36.4% of sites undergoing positive selection in the treatment groups were also found to be under positive selection in the database versus 21.1% in the control group. In addition, 36.8% of the sites undergoing positive selection in the HCV database matched sites that underwent positive selection in the ethanol and BSO treatment groups, compared to only 4.6% for the control group (FIG. 9). The majority of nonsynonymous amino acid substitutions in this study (97.4%) occurred at sites undergoing positive selection. No increase was found in positive selection at synonymous nt. substitution sites with treatments.

### Analysis of Mutation Sites Suggests Increased Propensity for Antiviral Resistance

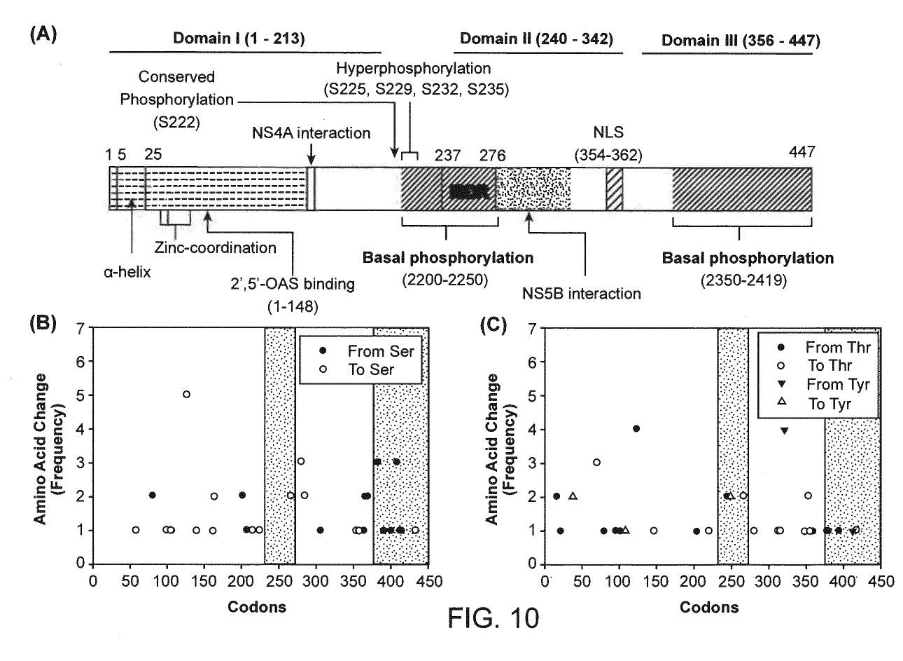

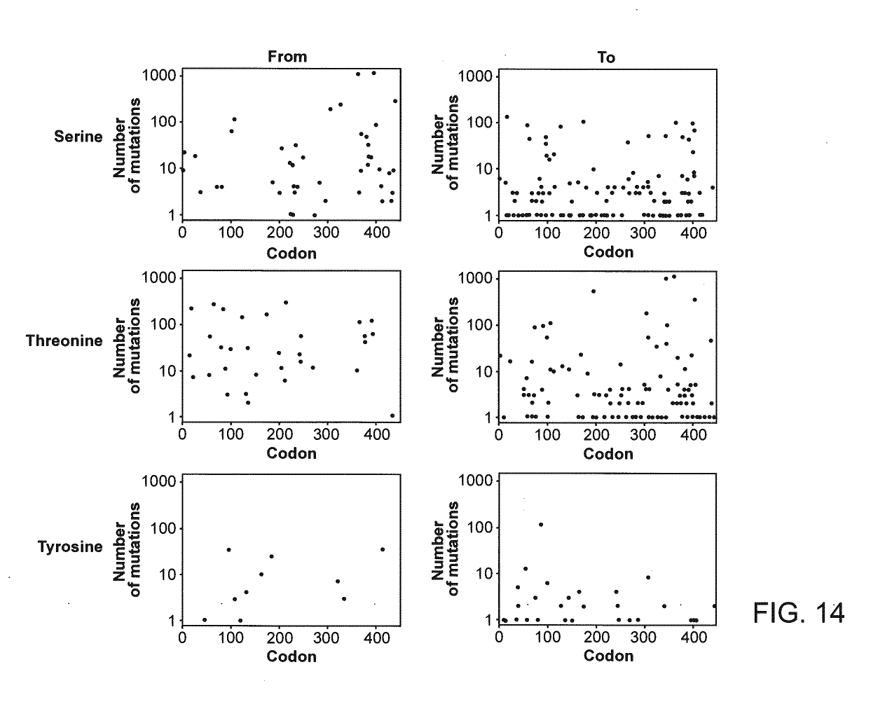

This example continued to analyze the sequences for changes occurring at any specific regions within NS5A. The C-terminal half of NS5A contains basal phosphorylation sites (FIG. 10A). Interestingly, it was found that 50.3% of all nonsynonymous mutations either added or removed serine, threonine, or tyrosine (STY) residues in the treatment groups, versus only 9.5% in the control group (P<0.05). Of these, mutations either adding or removing serine accounted for 26.3% of total amino acid substitutions in the treatment groups, compared to 0% in the control group (P<0.05). Mutations that either generated or removed threonine accounted for 21.0% of amino acid substitutions in the treatment groups versus 4.8% in the control group (P<0.05). In contrast, mutations involving tyrosine occurred less frequently, representing only 3.1% of all amino acid changes. The majority of mutations involving serine in the domain 3 of NS5A removed rather than generated serine, whereas mutations that generated serine were more frequent in domains 1 and 2 (FIGS. 10A and 10B). Mutations involving threonine or tyrosine did not show such obvious clustering except that certain mutations occurred more frequently at specific sites (FIG. 10C) (Tables III and IV). In comparison, STY substitutions represented 40.1% of all genotype 1b NS5A sequence variations in the database (FIG. 14). Furthermore, while the frequency of mutations occurring at any given site in this study tended to be low, some of the STY mutations occurred more frequently than others and underwent positive selection during treatment, and these mutations are listed in Table III. These sequence variations also occurred in genotype 1b HCV sequences in the database (Table III), with positive selection occurring at F127, T122, T379, and S401. A comprehensive list of amino acid variations compiled from 1,212 genotype 1b HCV NS5A sequences in the database, tabulated by position and by the type of amino acid substitutions, is found in Table V.

TABLE III
Frequent mutations involving serine/threonine/tyrosine residues in the NS5A region

| | Study | Database | | Type of substitutions, |
|---|---|---|---|---|
| Mutation | count ^{a} | count ^{b} | Accession Numbers ^{c} | database ^{d} |
| F127S | 5 | 79 | AB285036, AF207760, AF265148-50, AF265163-65, | F127S (79), F127C (1), |
| | | | AM402055-58, AM402060-78, AM402080-82, | F127L (4), F127Y (2) |
| | | | AM402084-90, AM402092-110, AM402112-116, | |
| | | | AM402118-122, AM402125, AM402127-129, | |
| | | | AY808022, DQ491974, DQ492015, EF407482, | |
| | | | EF407488, EU482883 | |
| T122A | 4 | 4 | DQ491961, DQ491964, DQ491966, DQ492018 | T122A (4), T122K (3), |
| | | | | T122R (37), T122V (91), |
| | | | | T122W (2) |
| Y321C | 4 | 3 | AM401892, AM401896, AM401974 | Y321C (3), Y321D (3), |
| | | | | Y321N (1) |
| N69T | 3 | 16 | AJ507172, AM402271, DQ491995-97, DQ491999, | N69T (16), N69A (1), |
| | | | DQ492000-07, EF407495, HM042050 | N69K (1), N69S (3) |
| S383P | 3 | 10 | AF165061, AF207769, AM401885, AM493449, | S383P (10), S383F (2) |
| | | | DQ244140, EF407461, EF407470, EF407482, | |
| | | | EU862837, FJ958916 | |
| S401P | 3 | 39 | AB285042, AF165061-62, AF265158, AF483269, | S401P (39), S401A (2), |
| | | | AJ000009, AJ507156, AJ507169, AJ507182, | S401F (6), S401L (39), |
| | | | AM493489-507, AY008261, AY808015, AY808026, | S401T (1) |
| | | | AY808030-31, AY808043, DQ492060, DQ492152, | |
| | | | EU155281, EU256062, EU256080 | |
| S408P | 3 | 3 | AM401886, AM402364, AM402374 | S408P (3), S408F (1), |
| | | | | S408T (5), S408Y (1) |
| T379A | 2 | 64 | AB049092, AB285040, AF165045-46, AF207755, | T379A (64), T379S (7) |
| | | | AM402055, AM402060, AM402066, AM402070-71, | |
| | | | AM402074-75, AM402081, AM402088, AM402090, | |
| | | | AM402096, AM402098, AM402101-102, AM402106, | |
| | | | AM402108, AM402111-112, AM402115, AM402125, | |
| | | | AM402127, AY460204, EF407470, EF407477, | |
| | | | EU155218, EU155381, EU862837, EU781832, HM042053 | |

^{a} Frequency (count) of substitutions occurring in the treatment groups.
^{b} Frequency of substitutions in the genotype 1b HCV sequences in the database compared to Con1 sequence used in the study.
^{c} Accession numbers for HCV sequences in column ^{b}.
^{d} Type of amino acid substitutions occurring at that position in the genotype 1b HCV database and frequencies (count), shown in parenthesis.

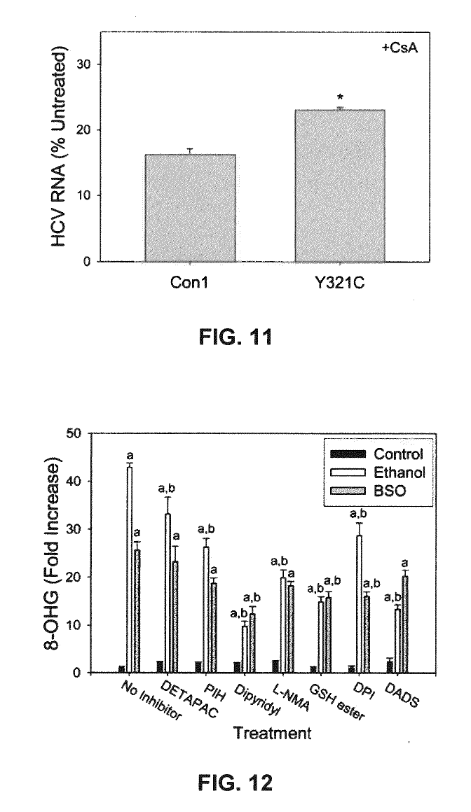

Most of all, it was found that some of the amino acid substitutions discovered in this study were mutations previously associated with antiviral resistance (Table IV). For example, F37L, which augments the binding of NS5A to 2′,5′-oligoadenylate synthetase, thereby inhibiting its action during IFN response, also occurred in this study with a dN/dS ratio>1. E442G, V279A, F284S, V445A, E442G, V410A, 1302V, and S414P, which were shown to confer or otherwise found to be strongly associated with resistance to ribavirin, interferon, cyclosporine, and/or Debio 025 were also identified (Table IV). In addition, Y321 N was previously shown to confer resistance to cyclosporine, and all of the nonsynonymous mutations removing tyrosine in this study occurred at this position (Y321C) (Table IV). When Y321C was introduced to the wild-type Con1(S1179I) sequence by site-directed mutagenesis, it was found that it conferred increased resistance to cyclosporine, as shown in FIG. 11; the HCV RNA titer was 46.7% higher with Y321 C mutant than the wild-type Con1(S1179I) after cyclosporine treatment. Ethanol and reactive species also generated mutations in all three of the CD8+ T cell-specific epitopes in NS5A previously associated with treatment response. Other nonsynonymous amino acid substitutions could also be found in binding regions for various viral and host proteins including double-stranded RNA-activated interferon induced protein kinase (PKR) and the putative interferon sensitivity determining regions (ISDR). Four sites within the ISDR which, in combinations, enhanced HCV RNA replication in the study by Kohashi et al. had a dN/dS ratio>1 in this study, but no single sequence in this study had more than two amino acid substitutions within the ISDR. It was also found that other amino acid substitutions that had been documented during in vivo infection of chimpanzees as well as those that affect the replication rate of HCV (Table IV). S437 (equivalent to S457 of JFH1), hyperphosphorylation sites, and zinc coordination motif were not changed.

TABLE IV
Functional changes associated with mutations found in this study^{a}

| Mutation in | | Occurrences | dN/dS >1 |
|---|---|---|---|
| Literature | Function^{b} | in Study^{c} | at codon^{d} |
| F37L | Inhibition of 2′,5′-oligoadenylate synthetase | F37L (4) | Yes |
| Y321N | Resistance to cyclosporine; conformational | Y321C (4) | Yes |
| | change in a major cyclophilin A binding region | | |
| V279A | Resistance to cyclosporine | V279A (2) | No |
| F284S | Resistance to cyclosporine | F284S (2), F284L (1) | Yes |
| D320E | Resistance to cyclosporine | D320H (1) | No |
| V445A | Resistance to Debio 025 | V445A (1) | Yes |
| E442G | Resistance to IFN | E422G (1), E422K (2), | Yes |
| | | E422V (1) | |
| V410A | Resistance to IFN | V410A (2) | Yes |
| I302V | Resistance to IFN | I302V (1) | Yes |
| S414P | Resistance to IFN | S414P (1) | No |
| W433R | Resistance to IFN | W433S (1), W433C (1) | Yes |
| S390R | Resistance to IFN | S390N (1) | No |
| K277E | Resistance to IFN | K277R (1) | No |
| E442G | Resistance to ribavirin | E442G (1) | Yes |
| G404S | Resistance to ribavirin | G404D (1) | No |
| T21A | Occurred during in vivo infection of chimpanzees | T21A (2) | Yes |
| M133V | Occurred during in vivo infection of chimpanzees | M133V (1) | Yes |
| R318H | Occurred during in vivo infection of chimpanzees | R318H (1) | Yes |
| N69T | Occurred during passage in HEK 293/Sip-L cells | N69T (3) | Yes |
| V177I | Occurred during passage in HEK 293/Sip-L cells | V177I (1) | No |
| N105Y | Occurred during passage in HEK 293/Sip-L cells | N105S (1), N105D (1), | Yes |
| | | N105G (1) | |
| A211E | Occurred during passage in HEK 293/Sip-L cells | A211V (1) | No |
| A252V/F | Increased HCV replication | A252V (3) | Yes |
| T244I | Increased HCV replication | T244A (2) | Yes |
| H247Y | Increased HCV replication | H247Y (2) | Yes |
| R246Q | Increased HCV replication | R246L (1) | Yes |
| K68R | Increased HCV replication | K68R (2) | No |
| L226S | Increased HCV replication | L226S (1) | No |
| S264P | Increased HCV replication | E264G (1) | No |
| M313A | Increased HCV replication | M313V (1) | Yes |
| G341A | Increased HCV replication | G341R (1) | No |

^{a}Mutations from literature were aligned to and listed by the codon numbers used in this study.
^{b}Functions attributed to mutations in the literature.
^{c}Amino acid substitutions occurring at the same position in this study with the number of occurrences shown in parenthesis. Mutations that occurred more frequently are listed in top rows.
^{d}Yes = positive selection occurring in this study; No = no positive selection.

There is increasing evidence that virus infections are associated with perturbation of the host redox status and increased generation of ROS/RNS. Reactive species have also been suggested to increase the mutability and/or virulence of RNA viruses. Mechanisms are thought to include modulation of the host immune system as well as modification of viral sequences. This study used HCV, an important hepatotropic human pathogen, plus ethanol, which is consumed in relatively high quantities and metabolized to generate reactive species and other reactive metabolites in the liver, to examine how these factors affect the genetic variability of viral RNA genome, either by acting as mutagen to the viral RNA or by selection. It was found that ethanol and endogenous reactive species could directly increase the sequence heterogeneity of HCV RNA in the context of hepatocytes alone, in the absence of the immune cells, within 48 hrs. Increased nt. and amino acid substitution rates of HCV RNA were associated with oxidative RNA damage and increased positive selection. Iron chelators, GSH ester, and inhibitors of ROS/RNS generating enzymes decreased RNA damage as well as nt. and amino acid substitutions. These findings raise an important possibility that ethanol and endogenous reactive species can affect the mutation rate of an RNA virus. These findings also suggest a role of human behavior in the evolution of viruses. To the applicant's knowledge, this is the first study pointing to a diverse yet targeted biochemical approach to attenuate the mutation rate of a virus, which may prove to be clinically relevant. Furthermore, as reactive species will modify RNAs as well as DNAs in general, rather than affect the HCV RNA specifically, and oxidative/nitrosative stress is documented in many different virus infections, findings of this study may apply to other viruses and their interactions with the physicochemical environment affecting viral evolution. It would be interesting also to test whether ethanol and reactive species affect the evolution of microbes in general, such as the resident bacteria of the gut.

During alcohol intake, alcohol concentration in the blood can exceed concentrations of 100 mM. The 0.5% (v/v) ethanol used is equivalent to 85.6 mM and lies in the toxic range but can be achieved physiologically, particularly in chronic alcohol users. In addition, ethanol is volatile and the concentration that remains will be significantly less than what is added to cell culture medium. The 0.1% ethanol (v/v), which also inflicted RNA damage with longer exposure, is equivalent to blood alcohol concentration of 17.2 mM, which is approximately the legal limit for driving under the influence in many countries. The cells express CYP2E1 and, therefore, can metabolize ethanol. In addition, acetaldehyde levels used in this study are physiologically attained concentrations. Regarding GSH, GSH is suppressed in hepatitis C patients but it is elevated by infectious HCV in cell culture. The reason for this discrepancy is unclear and can involve a number of factors including the status of p53, cell cycle, aging, and extent of oxidative/nitrosative stress, potentially exceeding the host antioxidant capacity in vivo. To mimic GSH depletion seen in vivo in this cell culture system, this example used BSO, an irreversible inhibitor of GSH de novo synthesis. BSO increased oxidative RNA damage and sequence heterogeneity of HCV RNA (FIG. 5). Moreover, infectious JFH1 induced RNA damage even in the absence of ethanol and under GSH-sufficient condition, and this was exacerbated by co-incubation with ethanol or BSO (FIG. 7). These data strongly suggest that HCV genome sequence would be affected by endogenous reactive species and GSH depletion occurring in vivo, and that reactive species' accelerating the mutation rate is a direct consequence of HCV infection. These effects, therefore, likely contributed to previous estimation of the mutation rate of HCV done in patients. Indeed, positive selection data from the HCV sequence database were closer to the treatment groups than the control group (FIG. 9), which is consistent with an important role of GSH depletion and endogenous and exogenous sources of reactive species in viral evolution in vivo. Differences, on the other hand, might be attributed to variable exposure to exogenous genotoxins, lack of complete viral replication cycle in this study (i.e., replicon model), diurnal/hormonal/age-dependent fluctuation of GSH, fluctuations in the level of oxidative/nitrosative stress with viral titer and stage of liver disease, as well as presence of additional selection pressures in vivo. Endogenous sources of ROS/RNS activated by HCV are likely to include the mitochondria, hepatocyte NAD(P)H oxidases, inducible nitric oxidase synthase, as well as CYP2E1, many of which can be targeted pharmacologically (FIG. 12).

Reactive species and acetaldehyde can induce chemical modification of the DNA, and similar modifications are likely to occur on the RNA. Oxidative modification of RNA, like corresponding lesions on the DNA, has been shown to be mutagenic, causing mispairing of the bases, which may have a significant consequence for viruses that rely on RNA genomes. On the other hand, evolution is affected by the environment, and both ethanol and reactive species are capable of altering intracellular environment of the virus via modification of cell metabolism and signaling, which may act as selection pressure. In this study, nonsynonymous mutations were found to be largely due to positive selection. Synonymous mutations did not increase with treatments. Pronounced changes in STY residues also suggested alteration of cell signaling followed by adaptation. These findings suggest that ethanol and reactive species increase the mutation rate of HCV, largely by positive selection. Indeed, HCV, like other RNA viruses, operate near error threshold and increasing its mutation rate beyond this threshold can lead to an error catastrophe. However, the extent to which ethanol and BSO pushed the mutation rate of HCV with respect to the error threshold in this study is yet unclear, and the ability to attenuate both the RNA damage and mutation rate of HCV with an iron chelator suggests a role of hydroxyl radical which, due to its high chemical reactivity, is thought to participate in irreversible, damaging reactions than cell signaling. Thus, chemical mutagenesis might have contributed to the generation of sequence variants, thereby enabling positive selection. Indeed, with more than 20 fold increases in the oxidative RNA damage (FIG. 5), some chemical mutagenesis of viral RNA would be expected. Also, although it has been debated, mutagens can impose selection pressure for the development of resistance, and ribavirin, which has been shown to exert antiviral effects on HCV at least in part by acting as a viral mutagen, can clearly lead to the emergence of drug resistance. In addition, mutations did not occur uniformly throughout the population of viral RNAs in this study and some RNAs contained only a few mutations. In such conditions, it has been argued that a few resistant strains could then outcompete and survive as the killed strains drop from the fitness curve. It should also be noted that reactive species can directly modify amino acids. Furthermore, although errors generated by the HCV viral polymerase are more likely to be transition mutations, similar to that of poliovirus, oxidative/nitrosative stress can induce transitions or transversions depending on the types of reactive species and polymerase involved. Thus, whether the nucleotide substitution trends in the treatment groups (Tables I and II) purely reflect the most common errors produced by HCV replicase plus positive selection/chemical mutagenesis of viral RNA or also other mechanisms, such as direct modification of the viral replicase by reactive species, remains to be determined. Previously, H₂O₂ was shown to suppress HCV RNA replication. This phenomenon was biochemically mediated by elevation of cytosolic calcium and not likely to be mediated by lethal mutagenesis.

In terms of biological effects of the mutations, alcohol consumption decreases the efficacy of the antiviral therapy and antioxidants generally improve the outcome of anti-HCV therapy. Also, although the overall complexity of quasispecies in HCV is typically more important in the success of viral adaptive response to selection pressures, there are several individual or combinations of sites that, when mutated, can confer resistance to various anti-HCV treatments (Table IV). NS5A is suggested to have diverse functions, including antiviral resistance/immune tolerance. The do/ds increased with ethanol and BSO treatments in this study, and specific sites previously implicated in antiviral resistance to interferon, ribavirin, cyclosporine, and Debio 025 could be demonstrated (Table IV). Additionally, it showed that Y321C confers a replicative fitness advantage to the virus over the wildtype in the presence of cyclosporine (FIG. 11). Therefore, although the frequency of these mutations was still low, these mutations demonstrate specific changes to the HCV genome that would increase its resistance to interferon and ribavirin-based therapy, cyclosporine, and its analogs. Previously, hepatic iron was found to inversely correlate with patients' response to interferon therapy. Iron depletion increased sustained response to interferon therapy, and N-acetylcysteine, which partly restored GSH, could enhance the response to interferon. It will be important to confirm these findings and determine whether iron chelator, GSH ester, and other inhibitors of ROS/RNS can affect the mutation rate of HCV in vitro and in vivo. Another possible consequence of increased mutations to HCV NS5A region is alteration of viral replication cycle. Previously, alcohol was suggested to affect HCV replication, and it was showed that alcohol and acetaldehyde potentiated HCV RNA replication through lipid metabolism and by elevating NADH/NAD+ ratio in the cell. NS5A is a phosphoprotein that can act as a molecular switch that regulates HCV replication, translation, assembly, and secretion. In this study, 50.3% of all nonsynonymous substitutions involved serine, threonine, or tyrosine residues. Therefore, although mutations that either added or removed serine residues still represented only a small percentage of total serine residues found in NS5A, it is possible these changes affect the phosphorylation state of specific micro or subdomains within NS5A. Removal of serine residues within the basal phosphorylation region of NS5A in ethanol and BSO groups suggests that the basal phosphorylation has a significant effect on average fitness of HCV so that mutations removing these sites were positively selected. In a recent study, CKII-induced basal phosphorylation of NS5A decreased the activity of NS5B and its association with viral RNA templates. On the other hand, removing NS5A basal phosphorylation sites besides S457 of genotype 2a JFH1 had no effect on HCV replication, and how these mutations involving serine, threonine, and tyrosine affect NS5A phosphorylation, HCV replication, and secretion remain to be tested. Whether the mutations induced by ethanol/reactive species also affect the virulence of HCV is unknown.

Lastly, it is interesting to note that so many of the mutations that are known to affect antiviral sensitivity, immune response, and viral replication cycle were generated by treating cells either with ethanol or BSO in this study. Although some of these mutations are most likely generated randomly, many were positively selected (Table IV), and these findings suggest an existence of shared biological processes so that adaptation to one variable affect the sensitivity to another. Cyclosporine, for example, was reported to inhibit HCV replication through oxidative stress, and some of the protein kinases implicated in NS5A phosphorylation are modulated by ethanol and/or oxidative stress. In this scheme, note that the present findings support, rather than counter, the previous findings that H₂O₂ tends to suppress HCV RNA replication, such that it can result in the selection of mutations conferring increased resistance to cyclosporine.

Therefore, this study showed that ethanol and endogenous reactive species could increase RNA damage and increase the mutation frequency of HCV in hepatoma cells in the absence of other components of the host immune system. RNA damage, positive selection, and sequence heterogeneity of HCV RNA could be decreased by strategies used to decrease ROS/RNS. These findings suggest another potential key mechanism of adverse interaction between ethanol and HCV, and another important consequence of chronic oxidative/nitrosative stress induced by HCV besides pathogenesis. This study also raises an interesting possibility that, by understanding the nature of such chemical-biological interactions driving viral evolution, it might be feasible to attenuate the antiviral resistance of an RNA virus, biochemically.

TABLE V
Amino acid variations in the HCV database.

| | Codon | Codon | AA | AA | |
|---|---|---|---|---|---|
| Pos | from | to | from | to | Accession #'s |
| 1 | tcc | gcc | S | A | AJ133096, EF407470, EF407500, EU155300, EU482833 |
| 1 | tcc | ccc | S | P | AM401884, AM401918, AM402082, AM402309 |
| 2 | ggc | gac | G | D | AJ507158, AM402310, AM402311, AM402312, AM402313, |
| | | | | | AM402314, AM402315, AM402316, AM402317, AM402318, |
| | | | | | AM402319, AM402320, AM402321, AM402322, AM402323, |
| | | | | | AM402325, AM402326, AM402327, AM402328, EU155263 |
| 2 | ggc | gat | G | D | EU862837 |
| 2 | ggc | aac | G | N | AM402258, AM402259, AM402260, AM402261, AM402262, |
| | | | | | AM402263, AM402264, AM402265, AM402266, AM402267, |
| | | | | | AM402268, AM402269, AM402270, AM402271, AM402272, |
| | | | | | AM402273, AM402274, AM402275, AM402276, AM402277, |
| | | | | | AM402278, AM402279, AM402280, AM402281, AM402282, |
| | | | | | AM402283, AM402284, AM402285, AM402286, AM402287, |
| | | | | | AM402288, AM402289, AM402290, AM402291, AM402292, |
| | | | | | AM402293, AM402294, AM402295, AM402296, AM402297, |
| | | | | | AM402298, AM402299, AM402300, AM402301, AM402302, |
| | | | | | AM402303, AM402304, AM402305, AM402306, AM402307, |
| | | | | | AM402308, AM402309, AM402324, AM493436, AM493437, |
| | | | | | AM493438, AM493439, AM493440, AM493441, AM493442, |
| | | | | | AM493443, AM493444, AM493445, AM493446, AM493447, |
| | | | | | AM493448, AM493449, AM493450, AM493451, EU155368 |
| 2 | ggc | agc | G | S | AJ507162, EF407486, EU155318, HM042058, M96362, U16362 |
| 3 | tcg | acg | S | T | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507, EU155376 |
| 3 | tcg | aca | S | T | EU155260, EU155306 |
| 5 | cta | cca | L | P | AM401862 |
| 6 | aga | aag | R | K | AB010249, AB285036, AB285037, AB285054, AB285055, |
| | | | | | AF034151, AF265163, AJ507163, AM402302, AY240968, |
| | | | | | AY240969, D11168, D90208, EU155281, FJ958928 |
| 6 | aga | tgg | R | W | AF265161 |
| 7 | gat | gaa | D | E | AF165061, EU155318 |
| 7 | gat | gag | D | E | M96362, U16362 |
| 7 | gat | ggc | D | G | AM402350, AM493498 |
| 8 | gtt | gct | V | A | DQ491949, FJ958920 |
| 8 | gtt | ata | V | I | AB285052 |
| 8 | gtt | atc | V | I | AF483269, AJ507156, AM402286, AY808021, EU155370, |
| | | | | | EU256089, GU451224 |
| 8 | gtt | att | V | I | AB080299, AB285067, AF165053, AF165054, AJ507172, |
| | | | | | AM494937, AY460204, AY808014, AY808033, D10934, D85516, |
| | | | | | DQ492093, DQ492163, DQ492164, DQ492165, DQ492166, |
| | | | | | DQ492167, DQ492168, DQ492169, DQ492170, DQ492171, |
| | | | | | DQ492172, DQ492173, DQ492174, DQ492175, DQ492176, |
| | | | | | DQ492177, DQ492178, DQ492179, DQ492180, DQ492181, |
| | | | | | DQ492182, DQ492183, DQ492184, DQ492185, DQ492186, |
| | | | | | DQ492187, DQ492188, DQ492189, DQ492190, DQ492191, |
| | | | | | DQ492192, DQ492193, DQ492194, DQ492195, DQ492196, |
| | | | | | DQ492197, EF638081, EU255960, EU255961, EU781830, |
| | | | | | FN435993, GU451221, GU451222, GU451223, HM042059, M96362, |
| | | | | | U16362 |
| 9 | tgg | agg | W | R | AM402291 |
| 9 | tgg | cgg | W | R | DQ492021, DQ492023, DQ492078, DQ492088, DQ492173 |
| 10 | gat | gaa | D | E | AJ507163, AJ507182, EU256090, EU482833, GQ913871, |
| | | | | | GU451224 |
| 10 | gat | ggc | D | G | AM401943, AM402115, DQ492095 |
| 10 | gat | tac | D | Y | AB285048 |
| 11 | tgg | tga | W | * | DQ492151 |
| 11 | tgg | cgg | W | R | AM402080 |
| 11 | tgg | agg | W | R | DQ492116 |
| 12 | ata | atg | I | M | AM493444 |
| 12 | ata | aca | I | T | DQ492000 |
| 12 | ata | gta | I | V | AF207761, DQ491944, DQ492141, DQ492180, DQ492194, |
| | | | | | DQ492195, EU155224 |
| 12 | ata | gtg | I | V | AB285054, AB285055, AM402329, AM402330, AM402331, |
| | | | | | AM402332, AM402333, AM402334, AM402335, AM402336, |
| | | | | | AM402337, AM402338, AM402339, AM402340, AM402341, |
| | | | | | AM402342, AM402343, AM402344, AM402345, AM402346, |
| | | | | | AM402347, AM402348, AM402349, AM402350, AM402351, |
| | | | | | AM402352, AM402353, AM402354, AM402355, AM402356, |
| | | | | | AM402357, AM402358, AM402359, AM402360, AM402361, |
| | | | | | AM402362, AM402363, AM402364, AM402365, AM402367, |
| | | | | | AM402369, AM402370, AM402371, AM402372, AM402373, |
| | | | | | AM402374, AM402375, AM402376, AM402377, AM402378, |
| | | | | | AM402379, AM402380, AM402381, AM402382, AM402383, |
| | | | | | AM402384, EU256102 |
| 13 | tgc | cgc | C | R | DQ491968 |
| 13 | tgc | tac | C | Y | AM402086 |
| 14 | acg | gcg | T | A | AB049092, AF207761, AM402265, AM402356, DQ491989, |
| | | | | | DQ492067, DQ492141, DQ492152 |
| 14 | acg | att | T | I | AY808022 |
| 14 | acg | atg | T | M | AB154195, AB154196, AB285078, DQ859970, EF638081, |
| | | | | | HM042052 |
| 14 | acg | tcg | T | S | AY808009, D10934, DQ492194, DQ492195, EF407471 |
| 14 | acg | gtg | T | V | EU155382 |
| 15 | gtg | gcg | V | A | AB285067, AY240968 |
| 15 | gtg | ata | V | I | EU155381 |
| 16 | ttg | atg | L | M | DQ491946, EF638081, EU155223 |
| 16 | ttg | ccg | L | P | DQ492158 |
| 16 | ttg | tcg | L | S | AM402379 |
| 16 | ttg | gtg | L | V | AJ507198, DQ492145, EU256089 |
| 17 | act | gcc | T | A | AF165063, AF165064, DQ492087, EF407499 |
| 17 | act | gct | T | A | AB010250, AB080299, AB154185, AB154186, AB154190, |
| | | | | | AB285047, AB285062, AB285066, AB285071, AB442221, |
| | | | | | AB442222, AF034151, AF165055, AF165056, AF207754, AF207760, |
| | | | | | AF265156, AJ507162, AJ507172, AJ507178, AJ507186, AJ507191, |
| | | | | | AM402269, AM402300, AM493503, AY587016, AY808010, |
| | | | | | AY808017, AY808022, D30613, D45172, D89815, DQ244140, |
| | | | | | DQ492066, DQ492067, DQ492070, DQ492073, DQ492074, |
| | | | | | DQ492078, DQ492079, DQ492080, DQ492081, DQ492082, |
| | | | | | DQ492083, DQ492084, DQ492085, DQ492086, DQ492088, |
| | | | | | DQ492089, DQ492090, DQ492091, DQ492092, EF407469, |
| | | | | | EF407485, EU155222, EU155223, EU155230, EU155255, |
| | | | | | EU155301, EU155307, EU155326, EU234061, EU255962, |
| | | | | | EU256081, EU256091, EU482880, EU857431, GQ913873, M84754 |
| 17 | act | att | T | I | AB049091, AB285045, AM401954, AM401955, DQ492010, |
| | | | | | EF407467, EU155259, EU155306 |
| 17 | act | atg | T | M | AB285079 |
| 17 | act | aac | T | N | EU155369 |
| 17 | act | agc | T | S | AB049100, AB154195, AB154196, AB285044, AB285052, |
| | | | | | AB285054, AB285055 |
| 17 | act | agt | T | S | AB285057, AB285059, AB285064, AB285072, AB285075, |
| | | | | | AB442219, AB442220, AF165047, AF165048, AF165051, AF165052, |
| | | | | | AF165059, AF207752, AF207753, AF207762, AF207763, AF207769, |
| | | | | | AF207770, AF207771, AF265142, AF265143, AF265144, AF265145, |
| | | | | | AF265146, AF265147, AF265153, AF265160, AF265161, AF265162, |
| | | | | | AF483269, AJ133096, AJ507156, AJ507160, AJ507181, AJ507198, |
| | | | | | AM401926, AM401939, AM494937, AY240968, AY808013, |
| | | | | | AY808019, AY808021, AY808026, AY808033, AY808036, D50481, |
| | | | | | D90208, DQ492094, DQ492095, DQ492096, DQ492097, DQ492098, |
| | | | | | DQ492099, DQ492100, DQ492101, DQ492102, DQ492103, |
| | | | | | DQ492104, DQ492105, DQ492106, DQ492107, DQ492108 |
| 17 | act | tca | T | S | DQ492109 |
| 17 | act | tcc | T | S | DQ492110, DQ492111, DQ492112, DQ492113, DQ492114, |
| | | | | | DQ492115, DQ492116, DQ492117 |
| 17 | act | tct | T | S | DQ492118, DQ492119, DQ492120, DQ492121, DQ492122, |
| | | | | | DQ492123, DQ492158, EF407459, EF407462, EF407468, |
| | | | | | EF407470, EF407475, EF407476, EF407480, EF407487, EF407488, |
| | | | | | EF407492, EF407500, EU155219, EU155220, EU155226, |
| | | | | | EU155232, EU155254, EU155256, EU155260, EU155262, |
| | | | | | EU155263, EU155303, EU155325, EU155327, EU155330, |
| | | | | | EU155334, EU155336, EU155361, EU155364, EU234062, |
| | | | | | EU239714, EU255960, EU256001, EU256059, EU256066, |
| | | | | | EU256083, EU256090, EU482875, EU482883, EU482885, |
| | | | | | EU781825, EU781827, EU781830, GQ913870, GU451218, |
| | | | | | GU451219, HM042053, HM042056, HM042058, HM042059, |
| | | | | | HM042060, HM042063 |
| 17 | act | gtt | T | V | AB049090 |
| 18 | gat | ggc | D | G | AM402359, DQ492106, DQ492134 |
| 18 | gat | aac | D | N | DQ492014 |
| 19 | ttc | ctc | F | L | AM402262, AM402307, DQ492197 |
| 19 | ttc | tta | F | L | AF034151, DQ492192 |
| 19 | ttc | ttg | F | L | AJ507165, DQ492011 |
| 19 | ttc | tcc | F | S | AM402065 |
| 20 | aag | gag | K | E | AM401859, AM401917, AM401954, AM401955, AM402074, |
| | | | | | AM493474, AM493495, FJ958914 |
| 20 | aag | atg | K | M | AM402275 |
| 20 | aag | cag | K | Q | DQ492192 |
| 20 | aag | agg | K | R | AM402127, AM402300, AM402333, EF407472, EU155329, |
| | | | | | HM042058 |
| 21 | acc | gcc | T | A | AF207754, AM401941, DQ492000, EF407485 |
| 21 | acc | atc | T | I | EU155281 |
| 21 | acc | aac | T | N | AY808032 |
| 21 | acc | tcc | T | S | AY808037 |
| 22 | tgg | ggg | W | G | DQ492110 |
| 22 | tgg | cgg | W | R | AM493506 |
| 23 | ctc | atc | L | I | AF207765 |
| 23 | ctc | ccc | L | P | DQ491958, DQ491969, DQ492021 |
| 24 | cag | aag | Q | K | AB049100, AB080299, AB442221, AF207756, D85516 |
| 24 | cag | cga | Q | R | AM401865, EU155361 |
| 24 | cag | cgg | Q | R | AF176573, AY808034, DQ492008, DQ492009, DQ492010, |
| | | | | | DQ492011, DQ492012, DQ492013, DQ492014, DQ492015, |
| | | | | | DQ492016, DQ492017, DQ492018, DQ492019, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ492168, |
| | | | | | DQ492174, EF407459, EU155225, EU256088 |
| 25 | tcc | gcc | S | A | EF407461 |
| 25 | tcc | ccc | S | P | DQ491970 |
| 25 | tcc | acc | S | T | AB285039, AB285046, AB285071, AF265148, AF265149, |
| | | | | | AF265150, AF265163, AF265164, AF265165, AJ507187, DQ491992, |
| | | | | | EF407477, EU155254, EU155263, EU862837, GQ913872 |
| 26 | aag | gag | K | E | AM401882, AM402261, AM402268, DQ491977, DQ492014, |
| | | | | | DQ492146 |
| 26 | aag | atg | K | M | AM402364 |
| 26 | aag | cag | K | Q | DQ492162 |
| 26 | aag | aga | K | R | D50481, DQ492057, EF407491 |
| 26 | aag | agg | K | R | AB285045, AJ507157, AM401853, D50485, DQ491961, DQ491964, |
| | | | | | DQ491966, DQ492154, DQ492155, DQ492156, EF407485, |
| | | | | | EU256001, EU781832, FJ958890, FJ958891, FJ958892, FJ958893, |
| | | | | | FJ958894, FJ958895, FJ958896, FJ958897, FJ958898, FJ958899, |
| | | | | | FJ958900, FJ958901, FJ958902, FJ958903, FJ958904, FJ958905, |
| | | | | | FJ958906, FJ958907, FJ958909, FJ958910, FJ958911, FJ958912, |
| | | | | | FJ958913, FJ958914, FJ958915, FJ958916, FJ958917, FJ958918, |
| | | | | | FJ958919, FJ958920, FJ958921, FJ958922, FJ958923, FJ958924, |
| | | | | | FJ958925, FJ958926, FJ958927, FJ958928, FJ958929, FJ958930, |
| | | | | | FJ958931, FJ958932, FJ958933, FJ958934, FJ958935 |
| 27 | ctc | atc | L | I | AF165059, AF165060, EU155255 |
| 27 | ctc | ccc | L | P | AM401851, DQ492132, DQ492135, DQ492139, DQ492148 |
| 27 | ctc | gtc | L | V | AB049090, AF207760, D50481, D50485, DQ492184, FJ958895, |
| | | | | | FJ958898, FJ958899, FJ958900, FJ958902, FJ958904, FJ958905, |
| | | | | | FJ958906, FJ958907, FJ958909, FJ958911, FJ958912, FJ958913, |
| | | | | | FJ958915, FJ958916, FJ958918, FJ958919, FJ958921, FJ958922, |
| | | | | | FJ958923, FJ958925, FJ958926, FJ958927, FJ958928, FJ958929, |
| | | | | | FJ958930, FJ958932, FJ958933 |
| 28 | ctg | atg | L | M | AB049100, AB080299, AB442221, AF165059, AF165060, |
| | | | | | AF207763, AJ507172, AJ507197, D85516, EU155372 |
| 28 | ctg | ccg | L | P | DQ492168, DQ492174 |
| 28 | ctg | cag | L | Q | DQ492084 |
| 28 | ctg | gtg | L | V | AF207756, EU256080, EU256084 |
| 29 | ccg | tca | P | S | FJ958897 |
| 29 | ccg | tcg | P | S | AM402324, AM402346 |
| 30 | cga | aaa | R | K | AB010249, D11168, D16435, D89872, EU155219, EU155226 |
| 30 | cga | aag | R | K | AB285067 |
| 30 | cga | ctg | R | L | AB442221, AY808034, DQ492008, DQ492009, DQ492010, |
| | | | | | DQ492011, DQ492012, DQ492013, DQ492014, DQ492015, |
| | | | | | DQ492016, DQ492017, DQ492018, DQ492019, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039 |
| 30 | cga | atg | R | M | AF265154, AF265155, AF265156, EU482879 |
| 30 | cga | caa | R | Q | AB080299, AB285069, AJ507183, AY808028, DQ491979, |
| | | | | | DQ491980, DQ491981, DQ491982, DQ491983, DQ491984, |
| | | | | | DQ491985, DQ491986, DQ491987, DQ491988, DQ491989, |
| | | | | | DQ491990, DQ491991, DQ491992, DQ491993, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, EU234062 |
| 30 | cga | cag | R | Q | AB049100, AB285065, AF207756, AF333324, AM402060, D63857, |
| | | | | | DQ492131, EF407472, EF407487, EF407501, EF407502, |
| | | | | | EU155228, EU155258, EU155262, EU155361, EU155374, |
| | | | | | EU256085, EU256088, EU256091, EU862835, GU451220, M58335 |
| 30 | cga | tgg | R | W | AM402377 |
| 31 | ttg | ttt | L | F | AY808037 |
| 31 | ttg | ata | L | I | AY808015, EU155373, EU234062 |
| 31 | ttg | atg | L | M | AB049089, AB285041, AB285042, AJ507158, AJ507163, AJ507182, |
| | | | | | AY808034, AY808035, DQ492008, DQ492009, DQ492010, |
| | | | | | DQ492011, DQ492012, DQ492013, DQ492014, DQ492015, |
| | | | | | DQ492016, DQ492017, DQ492018, DQ492019, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ492040, |
| | | | | | DQ492041, DQ492042, DQ492043, DQ492044, DQ492045, |
| | | | | | DQ492046, DQ492047, DQ492048, DQ492049, DQ492050, |
| | | | | | DQ492051, DQ492052, DQ492053, DQ492054, DQ492055, |
| | | | | | DQ492056, DQ492057, DQ492058, DQ492059, DQ492060, |
| | | | | | DQ492061, DQ492062, DQ492194, DQ492195, EF407478, |
| | | | | | EU155219, EU155262, EU155315, EU155327, EU256061, |
| | | | | | EU256065, EU256101, EU482833, EU482885, FJ024279, FJ390397 |
| 31 | ttg | tcg | L | S | DQ492001 |
| 31 | ttg | tgg | L | W | DQ492078, DQ492088 |
| 32 | ccg | tcg | P | S | AM401883, DQ492093 |
| 33 | gga | aga | G | R | AM401968 |
| 34 | gtc | gcc | V | A | AJ507170, AJ507182, AM401879, AM402058, DQ492009 |
| 34 | gtc | gac | V | D | DQ491947, DQ492136, X61596 |
| 34 | gtc | atc | V | I | AB285042, AB285043, AB285045, AB285047, AB285050, |
| | | | | | AB285061, AB285063, AB285065, AB285069, AB442222, |
| | | | | | AF165053, AF165054, AF165055, AF165056, AJ000009, AJ507189, |
| | | | | | AJ507194, AJ507197, AJ507199, AM494937, AY808027, AY808028, |
| | | | | | AY808033, D50482, D50484, D63857, DQ491979, DQ491980, |
| | | | | | DQ491981, DQ491982, DQ491983, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, EF407472, EF407480, EF407488, |
| | | | | | EF407491, EU155227, EU155235, EU155253, EU255961, |
| | | | | | EU256101, GQ913869, HM042054, HM042055, HM042056, |
| | | | | | HM042058, HM042061 |
| 34 | gtc | att | V | I | AB285078, EU155316, EU256065, HM042063 |
| 34 | gtc | ctc | V | L | AB285044, AB285059, AF165051, AF165052, AJ507163, AJ507167, |
| | | | | | AJ507172, AM402056, D90208, EF407483, EF407500, EF407501, |
| | | | | | EU155220, EU155228, EU155300, EU155362, EU239714, |
| | | | | | EU256000, EU256066, EU256079, EU482880, GQ913870, |
| | | | | | HM042053, HM042059, HM042060 |
| 36 | ttc | ctc | F | L | AB285040, DQ491997, DQ492017, EU256078 |
| 36 | ttc | ctt | F | L | AM401947 |
| 36 | ttc | tac | F | Y | DQ492128 |
| 37 | ttc | atc | F | I | AB442222, AJ507163, AJ507182, AJ507188, AJ507197, AY808023, |
| | | | | | EF032892, EF032893, EF032894, EF407461, EF407468, EU155254, |
| | | | | | EU155256, EU155257, EU155279, EU155316, EU256064, |
| | | | | | EU482833, EU660388, EU862837, FJ390396 |
| 37 | ttc | att | F | I | AY808036 |
| 37 | ttc | cta | F | L | AJ507177, AM402055, AM402056, AM402057, AM402058, |
| | | | | | AM402059, AM402060, AM402061, AM402062, AM402063, |
| | | | | | AM402064, AM402065, AM402066, AM402067, AM402068, |
| | | | | | AM402069, AM402070, AM402071, AM402072, AM402073, |
| | | | | | AM402074, AM402075, AM402076, AM402077, AM402078, |
| | | | | | AM402079, AM402080, AM402081, AM402082, AM402083, |
| | | | | | AM402084, AM402085, AM402086, AM402087, AM402088, |
| | | | | | AM402089, AM402090, AM402091, AM402092, AM402093, |
| | | | | | AM402094, AM402095, AM402096, AM402097, AM402098, |
| | | | | | AM402100, AM402101, AM402102, AM402103, AM402104, |
| | | | | | AM402105, AM402106, AM402107, AM402108, AM402109, |
| | | | | | AM402110, AM402111, AM402112, AM402113, AM402114, |
| | | | | | AM402115, AM402116, AM402117, AM402118, AM402119, |
| | | | | | AM402120, AM402121, AM402122, AM402123, AM402124, |
| | | | | | AM402125, AM402126, AM402127, AM402128, AM402129, |
| | | | | | AY808016, AY808030, EU155221, EU155307, EU155365, |
| | | | | | EU256066, EU256085, EU529682, EU862835, FJ390397 |
| 37 | ttc | ctc | F | L | AB049090, AB049095, AB154177, AB154178, AB154179, |
| | | | | | AB154180, AB154183, AB154184, AB154185, AB154186, |
| | | | | | AB154187, AB154188, AB154189, AB154190, AB154191, |
| | | | | | AB154192, AB154193, AB154195, AB154196, AB154197, |
| | | | | | AB154198, AB154199, AB154200, AB154201, AB154202, |
| | | | | | AB154203, AB154204, AB154205, AB154206, AB285037, |
| | | | | | AB285038, AB285039, AB285040, AB285042, AB285051, |
| | | | | | AB285062, AB285069, AB285072, AB285077, AF033373, |
| | | | | | AF034151, AF165047, AF165048, AF165051, AF165052, AF165059, |
| | | | | | AF165060, AF165063, AF165064, AF176573, AF207752, AF207753, |
| | | | | | AF207760, AF207762, AF207766, AF207770, AF207772, AF265142, |
| | | | | | AF265143, AF265144, AF265145, AF265146, AF265147, AF265148, |
| | | | | | AF265149, AF265150, AF265158, AF265159, AF265160, AF265161, |
| | | | | | AF265162, AF265163, AF265164, AF265165, AF313916, AJ133096, |
| | | | | | AJ507156, AJ507160, AJ507162, AJ507166, AJ507167, AJ507168, |
| | | | | | AJ507175, AJ507178, AJ507179, AJ507191, AJ507192, AJ507194, |
| | | | | | AJ507195, AJ507196, AJ507199, AM401881, AM401883, |
| | | | | | AM401890, AM401895, AM402269, AY240969, AY460204, |
| | | | | | AY587844, AY808010, AY808012, AY808014, AY808015, |
| | | | | | AY808025, AY808026, AY808027, AY808029, AY808031, |
| | | | | | AY808033, AY808040, AY808041, AY808043, D50481, D50485, |
| | | | | | DQ491943, DQ491944, DQ491945, DQ491946, DQ491947, |
| | | | | | DQ491948, DQ491949, DQ491950, DQ491951, DQ491952, |
| | | | | | DQ491953, DQ491954, DQ491955, DQ491956, DQ491957, |
| | | | | | DQ491958, DQ491959, DQ491960, DQ491961, DQ491962, |
| | | | | | DQ491963, DQ491964, DQ491965, DQ491966, DQ491967, |
| | | | | | DQ491968, DQ491969, DQ492019, DQ492063, DQ492064, |
| | | | | | DQ492065, DQ492066, DQ492067, DQ492068, DQ492069, |
| | | | | | DQ492070, DQ492071, DQ492072, DQ492073, DQ492074, |
| | | | | | DQ492075, DQ492076, DQ492077, DQ492078, DQ492079, |
| | | | | | DQ492080, DQ492081, DQ492082, DQ492083, DQ492084, |
| | | | | | DQ492085, DQ492086, DQ492087, DQ492088, DQ492089, |
| | | | | | DQ492090, DQ492091, DQ492092, DQ492093, DQ492163, |
| | | | | | DQ492164, DQ492165, DQ492166, DQ492167, DQ492168, |
| | | | | | DQ492169, DQ492170, DQ492171, DQ492172, DQ492173, |
| | | | | | DQ492174, DQ492175, DQ492176, DQ492177, DQ492178, |
| | | | | | DQ492179, DQ492180, DQ492181, DQ492182, DQ492183, |
| | | | | | DQ492184, DQ492185, DQ492186, DQ492187, DQ492188, |
| | | | | | DQ492189, DQ492190, DQ492191, DQ492192, DQ492193, |
| | | | | | DQ492194, DQ492195, DQ492196, DQ492197, EF407459, |
| | | | | | EF407460, EF407462, EF407464, EF407465, EF407466, EF407471, |
| | | | | | EF407476, EF407478, EF407479, EF407480, EF407483, EF407485, |
| | | | | | EF407486, EF407488, EF407499, EF407500, EF407502, EF407504, |
| | | | | | EF638081, EU155218, EU155220, EU155222, EU155224, |
| | | | | | EU155225, EU155227, EU155228, EU155229, EU155231, |
| | | | | | EU155235, EU155255, EU155259, EU155263, EU155264, |
| | | | | | EU155281, EU155300, EU155305, EU155308, EU155315, |
| | | | | | EU155318, EU155325, EU155327, EU155334, EU155335, |
| | | | | | EU155336, EU155337, EU155356, EU155357, EU155358, |
| | | | | | EU155359, EU155360, EU155362, EU155368, EU155372, |
| | | | | | EU155374, EU155375, EU155376, EU234061, EU234062, |
| | | | | | EU255962, EU256000, EU256001, EU256045, EU256059, |
| | | | | | EU256061, EU256062, EU256075, EU256076, EU256079, |
| | | | | | EU256081, EU256082, EU256083, EU256088, EU256089, |
| | | | | | EU256091, EU256098, EU256099, EU256102, EU256103, |
| | | | | | EU482839, EU482849, EU482859, EU482879, EU482888, |
| | | | | | EU781825, EU781827, EU781830, EU781832, FJ024277, FJ024279, |
| | | | | | FJ390398, FJ478453, FJ958890, FJ958891, FJ958893, FJ958895, |
| | | | | | FJ958896, FJ958897, FJ958898, FJ958899, FJ958900, FJ958901, |
| | | | | | FJ958902, FJ958903, FJ958904, FJ958905, FJ958906, FJ958907, |
| | | | | | FJ958909, FJ958910, FJ958911, FJ958912, FJ958913, FJ958914, |
| | | | | | FJ958915, FJ958916, FJ958917, FJ958918, FJ958919, FJ958920, |
| | | | | | FJ958921, FJ958922, FJ958923, FJ958924, FJ958925, FJ958926, |
| | | | | | FJ958927, FJ958928, FJ958929, FJ958930, FJ958931, FJ958932, |
| | | | | | FJ958933, FJ958934, FJ958935, GQ913868, GQ913869, |
| | | | | | GQ913870, GQ913871, GQ913873, GU451218, GU451219, |
| | | | | | GU451221, GU451222, GU451223, GU451224, HM042051, |
| | | | | | HM042053, HM042054, HM042055, HM042056, HM042057, |
| | | | | | HM042058, HM042059, HM042060, HM042061, HM042063, L02836, |
| | | | | | U45476 |
| 37 | ttc | ctg | F | L | AF207758, D90208, EU155302, EU256084 |
| 37 | ttc | ctt | F | L | AB036519, AB036520, AB036521, AB109543, AB154194, |
| | | | | | AB191333, AB285035, AB285043, AB285047, AB285050, |
| | | | | | AB285056, AB285078, AB442220, AF165053, AF165054, |
| | | | | | AF165055, AF165056, AF207757, AF207771, AJ507172, AM494937, |
| | | | | | AY044867, D63857, EF407470, EU155364, EU239714, EU256065, |
| | | | | | EU256080, HM042062 |
| 37 | ttc | ttg | F | L | AJ507174 |
| 37 | ttc | atg | F | M | AJ507171 |
| 37 | ttc | cca | F | P | AM402099 |
| 37 | ttc | ccc | F | P | DQ491970 |
| 37 | ttc | tcc | F | S | AM401869, DQ492094, DQ492109 |
| 37 | ttc | gta | F | V | EF407489 |
| 37 | ttc | gtc | F | V | EU155217, EU155304, EU155367, EU660386 |
| 37 | ttc | tac | F | Y | AJ000009, AJ507159, AJ507190, AJ507198, EU256101 |
| 38 | tca | tta | S | L | AM401851 |
| 38 | tca | ttg | S | L | FJ958915 |
| 38 | tca | cca | S | P | AM402086 |
| 39 | tgt | cgc | C | R | AM402374, DQ492192 |
| 39 | tgt | cgt | C | R | DQ491977 |
| 39 | tgt | tac | C | Y | DQ492161, DQ492165 |
| 40 | caa | cac | Q | H | AM493496 |
| 41 | cgt | cac | R | H | AM401908 |
| 41 | cgt | cat | R | H | AM401920, AM401949, AM401954, AM401977, DQ492027, |
| | | | | | DQ492028, DQ492034, DQ492036, DQ492037, DQ492039, |
| | | | | | EU155235 |
| 41 | cgt | agc | R | S | EU256089 |
| 42 | ggg | gag | G | E | AM401910, DQ492083 |
| 42 | ggg | agg | G | R | AM402066, DQ492044, DQ492045, DQ492051, DQ492052 |
| 43 | tac | tgc | Y | C | DQ492085 |
| 44 | aag | gag | K | E | AM401900, AM401903, AM402380, DQ492152, DQ492168, |
| | | | | | DQ492174 |
| 44 | aag | aga | K | R | AB285048, AB285052, AY808037, EU155362 |
| 44 | aag | agg | K | R | AB049094, AB049101, AB154183, AB154184, AB154185, |
| | | | | | AB154186, AB154195, AB154196, AB154197, AB154198, |
| | | | | | AB154205, AB154206, AB285044, AB285059, AB285070, |
| | | | | | AB285078, AB442219, AB442222, AF165055, AF165056, |
| | | | | | AF207764, AF265142, AF265143, AF265144, AF265160, AF265161, |
| | | | | | AF356827, AJ133096, AJ507159, AJ507164, AJ507165, AJ507168, |
| | | | | | AJ507169, AJ507170, AJ507184, AJ507185, AJ507194, AM401896, |
| | | | | | AM401916, AM401917, AM401918, AM401919, AM401920, |
| | | | | | AM401921, AM401922, AM401923, AM401924, AM401925, |
| | | | | | AM401926, AM401927, AM401928, AM401929, AM401930, |
| | | | | | AM401931, AM401932, AM401933, AM401934, AM401935, |
| | | | | | AM401936, AM401937, AM401938, AM401939, AM401940, |
| | | | | | AM401941, AM401942, AM401943, AM401944, AM401945, |
| | | | | | AM401946, AM401947, AM401948, AM401949, AM401950, |
| | | | | | AM401951, AM401952, AM401953, AM401954, AM401955, |
| | | | | | AM401956, AM401957, AM401958, AM401959, AM401960, |
| | | | | | AM401961, AM401962, AM401963, AM401964, AM401965, |
| | | | | | AM401966, AM401967, AM401968 AM401969, AM401970, |
| | | | | | AM401971, AM401972, AM401973, AM401974, AM401975, |
| | | | | | AM401976, AM401977, AM401978, AM401979, AM401980, |
| | | | | | AM493471, AM493472, AM493473, AM493474, AM493475, |
| | | | | | AM493476, AM493477, AM493478, AM493479, AM493480, |
| | | | | | AM493481, AM493482, AM493483, AM493484, AM493485, |
| | | | | | AM493486, AM493487, AM493488, AY808010, AY808021, |
| | | | | | AY808031, AY808034, AY808035, AY808042, AY808043, D50480, |
| | | | | | D50483, D85516, DQ491971, DQ491972, DQ491973, DQ491974, |
| | | | | | DQ491975, DQ491976, DQ491977, DQ491978, DQ491984, |
| | | | | | DQ491992, DQ492008, DQ492009, DQ492010, DQ492011, |
| | | | | | DQ492012, DQ492013, DQ492014, DQ492016, DQ492017, |
| | | | | | DQ492018, DQ492019, DQ492020, DQ492021, DQ492022, |
| | | | | | DQ492023, DQ492024, DQ492025, DQ492026, DQ492027, |
| | | | | | DQ492028, DQ492029, DQ492030, DQ492031, DQ492032, |
| | | | | | DQ492033, DQ492034, DQ492035, DQ492036, DQ492037, |
| | | | | | DQ492038, DQ492039, DQ492040, DQ492041, DQ492042, |
| | | | | | DQ492043, DQ492044 DQ492045, DQ492046, DQ492047, |
| | | | | | DQ492048, DQ492049 DQ492050, DQ492051, DQ492052, |
| | | | | | DQ492053, DQ492054 DQ492055, DQ492056, DQ492057, |
| | | | | | DQ492058, DQ492059 DQ492060, DQ492061, DQ492062, |
| | | | | | DQ492111, DQ492112 DQ492115, DQ492116 EF407467, |
| | | | | | EF407471, EF407491 EF407498, EU155222, EU155228, |
| | | | | | EU155256, EU155261 EU155316, EU155330, EU155332, |
| | | | | | EU155337, EU155370 EU155371, EU255960, EU256062, |
| | | | | | EU256077, EU256080 EU256084, EU256090, EU256092, |
| | | | | | EU256101, EU256103, EU482877, EU660388, FJ390398, FJ958914, |
| | | | | | GQ913868, GQ913872, HM042062, U45476, X61596 |
| 45 | gga | gag | G | E | AM401979 |
| 45 | gga | aga | G | R | AM401904, AM402058, DQ492115 |
| 46 | gtc | gcc | V | A | AB285039, AF165063, AF165064, DQ491982 |
| 46 | gtc | ttt | V | F | AB285076 |
| 46 | gtc | ata | V | I | AB154201, AB154202 |
| 46 | gtc | atc | V | I | AB049098, AB154177, AB154178, AB154179, AB154180, |
| | | | | | AB154181, AB154182, AB154183, AB154187, AB154188, |
| | | | | | AB154191, AB154192, AB154193, AB154194, AB154197, |
| | | | | | AB154198, AB154199, AB154200, AB154203, AB154204, |
| | | | | | AB154205, AB154206, AF207760, AF207761, AF207769, |
| | | | | | AM401888, AM401973, AY008261, DQ492090, EF407461, |
| | | | | | EF407468, EF407469, EF407488, EU155306, EU256085, |
| | | | | | EU482888, GQ913873 |
| 46 | gtc | att | V | I | AB154184, AB154195, AB154196, AB285040, AJ507168, |
| | | | | | AY808026, AY808038 |
| 47 | tgg | agg | W | R | AM402294 |
| 47 | tgg | cgg | W | R | DQ492087, DQ492106 |
| 48 | cgg | ctg | R | L | AB049087, AM493471, AM493472, AM493473, AM493474, |
| | | | | | AM493475, AM493476, AM493477, AM493478, AM493479, |
| | | | | | AM493480, AM493481, AM493482, AM493483, AM493484, |
| | | | | | AM493485, AM493486, AM493487, AM493488 |
| 48 | cgg | ccg | R | P | AM402294, AM402302 |
| 48 | cgg | cag | R | Q | AB154203, AB154204, AB285051, AM402258, AM402259, |
| | | | | | AM402260, AM402261, AM402262, AM402263, AM402264, |
| | | | | | AM402265, AM402266, AM402267, AM402268, AM402269, |
| | | | | | AM402270, AM402271, AM402272, AM402273, AM402274, |
| | | | | | AM402275, AM402276, AM402277, AM402278, AM402279, |
| | | | | | AM402280, AM402281, AM402282, AM402283, AM402284, |
| | | | | | AM402285, AM402286, AM402287, AM402288, AM402289, |
| | | | | | AM402290, AM402291, AM402292, AM402293, AM402295, |
| | | | | | AM402296, AM402297, AM402298, AM402299, AM402300, |
| | | | | | AM402301, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402326, |
| | | | | | AM402327, AM402328, AM493436, AM493437, AM493438, |
| | | | | | AM493439, AM493440, AM493441, AM493442, AM493443, |
| | | | | | AM493444, AM493445, AM493446, AM493447, AM493448, |
| | | | | | AM493449, AM493450, AM493451, AM494937, AY808027, |
| | | | | | DQ492056, DQ492157, EF407463, EU482875 |
| 49 | ggc | gcc | G | A | HM042062 |
| 49 | ggc | gcg | G | A | EU155306, HM042056, HM042058 |
| 49 | ggc | gaa | G | E | DQ491971, DQ491993, EU155381 |
| 49 | ggc | gag | G | E | AB049100, AF265142, AF265143, AF265144, AF265160, AF265161, |
| | | | | | AF265162 |
| 49 | ggc | tca | G | S | AJ507197 |
| 50 | gac | gcc | D | A | AM402115 |
| 50 | gac | gaa | D | E | AB049099, AB285036, AB285038, D14484 |
| 50 | gac | gag | D | E | EU256099 |
| 50 | gac | aac | D | N | DQ492064 |
| 51 | ggc | gac | G | D | AM402303 |
| 51 | ggc | agt | G | S | AM402384 |
| 52 | atc | gcc | I | A | DQ492151 |
| 52 | atc | atg | I | M | DQ244140, EU155261 |
| 52 | atc | acc | I | T | AB285038, DQ491996, DQ492024, DQ492103 |
| 52 | atc | gtc | I | V | AB036519, AB036520, AB036521, AB049087, AB049092, |
| | | | | | AB049094, AB049095, AB049096, AB109543, AB191333, |
| | | | | | AB285040, AB285043, AB285045, AB285047, AB285048, |
| | | | | | AB285049, AB285050, AB285051, AB285052, AB285053, |
| | | | | | AB285057, AB285064, AB285068, AB285073, AB285075, |
| | | | | | AB285078, AB285079, AB442219, AB442220, AF165047, |
| | | | | | AF165048, AF165053, AF165054, AF165055, AF165056, AF207757, |
| | | | | | AF207768, AF207772, AF265154, AF265155, AJ507175, AJ507189, |
| | | | | | AM401864, AM401869, AM401871, AM401872, AM401874, |
| | | | | | AM401875, AM401877, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, |
| | | | | | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507, AM494937, |
| | | | | | AY808011, AY808016, AY808023, AY808036, D50482, D50484, |
| | | | | | DQ492124, DQ492125, DQ492126, DQ492127, DQ492128, |
| | | | | | DQ492129, DQ492130, DQ492131, DQ492132, DQ492133, |
| | | | | | DQ492134, DQ492135, DQ492136, DQ492139, DQ492140, |
| | | | | | DQ492141, DQ492142, DQ492144, DQ492145, DQ492147, |
| | | | | | DQ492148, DQ492149, DQ492150, DQ492152, DQ492153, |
| | | | | | DQ492154, DQ492155, DQ492156, DQ492157, DQ492158, |
| | | | | | DQ492159, DQ492160, DQ492162, EU155224, EU256088, |
| | | | | | EU482859, EU529682, GU451218, GU451219, HM042061, X61596 |
| 52 | atc | gtt | I | V | AB049089, AB285054, AB285055, EU256102, FJ958914 |
| 53 | atg | ata | M | I | AM402107, AM402108, DQ491967 |
| 53 | atg | acg | M | T | DQ492062, DQ492076, DQ492193 |
| 53 | atg | gtg | M | V | AF165047, AF165048, AM401944, DQ492064, EU155373, |
| | | | | | EU482875, HM042053 |
| 54 | caa | tgt | Q | C | AY808012 |
| 54 | caa | cac | Q | H | AB036519, AB036520, AB036521, AB049089, AB049098, |
| | | | | | AB109543, AB154184, AB154193, AB191333, AB285036, |
| | | | | | AB285043, AB285047, AB285053, AB285057, AB285062, |
| | | | | | AB285073, AB285075, AB285081, AB429050, AF165047, |
| | | | | | AF165048, AF165055, AF165056, AF165061, AF165062, AF207761, |
| | | | | | AF207762, AF207764, AF265142, AF265143, AF265144, AF265160, |
| | | | | | AF265161, AF265162, AF483269, AJ133096, AJ507160, AJ507162, |
| | | | | | AJ507163, AJ507164, AJ507168, AJ507170, AJ507174, AJ507175, |
| | | | | | AJ507178, AJ507184, AJ507199, AM402266, AM493489, |
| | | | | | AM493490, AM493491, AM493492, AM493493, AM493494, |
| | | | | | AM493495, AM493496, AM493497, AM493498, AM493499, |
| | | | | | AM493500, AM493501, AM493502, AM493503, AM493504, |
| | | | | | AM493505, AM493506, AM493507, AM494937, AY808023, |
| | | | | | AY808026, AY808027, AY808039, AY808040, AY808043, D50482, |
| | | | | | D50484, D85516, DQ491943, DQ491944, DQ491945, DQ491946, |
| | | | | | DQ491947, DQ491948, DQ491949, DQ491950, DQ491951, |
| | | | | | DQ491952, DQ491953, DQ491954, DQ491955, DQ491956, |
| | | | | | DQ491957, DQ492076, EF407460, EF407464, EF407468, |
| | | | | | EF407478, EF407483, EF407498, EU155218, EU155220, |
| | | | | | EU155222, EU155225, EU155232, EU155235, EU155256, |
| | | | | | EU155261, EU155281, EU155325, EU155330, EU155337, |
| | | | | | EU155356, EU155360, EU239714, EU256000, EU256062, |
| | | | | | EU256076, EU256077, EU256079, EU256083, EU256090, |
| | | | | | EU482839, EU482860, EU482875, EU482877, EU482886, |
| | | | | | EU482888, EU781825, EU781830, FJ024279, FJ390398, FJ958929, |
| | | | | | GQ913869, GQ913873, HM042051, HM042052, HM042054, |
| | | | | | HM042055, HM042056, HM042057, HM042058, HM042059, |
| | | | | | HM042063, M96362, U16362, U45476 |
| 54 | caa | cat | Q | H | AB049091, AB049092, AB154203, AB154204, AB285037, |
| | | | | | AB285040, AB285041, AB285048, AB285060, AB285064, |
| | | | | | AF207766, AF207769, AF207770, AJ507166, AJ507167, AJ507169, |
| | | | | | AJ507177, AJ507179, AJ507182, AJ507192, AM402258, AM402259, |
| | | | | | AM402260, AM402261, AM402262, AM402263, AM402264, |
| | | | | | AM402265, AM402267, AM402268, AM402269, AM402270, |
| | | | | | AM402271, AM402272, AM402273, AM402274, AM402275, |
| | | | | | AM402276, AM402277, AM402278, AM402279, AM402280, |
| | | | | | AM402281, AM402282, AM402283, AM402284, AM402285, |
| | | | | | AM402286, AM402287, AM402288, AM402289, AM402290, |
| | | | | | AM402291, AM402292, AM402293, AM402294, AM402295, |
| | | | | | AM402296, AM402297, AM402298, AM402299, AM402300, |
| | | | | | AM402301, AM402302, AM402303, AM402304, AM402305, |
| | | | | | AM402306, AM402307, AM402308, AM402309, AM402310, |
| | | | | | AM402311, AM402312, AM402313, AM402314, AM402315, |
| | | | | | AM402316, AM402317, AM402318, AM402319, AM402320, |
| | | | | | AM402321, AM402322, AM402323, AM402324, AM402326, |
| | | | | | AM402327, AM402328, AM493436, AM493437, AM493438, |
| | | | | | AM493439, AM493440, AM493441, AM493442, AM493443, |
| | | | | | AM493444, AM493445, AM493446, AM493447, AM493448, |
| | | | | | AM493449, AM493450, AM493451, AY008261, AY808009, |
| | | | | | AY808015, AY808017, AY808021, AY808024, AY808036, |
| | | | | | AY808037, DQ244140, DQ244141, EF407462, EF407467, |
| | | | | | EF407481, EF407482, EF407484, EF407488, EF407497, EF407500, |
| | | | | | EF407501, EU155219, EU155228, EU155259, EU155279, |
| | | | | | EU155300, EU155303, EU155304, EU155306, EU155327, |
| | | | | | EU155332, EU155333, EU155357, EU155359, EU155369, |
| | | | | | EU155372, EU155373, EU155381, EU256089, EU256091, |
| | | | | | EU256102, EU482874, EU482881, EU482885, EU660386, |
| | | | | | EU781827, FJ024277, FJ390396, FJ958890, FJ958896, FJ958897, |
| | | | | | FJ958903, FJ958905, FJ958906, FJ958910, FJ958914, FJ958917, |
| | | | | | FJ958920, FJ958924, FJ958934, HM042061, HM042062 |
| 54 | caa | cta | Q | L | AY587844, AY808032, D63857, EF407489 |
| 54 | caa | ctc | Q | L | EU155358 |
| 54 | caa | aac | Q | N | AF034151, EU255962, FJ958892, FJ958894 |
| 54 | caa | aat | Q | N | EF407476, EU256066, FJ958891, FJ958893, FJ958895, FJ958898, |
| | | | | | FJ958899, FJ958900, FJ958901, FJ958902, FJ958904, FJ958907, |
| | | | | | FJ958909, FJ958911, FJ958912, FJ958913, FJ958915, FJ958916, |
| | | | | | FJ958918, FJ958919, FJ958921, FJ958922, FJ958923, FJ958925, |
| | | | | | FJ958926, FJ958927, FJ958928, FJ958930, FJ958931, FJ958932, |
| | | | | | FJ958933, FJ958935 |
| 54 | caa | ccg | Q | P | AM493484 |
| 54 | caa | cgg | Q | R | AM401932 |
| 54 | caa | cgt | Q | R | AM402325 |
| 54 | caa | tac | Q | Y | EU256064, EU256078 |
| 54 | caa | tat | Q | Y | AB010250, AB285065, AF165063, AF165064, AJ507171, AY587016, |
| | | | | | D30613, D45172, D89815, EU155368, EU857431 |
| 55 | acc | gcc | T | A | AB285038, AJ507169, AJ507179, AM402296, DQ492034, |
| | | | | | DQ492039, GQ913873, AJ507162, AM401848, AM401973, |
| | | | | | AM493484, DQ492024, EU482879 |
| 55 | acc | gct | T | A | DQ492163 |
| 56 | acc | atc | T | I | AB285038, AB285041, AB285046, AB285049, AB285066, |
| | | | | | AB285068, AB285075, AB442219, AF165045, AF165046, |
| | | | | | AF207755, AF207760, AF265152, AF265153, AJ507190, AM401958, |
| | | | | | AY808016, D50482, D50484, EF407499, EU155219 |
| 56 | acc | att | T | I | EF407469 |
| 56 | acc | aac | T | N | EU256064 |
| 56 | acc | gtc | T | V | AY808035, DQ492040, DQ492041, DQ492042, DQ492043, |
| | | | | | DQ492044, DQ492045, DQ492046, DQ492047, DQ492048, |
| | | | | | DQ492049, DQ492050, DQ492051, DQ492052, DQ492053, |
| | | | | | DQ492054, DQ492055, DQ492056, DQ492057, DQ492058, |
| | | | | | DQ492059, DQ492060, DQ492061, DQ492062, EU256088 |
| 57 | tgc | cgc | C | R | AM402334, DQ492017, DQ492080, DQ492193 |
| 57 | tgc | agc | C | S | DQ492164 |
| 57 | tgc | tgg | C | W | DQ491967 |
| 57 | tgc | tac | C | Y | DQ492101 |
| 58 | cca | gca | P | A | FJ390396 |
| 58 | cca | cta | P | L | AB154197, AB154198, EU155225 |
| 58 | cca | ctg | P | L | AJ507179 |
| 58 | cca | caa | P | Q | AF165049, AF165050, AY808033 |
| 58 | cca | cag | P | Q | AY808032 |
| 58 | cca | tca | P | S | AB154195, AB154196, AB285037, AB285045, AB285048, |
| | | | | | AB285052, AB285057, AB285077, AF483269, AJ507180, |
| | | | | | AM401916, AM401917, AM401918, AM401919, AM401920, |
| | | | | | AM401921, AM401922, AM401923, AM401924, AM401925, |
| | | | | | AM401926, AM401927, AM401928, AM401929, AM401930, |
| | | | | | AM401931, AM401932, AM401933, AM401934, AM401935, |
| | | | | | AM401936, AM401937, AM401938, AM401939, AM401940, |
| | | | | | AM401941, AM401942, AM401943, AM401944, AM401945, |
| | | | | | AM401946, AM401947, AM401948, AM401949, AM401950, |
| | | | | | AM401951, AM401952, AM401953, AM401954, AM401955, |
| | | | | | AM401956, AM401957, AM401958, AM401959, AM401960, |
| | | | | | AM401961, AM401962, AM401963, AM401964, AM401965, |
| | | | | | AM401966, AM401967, AM401968, AM401969, AM401970, |
| | | | | | AM401971, AM401972, AM401973, AM401974, AM401975, |
| | | | | | AM401976, AM401977, AM401978, AM401979, AM401980, |
| | | | | | AM493491, AM493492, AY808012, DQ492050, EF407459, |
| | | | | | EF407488, EU155235, EU155261, EU155317, EU155330, |
| | | | | | EU155382, EU256090, EU482859 |
| 58 | cca | tct | P | S | EU155253 |
| 58 | cca | aca | P | T | EF407482, EF407497, EF407500, EU155279, EU155303, EU482881 |
| 58 | cca | acg | P | T | FJ024277 |
| 59 | tgt | cgt | C | R | AM401854, AM402351, AM402358, DQ491970, DQ492002 |
| 60 | gga | gca | G | A | AF034151 |
| 60 | gga | gag | G | E | DQ492008 |
| 60 | gga | aga | G | R | AM402315 |
| 61 | gca | gga | A | G | AF207765, AM401928, AM401932, AY808042, EF407482, |
| | | | | | EF407497, EF407500, EU155262, EU155279, EU155303, |
| | | | | | FJ024277, FJ390396 |
| 61 | gca | aca | A | T | AM402332 |
| 61 | gca | gta | A | V | AM401853, AM401858 |
| 62 | cag | gac | Q | D | AB049089 |
| 62 | cag | gaa | Q | E | AB285053, AJ133096, AM494937, EF407462, EU155256, |
| | | | | | EU155337, EU781827 |
| 62 | cag | gag | Q | E | EF407481, EF407497 |
| 62 | cag | cac | Q | H | EU256080, EU256084 |
| 62 | cag | cat | Q | H | AF207760 |
| 62 | cag | aaa | Q | K | EF407501, EU155235 |
| 62 | cag | cga | Q | R | DQ492151 |
| 62 | cag | cgg | Q | R | AB154195, AB154196, DQ859970, EU862835 |
| 62 | cag | tcg | Q | S | AB442221 |
| 63 | atc | ctc | I | L | AB285072, EU255961, M84754 |
| 63 | atc | ctg | I | L | EU155261 |
| 63 | atc | ctt | I | L | EF407467 |
| 63 | atc | ttg | I | L | GU451219 |
| 63 | atc | atg | I | M | AM401865, AM401866, AM401870, U45476 |
| 63 | atc | aac | I | N | DQ492133 |
| 63 | atc | acc | I | T | DQ492072, DQ492112, DQ492142 |
| 63 | atc | gtc | I | V | EU155365, EU256001 |
| 64 | acc | gca | T | A | AF265154, AF265155, AF265156, EU482879 |
| 64 | acc | gcc | T | A | AB049090, AB049091, AB049095, AB049099, AB285036, |
| | | | | | AB285038, AB285046, AB285049, AB285050, AB285068, |
| | | | | | AB285077, AF176573, AF207758, AF207760, AF207762, AF207772, |
| | | | | | AF265145, AF265146, AF265147, AF265148, AF265149, AF265150, |
| | | | | | AF265152, AF265153, AF265158, AF265159, AF265163, AF265164, |
| | | | | | AF265165, AJ507156, AJ507166, AJ507195, AJ507196, AM401967, |
| | | | | | AM402055, AM402056, AM402057, AM402058, AM402059, |
| | | | | | AM402060, AM402061, AM402062, AM402063, AM402064, |
| | | | | | AM402065, AM402066, AM402067, AM402068, AM402069, |
| | | | | | AM402070, AM402071, AM402072, AM402073, AM402074, |
| | | | | | AM402075, AM402076, AM402077, AM402078, AM402079, |
| | | | | | AM402080, AM402081, AM402082, AM402083, AM402084, |
| | | | | | AM402085, AM402086, AM402087, AM402088, AM402089, |
| | | | | | AM402090, AM402091, AM402092, AM402093, AM402094, |
| | | | | | AM402095, AM402096, AM402097, AM402098, AM402099, |
| | | | | | AM402100, AM402101, AM402102, AM402103, AM402104, |
| | | | | | AM402105, AM402106, AM402107, AM402108, AM402109, |
| | | | | | AM402110, AM402111, AM402112, AM402113, AM402114, |
| | | | | | AM402115, AM402116, AM402117, AM402118, AM402119, |
| | | | | | AM402120, AM402121, AM402122, AM402123, AM402124, |
| | | | | | AM402125, AM402126, AM402127, AM402128, AM402129, |
| | | | | | AY808016, AY808025, AY808030, AY808035, AY808041, D14484, |
| | | | | | D50482, D50484, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, DQ492040, |
| | | | | | DQ492041, DQ492042, DQ492043, DQ492044, DQ492045, |
| | | | | | DQ492046, DQ492047, DQ492048, DQ492049, DQ492050, |
| | | | | | DQ492051, DQ492052, DQ492053, DQ492054, DQ492055, |
| | | | | | DQ492056, DQ492057, DQ492058, DQ492059, DQ492060, |
| | | | | | DQ492061, DQ492062, EF407465, EF407471, EF407479, |
| | | | | | EF407485, EF407486, EF407502, EF407504, EU155217, |
| | | | | | EU155218, EU155221, EU155231, EU155263, EU155302, |
| | | | | | EU155305, EU155307, EU155315, EU155318, EU155335, |
| | | | | | EU155336, EU155365, EU155367, EU155374, EU155376, |
| | | | | | EU256045, EU256059, EU256061, EU256075, EU256082, |
| | | | | | EU256085, EU256088, EU256098, EU256099, EU256103, |
| | | | | | EU482859, EU482883, EU529682, EU660388, EU862835, |
| | | | | | GQ913870, GU451218, HM042054, HM042060, HM042061, U45476 |
| 64 | acc | gct | T | A | AB049094, AB285039, AB285043, AB285051, AB285052, |
| | | | | | AB285056, AB442220, AF165045, AF165046, AF165053, AF165054, |
| | | | | | AF207755, AF207757, AF207771, AJ507189, EF407459, EF407461, |
| | | | | | EF407464 |
| 64 | acc | agc | T | S | AB049096, AB285042, AB285045, AB285054, AB285055, |
| | | | | | AB285079, AB442219 |
| 64 | acc | agt | T | S | AB285035, AB429050 |
| 64 | acc | tca | T | S | EF407466 |
| 64 | acc | tcc | T | S | AB049087, AB049088, AB049092, AF165058, AF207769, |
| | | | | | AY240968, AY240969, AY808029, DQ492063, DQ492064, |
| | | | | | DQ492065, DQ492068, DQ492069, DQ492071, DQ492072, |
| | | | | | DQ492077, DQ492083, EF032892, EF032893, EF032894, |
| | | | | | EF407467, EF407499, EU155229, EU155254, EU155260, |
| | | | | | EU155363, EU234062, EU255961, EU256065, EU482849 |
| 64 | acc | tct | T | S | AB285069, AF165057, AF356827, AY808027, EU155253 |
| 66 | cat | att | H | I | GU451219 |
| 66 | cat | caa | H | Q | DQ491972, DQ491973 |
| 66 | cat | cgt | H | R | DQ492083, EF638081 |
| 67 | gtg | gcc | V | A | AM401864 |
| 67 | gtg | gac | V | D | DQ492098 |
| 67 | gtg | ttc | V | F | D50482, D50484 |
| 68 | aaa | gaa | K | E | AM402354, DQ491979 |
| 68 | aaa | agg | K | R | DQ492114, DQ492120 |
| 68 | aaa | aca | K | T | AY044867 |
| 69 | aac | gcc | N | A | DQ491998 |
| 69 | aac | aag | N | K | AB285064 |
| 69 | aac | agt | N | S | AM402350, AM402352, AM402353 |
| 69 | aac | acc | N | T | AJ507172, AM402271, DQ491995, DQ491996, DQ491997, |
| | | | | | DQ491999, DQ492000, DQ492001, DQ492002, DQ492003, |
| | | | | | DQ492004, DQ492005, DQ492006, DQ492007, EF407495 |
| 69 | aac | act | N | T | HM042050 |
| 70 | ggt | agt | G | S | AM402262, DQ491955 |
| 71 | tcc | ttc | S | F | DQ492182 |
| 71 | tcc | ccc | S | P | DQ859970 |
| 71 | tcc | acc | S | T | AM401881 |
| 71 | tcc | act | S | T | EU256103 |
| 72 | atg | aag | M | K | DQ492040 |
| 72 | atg | acg | M | T | DQ492109 |
| 72 | atg | gtg | M | V | AM402356 |
| 73 | agg | ggg | R | G | DQ492145 |
| 73 | agg | aag | R | K | AB049087, AB049094, AB285044, AB285057, AF165045, |
| | | | | | AF165046, AF165053, AF165054, AF207755, AF207771, D50482, |
| | | | | | D50484, HM042059 |
| 74 | atc | ctc | I | L | EU482860, HM042056 |
| 74 | atc | ctt | I | L | AB285062 |
| 74 | atc | acc | I | T | DQ491965, DQ492046, FJ958904 |
| 74 | atc | gtc | I | V | AF265153, AM401896, AM401901, AY808035, DQ492040, |
| | | | | | DQ492041, DQ492042, DQ492043, DQ492044, DQ492045, |
| | | | | | DQ492047, DQ492048, DQ492049, DQ492051, DQ492052, |
| | | | | | DQ492053, DQ492054, DQ492055, DQ492056, DQ492057, |
| | | | | | DQ492058, DQ492059, DQ492060, DQ492061, DQ492062, |
| | | | | | EF407464, EF407498 |
| 75 | gtg | gcc | V | A | EU660386 |
| 75 | gtg | gct | V | A | AB049096, AB080299, AB285047, AB285048, AB285058, |
| | | | | | AB285064, AJ507155, AJ507163, AY240969, AY808009, AY808036, |
| | | | | | DQ244140, DQ244141, DQ492092, EU155235, EU155281, |
| | | | | | EU155356, EU155360, HM042055 |
| 75 | gtg | ttc | V | F | AJ507169, GQ913873 |
| 75 | gtg | ttt | V | F | AJ238800 |
| 75 | gtg | ggt | V | G | AM402382 |
| 75 | gtg | ata | V | I | EU482879 |
| 75 | gtg | atc | V | I | EU155227 |
| 75 | gtg | att | V | I | AB285044, AF208024, EU155263, EU155365, EU256103 |
| 75 | gtg | aca | V | T | AF207758, EU155302 |
| 75 | gtg | acc | V | T | AB285062, EU155367, EU482883 |
| 75 | gtg | act | V | T | AB154197, AB154198, AF265154, AF265155, AF265156, AJ507170, |
| | | | | | AM402055, AM402056, AM402057, AM402058, AM402059, |
| | | | | | AM402060, AM402061, AM402062, AM402063, AM402064, |
| | | | | | AM402065, AM402066, AM402067, AM402068, AM402069, |
| | | | | | AM402070, AM402071, AM402072, AM402073, AM402074, |
| | | | | | AM402075, AM402076, AM402077, AM402078, AM402079, |
| | | | | | AM402080, AM402081, AM402082, AM402083, AM402084, |
| | | | | | AM402085, AM402086, AM402087, AM402088, AM402089, |
| | | | | | AM402090, AM402091, AM402092, AM402093, AM402094, |
| | | | | | AM402095, AM402096, AM402097, AM402098, AM402099, |
| | | | | | AM402100, AM402101, AM402102, AM402103, AM402104, |
| | | | | | AM402105, AM402106, AM402107, AM402108, AM402109, |
| | | | | | AM402110, AM402111, AM402112, AM402113, AM402114, |
| | | | | | AM402115, AM402116, AM402117, AM402118, AM402119, |
| | | | | | AM402120, AM402121, AM402122, AM402123, AM402124, |
| | | | | | AM402125, AM402126, AM402127, AM402128, AM402129, |
| | | | | | AY808030, EU529682 |
| 75 | gtg | tgg | V | W | M84754 |
| 75 | gtg | tat | V | Y | AB285041, EF407468, HM042056 |
| 76 | ggg | gag | G | E | DQ492068 |
| 76 | ggg | agg | G | R | AM401892 |
| 77 | cct | ctc | P | L | DQ492009 |
| 77 | cct | ctt | P | L | FJ958892, FJ958894 |
| 77 | cct | tct | P | S | AB154190, AM402312 |
| 78 | agg | ggg | R | G | AM402120 |
| 78 | agg | aaa | R | K | AB010249, AB036519, AB036520, AB036521, AB049088, |
| | | | | | AB049089, AB049092, AB049094, AB049095, AB049098, |
| | | | | | AB049099, AB049101, AB080299, AB109543, AB191333, |
| | | | | | AB249644, AB285035, AB285036, AB285037, AB285039, |
| | | | | | AB285040, AB285043, AB285044, AB285045, AB285046, |
| | | | | | AB285047, AB285048, AB285049, AB285050, AB285051, |
| | | | | | AB285052, AB285053, AB285054, AB285055, AB285056, |
| | | | | | AB285057, AB285058, AB285060, AB285061, AB285062, |
| | | | | | AB285063, AB285064, AB285069, AB285070, AB285071, |
| | | | | | AB285072, AB285073, AB285074, AB285075, AB285076, |
| | | | | | AB285077, AB285079, AB285081, AB426117, AB429050, |
| | | | | | AB442219, AB442220, AB442221, AF033373, AF034151, |
| | | | | | AF165045, AF165046, AF165047, AF165048, AF165053, AF165055, |
| | | | | | AF165056, AF165057, AF165058, AF165059, AF165060, AF165061, |
| | | | | | AF165062, AF207752, AF207753, AF207755, AF207756, AF207758, |
| | | | | | AF207760, AF207761, AF207762, AF207763, AF207765, AF207768, |
| | | | | | AF207770, AF207771, AF207774, AF265142, AF265143, AF265144, |
| | | | | | AF265160, AF265161, AF265162, AF483269, AJ000009, AJ507155, |
| | | | | | AJ507158, AJ507160, AJ507162, AJ507164, AJ507167, AJ507168, |
| | | | | | AJ507171, AJ507172, AJ507174, AJ507175, AJ507178, AJ507186, |
| | | | | | AJ507188, AJ507190, AJ507192, AJ507193, AJ507194, AJ507197, |
| | | | | | AJ507198, AJ507199, AM401867, AM401868, AM401876, |
| | | | | | AM401878, AM401880, AM401916, AM401917, AM401918, |
| | | | | | AM401919, AM401920, AM401921, AM401922, AM401923, |
| | | | | | AM401924, AM401925, AM401926, AM401927, AM401928, |
| | | | | | AM401929, AM401930, AM401931, AM401932, AM401933, |
| | | | | | AM401934, AM401935, AM401936, AM401937, AM401938, |
| | | | | | AM401939, AM401940, AM401941, AM401942, AM401943, |
| | | | | | AM401944, AM401945, AM401946, AM401947, AM401948, |
| | | | | | AM401954, AM401955, AM401957, AM401959, AM401961, |
| | | | | | AM401962, AM401963, AM401964, AM401965, AM401966, |
| | | | | | AM401967, AM401968, AM401969, AM401970, AM401971, |
| | | | | | AM401972, AM401973, AM401974, AM401975, AM401976, |
| | | | | | AM401977, AM401978, AM401979, AM401980, AM402258, |
| | | | | | AM402259, AM402260, AM402261, AM402262, AM402264, |
| | | | | | AM402265, AM402266, AM402267, AM402268, AM402270, |
| | | | | | AM402271, AM402272, AM402273, AM402274, AM402275, |
| | | | | | AM402276, AM402277, AM402278, AM402279, AM402280, |
| | | | | | AM402281, AM402282, AM402283, AM402284, AM402285, |
| | | | | | AM402286, AM402287, AM402288, AM402289, AM402290, |
| | | | | | AM402291, AM402292, AM402293, AM402294, AM402295, |
| | | | | | AM402296, AM402297, AM402298, AM402299, AM402300, |
| | | | | | AM402301, AM402302, AM402303, AM402304, AM402305, |
| | | | | | AM402306, AM402307, AM402308, AM402309, AM493436, |
| | | | | | AM493437, AM493438, AM493439, AM493440, AM493441, |
| | | | | | AM493442, AM493443, AM493444, AM493445, AM493446, |
| | | | | | AM493447, AM493448, AM493449, AM493450, AM493451, |
| | | | | | AM493489, AM493490, AM493491, AM493492, AM493493, |
| | | | | | AM493494, AM493496, AM493497, AM493498, AM493499, |
| | | | | | AM493500, AM493501, AM493502, AM493503, AM493504, |
| | | | | | AM493505, AM493506, AM493507, AY044867, AY045702, |
| | | | | | AY460204, AY808010, AY808011, AY808014, AY808015, |
| | | | | | AY808017, AY808020, AY808021, AY808022, AY808023, |
| | | | | | AY808024, AY808025, AY808027, AY808032, AY808035, |
| | | | | | AY808036, AY808037, AY808038, AY808039, AY808041, D10934, |
| | | | | | D11168, D16435, D50482, D50484, D63857, D85516, D89872, |
| | | | | | D90208, DQ071885, DQ244141, DQ491958, DQ491959, DQ491960, |
| | | | | | DQ491961, DQ491962, DQ491963, DQ491964, DQ491965, |
| | | | | | DQ491966, DQ491967, DQ491968, DQ491969, DQ491970, |
| | | | | | DQ492040, DQ492041, DQ492042, DQ492043, DQ492044, |
| | | | | | DQ492045, DQ492046, DQ492047, DQ492048, DQ492049, |
| | | | | | DQ492050, DQ492051, DQ492052, DQ492053, DQ492054, |
| | | | | | DQ492055, DQ492056, DQ492057, DQ492058, DQ492059, |
| | | | | | DQ492060, DQ492061, DQ492062, DQ492076, DQ492093, |
| | | | | | DQ492103, DQ492105, DQ492106, DQ492124, DQ492125, |
| | | | | | DQ492126, DQ492128, DQ492129, DQ492130, DQ492131, |
| | | | | | DQ492132, DQ492133, DQ492134, DQ492135, DQ492136, |
| | | | | | DQ492137, DQ492138, DQ492139, DQ492140, DQ492141, |
| | | | | | DQ492142, DQ492143, DQ492144, DQ492145, DQ492146, |
| | | | | | DQ492147, DQ492148, DQ492149, DQ492150, DQ492151, |
| | | | | | DQ492152, DQ492153, DQ492154, DQ492155, DQ492156, |
| | | | | | DQ492157, DQ492158, DQ492159, DQ492160, DQ492162, |
| | | | | | DQ492163, DQ492164, DQ492165, DQ492166, DQ492167, |
| | | | | | DQ492168, DQ492169, DQ492170, DQ492171, DQ492172, |
| | | | | | DQ492173, DQ492174, DQ492175, DQ492176, DQ492177, |
| | | | | | DQ492178, DQ492179, DQ492180, DQ492181, DQ492182, |
| | | | | | DQ492183, DQ492184, DQ492185, DQ492186, DQ492187, |
| | | | | | DQ492188, DQ492189, DQ492190, DQ492191, DQ492192, |
| | | | | | DQ492193, DQ492194, DQ492195, DQ492196, DQ492197, |
| | | | | | EF407459, EF407462, EF407468, EF407472, EF407474, EF407475, |
| | | | | | EF407477, EF407480, EF407483, EF407486, EF407487, EF407489, |
| | | | | | EF407491, EF407494, EF407495, EF407497, EF407498, EF407504, |
| | | | | | EU155218, EU155219, EU155220, EU155222, EU155223, |
| | | | | | EU155224, EU155225, EU155226, EU155227, EU155228, |
| | | | | | EU155231, EU155258, EU155260, EU155262, EU155263, |
| | | | | | EU155279, EU155300, EU155303, EU155315, EU155316, |
| | | | | | EU155317, EU155318, EU155327, EU155329, EU155332, |
| | | | | | EU155335, EU155336, EU155356, EU155358, EU155361, |
| | | | | | EU155362, EU155368, EU155372, EU155373, EU155374, |
| | | | | | EU155381, EU155382, EU239714, EU255960, EU255962, |
| | | | | | EU256000, EU256001, EU256045, EU256061, EU256062, |
| | | | | | EU256064, EU256066, EU256075, EU256076, EU256078, |
| | | | | | EU256079, EU256080, EU256083, EU256084, EU256085, |
| | | | | | EU256099, EU256101, EU256102, EU256103, EU482839, |
| | | | | | EU482875, EU482879, EU482881, EU482883, EU482885, |
| | | | | | EU482888, EU660388, FJ390396, FJ958891, FJ958892, FJ958893, |
| | | | | | FJ958894, FJ958895, FJ958898, FJ958899, FJ958900, FJ958901, |
| | | | | | FJ958902, FJ958904, FJ958906, FJ958907, FJ958909, FJ958911, |
| | | | | | FJ958912, FJ958913, FJ958915, FJ958916, FJ958918, FJ958919, |
| | | | | | FJ958921, FJ958922, FJ958923, FJ958924, FJ958925, FJ958927, |
| | | | | | FJ958928, FJ958929, FJ958930, FJ958931, FJ958932, FJ958933, |
| | | | | | FJ958935, GQ913869, GQ913873, GU451218, GU451219, |
| | | | | | GU451220, HM042050, HM042051, HM042052, HM042053, |
| | | | | | HM042055, HM042056, HM042057, HM042058, HM042059, |
| | | | | | HM042061, HM042063, M84754, M96362, U01214, U16362, |
| | | | | | U45476, X61596 |
| 78 | agg | aag | R | K | AB049087, AB049091, AB049096, AB285066, AB285067, |
| | | | | | AB285068, AB285078, AF165051, AF165052, AF207757, AF207764, |
| | | | | | AF208024, AF265154, AF265155, AF265156, AF265158, AF265159, |
| | | | | | AF333324, AJ507157, AJ507165, AJ507166, AJ507169, AJ507173, |
| | | | | | AJ507177, AJ507181, AJ507182, AJ507183, AJ507184, AJ507189, |
| | | | | | AM401848, AM401849, AM401850, AM401851, AM401852, |
| | | | | | AM401853, AM401854, AM401855, AM401856, AM401857, |
| | | | | | AM401859, AM401860, AM401861, AM401862, AM401863, |
| | | | | | AM401864, AM401865, AM401866, AM401869, AM401870, |
| | | | | | AM401871, AM401872, AM401873, AM401874, AM401875, |
| | | | | | AM401877, AM401879, AM401881, AM401882, AM401883, |
| | | | | | AM401884, AM401885, AM401886, AM401887, AM401888, |
| | | | | | AM401889, AM401890, AM401891, AM401892, AM401893, |
| | | | | | AM401894, AM401895, AM401896, AM401897, AM401898, |
| | | | | | AM401899, AM401900, AM401901, AM401902, AM401903, |
| | | | | | AM401904, AM401905, AM401906, AM401907, AM401908, |
| | | | | | AM401909, AM401910, AM401911, AM401912, AM401913, |
| | | | | | AM401914, AM401915, AM493495, AY587844, AY808018, |
| | | | | | AY808019, AY808026, AY808028, AY808033, AY808042, D50480, |
| | | | | | D50481, D50483, D50485, DQ491971, DQ491972, DQ491973, |
| | | | | | DQ491974, DQ491975, DQ491976, DQ491977, DQ491978, |
| | | | | | DQ491979, DQ491980, DQ491981, DQ491982, DQ491983, |
| | | | | | DQ491984, DQ491985, DQ491986, DQ491987, DQ491988, |
| | | | | | DQ491989, DQ491990, DQ491991, DQ491992, DQ491993, |
| | | | | | DQ491994, DQ491995, DQ491996, DQ491997, DQ491998, |
| | | | | | DQ491999, DQ492000, DQ492001, DQ492002, DQ492003, |
| | | | | | DQ492004, DQ492005, DQ492006, DQ492007, DQ492082, |
| | | | | | DQ492094, DQ492095, DQ492096, DQ492097, DQ492098, |
| | | | | | DQ492099, DQ492100, DQ492101, DQ492102, DQ492104, |
| | | | | | DQ492107, DQ492108, DQ492109, DQ492110, DQ492111, |
| | | | | | DQ492112, DQ492113, DQ492114, DQ492115, DQ492116, |
| | | | | | DQ492117, DQ492118, DQ492119, DQ492120, DQ492121, |
| | | | | | DQ492122, DQ492123, DQ859970, EF407460, EF407463, |
| | | | | | EF407466, EF407470, EF407476, EF407478, EF407479, EF407482, |
| | | | | | EF407490, EF407492, EF407496, EF407503, EU155232, |
| | | | | | EU155261, EU155301, EU155306, EU155308, EU155325, |
| | | | | | EU155326, EU155328, EU155330, EU155357, EU155359, |
| | | | | | EU155360, EU155363, EU155364, EU155366, EU155370, |
| | | | | | EU155371, EU255961, EU256059, EU256065, EU256098, |
| | | | | | EU482833, EU482877, EU482880, EU862837, FJ958890, FJ958896, |
| | | | | | FJ958897, FJ958903, FJ958905, FJ958910, FJ958914, FJ958917, |
| | | | | | FJ958920, FJ958926, FJ958934, FN435993, GQ913872, GU451221, |
| | | | | | GU451222, GU451223, GU451224, HM042054, HM042062, L02836, |
| | | | | | M58335 |
| 78 | agg | aac | R | N | EU482886 |
| 78 | agg | agt | R | S | DQ491944 |
| 78 | agg | aca | R | T | DQ492127 |
| 79 | acc | gcc | T | A | AB285041, AF207768, AF265153, AM401882, AM401911, |
| | | | | | AY008261, AY808020, AY808022, EF407481, EU155227, |
| | | | | | EU155333, EU155337, EU155362, EU256089, EU256090, |
| | | | | | EU781827, FJ390398 |
| 79 | acc | gct | T | A | AB285072 |
| 79 | acc | att | T | I | AM402290, AM402291, AM402293, AM402294, AM402296, |
| | | | | | AM402298, AM402299, AM402300, AM402303, AM402304, |
| | | | | | AM402305 |
| 79 | acc | atg | T | M | AB285037 |
| 79 | acc | tcc | T | S | AF356827 |
| 80 | tgt | ggc | C | G | DQ492197 |
| 80 | tgt | cgc | C | R | DQ491954 |
| 80 | tgt | tgg | C | W | DQ491992 |
| 80 | tgt | tac | C | Y | AM493498 |
| 81 | agt | gcc | S | A | FN435993 |
| 81 | agt | tgc | S | C | AB285074 |
| 81 | agt | ggc | S | G | AM402359, DQ491952 |
| 82 | aac | gac | N | D | DQ491971, DQ492082, DQ492168, DQ492174 |
| 82 | aac | agc | N | S | AM402095, AY045702, DQ492131 |
| 83 | acg | gcg | T | A | DQ491981, DQ491984, DQ491988, DQ491989, DQ491992 |
| 83 | acg | atg | T | M | AB285041, AB285047, AF033373, AF165063, AF165064, AJ507155, |
| | | | | | AJ507179, AJ507186, AJ507188, AJ507198, AM402258, AM402259, |
| | | | | | AM402260, AM402261, AM402262, AM402263, AM402264, |
| | | | | | AM402265, AM402266, AM402267, AM402268, AM402269, |
| | | | | | AM402270, AM402271, AM402272, AM402273, AM402274, |
| | | | | | AM402275, AM402276, AM402277, AM402278, AM402279, |
| | | | | | AM402280, AM402281, AM402282, AM402283, AM402284, |
| | | | | | AM402285, AM402286, AM402287, AM402288, AM402289, |
| | | | | | AM402290, AM402291, AM402292, AM402293, AM402294, |
| | | | | | AM402295, AM402296, AM402297, AM402298, AM402299, |
| | | | | | AM402300, AM402301, AM402302, AM402303, AM402304, |
| | | | | | AM402305, AM402306, AM402307, AM402308, AM402309, |
| | | | | | AM402310, AM402311, AM402312, AM402313, AM402314, |
| | | | | | AM402315, AM402316, AM402317, AM402318, AM402319, |
| | | | | | AM402320, AM402321, AM402322, AM402323, AM402324, |
| | | | | | AM402325, AM402326, AM402327, AM402328, AM493436, |
| | | | | | AM493437, AM493438, AM493439, AM493440, AM493441, |
| | | | | | AM493442, AM493443, AM493444, AM493445, AM493446, |
| | | | | | AM493447, AM493448, AM493449, AM493450, AM493451, |
| | | | | | AM494937, AY240967, AY240968, AY240969, AY808009, |
| | | | | | AY808011, AY808014, AY808032, DQ492040, DQ492048, |
| | | | | | DQ492085, DQ492093, DQ492124, DQ492125, DQ492126, |
| | | | | | DQ492127, DQ492128, DQ492129, DQ492130, DQ492131, |
| | | | | | DQ492132, DQ492133, DQ492134, DQ492135, DQ492136, |
| | | | | | DQ492137, DQ492138, DQ492139, DQ492140, DQ492141, |
| | | | | | DQ492142, DQ492143, DQ492144, DQ492145, DQ492146, |
| | | | | | DQ492147, DQ492148, DQ492149, DQ492150, DQ492151, |
| | | | | | DQ492152, DQ492153, DQ492154, DQ492155, DQ492156, |
| | | | | | DQ492157, DQ492158, DQ492159, DQ492160, DQ492161, |
| | | | | | DQ492162, DQ492163, DQ492164, DQ492165, DQ492166, |
| | | | | | DQ492167, DQ492168, DQ492169, DQ492170, DQ492171, |
| | | | | | DQ492172, DQ492173, DQ492174, DQ492175, DQ492176, |
| | | | | | DQ492177, DQ492178, DQ492179, DQ492180, DQ492181, |
| | | | | | DQ492182, DQ492183, DQ492185, DQ492186, DQ492187, |
| | | | | | DQ492188, DQ492189, DQ492190, DQ492191, DQ492192, |
| | | | | | DQ492193, DQ492194, DQ492195, DQ492196, DQ492197, |
| | | | | | EF407468, EF407496, EF407503, EU155220, EU155223, |
| | | | | | EU155226, EU155230, EU155300, EU155301, EU155358, |
| | | | | | EU255961, GU451221, GU451222, GU451223, GU451224, |
| | | | | | HM042051 |
| 83 | acg | gtg | T | V | AB049099, DQ491980, DQ492184 |
| 84 | tgg | tgt | W | C | AY044867 |
| 84 | tgg | cgg | W | R | AM402121, AM402382, DQ492060, DQ492151 |
| 85 | cat | tgc | H | C | AY460204, EF638081, GU451221, GU451222, GU451223, |
| | | | | | GU451224 |
| 85 | cat | tgt | H | C | AB049092, AF265154, AF265155, AF265156, EU482859 |
| 85 | cat | gac | H | D | D50482, D50484 |
| 85 | cat | ctc | H | L | AY008261 |
| 85 | cat | aat | H | N | AY808039, DQ492150 |
| 85 | cat | caa | H | Q | AB049096, AJ507188 |
| 85 | cat | cag | H | Q | AB285074 |
| 85 | cat | cgc | H | R | AB285040, AM493477 |
| 85 | cat | cgt | H | R | AB285052, AM401911, AM401918, AY808041, DQ491958, |
| | | | | | DQ491959, DQ491960, DQ491961, DQ491962, DQ491963, |
| | | | | | DQ491964, DQ491965, DQ491966, DQ491967, DQ491968, |
| | | | | | DQ491969, DQ491970, DQ492128, DQ492186, EF407468, |
| | | | | | EU155300, HM042051, HM042062 |
| 85 | cat | agt | H | S | AF265148, AF265149, AF265150, AF265163, AF265164, AF265165 |
| 85 | cat | tac | H | Y | AB285047, AJ507174, AM402258, AM402259, AM402260, |
| | | | | | AM402261, AM402262, AM402263, AM402264, AM402265, |
| | | | | | AM402266, AM402267, AM402268, AM402269, AM402270, |
| | | | | | AM402271, AM402272, AM402273, AM402274, AM402275, |
| | | | | | AM402276, AM402277, AM402278, AM402279, AM402280, |
| | | | | | AM402281, AM402282, AM402283, AM402284, AM402285, |
| | | | | | AM402286, AM402287, AM402288, AM402289, AM402290, |
| | | | | | AM402291, AM402292, AM402293, AM402294, AM402295, |
| | | | | | AM402296, AM402297, AM402298, AM402299, AM402300, |
| | | | | | AM402301, AM402302, AM402303, AM402304, AM402305, |
| | | | | | AM402306, AM402307, AM402308, AM402309, AM402310, |
| | | | | | AM402311, AM402312, AM402313, AM402314, AM402315, |
| | | | | | AM402316, AM402317, AM402318, AM402319, AM402320, |
| | | | | | AM402321, AM402322, AM402323, AM402324, AM402325, |
| | | | | | AM402326, AM402327, AM402328, AM493436, AM493437, |
| | | | | | AM493438, AM493439, AM493440, AM493441, AM493442, |
| | | | | | AM493443, AM493444, AM493445, AM493446, AM493447, |
| | | | | | AM493448, AM493449, AM493450, AM493451, AM494937, |
| | | | | | AY808026, M96362, U16362 |
| 85 | cat | tat | H | Y | AJ507164, AJ507170, AJ507199, DQ492046, DQ492100, |
| | | | | | EU155220, EU155228, EU239714, EU256077, FJ958890, FJ958892, |
| | | | | | FJ958894, FJ958896, FJ958897, FJ958910, FJ958914, FJ958920, |
| | | | | | GQ913869, HM042054, HM042061, HM042063, U45476 |
| 86 | gga | gaa | G | E | DQ492061 |
| 87 | aca | gca | T | A | AB154196, AJ507170, AM401937, DQ491962, EU155228, |
| | | | | | EU256062, AJ507164, AM401879 |
| 87 | aca | ata | T | I | DQ492017 |
| 87 | aca | aga | T | R | DQ492167 |
| 87 | aca | tca | T | S | U45476 |
| 88 | ttc | ctc | F | L | DQ492097 |
| 88 | ttc | ctt | F | L | DQ491968 |
| 88 | ttc | tcc | F | S | AM402350, DQ492057, DQ492171, FJ958915 |
| 89 | ccc | ctc | P | L | AM402315 |
| 89 | ccc | tcc | P | S | AY240968, AY240969 |
| 90 | att | acc | I | T | AF034151, AM401896, DQ492111, FJ958931 |
| 90 | att | gtc | I | V | AB049089, AB285037, AB285063, AF207766, AJ507171, AY808021, |
| | | | | | DQ492035, DQ492048, DQ492188, EF407494 |
| 91 | aac | gac | N | D | AM401975, DQ492008 |
| 91 | aac | agc | N | S | AM402315, DQ491981, DQ492087 |
| 92 | gcg | gga | A | G | D50482, D50484 |
| 92 | gcg | aca | A | T | AB429050, AM402258, AM402259, AM402260, AM402261, |
| | | | | | AM402262, AM402263, AM402264, AM402265, AM402266, |
| | | | | | AM402267, AM402268, AM402269, AM402270, AM402271, |
| | | | | | AM402272, AM402273, AM402274, AM402275, AM402276, |
| | | | | | AM402277, AM402278, AM402279, AM402280, AM402281, |
| | | | | | AM402282, AM402283, AM402284, AM402285, AM402286, |
| | | | | | AM402287, AM402288, AM402289, AM402290, AM402291, |
| | | | | | AM402292, AM402293, AM402294, AM402295, AM402296, |
| | | | | | AM402297, AM402298, AM402299, AM402300, AM402301, |
| | | | | | AM402302, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402326, |
| | | | | | AM402327, AM402328, AM493436, AM493437, AM493438, |
| | | | | | AM493439, AM493440, AM493441, AM493442, AM493443, |
| | | | | | AM493444, AM493445, AM493446, AM493447, AM493448, |
| | | | | | AM493449, AM493450, AM493451, AY808039, DQ492196, |
| | | | | | EF407496 |
| 92 | gcg | acg | A | T | AB285042, AJ507184, AM401857, AM493475, AY587844, |
| | | | | | AY808015, EU155357 |
| 92 | gcg | gta | A | V | DQ244141, EF407489, AJ507188, AM401869, AM402059, |
| | | | | | EU155281, EU155358 |
| 93 | tac | tgc | Y | C | AY240968, DQ491979 |
| 93 | tac | cac | Y | H | AB049101, AB154189, AB154190, AB285065, AB285073, |
| | | | | | AB285075, AF207770, AJ507157, AJ507162, AJ507163, AY808032, |
| | | | | | AY808038, DQ491981, DQ491983, DQ491990, DQ491991, |
| | | | | | DQ491994, DQ492048, DQ492050, EF407460, EF407500, |
| | | | | | EF407501, EU155232, EU155256, EU155373, EU155374, |
| | | | | | EU256083, EU482874, EU781825, EU781827, HM042051, |
| | | | | | HM042055 |
| 94 | acc | atc | T | I | AM402088, AM493437, DQ492015 |
| 95 | acg | gcg | T | A | AB285066, DQ492116 |
| 95 | acg | atg | T | M | AM493477 |
| 96 | ggc | gac | G | D | AM493450, AY044867, DQ492164 |
| 96 | ggc | agc | G | S | AM401950 |
| 97 | ccc | cac | P | H | AF207769 |
| 97 | ccc | cgt | P | R | AY808038 |
| 97 | ccc | tcc | P | S | AB036519, AB036520, AB036521, AB109543, AB154179, |
| | | | | | AB191333, AB285068, AB285071, AJ507161, AJ507163, AJ507196, |
| | | | | | AM493487, AM493489, AM493491, AM493492, AM493493, |
| | | | | | AM493494, AM493495, AM493496, AM493498, AM493499, |
| | | | | | AM493501, AM493502, AM493503, AM493504, AM493505, |
| | | | | | AM493506, AM493507, D50482, D50484, EU155368, EU155372, |
| | | | | | EU256083 |
| 97 | ccc | tct | P | S | AF207762 |
| 98 | tgc | ggc | C | G | DQ492026, DQ492030, DQ492038, DQ492039 |
| 98 | tgc | aac | C | N | DQ492015 |
| 98 | tgc | agc | C | S | AF207762, AJ507191, AY808034, D50482, D50484, DQ492008, |
| | | | | | DQ492009, DQ492010, DQ492011, DQ492012, DQ492013, |
| | | | | | DQ492014, DQ492016, DQ492017, DQ492018, DQ492019, |
| | | | | | DQ492020, DQ492021, DQ492022, DQ492023, DQ492024, |
| | | | | | DQ492025, DQ492027, DQ492028, DQ492029, DQ492031, |
| | | | | | DQ492032, DQ492033, DQ492034, DQ492035, DQ492036, |
| | | | | | DQ492037, EF407477, EU155232, EU155262, FJ958890, FJ958896, |
| | | | | | FJ958897, FJ958903, FJ958905, FJ958910, FJ958920, FJ958926, |
| | | | | | FJ958934 |
| 98 | tgc | agt | C | S | FJ958914 |
| 98 | tgc | tcc | C | S | AB285049, AF207774, AJ507193, EU155369 |
| 98 | tgc | tgg | C | W | AY460204 |
| 98 | tgc | tac | C | Y | AM402113, AM402121, DQ492078, DQ492088, DQ492168, |
| | | | | | DQ492174 |
| 99 | acg | gca | T | A | AY045702, EU155260 |
| 99 | acg | gcc | T | A | AM402322 |
| 99 | acg | atg | T | M | AJ507162 |
| 99 | acg | tca | T | S | EU155224, EU256065, EU256103 |
| 99 | acg | tcg | T | S | AJ507166, AY808041, D14484, DQ491958, DQ491959, DQ491960, |
| | | | | | DQ491961, DQ491962, DQ491963, DQ491964, DQ491965, |
| | | | | | DQ491966, DQ491967, DQ491968, DQ491969, DQ491970 |
| 99 | acg | gta | T | V | AB285071, AJ507161 |
| 99 | acg | gtg | T | V | AF207765, AF265158, AF265159, EU862837 |
| 100 | ccc | gcc | P | A | EU155222, HM042052 |
| 100 | ccc | ctc | P | L | AM402259 |
| 100 | ccc | ctt | P | L | DQ492155 |
| 101 | tcc | gcc | S | A | AB285069, AF356827, AY808013, EU155356, GQ913873, U01214 |
| 101 | tcc | cca | S | P | FJ958928 |
| 101 | tcc | ccc | S | P | AM402373, DQ492001 |
| 101 | tcc | acc | S | T | AB049098, AB285036, AB285038, AF165062, AF207757, |
| | | | | | AF207762, AF265152, AF265153, AY587844, AY808009, |
| | | | | | AY808014, AY808025, AY808032, AY808039, D50482, D50484, |
| | | | | | DQ492093, DQ492163, DQ492164, DQ492165, DQ492166, |
| | | | | | DQ492167, DQ492168, DQ492169, DQ492170, DQ492171, |
| | | | | | DQ492172, DQ492173, DQ492174, DQ492175, DQ492176, |
| | | | | | DQ492177, DQ492178, DQ492179, DQ492180, DQ492181, |
| | | | | | DQ492182, DQ492183, DQ492184, DQ492185, DQ492186, |
| | | | | | DQ492187, DQ492188, DQ492189, DQ492190, DQ492191, |
| | | | | | DQ492193, DQ492194, DQ492195, DQ492196, DQ492197, |
| | | | | | EF407473, EU155218, EU155332 |
| 101 | tcc | gtc | S | V | EU155306 |
| 102 | ccg | ctg | P | L | DQ491979 |
| 102 | ccg | acg | P | T | DQ492048 |
| 103 | gcg | acg | A | T | EU255961, U01214 |
| 104 | cca | tca | P | S | AM402083, D50482, D50484 |
| 105 | aat | gac | N | D | AF165057, AF165058, AM401853 |
| 105 | aat | agc | N | S | AB285057, AM401923, AM401976, AM493444, AY240968, |
| | | | | | AY587844, AY808037, DQ491958, DQ491969, EU256045, |
| | | | | | EU256075, EU482888 |
| 105 | aat | agt | N | S | AB285048, AB285059, EF407494, EU660388 |
| 106 | tat | tgt | Y | C | AM401885, DQ492156 |
| 106 | tat | cat | Y | H | DQ492192 |
| 107 | tct | gcc | S | A | DQ244141, EU155363 |
| 107 | tct | ttt | S | F | AJ132996, AJ507180 |
| 107 | tct | ccc | S | P | DQ492069, FJ958891 |
| 107 | tct | acc | S | T | AB285042, AB285053, AF207771, AF265145, AF265146, AF265147, |
| | | | | | AM401906, AM401916, AM401917, AM401918, AM401919, |
| | | | | | AM401920, AM401921, AM401922, AM401923, AM401924, |
| | | | | | AM401925, AM401926, AM401927, AM401928, AM401929, |
| | | | | | AM401930, AM401931, AM401932, AM401933, AM401934, |
| | | | | | AM401935, AM401936, AM401937, AM401938, AM401939, |
| | | | | | AM401940, AM401941, AM401942, AM401943, AM401944, |
| | | | | | AM401945, AM401946, AM401947, AM401948, AM401949, |
| | | | | | AM401950, AM401951, AM401952, AM401953, AM401954, |
| | | | | | AM401955, AM401956, AM401957, AM401958, AM401959, |
| | | | | | AM401960, AM401961, AM401962, AM401963, AM401964, |
| | | | | | AM401965, AM401966, AM401967, AM401968, AM401969, |
| | | | | | AM401970, AM401971, AM401972, AM401973, AM401974, |
| | | | | | AM401975, AM401976, AM401977, AM401978, AM401979, |
| | | | | | AM401980, AY808019, DQ492094, DQ492095, DQ492096, |
| | | | | | DQ492097, DQ492098, DQ492099, DQ492100, DQ492101, |
| | | | | | DQ492102, DQ492103, DQ492104, DQ492105, DQ492106, |
| | | | | | DQ492107, DQ492108, DQ492109, DQ492110, DQ492111, |
| | | | | | DQ492112, DQ492113, DQ492114, DQ492115, DQ492116, |
| | | | | | DQ492117, DQ492118, DQ492119, DQ492120, DQ492121, |
| | | | | | DQ492122, DQ492123, EF407485, EF407495, EU155370, |
| | | | | | EU155371, FN435993 |
| 107 | tct | act | S | T | AF483269, AY808043 |
| 108 | agg | ggg | R | G | AM401912, AM402363, AM402372 |
| 108 | agg | aaa | R | K | AF207766, AJ507171 |
| 108 | agg | aag | R | K | AB049089, AB049092, AB049095, AB049098, AB049099, |
| | | | | | AB285043, AB285045, AB285050, AB285053, AB285058, |
| | | | | | AB285070, AF033373, AF165058, AF207758, AF207762, AF207763, |
| | | | | | AF207764, AF207765, AF207772, AF265158, AF265159, AJ507166, |
| | | | | | AJ507168, AJ507173, AJ507180, AJ507190, AJ507197, AM402339, |
| | | | | | AY460204, AY808039, D50482, D50484, DQ071885, DQ244140, |
| | | | | | DQ492168, DQ492174, EF407480, EU155257, EU155302, |
| | | | | | EU155306, EU155369, EU256045, EU256103, EU482849, |
| | | | | | HM042054, HM042055, HM042056, M96362, U16362 |
| 108 | agg | atg | R | M | DQ492120 |
| 108 | agg | aat | R | N | AY240968 |
| 108 | agg | aca | R | T | EF407460, EU155362 |
| 108 | agg | acg | R | T | AY808029, DQ492063, DQ492064, DQ492068, DQ492069, |
| | | | | | DQ492071, DQ492072, DQ492077, EU155365 |
| 109 | gcg | ggg | A | G | AB049100 |
| 109 | gcg | ggc | A | G | AY008261 |
| 109 | gcg | gtg | A | V | AM402325 |
| 110 | ctg | ccg | L | P | AM401858, AM402109, AM402311 |
| 110 | ctg | cgg | L | R | DQ492159 |
| 111 | tgg | cgg | W | R | AM402373, DQ492050, DQ492180 |
| 111 | tgg | tcg | W | S | DQ492058 |
| 112 | cgg | cag | R | Q | DQ492063, DQ492153 |
| 112 | cgg | tgg | R | W | AM402301, AM402308, DQ492056 |
| 113 | gtg | gcg | V | A | AM402128, DQ492082 |
| 113 | gtg | atg | V | M | AJ507192 |
| 114 | gct | agt | A | S | AJ507197 |
| 114 | gct | tcc | A | S | DQ491946, DQ491956, EU155369 |
| 114 | gct | tct | A | S | AB285041, AF207770, AY808040, DQ491943, DQ491944, |
| | | | | | DQ491945, DQ491947, DQ491948, DQ491949, DQ491950, |
| | | | | | DQ491951, DQ491952, DQ491953, DQ491954, DQ491955, |
| | | | | | DQ491957, EU256076 |
| 114 | gct | aca | A | T | DQ492035 |
| 114 | gct | act | A | T | AB154177, AB154178, AB154185, AB154186, AB154191, |
| | | | | | AB154192, AB285044, AB442219, AF313916 |
| 114 | gct | gtt | A | V | D50482, D50484 |
| 115 | gct | ttt | A | F | D50482, D50484 |
| 115 | gct | cct | A | P | AB285075, L02836 |
| 115 | gct | tct | A | S | AJ507185, AM401887, AM401894, AM402107 |
| 115 | gct | gtt | A | V | DQ492015, DQ492042 |
| 116 | gag | gat | E | D | AJ507162, DQ492120, EU155307 |
| 116 | gag | ggg | E | G | AM402311, DQ492101 |
| 117 | gag | gac | E | D | AB049096, AB285076, EU155281 |
| 117 | gag | ggg | E | G | AM402112 |
| 117 | gag | aag | E | K | DQ492089, DQ492096 |
| 117 | gag | gtg | E | V | DQ492041, DQ492043 |
| 118 | tac | tgc | Y | C | AM402384 |
| 119 | gtg | gcg | V | A | AM401889, AM493438, DQ491979, DQ492176, DQ492183 |
| 119 | gtg | ttg | V | L | AF313916 |
| 120 | gag | gat | E | D | DQ492187 |
| 120 | gag | ggg | E | G | AM401888, AM402333 |
| 121 | gtt | gcc | V | A | AM402092 |
| 121 | gtt | atc | V | I | AB285054, AB285055, AB442220, AM402346, AM402351, |
| | | | | | AY808027, EU256001, EU256078, EU482833 |
| 121 | gtt | att | V | I | AB010249, AB049090, AF176573, AF207760, AF265145, AF265146, |
| | | | | | AF265147, D11168, D16435, D89872, EF407491, EF407498, |
| | | | | | EU155223, EU155332, EU862837 |
| 122 | acg | gcg | T | A | AF165055, AF165056, AF165062, AM401942, AM401946, |
| | | | | | DQ491961, DQ491964, DQ491966, DQ492018 |
| 122 | acg | aag | T | K | AB154195, AB154196, AB285076 |
| 122 | acg | ccg | T | P | FJ958914 |
| 122 | acg | agg | T | R | AJ507166, AY808029, DQ492063, DQ492064, DQ492065, |
| | | | | | DQ492066, DQ492067, DQ492068, DQ492069, DQ492070, |
| | | | | | DQ492071, DQ492072, DQ492073, DQ492074, DQ492075, |
| | | | | | DQ492076, DQ492077, DQ492079, DQ492080, DQ492081, |
| | | | | | DQ492082, DQ492083, DQ492084, DQ492085, DQ492086, |
| | | | | | DQ492087, DQ492089, DQ492090, DQ492091, DQ492092, |
| | | | | | EF407460, EU155224, EU234062, EU482849, EU862835, M84754 |
| 122 | acg | gtg | T | V | AF207769, AM402258, AM402259, AM402260, AM402261, |
| | | | | | AM402262, AM402263, AM402264, AM402265, AM402266, |
| | | | | | AM402267, AM402268, AM402269, AM402270, AM402271, |
| | | | | | AM402272, AM402273, AM402274, AM402275, AM402276, |
| | | | | | AM402277, AM402278, AM402279, AM402280, AM402281, |
| | | | | | AM402282, AM402283, AM402284, AM402285, AM402286, |
| | | | | | AM402287, AM402288, AM402289, AM402290, AM402291, |
| | | | | | AM402292, AM402293, AM402294, AM402295, AM402296, |
| | | | | | AM402297, AM402298, AM402299, AM402300, AM402301, |
| | | | | | AM402302, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402326, |
| | | | | | AM402327, AM402328, AM493436, AM493437, AM493438, |
| | | | | | AM493439, AM493440, AM493441, AM493442, AM493443, |
| | | | | | AM493444, AM493445, AM493446, AM493447, AM493448, |
| | | | | | AM493449, AM493450, AM493451, EF407492, EU155306, |
| | | | | | EU155376 |
| 122 | acg | tgg | T | W | DQ492078, DQ492088 |
| 123 | cgg | caa | R | Q | AM401924 |
| 123 | cgg | cag | R | Q | AB154177, AB154194, AF313916, AJ507177, EU155221, |
| | | | | | EU155381, EU256045, EU482860, EU660388 |
| 123 | cgg | tgg | R | W | DQ492011, DQ492012, DQ492013 |
| 124 | gtg | ttg | V | L | AJ507162, AJ507195, EU256061 |
| 124 | gtg | atg | V | M | AF165045, AF165046, AF207755, AY808034, DQ492008, |
| | | | | | DQ492009, DQ492010, DQ492011, DQ492012, DQ492013, |
| | | | | | DQ492014, DQ492015, DQ492016, DQ492017, DQ492018, |
| | | | | | DQ492019, DQ492020, DQ492021, DQ492022, DQ492023, |
| | | | | | DQ492024, DQ492025, DQ492026, DQ492027, DQ492028, |
| | | | | | DQ492029, DQ492030, DQ492031, DQ492032, DQ492033, |
| | | | | | DQ492034, DQ492035, DQ492036, DQ492037, DQ492038, |
| | | | | | DQ492039, FJ958902, FJ958935 |
| 125 | ggg | gcg | G | A | AM493499 |
| 125 | ggg | agg | G | R | AM402285 |
| 126 | gat | gaa | D | E | AB049087 |
| 126 | gat | gag | D | E | AB049094, AF165053 |
| 126 | gat | ggt | D | G | DQ492034 |
| 127 | ttc | tgc | F | C | AY808027 |
| 127 | ttc | ctc | F | L | DQ491976, DQ492038, DQ492081, DQ492126, DQ492127, |
| | | | | | DQ492131 |
| 127 | ttc | tcc | F | S | AB285036, AF207760, AF265148, AF265149, AF265150, AF265163, |
| | | | | | AF265164, AF265165, AM402055, AM402056, AM402057, |
| | | | | | AM402058, AM402060, AM402061, AM402062, AM402063, |
| | | | | | AM402064, AM402066, AM402067, AM402068, AM402069, |
| | | | | | AM402070, AM402071, AM402072, AM402073, AM402074, |
| | | | | | AM402075, AM402076, AM402077, AM402078, AM402080, |
| | | | | | AM402081, AM402082, AM402084, AM402085, AM402086, |
| | | | | | AM402087, AM402088, AM402089, AM402090, AM402092, |
| | | | | | AM402093, AM402094, AM402095, AM402096, AM402097, |
| | | | | | AM402098, AM402099, AM402100, AM402101, AM402102, |
| | | | | | AM402103, AM402104, AM402105, AM402106, AM402107, |
| | | | | | AM402108, AM402109, AM402110, AM402112, AM402113, |
| | | | | | AM402114, AM402115, AM402116, AM402118, AM402119, |
| | | | | | AM402120, AM402121, AM402122, AM402125, AM402127, |
| | | | | | AM402128, AM402129, AY808022, DQ491974, DQ492015, |
| | | | | | EF407482, EF407488, EU482883 |
| 127 | ttc | tac | F | Y | AB049091, AB285039 |
| 128 | cac | ctc | H | L | DQ492047 |
| 128 | cac | cgc | H | R | DQ492103, DQ492179 |
| 129 | tac | tgc | Y | C | DQ492188 |
| 129 | tac | cac | Y | H | AY044867, DQ492179 |
| 129 | tac | tcc | Y | S | AM402275 |
| 130 | gtg | gcg | V | A | DQ492091 |
| 130 | gtg | ata | V | I | AF207774, AF265164, AJ507159, AY808038, D10934, EU155256 |
| 130 | gtg | atc | V | I | AB049091, EU256061 |
| 130 | gtg | ctg | V | L | DQ492170 |
| 131 | acg | gcg | T | A | DQ492016 |
| 131 | acg | ata | T | I | AF165062 |
| 131 | acg | atg | T | M | AM401953 |
| 132 | ggc | gcc | G | A | AM401914 |
| 132 | ggc | gac | G | D | AY240968, AY240969 |
| 132 | ggc | agt | G | S | AJ507164 |
| 133 | atg | ata | M | I | AB285036, AB285046, AF265142, AF265143, AF265144, AF265160, |
| | | | | | AJ507155, EF407473, EF407492, EU256088 |
| 133 | atg | atc | M | I | DQ492197, EU155336, EU256092 |
| 133 | atg | att | M | I | EU155230 |
| 133 | atg | ttg | M | L | AB049092 |
| 133 | atg | aca | M | T | AF265162 |
| 133 | atg | acg | M | T | AM401882, AM402373, AM402377, AM402382, AM402384, |
| | | | | | DQ491988, DQ492127, DQ492130, DQ492131, DQ492140, |
| | | | | | DQ492155, EU155259 |
| 133 | atg | gta | M | V | AF265161, EU155222, EU256090 |
| 133 | atg | gtg | M | V | AB154194, AB285041, AF165063, AF165064, AJ507179, |
| | | | | | AM401916, AM401917, AM401918, AM401919, AM401920, |
| | | | | | AM401921, AM401922, AM401923, AM401924, AM401925, |
| | | | | | AM401926, AM401928, AM401929, AM401930, AM401931, |
| | | | | | AM401932, AM401933, AM401934, AM401935, AM401936, |
| | | | | | AM401937, AM401938, AM401939, AM401940, AM401941, |
| | | | | | AM401942, AM401943, AM401944, AM401945, AM401946, |
| | | | | | AM401947, AM401948, AM401949, AM401950, AM401951, |
| | | | | | AM401952, AM401953, AM401954, AM401955, AM401956, |
| | | | | | AM401957, AM401958, AM401959, AM401960, AM401961, |
| | | | | | AM401962, AM401963, AM401964, AM401965, AM401966, |
| | | | | | AM401967, AM401968, AM401969, AM401970, AM401971, |
| | | | | | AM401972, AM401973, AM401974, AM401975, AM401976, |
| | | | | | AM401977, AM401978, AM401979, AM401980, AY808017, |
| | | | | | AY808041, D14484, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, EF407461, |
| | | | | | EF407471, EU155365, EU256061, EU256085, EU482875, |
| | | | | | EU660388, EU862835, HM042056, HM042058 |
| 133 | atg | gtt | M | V | AY808010 |
| 134 | acc | gcc | T | A | DQ492172, FJ958897 |
| 135 | act | gcc | T | A | AF265154, AF265155, AF265156 |
| 135 | act | gct | T | A | AB285035, AB285038, AB285046, AB285063, AB442220, |
| | | | | | AF207757, AF207772, AJ507194, AM401906, AY808025, |
| | | | | | AY808043, DQ491994, DQ492041, DQ492043, DQ492090, |
| | | | | | EU155224, EU155259, EU155382, EU256079 |
| 135 | act | att | T | I | AJ507178 |
| 135 | act | aac | T | N | AY808033 |
| 135 | act | aat | T | N | AF165051, AF165052 |
| 135 | act | tcc | T | S | EF407459 |
| 135 | act | gtc | T | V | EU155279 |
| 135 | act | gtt | T | V | AJ507168, EU155361 |
| 136 | gac | ggc | D | G | FJ958920 |
| 136 | gac | aac | D | N | DQ492184, EU155306 |
| 137 | aac | gac | N | D | EU482839 |
| 137 | aac | tac | N | Y | DQ491953 |
| 138 | gta | ata | V | I | AB036519, AB036520, AB036521, AB049090, AB049091, |
| | | | | | AB049095, AB109543, AB154203, AB154204, AB191333, |
| | | | | | AB285037, AB285044, AB285051, AB285057, AB285060, |
| | | | | | AB285062, AB285077, AB285078, AF165051, AF165052, |
| | | | | | AF165056, AF165062, AF165063, AF165064, AF207757, AF207760, |
| | | | | | AF207765, AF265142, AF265143, AF265144, AF265148, AF265149, |
| | | | | | AF265150, AF265158, AF265159, AF265160, AF265161, AF265162, |
| | | | | | AF265163, AF265164, AF265165, AJ133096, AJ507159, AJ507164, |
| | | | | | AJ507166, AJ507167, AJ507168, AJ507170, AJ507174, AJ507178, |
| | | | | | AJ507179, AJ507186, AJ507189, AJ507196, AJ507198, AJ507199, |
| | | | | | AM402057, AM402058, AM402063, AM402069, AM402071, |
| | | | | | AM402085, AM402086, AM402089, AM402098, AM402103, |
| | | | | | AM402120, AM402121, AM402129, AM494937, AY240967, |
| | | | | | AY808012, AY808026, AY808033, AY808037, AY808040, D10934, |
| | | | | | DQ071885, DQ244140, DQ491943, DQ491944, DQ491945, |
| | | | | | DQ491946, DQ491947, DQ491948, DQ491949, DQ491950, |
| | | | | | DQ491951, DQ491952, DQ491953, DQ491954, DQ491955, |
| | | | | | DQ491956, DQ491957, EF407460, EF407464, EF407467, |
| | | | | | EF407468, EF407470, EF407478, EF407483, EF407485, EF407486, |
| | | | | | EF407488, EF407491, EF407497, EF407500, EF407501, EU155219, |
| | | | | | EU155220, EU155221, EU155222, EU155224, EU155228, |
| | | | | | EU155231, EU155235, EU155279, EU155302, EU155303, |
| | | | | | EU155316, EU155318, EU155325, EU155327, EU155332, |
| | | | | | EU155333, EU155356, EU155359, EU155367, EU255960, |
| | | | | | EU256000, EU256045, EU256061, EU256062, EU256064, |
| | | | | | EU256066, EU256076, EU256077, EU256085, EU256091, |
| | | | | | EU256103, EU482839, EU482859, EU482875, EU482877, |
| | | | | | EU482885, EU660388, EU781825, EU781830, EU862837, |
| | | | | | FJ024277, FJ024279, FJ390396, FJ390398, GQ913869, HM042051, |
| | | | | | HM042052, HM042055, HM042056, HM042057, HM042058, |
| | | | | | HM042059, HM042061, HM042063, U45476 |
| 138 | gta | atc | V | I | AF207771, AJ507169, AJ507184 |
| 138 | gta | att | V | I | AY808041, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, EU155225, |
| | | | | | EU155281 |
| 138 | gta | cta | V | L | AB285072, AY808032, D50481, EU155373, EU529682 |
| 138 | gta | ctg | V | L | AB285041, EU155357, EU155375, EU482874 |
| 138 | gta | tta | V | L | AB049088, AB285035, AB285040, AB285066, AB285076, |
| | | | | | AF165061, AF207769, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, |
| | | | | | AY008261, AY240968, AY808028, D50485, DQ491979, DQ491980, |
| | | | | | DQ491981, DQ491982, DQ491983, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, EF407474, EF407487, EF407498, |
| | | | | | EU155217, EU155226, EU155304, EU256079, EU482880, |
| | | | | | GQ913873, HM042062 |
| 138 | gta | ttg | V | L | AB285063, AB442221, AM402258, AM402259, AM402260, |
| | | | | | AM402261, AM402262, AM402263, AM402264, AM402265, |
| | | | | | AM402266, AM402267, AM402268, AM402269, AM402270, |
| | | | | | AM402271, AM402272, AM402273, AM402274, AM402275, |
| | | | | | AM402276, AM402277, AM402278, AM402279, AM402280, |
| | | | | | AM402281, AM402282, AM402283, AM402284, AM402285, |
| | | | | | AM402286, AM402287, AM402288, AM402289, AM402290, |
| | | | | | AM402291, AM402292, AM402293, AM402294, AM402295, |
| | | | | | AM402296, AM402297, AM402298, AM402299, AM402300, |
| | | | | | AM402301, AM402302, AM402303, AM402304, AM402305, |
| | | | | | AM402306, AM402307, AM402308, AM402309, AM402310, |
| | | | | | AM402311, AM402312, AM402313, AM402314, AM402315, |
| | | | | | AM402316, AM402317, AM402318, AM402319, AM402320, |
| | | | | | AM402321, AM402322, AM402323, AM402324, AM402325, |
| | | | | | AM402326, AM402327, AM402328, AM493436, AM493437, |
| | | | | | AM493438, AM493439, AM493440, AM493441, AM493442, |
| | | | | | AM493443, AM493444, AM493445, AM493446, AM493447, |
| | | | | | AM493448, AM493449, AM493450, AM493451, EU155361 |
| 139 | aag | gaa | K | E | AJ507183 |
| 139 | aag | gag | K | E | DQ492190 |
| 139 | aag | aga | K | R | AB049092, AB049095, AB285054, AB285055, AB429050 |
| 139 | aag | agg | K | R | AB285045, AM401849, AY808039, DQ492181 |
| 140 | tgc | cgc | C | R | DQ492021 |
| 142 | tgt | ggt | C | G | DQ492103 |
| 142 | tgt | cgc | C | R | AM401892 |
| 142 | tgt | tac | C | Y | AM402309, DQ492144 |
| 142 | tgt | tat | C | Y | DQ492120 |
| 143 | cag | cgg | Q | R | AM401895, AM402112, AM402300, AM402346, DQ492127 |
| 144 | gtt | gct | V | A | AM493451, DQ491978, DQ491989, DQ492056, DQ492067, |
| | | | | | DQ492158 |
| 145 | ccg | ctg | P | L | AM401894, DQ492010 |
| 145 | ccg | tcg | P | S | DQ492018 |
| 146 | gcc | ggc | A | G | DQ492047, DQ492050, EU256059, GQ913872 |
| 146 | gcc | ccc | A | P | D50480, D50482, D50483, D50484 |
| 146 | gcc | agc | A | S | AB049087, EU155260 |
| 146 | gcc | tcc | A | S | AB285044, DQ071885, EF407480 |
| 146 | gcc | acc | A | T | AB049088, AB285042, AB285053, AB442221, AB442222, |
| | | | | | AJ507168, AM402268, AM493476, EU155254, EU155357 |
| 146 | gcc | act | A | T | AB285039 |
| 146 | gcc | gtc | A | V | AM401858 |
| 147 | ccc | ctc | P | L | AM402103, DQ492097 |
| 147 | ccc | ctt | P | L | AJ238800, FJ958934 |
| 148 | gaa | gat | E | D | AB154193 |
| 148 | gaa | gta | E | V | DQ492019 |
| 149 | ttc | atc | F | I | DQ492039 |
| 149 | ttc | ctc | F | L | DQ492035 |
| 149 | ttc | tta | F | L | DQ492195 |
| 149 | ttc | tcc | F | S | DQ491949 |
| 150 | ttc | ctc | F | L | AM401874 |
| 150 | ttc | tcc | F | S | DQ491951, DQ492010 |
| 150 | ttc | tac | F | Y | EU256079 |
| 151 | aca | gcg | T | A | AM402100, AM402369, DQ492103 |
| 151 | aca | aaa | T | K | D50481, D50485 |
| 151 | aca | atg | T | M | DQ491975 |
| 151 | aca | tcg | T | S | AF333324, M58335 |
| 152 | gaa | gac | E | D | AM402316 |
| 152 | gaa | gga | E | G | DQ492019 |
| 152 | gaa | caa | E | Q | EU256085 |
| 153 | gtg | gcg | V | A | AM401869, AM493507 |
| 153 | gtg | ata | V | I | EU255962, GQ913869 |
| 153 | gtg | cta | V | L | AB049090 |
| 153 | gtg | ctc | V | L | AF207768 |
| 153 | gtg | ctg | V | L | AB049094, AB285073, AF207760, AF207769, AF207771, AJ133096, |
| | | | | | AJ507186, AJ507189, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, D50481, |
| | | | | | D50485, DQ244140, EF407467, EF407469, EF407485, EU155223, |
| | | | | | EU155224, EU155226, EU155255, EU155279, EU155281, |
| | | | | | EU155326, EU155332, EU256001, EU529682, EU862837, |
| | | | | | FJ958890, FJ958891, FJ958892, FJ958893, FJ958894, FJ958895, |
| | | | | | FJ958896, FJ958897, FJ958898, FJ958899, FJ958900, FJ958901, |
| | | | | | FJ958902, FJ958903, FJ958904, FJ958905, FJ958906, FJ958907, |
| | | | | | FJ958909, FJ958910, FJ958911, FJ958912, FJ958913, FJ958914, |
| | | | | | FJ958915, FJ958916, FJ958917, FJ958918, FJ958919, FJ958920, |
| | | | | | FJ958921, FJ958922, FJ958923, FJ958924, FJ958925, FJ958926, |
| | | | | | FJ958927, FJ958928, FJ958929, FJ958930, FJ958931, FJ958932, |
| | | | | | FJ958933, FJ958934, FJ958935 |
| 153 | gtg | ttg | V | L | AB010249, AB010250, AB036519, AB036520, AB036521, |
| | | | | | AB049089, AB049092, AB049095, AB049096, AB109543, |
| | | | | | AB191333, AB285035, AB285038, AB285042, AB285043, |
| | | | | | AB285045, AB285047, AB285048, AB285049, AB285050, |
| | | | | | AB285052, AB285054, AB285055, AB285057, AB285060, |
| | | | | | AB285064, AB285068, AB285075, AB285078, AB285079, |
| | | | | | AB429050, AB442219, AB442220, AF165047, AF165048, |
| | | | | | AF165053, AF165054, AF165055, AF165056, AF165063, AF165064, |
| | | | | | AF207757, AF207762, AF207763, AF207764, AF207772, AJ507172, |
| | | | | | AJ507178, AY240968, AY587016, AY808013, AY808036, D11168, |
| | | | | | D14484, D16435, D30613, D45172, D50482, D50484, D89815, |
| | | | | | D89872, DQ071885, DQ244141, EF407498, EU256080, EU256084, |
| | | | | | EU256085, EU482874, EU482880, EU857431, GU451218, |
| | | | | | GU451219, GU451220 |
| 153 | gtg | atg | V | M | AM401875 |
| 154 | gat | ggc | D | G | AM401937 |
| 154 | gat | ggt | D | G | AM401872 |
| 155 | ggg | gag | G | E | AM402330 |
| 155 | ggg | agg | G | R | AM402277, DQ491974 |
| 156 | gtg | gcg | V | A | AM401870, AM402322, DQ492189 |
| 156 | gtg | ata | V | I | AF207761, EF407488, EU155222, EU256061, EU256062, |
| | | | | | EU256064, HM042053 |
| 157 | cgg | ggg | R | G | DQ492144 |
| 157 | cgg | ctg | R | L | AM401879 |
| 157 | cgg | cag | R | Q | AB080299, AB154183, AB154184, AM493472, D85516, EU155300 |
| 157 | cgg | tgg | R | W | AM401891 |
| 158 | ttg | ata | L | I | AB285063 |
| 158 | ttg | atg | L | M | DQ491991, EU155336 |
| 158 | ttg | ccg | L | P | AM401871, AM402298, DQ491967, DQ492037, DQ492073 |
| 158 | ttg | gta | L | V | EU155222 |
| 158 | ttg | gtg | L | V | EU155304 |
| 159 | cac | cgc | H | R | AM402300, AM402312, DQ492008, DQ492141 |
| 160 | agg | gga | R | G | AM402323 |
| 160 | agg | ggg | R | G | AM493488 |
| 160 | agg | tgg | R | W | AM401886 |
| 161 | tac | tgc | Y | C | AM402318 |
| 161 | tac | tgt | Y | C | DQ491981 |
| 161 | tac | ttc | Y | F | AB154193, AB154194, EF407480 |
| 161 | tac | cac | Y | H | DQ492158 |
| 161 | tac | cat | Y | H | AM493480 |
| 161 | tac | aac | Y | N | D50480, D50483, DQ492022 |
| 162 | gct | act | A | T | AM401866, AM401977, AM402088 |
| 162 | gct | gtt | A | V | AB285042, AB285075 |
| 163 | cca | ctg | P | L | DQ492160 |
| 163 | cca | cag | P | Q | AM402290 |
| 164 | gcg | gag | A | E | EF407463 |
| 164 | gcg | cca | A | P | AB154189, AB154190, AB154200, AB154203, AB154204 |
| 164 | gcg | ccg | A | P | AB154177, AB154178, AB154179, AB154180, AB154181, |
| | | | | | AB154182, AB154183, AB154184, AB154185, AB154186, |
| | | | | | AB154187, AB154188, AB154191, AB154192, AB154193, |
| | | | | | AB154194, AB154195, AB154196, AB154197, AB154198, |
| | | | | | AB154199, AB154205, AB154206, AB285077, AF313916, |
| | | | | | EU155224, EU660388, EU862837 |
| 164 | gcg | aca | A | T | AB429050 |
| 164 | gcg | acg | A | T | DQ492038, EU781827 |
| 164 | gcg | gtg | A | V | AB049095, AB049101, AB285043, AB285049, AB285051, |
| | | | | | AB285078, AB442221, AF033373, AF165053, AF165054, AF176573, |
| | | | | | AF207756, AF207763, AF207764, AJ507196, AJ507199, AM402087, |
| | | | | | AM402119, AM493471, AM493472, AM493473, AM493474, |
| | | | | | AM493475, AM493476, AM493477, AM493478, AM493479, |
| | | | | | AM493480, AM493481, AM493482, AM493483, AM493484, |
| | | | | | AM493485, AM493486, AM493487, AM493488, D50482, D50484, |
| | | | | | D90208, DQ244140, DQ492061, EF407480, EU155219, EU155308, |
| | | | | | EU256075, EU256084, HM042054, HM042059, HM042060 |
| 165 | tgc | cgc | C | R | AM401879, AM402349, DQ492069, DQ492120 |
| 165 | tgc | agc | C | S | AB285043 |
| 165 | tgc | tcc | C | S | D50481, D50482, D50484, D50485 |
| 165 | tgc | tac | C | Y | AM401884, AM402301, DQ492158, EF407482 |
| 166 | aaa | gaa | K | E | DQ492111 |
| 166 | aaa | gag | K | E | AM402085 |
| 166 | aaa | gga | K | G | AM401964, D50480, D50483 |
| 166 | aaa | ata | K | I | DQ492126 |
| 166 | aaa | atg | K | M | AM493504 |
| 166 | aaa | aga | K | R | AB010249, AB154180, AB154181, AB154182, AB154187, |
| | | | | | AB154188, AB154200, AB154205, AB249644, AB285061, |
| | | | | | AB285067, AB285074, AB426117, AF207768, AF207774, AJ132997, |
| | | | | | AJ507192, AM401916, AM401917, AM401918, AM401919, |
| | | | | | AM401920, AM401921, AM401922, AM401923, AM401924, |
| | | | | | AM401925, AM401926, AM401927, AM401928, AM401929, |
| | | | | | AM401930, AM401931, AM401932, AM401933, AM401934, |
| | | | | | AM401935, AM401936, AM401937, AM401938, AM401939, |
| | | | | | AM401940, AM401941, AM401942, AM401943, AM401945, |
| | | | | | AM401946, AM401948, AM401949, AM401950, AM401951, |
| | | | | | AM401952, AM401953, AM401954, AM401955, AM401956, |
| | | | | | AM401957, AM401958, AM401959, AM401960, AM401961, |
| | | | | | AM401962, AM401963, AM401965, AM401966, AM401967, |
| | | | | | AM401968, AM401969, AM401970, AM401971, AM401972, |
| | | | | | AM401973, AM401974, AM401975, AM401976, AM401977, |
| | | | | | AM401978, AM401979, AM401980, AY460204, AY808013, |
| | | | | | AY808014, AY808038, D11168, D16435, D63857, D89872, |
| | | | | | DQ491950, DQ492093, DQ492125, DQ492163, DQ492164, |
| | | | | | DQ492165, DQ492166, DQ492167, DQ492168, DQ492169, |
| | | | | | DQ492170, DQ492171, DQ492172, DQ492173, DQ492174, |
| | | | | | DQ492175, DQ492176, DQ492177, DQ492178, DQ492179, |
| | | | | | DQ492180, DQ492181, DQ492182, DQ492183, DQ492186, |
| | | | | | DQ492187, DQ492188, DQ492189, DQ492190, DQ492191, |
| | | | | | DQ492192, DQ492193, DQ492194, DQ492195, DQ492196, |
| | | | | | DQ492197, EF407469, EF407477, EF407480, EF638081, |
| | | | | | EU155220, EU155226, EU155227, EU155262, EU155373, |
| | | | | | EU155375, EU256100, EU482881, FJ390396, M96362, U16362 |
| 166 | aaa | agg | K | R | AB154206, AF165063, AF165064, AF333324, AJ507155, |
| | | | | | AM402102, DQ492184, DQ492185, EF407465, EU155257, |
| | | | | | EU256092, EU482833, FJ390397, GU451221, GU451222, |
| | | | | | GU451223, GU451224, M58335 |
| 166 | aaa | cga | K | R | AM401944, AM401947 |
| 167 | ccc | gcc | P | A | AJ507168, EU155316 |
| 167 | ccc | ctc | P | L | AM401960 |
| 167 | ccc | ctt | P | L | DQ492040 |
| 167 | ccc | tct | P | S | EF407496 |
| 167 | ccc | act | P | T | U01214 |
| 168 | ctc | ccc | L | P | AM401934, DQ492080, FJ958911 |
| 168 | ctc | gtc | L | V | D10934 |
| 169 | cta | ata | L | I | AB154188, AF265155, EU482879 |
| 169 | cta | atg | L | M | AF265154, AF265156, EU155327 |
| 170 | cgg | cac | R | H | AF176573 |
| 170 | cgg | ccg | R | P | AY240968 |
| 170 | cgg | tgg | R | W | AM401898, AM401905, AM402330 |
| 171 | gag | gac | E | D | AB285044, AB285047, AB285066, AB285073, AJ132997, AJ133096, |
| | | | | | AJ507164, AJ507167, AJ507169, AJ507170, AJ507178, AJ507184, |
| | | | | | AJ507199, AM402055, AM402059, AM402060, AM402061, |
| | | | | | AM402065, AM402066, AM402070, AM402073, AM402074, |
| | | | | | AM402075, AM402077, AM402079, AM402081, AM402083, |
| | | | | | AM402084, AM402085, AM402088, AM402090, AM402091, |
| | | | | | AM402093, AM402094, AM402099, AM402102, AM402104, |
| | | | | | AM402106, AM402108, AM402111, AM402112, AM402113, |
| | | | | | AM402115, AM402117, AM402123, AM402124, AM402125, |
| | | | | | AM402126, AM402127, AY008261, AY240968, AY460204, |
| | | | | | AY808017, AY808030, AY808041, AY808042, DQ491958, |
| | | | | | DQ491959, DQ491960, DQ491961, DQ491962, DQ491963, |
| | | | | | DQ491964, DQ491965, DQ491966, DQ491967, DQ491968, |
| | | | | | DQ491969, DQ491970, DQ491971, DQ491972, DQ491973, |
| | | | | | DQ491974, DQ491975, DQ491976, DQ491977, DQ491978, |
| | | | | | EF407472, EF407480, EF407482, EF407488, EF638081, EU155222, |
| | | | | | EU155227, EU155330, EU155365, EU155366, EU155367, |
| | | | | | EU155368, EU256000, EU256001, EU256062, EU256066, |
| | | | | | EU256091, EU256102, EU482881, EU482885, EU862835, |
| | | | | | GQ913869, GQ913873, GU451221, GU451222, GU451223, |
| | | | | | GU451224, HM042052, HM042054, HM042056, HM042057, |
| | | | | | HM042058, HM042061, HM042063 |
| 171 | gag | gat | E | D | AB010250, AB036519, AB036520, AB036521, AB049087, |
| | | | | | AB049089, AB049090, AB049092, AB049094, AB049095, |
| | | | | | AB049096, AB049098, AB049099, AB049100, AB049101, |
| | | | | | AB080299, AB109543, AB154181, AB154182, AB154205, |
| | | | | | AB154206, AB191333, AB285035, AB285036, AB285037, |
| | | | | | AB285038, AB285040, AB285041, AB285042, AB285043, |
| | | | | | AB285045, AB285048, AB285049, AB285050, AB285052, |
| | | | | | AB285053, AB285054, AB285055, AB285056, AB285057, |
| | | | | | AB285059, AB285060, AB285064, AB285068, AB285072, |
| | | | | | AB285075, AB285076, AB285078, AB285079, AB285081, |
| | | | | | AB429050, AB442219, AB442220, AF034151, AF165045, |
| | | | | | AF165046, AF165053, AF165054, AF165061, AF165062, AF265152, |
| | | | | | AF265153, AJ000009, AJ507159, AJ507160, AJ507163, AJ507171, |
| | | | | | AJ507172, AJ507174, AJ507185, AJ507186, AJ507189, AJ507190, |
| | | | | | AM402056, AM402057, AM402058, AM402062, AM402063, |
| | | | | | AM402064, AM402067, AM402068, AM402069, AM402071, |
| | | | | | AM402072, AM402076, AM402078, AM402080, AM402082, |
| | | | | | AM402086, AM402087, AM402089, AM402092, AM402095, |
| | | | | | AM402096, AM402097, AM402098, AM402100, AM402101, |
| | | | | | AM402103, AM402105, AM402107, AM402109, AM402110, |
| | | | | | AM402114, AM402116, AM402118, AM402119, AM402120, |
| | | | | | AM402121, AM402122, AM402128, AM402129, AM493471, |
| | | | | | AM493472, AM493473, AM493474, AM493475, AM493476, |
| | | | | | AM493477, AM493478, AM493479, AM493480, AM493481, |
| | | | | | AM493482, AM493483, AM493484, AM493485, AM493486, |
| | | | | | AM493487, AM493488, AM493489, AM493490, AM493491, |
| | | | | | AM493492, AM493493, AM493494, AM493495, AM493496, |
| | | | | | AM493497, AM493498, AM493499, AM493500, AM493501, |
| | | | | | AM493502, AM493503, AM493504, AM493505, AM493506, |
| | | | | | AM493507, AM494937, AY240967, AY240969, AY587016, |
| | | | | | AY808013, AY808023, AY808026, AY808027, AY808032, |
| | | | | | AY808033, AY808037, AY808039, D14484, D30613, D45172, |
| | | | | | D50481, D50482, D50484, D50485, D85516, D89815, DQ071885, |
| | | | | | DQ244141, DQ859970, EF407459, EF407461, EF407463, |
| | | | | | EF407466, EF407467, EF407468, EF407469, EF407471, EF407477, |
| | | | | | EF407478, EF407479, EF407485, EF407487, EF407491, EF407498, |
| | | | | | EF407504, EU155217, EU155218, EU155219, EU155223, |
| | | | | | EU155228, EU155229, EU155230, EU155256, EU155307, |
| | | | | | EU155326, EU155329, EU155333, EU155335, EU155336, |
| | | | | | EU155356, EU155359, EU155360, EU155364, EU155370, |
| | | | | | EU155371, EU155372, EU155374, EU239714, EU255962, |
| | | | | | EU256075, EU256076, EU256077, EU256080, EU256081, |
| | | | | | EU256084, EU256085, EU256088, EU256092, EU256099, |
| | | | | | EU482833, EU482859, EU482877, EU482880, EU482883, |
| | | | | | EU529682, EU660386, EU857431, FJ390396, FJ958890, FJ958891, |
| | | | | | FJ958892, FJ958893, FJ958894, FJ958895, FJ958896, FJ958897, |
| | | | | | FJ958898, FJ958899, FJ958900, FJ958901, FJ958902, FJ958903, |
| | | | | | FJ958904, FJ958905, FJ958906, FJ958907, FJ958909, FJ958910, |
| | | | | | FJ958911, FJ958912, FJ958913, FJ958914, FJ958915, FJ958916, |
| | | | | | FJ958917, FJ958918, FJ958919, FJ958920, FJ958921, FJ958922, |
| | | | | | FJ958923, FJ958924, FJ958925, FJ958926, FJ958927, FJ958928, |
| | | | | | FJ958929, FJ958930, FJ958931, FJ958932, FJ958933, FJ958934, |
| | | | | | FJ958935, GU451218, GU451219, GU451220, HM042051, |
| | | | | | HM042055, HM042059, HM042062, U45476, X61596 |
| 171 | gag | aag | E | K | AY808043, DQ492152, EU256059 |
| 171 | gag | agt | E | S | EU155226 |
| 171 | gag | acg | E | T | AY808035, DQ492040, DQ492041, DQ492042, DQ492043, |
| | | | | | DQ492044, DQ492045, DQ492046, DQ492047, DQ492048, |
| | | | | | DQ492049, DQ492050, DQ492051, DQ492052, DQ492053, |
| | | | | | DQ492054, DQ492055, DQ492056, DQ492057, DQ492058, |
| | | | | | DQ492059, DQ492060, DQ492061, DQ492062 |
| 171 | gag | gtg | E | V | AB010249, D16435, D89872 |
| 172 | gag | gac | E | D | AF207765, AM402055, AM402056, AM402057, AM402058, |
| | | | | | AM402060, AM402061, AM402062, AM402063, AM402064, |
| | | | | | AM402066, AM402067, AM402068, AM402069, AM402070, |
| | | | | | AM402071, AM402072, AM402073, AM402074, AM402075, |
| | | | | | AM402076, AM402077, AM402078, AM402080, AM402081, |
| | | | | | AM402082, AM402084, AM402085, AM402086, AM402087, |
| | | | | | AM402088, AM402089, AM402090, AM402092, AM402093, |
| | | | | | AM402094, AM402095, AM402096, AM402097, AM402098, |
| | | | | | AM402099, AM402100, AM402101, AM402102, AM402103, |
| | | | | | AM402104, AM402105, AM402106, AM402107, AM402108, |
| | | | | | AM402109, AM402110, AM402111, AM402112, AM402113, |
| | | | | | AM402114, AM402115, AM402116, AM402117, AM402118, |
| | | | | | AM402119, AM402120, AM402121, AM402122, AM402125, |
| | | | | | AM402126, AM402127, AM402128, AM402129, AM493471, |
| | | | | | AM493472, AM493473, AM493474, AM493475, AM493476, |
| | | | | | AM493477, AM493478, AM493479, AM493480, AM493481, |
| | | | | | AM493482, AM493483, AM493484, AM493485, AM493486, |
| | | | | | AM493487, AM493488, EU155376 |
| 172 | gag | gat | E | D | AB010249, AF176573, AJ507196, AM402059, AM402065, |
| | | | | | AM402079, AM402083, AM402091, AM402123, AM402124, D11168, |
| | | | | | D16435, D89872, EU256085, EU660388 |
| 172 | gag | ggg | E | G | AY044867, DQ492071 |
| 173 | gtc | gcc | V | A | AB049091, EF407503, EU256103, EU482874 |
| 173 | gtc | atc | V | I | FJ958895 |
| 173 | gtc | acc | V | T | AY808043 |
| 174 | aca | gca | T | A | AJ507196, EU155318, EU155359, EU155368, GU451221, |
| | | | | | GU451222, GU451223, GU451224 |
| 174 | aca | gac | T | D | EU155306 |
| 174 | aca | gat | T | D | AY240967, D10934 |
| 174 | aca | gga | T | G | EU256090 |
| 174 | aca | aaa | T | K | D63857 |
| 174 | aca | tta | T | L | DQ244140, EU862835 |
| 174 | aca | atg | T | M | AB285062, AJ507172, AM401867, AM401868, AM401876, |
| | | | | | AM401878, AM401880, EU155264, EU155307 |
| 174 | aca | aac | T | N | EU155377 |
| 174 | aca | agt | T | S | AB285069 |
| 174 | aca | tca | T | S | AB049099, AB285036, AB285038, AB285068, AB285081, |
| | | | | | AF165049, AF165050, AJ507155, AJ507182, AM402056, |
| | | | | | AM402057, AM402058, AM402060, AM402062, AM402063, |
| | | | | | AM402064, AM402066, AM402067, AM402068, AM402069, |
| | | | | | AM402070, AM402071, AM402072, AM402073, AM402074, |
| | | | | | AM402075, AM402076, AM402077, AM402078, AM402080, |
| | | | | | AM402081, AM402082, AM402084, AM402085, AM402086, |
| | | | | | AM402087, AM402088, AM402089, AM402090, AM402092, |
| | | | | | AM402093, AM402094, AM402095, AM402096, AM402097, |
| | | | | | AM402098, AM402099, AM402100, AM402101, AM402102, |
| | | | | | AM402103, AM402104, AM402105, AM402106, AM402107, |
| | | | | | AM402108, AM402109, AM402110, AM402111, AM402112, |
| | | | | | AM402114, AM402115, AM402116, AM402117, AM402118, |
| | | | | | AM402119, AM402120, AM402121, AM402122, AM402125, |
| | | | | | AM402126, AM402127, AM402128, AM402129, AY808016, |
| | | | | | AY808023, AY808030, D14484, EF407467, EU155221, EU155336, |
| | | | | | EU155365, EU155367, EU155381, EU256064, EU256066, |
| | | | | | EU256089, EU256091, EU256099, EU862837, FJ390397, |
| | | | | | HM042056, M84754 |
| 174 | aca | tcg | T | S | AM402055, AM402059, AM402061, AM402065, AM402079, |
| | | | | | AM402083, AM402091, AM402113, AM402123, AM402124, |
| | | | | | EU482875, U45476 |
| 174 | aca | tct | T | S | HM042053 |
| 174 | aca | gta | T | V | AB049088, AB049091, AB154181, AB154182, AB285041, |
| | | | | | AB285058, AB285070, AB442221, AF034151, AF207752, |
| | | | | | AF207758, AY460204, D90208, EF638081, M96362, U16362 |
| 174 | aca | gtc | T | V | AY808013 |
| 174 | aca | gtg | T | V | AB285061, AB285077, AB442222, AF207772, AJ507158, AY808038, |
| | | | | | EF407465, EF407472, EF407498, EU155280, EU155302, |
| | | | | | EU781832, L02836 |
| 174 | aca | gtt | T | V | AF207774 |
| 175 | ttc | ctc | F | L | DQ491952, DQ492064 |
| 175 | ttc | tcc | F | S | DQ492050 |
| 175 | ttc | tac | F | Y | AF207762, AF207764 |
| 176 | ctg | tgt | L | C | EU256085 |
| 176 | ctg | gag | L | E | EU155279 |
| 176 | ctg | cat | L | H | HM042059 |
| 176 | ctg | atg | L | M | AB154189, AB154190, AF033373, AJ507188, AM402059, |
| | | | | | AM402065, AM402073, AM402079, AM402083, AM402091, |
| | | | | | AM402093, AM402111, AM402117, AM402123, AM402124, |
| | | | | | AM402126, AY808015, AY808036, EU234062, EU482849, |
| | | | | | EU862837 |
| 176 | ctg | caa | L | Q | AB285063, AB285079, AF207769, AJ507185, AY240968, |
| | | | | | EU155224, EU155318, GQ913872 |
| 176 | ctg | cag | L | Q | AB010249, AB010250, AB036519, AB036520, AB036521, |
| | | | | | AB049087, AB049088, AB049089, AB049090, AB049091, |
| | | | | | AB049092, AB049094, AB049095, AB049096, AB049098, |
| | | | | | AB049099, AB049100, AB049101, AB080299, AB109543, |
| | | | | | AB154177, AB154178, AB154179, AB154180, AB154181, |
| | | | | | AB154182, AB154183, AB154184, AB154185, AB154186, |
| | | | | | AB154187, AB154188, AB154191, AB154192, AB154193, |
| | | | | | AB154194, AB154195, AB154196, AB154197, AB154198, |
| | | | | | AB154199, AB154200, AB154201, AB154202, AB154203, |
| | | | | | AB154204, AB154205, AB154206, AB191333, AB249644, |
| | | | | | AB285035, AB285036, AB285037, AB285038, AB285039, |
| | | | | | AB285040, AB285041, AB285042, AB285043, AB285044, |
| | | | | | AB285045, AB285046, AB285047, AB285048, AB285049, |
| | | | | | AB285050, AB285051, AB285052, AB285053, AB285054, |
| | | | | | AB285055, AB285056, AB285057, AB285058, AB285059, |
| | | | | | AB285060, AB285061, AB285062, AB285064, AB285065, |
| | | | | | AB285066, AB285067, AB285068, AB285069, AB285070, |
| | | | | | AB285071, AB285072, AB285073, AB285074, AB285075, |
| | | | | | AB285076, AB285077, AB285078, AB285081, AB426117, |
| | | | | | AB429050, AB442219, AB442220, AB442221, AB442222, |
| | | | | | AF034151, AF165045, AF165046, AF165047, AF165048, AF165049, |
| | | | | | AF165050, AF165051, AF165052, AF165053, AF165054, AF165055, |
| | | | | | AF165056, AF165057, AF165058, AF165059, AF165060, AF165061, |
| | | | | | AF165062, AF165063, AF165064, AF176573, AF207752, AF207753, |
| | | | | | AF207754, AF207755, AF207756, AF207757, AF207758, AF207760, |
| | | | | | AF207761, AF207762, AF207763, AF207764, AF207765, AF207766, |
| | | | | | AF207768, AF207770, AF207771, AF207772, AF207774, AF208024, |
| | | | | | AF265142, AF265143, AF265144, AF265145, AF265146, AF265147, |
| | | | | | AF265152, AF265153, AF265154, AF265155, AF265156, AF265158, |
| | | | | | AF265159, AF265160, AF265161, AF265162, AF313916, AF333324, |
| | | | | | AF356827, AF483269, AJ000009, AJ132996, AJ132997, AJ507155, |
| | | | | | AJ507157, AJ507158, AJ507159, AJ507160, AJ507161, AJ507162, |
| | | | | | AJ507163, AJ507164, AJ507165, AJ507166, AJ507168, AJ507169, |
| | | | | | AJ507170, AJ507171, AJ507172, AJ507173, AJ507174, AJ507175, |
| | | | | | AJ507177, AJ507178, AJ507179, AJ507180, AJ507181, AJ507182, |
| | | | | | AJ507183, AJ507184, AJ507186, AJ507187, AJ507189, AJ507190, |
| | | | | | AJ507191, AJ507192, AJ507193, AJ507194, AJ507195, AJ507196, |
| | | | | | AJ507197, AJ507198, AJ507199, AM401848, AM401849, |
| | | | | | AM401850, AM401851, AM401852, AM401853, AM401854, |
| | | | | | AM401855, AM401856, AM401857, AM401858, AM401859, |
| | | | | | AM401860, AM401861, AM401862, AM401863, AM401864, |
| | | | | | AM401865, AM401866, AM401867, AM401868, AM401869, |
| | | | | | AM401870, AM401871, AM401872, AM401873, AM401874, |
| | | | | | AM401875, AM401876, AM401877, AM401878, AM401879, |
| | | | | | AM401880, AM401881, AM401882, AM401883, AM401884, |
| | | | | | AM401885, AM401886, AM401887, AM401888, AM401889, |
| | | | | | AM401890, AM401891, AM401892, AM401893, AM401894, |
| | | | | | AM401895, AM401896, AM401897, AM401898, AM401899, |
| | | | | | AM401900, AM401901, AM401902, AM401903, AM401904, |
| | | | | | AM401905, AM401906, AM401907, AM401908, AM401909, |
| | | | | | AM401910, AM401911, AM401912, AM401913, AM401914, |
| | | | | | AM401915, AM401916, AM401917, AM401918, AM401919, |
| | | | | | AM401920, AM401921, AM401922, AM401923, AM401924, |
| | | | | | AM401925, AM401926, AM401927, AM401928, AM401929, |
| | | | | | AM401930, AM401931, AM401932, AM401933, AM401934, |
| | | | | | AM401935, AM401936, AM401937, AM401938, AM401939, |
| | | | | | AM401940, AM401941, AM401942, AM401943, AM401944, |
| | | | | | AM401945, AM401946, AM401947, AM401948, AM401949, |
| | | | | | AM401950, AM401951, AM401952, AM401953, AM401954, |
| | | | | | AM401955, AM401956, AM401957, AM401958, AM401959, |
| | | | | | AM401960, AM401961, AM401962, AM401963, AM401964, |
| | | | | | AM401965, AM401966, AM401967, AM401968, AM401969, |
| | | | | | AM401970, AM401971, AM401972, AM401973, AM401974, |
| | | | | | AM401975, AM401976, AM401977, AM401978, AM401979, |
| | | | | | AM401980, AM402258, AM402259, AM402260, AM402261, |
| | | | | | AM402262, AM402263, AM402264, AM402265, AM402266, |
| | | | | | AM402267, AM402268, AM402269, AM402270, AM402271, |
| | | | | | AM402272, AM402273, AM402274, AM402275, AM402276, |
| | | | | | AM402277, AM402278, AM402279, AM402280, AM402281, |
| | | | | | AM402282, AM402283, AM402284, AM402285, AM402286, |
| | | | | | AM402287, AM402288, AM402289, AM402290, AM402291, |
| | | | | | AM402292, AM402293, AM402294, AM402295, AM402296, |
| | | | | | AM402297, AM402298, AM402299, AM402300, AM402301, |
| | | | | | AM402302, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402326, |
| | | | | | AM402327, AM402328, AM493436, AM493437, AM493438, |
| | | | | | AM493439, AM493440, AM493441, AM493442, AM493443, |
| | | | | | AM493444, AM493445, AM493446, AM493447, AM493448, |
| | | | | | AM493449, AM493450, AM493451, AM493471, AM493472, |
| | | | | | AM493473, AM493474, AM493475, AM493476, AM493477, |
| | | | | | AM493478, AM493479, AM493480, AM493481, AM493482, |
| | | | | | AM493483, AM493484, AM493485, AM493486, AM493487, |
| | | | | | AM493488, AM493489, AM493490, AM493491, AM493492, |
| | | | | | AM493493, AM493494, AM493495, AM493496, AM493497, |
| | | | | | AM493498, AM493499, AM493500, AM493501, AM493502, |
| | | | | | AM493503, AM493504, AM493505, AM493506, AM493507, |
| | | | | | AM494937, AY045702, AY240967, AY240969, AY460204, |
| | | | | | AY587016, AY587844, AY808009, AY808011, AY808012, |
| | | | | | AY808013, AY808014, AY808016, AY808017, AY808018, |
| | | | | | AY808020, AY808021, AY808022, AY808024, AY808025, |
| | | | | | AY808026, AY808027, AY808028, AY808029, AY808030, |
| | | | | | AY808032, AY808033, AY808034, AY808035, AY808037, |
| | | | | | AY808038, AY808039, AY808040, AY808041, AY808042, D10934, |
| | | | | | D11168, D14484, D16435, D30613, D45172, D50480, D50481, |
| | | | | | D50482, D50483, D50484, D50485, D63857, D85516, D89815, |
| | | | | | D89872, D90208, DQ071885, DQ244140, DQ244141, DQ491943, |
| | | | | | DQ491944, DQ491945, DQ491946, DQ491947, DQ491948, |
| | | | | | DQ491949, DQ491950, DQ491951, DQ491952, DQ491953, |
| | | | | | DQ491954, DQ491955, DQ491956, DQ491957, DQ491958, |
| | | | | | DQ491959, DQ491960, DQ491961, DQ491962, DQ491963, |
| | | | | | DQ491964, DQ491965, DQ491966, DQ491967, DQ491968, |
| | | | | | DQ491969, DQ491970, DQ491971, DQ491972, DQ491973, |
| | | | | | DQ491974, DQ491975, DQ491976, DQ491977, DQ491978, |
| | | | | | DQ491979, DQ491980, DQ491981, DQ491982, DQ491983, |
| | | | | | DQ491984, DQ491985, DQ491986, DQ491987, DQ491988, |
| | | | | | DQ491989, DQ491990, DQ491991, DQ491992, DQ491993, |
| | | | | | DQ491994, DQ491995, DQ491996, DQ491997, DQ491998, |
| | | | | | DQ491999, DQ492000, DQ492001, DQ492002, DQ492003, |
| | | | | | DQ492004, DQ492005, DQ492006, DQ492007, DQ492008, |
| | | | | | DQ492009, DQ492010, DQ492011, DQ492012, DQ492013, |
| | | | | | DQ492014, DQ492015, DQ492016, DQ492017, DQ492018, |
| | | | | | DQ492019, DQ492020, DQ492021, DQ492022, DQ492023, |
| | | | | | DQ492024, DQ492025, DQ492026, DQ492027, DQ492028, |
| | | | | | DQ492029, DQ492030, DQ492031, DQ492032, DQ492033, |
| | | | | | DQ492034, DQ492035, DQ492036, DQ492037, DQ492038, |
| | | | | | DQ492039, DQ492040, DQ492041, DQ492042, DQ492043, |
| | | | | | DQ492044, DQ492045, DQ492046, DQ492047, DQ492048, |
| | | | | | DQ492049, DQ492050, DQ492051, DQ492052, DQ492053, |
| | | | | | DQ492054, DQ492055, DQ492056, DQ492057, DQ492058, |
| | | | | | DQ492059, DQ492060, DQ492061, DQ492062, DQ492063, |
| | | | | | DQ492064, DQ492065, DQ492066, DQ492067, DQ492068, |
| | | | | | DQ492069, DQ492070, DQ492071, DQ492072, DQ492073, |
| | | | | | DQ492074, DQ492075, DQ492076, DQ492077, DQ492078, |
| | | | | | DQ492079, DQ492080, DQ492081, DQ492082, DQ492083, |
| | | | | | DQ492084, DQ492085, DQ492086, DQ492087, DQ492088, |
| | | | | | DQ492089, DQ492090, DQ492091, DQ492092, DQ492093, |
| | | | | | DQ492124, DQ492125, DQ492126, DQ492127, DQ492128, |
| | | | | | DQ492129, DQ492130, DQ492131, DQ492132, DQ492133, |
| | | | | | DQ492134, DQ492135, DQ492136, DQ492137, DQ492138, |
| | | | | | DQ492139, DQ492140, DQ492141, DQ492142, DQ492143, |
| | | | | | DQ492144, DQ492145, DQ492146, DQ492147, DQ492148, |
| | | | | | DQ492149, DQ492150, DQ492151, DQ492152, DQ492153, |
| | | | | | DQ492154, DQ492155, DQ492156, DQ492157, DQ492158, |
| | | | | | DQ492159, DQ492160, DQ492161, DQ492162, DQ492163, |
| | | | | | DQ492164, DQ492165, DQ492166, DQ492167, DQ492168, |
| | | | | | DQ492169, DQ492170, DQ492171, DQ492172, DQ492173, |
| | | | | | DQ492174, DQ492175, DQ492176, DQ492177, DQ492178, |
| | | | | | DQ492179, DQ492180, DQ492181, DQ492182, DQ492183, |
| | | | | | DQ492184, DQ492185, DQ492186, DQ492187, DQ492188, |
| | | | | | DQ492189, DQ492190, DQ492191, DQ492192, DQ492193, |
| | | | | | DQ492194, DQ492195, DQ492196, DQ492197, DQ859970, |
| | | | | | EF032892, EF032893, EF032894, EF407459, EF407460, EF407461, |
| | | | | | EF407462, EF407463, EF407465, EF407466, EF407467, EF407468, |
| | | | | | EF407469, EF407470, EF407471, EF407472, EF407473, EF407475, |
| | | | | | EF407476, EF407477, EF407478, EF407479, EF407480, EF407481, |
| | | | | | EF407482, EF407483, EF407484, EF407485, EF407486, EF407487, |
| | | | | | EF407488, EF407489, EF407490, EF407491, EF407492, EF407494, |
| | | | | | EF407495, EF407496, EF407497, EF407498, EF407499, EF407500, |
| | | | | | EF407501, EF407502, EF407503, EF407504, EF638081, EU155217, |
| | | | | | EU155218, EU155219, EU155220, EU155221, EU155222, |
| | | | | | EU155223, EU155225, EU155226, EU155227, EU155228, |
| | | | | | EU155229, EU155230, EU155231, EU155232, EU155235, |
| | | | | | EU155253, EU155254, EU155255, EU155256, EU155257, |
| | | | | | EU155259, EU155260, EU155261, EU155262, EU155263, |
| | | | | | EU155280, EU155281, EU155300, EU155301, EU155302, |
| | | | | | EU155303, EU155305, EU155306, EU155307, EU155308, |
| | | | | | EU155315, EU155316, EU155317, EU155324, EU155325, |
| | | | | | EU155326, EU155327, EU155328, EU155329, EU155330, |
| | | | | | EU155331, EU155332, EU155334, EU155335, EU155336, |
| | | | | | EU155337, EU155356, EU155357, EU155358, EU155359, |
| | | | | | EU155360, EU155361, EU155362, EU155363, EU155364, |
| | | | | | EU155365, EU155366, EU155367, EU155368, EU155369, |
| | | | | | EU155370, EU155371, EU155372, EU155373, EU155374, |
| | | | | | EU155375, EU155376, EU155377, EU155381, EU155382, |
| | | | | | EU234061, EU239714, EU255960, EU255961, EU255962, |
| | | | | | EU256000, EU256001, EU256045, EU256059, EU256061, |
| | | | | | EU256062, EU256064, EU256065, EU256066, EU256075, |
| | | | | | EU256076, EU256078, EU256079, EU256080, EU256081, |
| | | | | | EU256082, EU256083, EU256084, EU256088, EU256089, |
| | | | | | EU256090, EU256092, EU256098, EU256099, EU256100, |
| | | | | | EU256101, EU256102, EU256103, EU482833, EU482859, |
| | | | | | EU482860, EU482875, EU482877, EU482879, EU482880, |
| | | | | | EU482881, EU482883, EU482886, EU482888, EU529682, |
| | | | | | EU660386, EU660388, EU781825, EU781827, EU781830, |
| | | | | | EU781832, EU857431, EU862835, FJ024277, FJ024279, FJ390396, |
| | | | | | FJ390397, FJ390398, FJ478453, FJ958890, FJ958891, FJ958892, |
| | | | | | FJ958893, FJ958894, FJ958895, FJ958896, FJ958897, FJ958898, |
| | | | | | FJ958899, FJ958900, FJ958901, FJ958902, FJ958903, FJ958904, |
| | | | | | FJ958905, FJ958906, FJ958907, FJ958909, FJ958910, FJ958911, |
| | | | | | FJ958912, FJ958913, FJ958914, FJ958915, FJ958916, FJ958917, |
| | | | | | FJ958918, FJ958919, FJ958920, FJ958921, FJ958922, FJ958923, |
| | | | | | FJ958924, FJ958925, FJ958926, FJ958927, FJ958928, FJ958929, |
| | | | | | FJ958930, FJ958931, FJ958932, FJ958933, FJ958934, FJ958935, |
| | | | | | FN435993, GQ913868, GQ913869, GQ913870, GQ913871, |
| | | | | | GQ913873, GU451218, GU451219, GU451220, GU451221, |
| | | | | | GU451222, GU451223, GU451224, HM042050, HM042051, |
| | | | | | HM042052, HM042054, HM042055, HM042056, HM042057, |
| | | | | | HM042058, HM042060, HM042061, HM042062, HM042063, L02836, |
| | | | | | M58335, M84754, M96362, U01214, U16362, U45476, X61596 |
| 176 | ctg | cgg | L | R | EU482839 |
| 176 | ctg | acg | L | T | AY808023 |
| 176 | ctg | tgg | L | W | EU155264 |
| 177 | gtc | gcc | V | A | AM402110, DQ492137 |
| 177 | gtc | atc | V | I | DQ492040, EF407487, EU155303 |
| 178 | ggg | gaa | G | E | AM402320 |
| 178 | ggg | cgg | G | R | AY240968 |
| 179 | ctc | tgc | L | C | DQ244140, EU155280 |
| 179 | ctc | tgt | L | C | EU482875 |
| 179 | ctc | ttc | L | F | AB285068, AM402283, FJ958923 |
| 179 | ctc | cac | L | H | AM493442, DQ492027 |
| 179 | ctc | ccc | L | P | AM401957, DQ491994, DQ492046, DQ492067, DQ492097, |
| | | | | | DQ492151 |
| 180 | aat | gac | N | D | AM401859 |
| 180 | aat | cac | N | H | AB285041, AB285059, AJ133096, AJ507158, AJ507177, AM402055, |
| | | | | | AM402056, AM402057, AM402058, AM402059, AM402060, |
| | | | | | AM402061, AM402062, AM402063, AM402064, AM402065, |
| | | | | | AM402066, AM402067, AM402068, AM402069, AM402070, |
| | | | | | AM402071, AM402072, AM402073, AM402074, AM402075, |
| | | | | | AM402076, AM402077, AM402078, AM402079, AM402080, |
| | | | | | AM402081, AM402082, AM402083, AM402084, AM402085, |
| | | | | | AM402086, AM402087, AM402088, AM402089, AM402090, |
| | | | | | AM402091, AM402092, AM402093, AM402094, AM402095, |
| | | | | | AM402096, AM402097, AM402098, AM402099, AM402100, |
| | | | | | AM402101, AM402102, AM402103, AM402104, AM402105, |
| | | | | | AM402106, AM402107, AM402108, AM402109, AM402110, |
| | | | | | AM402111, AM402112, AM402113, AM402114, AM402115, |
| | | | | | AM402116, AM402117, AM402118, AM402119, AM402120, |
| | | | | | AM402121, AM402122, AM402123, AM402124, AM402125, |
| | | | | | AM402126, AM402127, AM402128, AM402129, AY808016, |
| | | | | | AY808030, EF407491, EU155221, EU155307, EU155331, |
| | | | | | EU155363, EU155374, EU155381, EU234062, EU256066, |
| | | | | | EU256085, EU256100, EU529682, EU862835, FJ390397, |
| | | | | | GU451221, GU451222, GU451223, GU451224, HM042051, |
| | | | | | M96362, U16362 |
| 180 | aat | cat | N | H | EU155328, EU155330, EU155365, EU155367 |
| 180 | aat | agc | N | S | AM402283, AM402352, DQ492166, FJ958920 |
| 181 | caa | gaa | Q | E | AM402056, AM402057, AM402058, AM402062, AM402063, |
| | | | | | AM402064, AM402067, AM402068, AM402069, AM402071, |
| | | | | | AM402072, AM402076, AM402078, AM402080, AM402082, |
| | | | | | AM402086, AM402087, AM402089, AM402092, AM402095, |
| | | | | | AM402096, AM402097, AM402098, AM402099, AM402101, |
| | | | | | AM402103, AM402105, AM402107, AM402110, AM402114, |
| | | | | | AM402116, AM402118, AM402119, AM402120, AM402121, |
| | | | | | AM402122, AM402128, AM402129, AY808026 |
| 181 | caa | gag | Q | E | AM402100 |
| 181 | caa | cac | Q | H | AB442219, U45476 |
| 181 | caa | cat | Q | H | AB285079 |
| 181 | caa | aag | Q | K | AM402109 |
| 181 | caa | cta | Q | L | DQ492131, DQ492146 |
| 181 | caa | cga | Q | R | AM402370, DQ492011, DQ492012, DQ492013, DQ492059, |
| | | | | | DQ492137, EU155257 |
| 181 | caa | cgc | Q | R | D50482, D50484 |
| 181 | caa | cgg | Q | R | AM494937, AY808023 |
| 182 | tac | tgc | Y | C | AM402278, DQ492084 |
| 182 | tac | ttc | Y | F | AB049095, AB049096, AB285035, AB285045, AB285048, |
| | | | | | AB285054, AB285055, AB285068, AB285071, AB285079, |
| | | | | | AB442219, AF034151, AF207763, AJ507189, EF407480, EU155219, |
| | | | | | X61596 |
| 182 | tac | ttt | Y | F | AB285049, AB285066, AB285072 |
| 182 | tac | cac | Y | H | DQ492003 |
| 182 | tac | cat | Y | H | DQ492138 |
| 182 | tac | aac | Y | N | AM494937 |
| 183 | ctg | gca | L | A | AB285047, EU155365, EU155366, EU256076, EU529682 |
| 183 | ctg | gcg | L | A | AF207760, AJ507160, AJ507164, AM402055, AM402060, |
| | | | | | AM402061, AM402066, AM402070, AM402074, AM402075, |
| | | | | | AM402077, AM402081, AM402084, AM402085, AM402088, |
| | | | | | AM402090, AM402094, AM402102, AM402106, AM402108, |
| | | | | | AM402111, AM402112, AM402113, AM402127, AY808030, D50484, |
| | | | | | EF407469, EU155222, EU155367, EU256062, EU256085, |
| | | | | | EU482849, EU482885, FJ958934, HM042059, HM042063 |
| 183 | ctg | ata | L | I | AF165063, AF165064 |
| 183 | ctg | cca | L | P | AB285073, AF165047, AF165048, AJ507171 |
| 183 | ctg | ccg | L | P | AB049087, AB049089, AB049092, AB049094, AB049095, |
| | | | | | AB049096, AB154181, AB154182, AB154205, AB154206, |
| | | | | | AB285035, AB285038, AB285040, AB285042, AB285043, |
| | | | | | AB285044, AB285045, AB285048, AB285049, AB285050, |
| | | | | | AB285052, AB285053, AB285054, AB285055, AB285056, |
| | | | | | AB285057, AB285059, AB285060, AB285068, AB285075, |
| | | | | | AB285078, AB285079, AB429050, AB442219, AB442220, |
| | | | | | AF034151, AF165045, AF165046, AF165053, AF165055, AF165056, |
| | | | | | AF207755, AF207757, AF207763, AF207768, AF207769, AF207771, |
| | | | | | AF265149, AJ507169, AJ507172, AJ507185, AJ507189, AY240967, |
| | | | | | AY240969, AY808013, AY808037, D10934, D50482, DQ071885, |
| | | | | | DQ244141, DQ492134, EF407467, EF407480, EF407491, |
| | | | | | EF407498, EU155227, EU155306, EU155307, EU155326, |
| | | | | | EU155330, EU155336, EU155359, EU155360, EU155368, |
| | | | | | EU256075, EU256077, EU256084, EU256088, EU256102, |
| | | | | | EU482859, EU482881, EU660386, EU862835, GU451219, |
| | | | | | GU451220, GU451221, GU451222, GU451223, GU451224, |
| | | | | | HM042062, U45476, X61596 |
| 183 | ctg | cct | L | P | AJ133096, EU256080, GU451218 |
| 183 | ctg | acg | L | T | AB010250, AF165054, AM402115, AM402125, AY587016, D30613, |
| | | | | | D45172, D89815, EU857431 |
| 183 | ctg | gta | L | V | AB285066, GQ913873 |
| 183 | ctg | gtg | L | V | AB036519, AB036520, AB036521, AB049090, AB109543, |
| | | | | | AB154195, AB154196, AB191333, AB285036, AB285041, |
| | | | | | AB285072, AB285076, AB285081, AJ000009, AJ507166, AJ507167, |
| | | | | | AJ507170, AJ507178, AJ507184, AJ507186, AJ507188, AJ507199, |
| | | | | | AM402056, AM402057, AM402058, AM402059, AM402062, |
| | | | | | AM402063, AM402064, AM402065, AM402067, AM402068, |
| | | | | | AM402069, AM402071, AM402072, AM402073, AM402076, |
| | | | | | AM402078, AM402079, AM402080, AM402082, AM402083, |
| | | | | | AM402086, AM402087, AM402089, AM402091, AM402092, |
| | | | | | AM402093, AM402095, AM402096, AM402097, AM402098, |
| | | | | | AM402099, AM402100, AM402101, AM402103, AM402104, |
| | | | | | AM402105, AM402107, AM402109, AM402110, AM402114, |
| | | | | | AM402116, AM402117, AM402118, AM402119, AM402120, |
| | | | | | AM402121, AM402122, AM402123, AM402124, AM402126, |
| | | | | | AM402128, AM402129, AY008261, AY808016, AY808023, |
| | | | | | AY808026, AY808027, AY808033, AY808041, D50481, D50485, |
| | | | | | DQ491958, DQ491959, DQ491960, DQ491961, DQ491962, |
| | | | | | DQ491963, DQ491964, DQ491965, DQ491966, DQ491967, |
| | | | | | DQ491968, DQ491969, DQ491970, EF407459, EF407461, |
| | | | | | EF407468, EF407477, EF407482, EF407487, EF407488, EU155217, |
| | | | | | EU155219, EU155223, EU155224, EU155228, EU155255, |
| | | | | | EU155318, EU155333, EU155356, EU155364, EU155370, |
| | | | | | EU155371, EU155376, EU155377, EU234062, EU255962, |
| | | | | | EU256001, EU256066, EU256090, EU256091, EU256099, |
| | | | | | EU482874, EU482883, FJ958890, FJ958891, FJ958892, FJ958893, |
| | | | | | FJ958894, FJ958895, FJ958896, FJ958897, FJ958898, FJ958899, |
| | | | | | FJ958900, FJ958901, FJ958902, FJ958903, FJ958904, FJ958905, |
| | | | | | FJ958906, FJ958907, FJ958909, FJ958910, FJ958911, FJ958912, |
| | | | | | FJ958913, FJ958914, FJ958915, FJ958916, FJ958917, FJ958918, |
| | | | | | FJ958919, FJ958920, FJ958921, FJ958922, FJ958923, FJ958924, |
| | | | | | FJ958925, FJ958926, FJ958927, FJ958928, FJ958929, FJ958930, |
| | | | | | FJ958931, FJ958932, FJ958933, FJ958935, GQ913869, HM042051, |
| | | | | | HM042052, HM042054, HM042055, HM042056, HM042057, |
| | | | | | HM042058, HM042061, M84754 |
| 184 | gtt | gct | V | A | DQ491954 |
| 184 | gtt | atc | V | I | AY808024 |
| 184 | gtt | att | V | I | AB285036, AJ507194 |
| 185 | ggg | gaa | G | E | AM402129 |
| 185 | ggg | gag | G | E | AM401894, AM401940, DQ491947 |
| 186 | tca | tta | S | L | DQ492011, DQ492012, DQ492013 |
| 186 | tca | cca | S | P | FJ958904 |
| 186 | tca | cct | S | P | AM402330 |
| 187 | cag | cgg | Q | R | AM401971, AM402119, AM402326, DQ492109, DQ492133, |
| | | | | | DQ492168, DQ492174 |
| 188 | ctc | ttc | L | F | AM401907 |
| 188 | ctc | cac | L | H | DQ492115 |
| 188 | ctc | ccc | L | P | FJ958935 |
| 189 | cca | cta | P | L | DQ491967 |
| 189 | cca | tca | P | S | DQ492173 |
| 190 | tgc | cgt | C | R | DQ492154 |
| 191 | gag | ggg | E | G | AM401860, AM402124, DQ492075, DQ492102, DQ492131 |
| 191 | gag | aag | E | K | DQ492162 |
| 191 | gag | gtg | E | V | AM402339 |
| 192 | ccc | gcc | P | A | DQ492000 |
| 192 | ccc | tcc | P | S | DQ492076 |
| 193 | gaa | gca | E | A | DQ492187 |
| 193 | gaa | gac | E | D | AM401879 |
| 193 | gaa | gat | E | D | AM401933 |
| 193 | gaa | gta | E | V | DQ492093 |
| 194 | ccg | ctg | P | L | AJ507174, AJ507193, AM402323, EU155301, EU155316 |
| 195 | gac | ggc | D | G | AJ132996, AM401886, AM493441 |
| 195 | gac | aac | D | N | AJ133096 |
| 195 | gac | aat | D | N | AM402061 |
| 196 | gta | gca | V | A | DQ492133 |
| 196 | gta | gcg | V | A | AM401907, AY808041, DQ491958, DQ491959, DQ491960, |
| | | | | | DQ491961, DQ491962, DQ491963, DQ491964, DQ491965, |
| | | | | | DQ491966, DQ491967, DQ491968, DQ491969, DQ491970 |
| 196 | gta | aca | V | T | AY808025 |
| 197 | gca | gaa | A | E | EU256079 |
| 197 | gca | gag | A | E | EF407491 |
| 197 | gca | ata | A | I | AB285035 |
| 197 | gca | atg | A | M | AB285048, AB285073, EF407467 |
| 197 | gca | caa | A | Q | EF407488 |
| 197 | gca | aga | A | R | EU256085 |
| 197 | gca | tca | A | S | AB285067, AB285074, AB285081, AJ507174, EU155220, |
| | | | | | EU155226, EU155259, EU155279, EU155358 |
| 197 | gca | tcg | A | S | EU155256 |
| 197 | gca | aca | A | T | AB010250, AB036519, AB036520, AB036521, AB049089, |
| | | | | | AB049090, AB049092, AB049094, AB049095, AB049096, |
| | | | | | AB049100, AB049101, AB109543, AB191333, AB285040, |
| | | | | | AB285042, AB285043, AB285045, AB285047, AB285049, |
| | | | | | AB285050, AB285053, AB285054, AB285055, AB285056, |
| | | | | | AB285057, AB285058, AB285060, AB285064, AB285068, |
| | | | | | AB285075, AB285078, AB429050, AB442219, AB442220, |
| | | | | | AF033373, AF165045, AF165046, AF165047, AF165048, AF165049, |
| | | | | | AF165050, AF165053, AF165055, AF165056, AF207755, AF207757, |
| | | | | | AF207760, AF207768, AF265145, AF265146, AF265147, AF265148, |
| | | | | | AF265149, AF265150, AF265154, AF265155, AF265156, AF265158, |
| | | | | | AF265159, AF265163, AF265164, AF265165, AF483269, AJ132996, |
| | | | | | AJ132997, AJ133096, AJ507155, AJ507157, AJ507161, AJ507165, |
| | | | | | AJ507168, AJ507169, AJ507171, AJ507175, AJ507177, AJ507181, |
| | | | | | AJ507182, AJ507186, AJ507187, AJ507189, AJ507190, AJ507191, |
| | | | | | AJ507193, AJ507194, AJ507196, AJ507198, AM402258, AM402259, |
| | | | | | AM402260, AM402261, AM402262, AM402263, AM402264, |
| | | | | | AM402265, AM402266, AM402267, AM402268, AM402269, |
| | | | | | AM402270, AM402271, AM402272, AM402273, AM402274, |
| | | | | | AM402275, AM402276, AM402277, AM402278, AM402279, |
| | | | | | AM402280, AM402281, AM402282, AM402283, AM402284, |
| | | | | | AM402285, AM402286, AM402287, AM402288, AM402289, |
| | | | | | AM402290, AM402291, AM402292, AM402293, AM402294, |
| | | | | | AM402295, AM402296, AM402297, AM402298, AM402299, |
| | | | | | AM402300, AM402301, AM402302, AM402303, AM402304, |
| | | | | | AM402305, AM402306, AM402307, AM402308, AM402309, |
| | | | | | AM402310, AM402311, AM402312, AM402313, AM402314, |
| | | | | | AM402315, AM402316, AM402317, AM402318, AM402319, |
| | | | | | AM402320, AM402321, AM402322, AM402323, AM402324, |
| | | | | | AM402325, AM402326, AM402327, AM402328, AM402329, |
| | | | | | AM402330, AM402331, AM402332, AM402333, AM402334, |
| | | | | | AM402335, AM402336, AM402337, AM402338, AM402339, |
| | | | | | AM402340, AM402341, AM402342, AM402343, AM402344, |
| | | | | | AM402345, AM402346, AM402347, AM402348, AM402349, |
| | | | | | AM402350, AM402351, AM402352, AM402353, AM402354, |
| | | | | | AM402355, AM402356, AM402357, AM402358, AM402359, |
| | | | | | AM402360, AM402361, AM402362, AM402363, AM402364, |
| | | | | | AM402365, AM402367, AM402369, AM402370, AM402371, |
| | | | | | AM402372, AM402373, AM402374, AM402375, AM402376, |
| | | | | | AM402377, AM402378, AM402379, AM402380, AM402381, |
| | | | | | AM402382, AM402383, AM402384, AM493436, AM493437, |
| | | | | | AM493438, AM493439, AM493440, AM493441, AM493442, |
| | | | | | AM493443, AM493444, AM493445, AM493446, AM493447, |
| | | | | | AM493448, AM493449, AM493450, AM493451, AY240969, |
| | | | | | AY587016, AY808010, AY808011, AY808013, AY808014, |
| | | | | | AY808015, AY808020, AY808022, AY808023, AY808024, |
| | | | | | AY808025, AY808026, AY808027, AY808028, AY808029, |
| | | | | | AY808036, AY808037, AY808039, AY808043, D30613, D45172, |
| | | | | | D50484, D89815, DQ244141, DQ491979, DQ491980, DQ491981, |
| | | | | | DQ491982, DQ491983, DQ491984, DQ491985, DQ491986, |
| | | | | | DQ491987, DQ491988, DQ491989, DQ491990, DQ491991, |
| | | | | | DQ491992, DQ491993, DQ491994, DQ491995, DQ491996, |
| | | | | | DQ491997, DQ491998, DQ491999, DQ492000, DQ492001, |
| | | | | | DQ492002, DQ492003, DQ492004, DQ492005, DQ492006, |
| | | | | | DQ492007, DQ492063, DQ492064, DQ492065, DQ492066, |
| | | | | | DQ492067, DQ492068, DQ492069, DQ492070, DQ492071, |
| | | | | | DQ492072, DQ492073, DQ492074, DQ492075, DQ492076, |
| | | | | | DQ492078, DQ492079, DQ492080, DQ492081, DQ492082, |
| | | | | | DQ492083, DQ492084, DQ492085, DQ492086, DQ492087, |
| | | | | | DQ492088, DQ492089, DQ492090, DQ492091, DQ492092, |
| | | | | | DQ492093, DQ492124, DQ492125, DQ492127, DQ492128, |
| | | | | | DQ492129, DQ492130, DQ492131, DQ492132, DQ492134, |
| | | | | | DQ492135, DQ492136, DQ492137, DQ492138, DQ492139, |
| | | | | | DQ492140, DQ492141, DQ492142, DQ492143, DQ492144, |
| | | | | | DQ492145, DQ492146, DQ492147, DQ492148, DQ492149, |
| | | | | | DQ492150, DQ492151, DQ492152, DQ492153, DQ492154, |
| | | | | | DQ492155, DQ492156, DQ492157, DQ492158, DQ492159, |
| | | | | | DQ492160, DQ492161, DQ492162, DQ492163, DQ492164, |
| | | | | | DQ492165, DQ492166, DQ492167, DQ492168, DQ492169, |
| | | | | | DQ492170, DQ492171, DQ492172, DQ492173, DQ492174, |
| | | | | | DQ492175, DQ492176, DQ492177, DQ492178, DQ492179, |
| | | | | | DQ492180, DQ492181, DQ492182, DQ492183, DQ492184, |
| | | | | | DQ492185, DQ492186, DQ492187, DQ492188, DQ492189, |
| | | | | | DQ492190, DQ492191, DQ492192, DQ492193, DQ492194, |
| | | | | | DQ492195, DQ492196, DQ492197, EF407460, EF407462, |
| | | | | | EF407466, EF407469, EF407470, EF407474, EF407475, EF407476, |
| | | | | | EF407477, EF407479, EF407485, EF407486, EF407489, EF407490, |
| | | | | | EF407499, EF407500, EF407502, EF407503, EF407504, EU155218, |
| | | | | | EU155223, EU155224, EU155225, EU155227, EU155230, |
| | | | | | EU155231, EU155255, EU155258, EU155280, EU155300, |
| | | | | | EU155301, EU155302, EU155307, EU155316, EU155318, |
| | | | | | EU155324, EU155325, EU155326, EU155327, EU155330, |
| | | | | | EU155335, EU155360, EU155361, EU155363, EU155368, |
| | | | | | EU155371, EU155373, EU155374, EU155375, EU255962, |
| | | | | | EU256001, EU256045, EU256059, EU256075, EU256077, |
| | | | | | EU256078, EU256082, EU256088, EU256089, EU256100, |
| | | | | | EU256103, EU482833, EU482849, EU482874, EU482875, |
| | | | | | EU482877, EU482879, EU482883, EU482888, EU857431, |
| | | | | | FJ024279, FJ390397, FJ958890, FJ958891, FJ958892, FJ958893, |
| | | | | | FJ958894, FJ958895, FJ958896, FJ958897, FJ958898, FJ958899, |
| | | | | | FJ958900, FJ958901, FJ958902, FJ958903, FJ958904, FJ958905, |
| | | | | | FJ958906, FJ958907, FJ958909, FJ958910, FJ958911, FJ958912, |
| | | | | | FJ958913, FJ958914, FJ958915, FJ958916, FJ958917, FJ958918, |
| | | | | | FJ958919, FJ958920, FJ958921, FJ958922, FJ958923, FJ958924, |
| | | | | | FJ958925, FJ958926, FJ958927, FJ958928, FJ958929, FJ958930, |
| | | | | | FJ958931, FJ958932, FJ958933, FJ958934, FJ958935, GQ913868, |
| | | | | | GU451218, GU451219, GU451220, HM042060, L02836, X61596 |
| 197 | gca | acc | A | T | AY044867, EU256083 |
| 197 | gca | acg | A | T | AB154177, AB154178, AB154192, AB285037, AB285052, |
| | | | | | AB285066, AB285079, AF165054, AJ507167, AJ507188, AY808034, |
| | | | | | D50480, EU155232, EU234062, EU482880, EU862837 |
| 197 | gca | act | A | T | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507 |
| 197 | gca | gta | A | V | AM402066, AM402119, EU155260, EU256092, EU256101 |
| 197 | gca | gtg | A | V | AF207771, AJ000009, DQ492025, DQ492033, EF407495 |
| 197 | gca | gtt | A | V | AY808041, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970 |
| 198 | gtg | gcg | V | A | AM402374, DQ491970, DQ492099, EU256059 |
| 198 | gtg | ata | V | I | EU155327 |
| 198 | gtg | atg | V | M | AM401914 |
| 198 | gtg | aca | V | T | EU256092 |
| 198 | gtg | acg | V | T | AF483269, EU482833 |
| 199 | ctc | gcc | L | A | AM402326 |
| 199 | ctc | ttc | L | F | AF207754, AM401941, DQ492179 |
| 199 | ctc | atc | L | I | AB049089, AB049096, AB285040, AB285042, AB285043, |
| | | | | | AB285056, AB285068, AB429050, AJ507171, D50484, EU256077, |
| | | | | | EU256085, EU256101 |
| 199 | ctc | att | L | I | AJ507167 |
| 199 | ctc | ccc | L | P | AF165050 |
| 199 | ctc | gtc | L | V | AB010250, AB049094, AB285035, AB285048, AB285050, |
| | | | | | AB285052, AB285064, AB285073, AB285078, AB285079, |
| | | | | | AB442219, AB442220, AF165045, AF165046, AF165053, AF165054, |
| | | | | | AF207755, AF207757, AF207771, AF265148, AF265149, AF265150, |
| | | | | | AF265154, AF265163, AF265164, AF265165, AJ000009, AJ507169, |
| | | | | | AJ507189, AM402258, AM402259, AM402260, AM402261, |
| | | | | | AM402262, AM402263, AM402264, AM402265, AM402266, |
| | | | | | AM402267, AM402268, AM402269, AM402270, AM402271, |
| | | | | | AM402272, AM402273, AM402274, AM402275, AM402276, |
| | | | | | AM402277, AM402278, AM402279, AM402280, AM402281, |
| | | | | | AM402282, AM402283, AM402284, AM402285, AM402286, |
| | | | | | AM402287, AM402288, AM402289, AM402290, AM402291, |
| | | | | | AM402292, AM402293, AM402294, AM402295, AM402296, |
| | | | | | AM402297, AM402298, AM402299, AM402300, AM402301, |
| | | | | | AM402302, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402327, |
| | | | | | AM402328, AM493436, AM493437, AM493438, AM493439, |
| | | | | | AM493440, AM493441, AM493442, AM493443, AM493444, |
| | | | | | AM493445, AM493446, AM493447, AM493448, AM493449, |
| | | | | | AM493450, AM493451, AY587016, D30613, D45172, D89815, |
| | | | | | DQ244141, EF407467, EF407502, EF407504, EU155260, |
| | | | | | EU155318, EU155335, EU155368, EU256088, EU482877, |
| | | | | | EU857431 |
| 199 | ctc | gtt | L | V | AB285049, EF407495 |
| 200 | act | gct | T | A | AB154177, AB154178, AF207774, AM402363, AY808041, |
| | | | | | DQ491957, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, DQ491977, |
| | | | | | DQ492079, DQ492173, EF407500 |
| 200 | act | tct | T | S | DQ492175 |
| 201 | tcc | ccc | S | P | DQ492039, DQ492095, DQ492144 |
| 202 | atg | ata | M | I | DQ491989, DQ492081 |
| 202 | atg | ctg | M | L | AB049101, AY808037 |
| 202 | atg | ttg | M | L | EU155225 |
| 202 | atg | acg | M | T | DQ492179 |
| 202 | atg | gtg | M | V | DQ492023, DQ492028, DQ492035, DQ492158 |
| 203 | ctc | ttc | L | F | AM402059, AM402062, AM402065, AM402079, AM402083, |
| | | | | | AM402091, AM402123, AM402124, AY808016, AY808030, |
| | | | | | AY808032, DQ492074, DQ492181, EU155365, EU155367, |
| | | | | | EU529682 |
| 203 | ctc | ttt | L | F | EU482833 |
| 203 | ctc | ccc | L | P | AM402086 |
| 204 | acc | gcc | T | A | AM402117, AY240969, DQ244140, DQ492068, FJ958905 |
| 204 | acc | atc | T | I | AF483269, EU155263 |
| 204 | acc | att | T | I | DQ492087 |
| 204 | acc | tcc | T | S | AF207769, AF265158, AF265159 |
| 205 | gac | ggc | D | G | AM493486, DQ492073 |
| 205 | gac | aac | D | N | DQ492068 |
| 207 | tcc | gcc | S | A | AY808041, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, EU155369, |
| | | | | | EU155374, EU256065, EU256066 |
| 207 | tcc | ccc | S | P | AB049092, AF207768, AF265154, AF265155, AF265156, |
| | | | | | AM493490, AY044867, EU155223 |
| 207 | tcc | cct | S | P | AM401871 |
| 208 | cac | cgc | H | R | DQ491945, DQ492137 |
| 209 | att | acc | I | T | AM401858, AM402129 |
| 209 | att | act | I | T | DQ492042 |
| 210 | acg | gca | T | A | AM401855, AM402372 |
| 210 | acg | gcc | T | A | AM402301, AM402308 |
| 210 | acg | ata | T | I | DQ491945, DQ492187 |
| 211 | gcg | gga | A | G | D50480, D50483 |
| 211 | gcg | gta | A | V | DQ491948 |
| 212 | gag | gac | E | D | EU255961 |
| 212 | gag | ggg | E | G | AM401882 |
| 212 | gag | cac | E | H | AM401868, AM401879 |
| 213 | acg | gca | T | A | FJ958907 |
| 213 | acg | gcg | T | A | AB010250, AB049089, AB049092, AB049098, AB154177, |
| | | | | | AB154178, AB154179, AB154181, AB154182, AB154185, |
| | | | | | AB154186, AB154187, AB154188, AB154191, AB154192, |
| | | | | | AB154193, AB154194, AB154197, AB154198, AB154199, |
| | | | | | AB154200, AB154201, AB154202, AB154205, AB154206, |
| | | | | | AB285037, AB285040, AB285042, AB285046, AB285047, |
| | | | | | AB285051, AB285054, AB285055, AB285060, AB285065, |
| | | | | | AB285066, AB285067, AB285071, AB285072, AB285073, |
| | | | | | AB285076, AB285081, AB429050, AB442222, AF033373, |
| | | | | | AF165055, AF207760, AF207768, AF208024, AF265142, AF265143, |
| | | | | | AF265144, AF265160, AF265162, AF313916, AJ132997, AJ507159, |
| | | | | | AJ507162, AJ507169, AJ507171, AJ507172, AJ507195, AM401916, |
| | | | | | AM401919, AM401920, AM401921, AM401923, AM401925, |
| | | | | | AM401926, AM401929, AM401933, AM401934, AM401935, |
| | | | | | AM401937, AM401938, AM401942, AM401946, AM401948, |
| | | | | | AM401955, AM401957, AM402055, AM402056, AM402057, |
| | | | | | AM402058, AM402059, AM402060, AM402061, AM402062, |
| | | | | | AM402063, AM402064, AM402065, AM402066, AM402067, |
| | | | | | AM402068, AM402069, AM402070, AM402071, AM402072, |
| | | | | | AM402073, AM402074, AM402075, AM402076, AM402077, |
| | | | | | AM402078, AM402079, AM402080, AM402081, AM402082, |
| | | | | | AM402083, AM402084, AM402085, AM402086, AM402087, |
| | | | | | AM402088, AM402089, AM402090, AM402091, AM402092, |
| | | | | | AM402093, AM402094, AM402095, AM402096, AM402097, |
| | | | | | AM402098, AM402099, AM402100, AM402101, AM402102, |
| | | | | | AM402103, AM402104, AM402105, AM402106, AM402107, |
| | | | | | AM402108, AM402109, AM402110, AM402111, AM402112, |
| | | | | | AM402113, AM402114, AM402115, AM402116, AM402117, |
| | | | | | AM402118, AM402119, AM402120, AM402121, AM402122, |
| | | | | | AM402123, AM402124, AM402125, AM402126, AM402127, |
| | | | | | AM402128, AM402129, AM402339, AM494937, AY587016, |
| | | | | | AY587844, AY808013, AY808014, AY808016, AY808023, |
| | | | | | AY808027, AY808030, D10934, D30613, D45172, D50480, D50485, |
| | | | | | D85516, D89815, DQ244141, DQ492093, DQ492163, DQ492164, |
| | | | | | DQ492165, DQ492166, DQ492167, DQ492168, DQ492169, |
| | | | | | DQ492170, DQ492171, DQ492172, DQ492173, DQ492174, |
| | | | | | DQ492175, DQ492176, DQ492177, DQ492178, DQ492179, |
| | | | | | DQ492180, DQ492181, DQ492182, DQ492183, DQ492184, |
| | | | | | DQ492185, DQ492186, DQ492187, DQ492188, DQ492189, |
| | | | | | DQ492190, DQ492191, DQ492192, DQ492193, DQ492194, |
| | | | | | DQ492195, DQ492196, DQ492197, EF407459, EF407492, |
| | | | | | EF407497, EF407503, EU155219, EU155221, EU155232, |
| | | | | | EU155254, EU155255, EU155300, EU155301, EU155307, |
| | | | | | EU155316, EU155317, EU155365, EU155367, EU155368, |
| | | | | | EU155369, EU155377, EU234062, EU256045, EU256075, |
| | | | | | EU256085, EU482849, EU482875, EU482886, EU529682, |
| | | | | | EU857431, EU862835, FJ390397, FJ390398, FJ958890, FJ958891, |
| | | | | | FJ958892, FJ958893, FJ958894, FJ958895, FJ958896, FJ958897, |
| | | | | | FJ958898, FJ958899, FJ958900, FJ958901, FJ958902, FJ958903, |
| | | | | | FJ958904, FJ958905, FJ958906, FJ958909, FJ958910, FJ958911, |
| | | | | | FJ958912, FJ958913, FJ958914, FJ958915, FJ958916, FJ958917, |
| | | | | | FJ958918, FJ958919, FJ958920, FJ958921, FJ958922, FJ958923, |
| | | | | | FJ958924, FJ958925, FJ958926, FJ958927, FJ958928, FJ958929, |
| | | | | | FJ958930, FJ958931, FJ958932, FJ958933, FJ958934, FJ958935, |
| | | | | | FN435993, GQ913870, HM042060 |
| 213 | acg | atg | T | M | AM493481, DQ492028 |
| 213 | acg | tcg | T | S | EU155217 |
| 213 | acg | gtg | T | V | AY044867 |
| 214 | gct | gtt | A | V | AM402115, DQ492050 |
| 215 | aag | gcg | K | A | AJ507156 |
| 215 | aag | gaa | K | E | DQ492132, DQ492135 |
| 215 | aag | gga | K | G | DQ492197 |
| 215 | aag | ggc | K | G | AY808015 |
| 215 | aag | ggg | K | G | AB285045, AY808014, AY808019, AY808021, AY808041, |
| | | | | | DQ491958, DQ491959, DQ491960, DQ491961, DQ491962, |
| | | | | | DQ491963, DQ491964, DQ491965, DQ491966, DQ491967, |
| | | | | | DQ491968, DQ491969, DQ491970, DQ492093, DQ492094, |
| | | | | | DQ492095, DQ492096, DQ492097, DQ492098, DQ492099, |
| | | | | | DQ492100, DQ492101, DQ492102, DQ492103, DQ492104, |
| | | | | | DQ492105, DQ492106, DQ492107, DQ492108, DQ492109, |
| | | | | | DQ492110, DQ492111, DQ492112, DQ492113, DQ492114, |
| | | | | | DQ492115, DQ492116, DQ492117, DQ492118, DQ492119, |
| | | | | | DQ492120, DQ492121, DQ492122, DQ492123, DQ492163, |
| | | | | | DQ492164, DQ492165, DQ492166, DQ492167, DQ492168, |
| | | | | | DQ492169, DQ492170, DQ492171, DQ492172, DQ492173, |
| | | | | | DQ492174, DQ492175, DQ492176, DQ492177, DQ492178, |
| | | | | | DQ492179, DQ492180, DQ492181, DQ492182, DQ492183, |
| | | | | | DQ492184, DQ492185, DQ492186, DQ492187, DQ492188, |
| | | | | | DQ492189, DQ492190, DQ492191, DQ492192, DQ492193, |
| | | | | | DQ492194, DQ492195, DQ492196, EF407473, EF407489, |
| | | | | | EU155224, EU155377, EU256075 |
| 215 | aag | ata | K | I | DQ492133 |
| 215 | aag | aga | K | R | AB285049, AB442222 |
| 215 | aag | agg | K | R | AB010250, AB049087, AB049092, AB049094, AB049099, |
| | | | | | AB049101, AB154185, AB154186, AB285040, AB285042, |
| | | | | | AB285046, AB285052, AB285058, AB285064, AB285073, |
| | | | | | AB285076, AB285078, AB285079, AB442219, AF165051, |
| | | | | | AF165052, AF165053, AF165054, AF165061, AF165062, AF207762, |
| | | | | | AF207764, AF207768, AF207769, AF207772, AF265154, AF265155, |
| | | | | | AF265156, AJ507171, AJ507189, AM401876, AM402330, |
| | | | | | AM402332, AM402333, AM402335, AM402337, AM402338, |
| | | | | | AM402345, AM402346, AM402347, AM402348, AM402350, |
| | | | | | AM402352, AM402353, AM402354, AM402355, AM402356, |
| | | | | | AM402357, AM402358, AM402359, AM402360, AM402361, |
| | | | | | AM402363, AM402364, AM402365, AM402367, AM402369, |
| | | | | | AM402370, AM402372, AM402373, AM402374, AM402375, |
| | | | | | AM402376, AM402377, AM402378, AM402381, AM402382, |
| | | | | | AM402383, AM402384, AY587016, AY808025, D30613, D45172, |
| | | | | | D89815, DQ244141, EF407467, EF407485, EF407498, EF407502, |
| | | | | | EU155219, EU155227, EU155231, EU155255, EU155382, |
| | | | | | EU256079, EU482879, EU857431, HM042058, HM042059 |
| 216 | cgt | cac | R | H | DQ491954, DQ492161 |
| 216 | cgt | ccc | R | P | DQ492157 |
| 217 | agg | aaa | R | K | AB049094, GQ913869 |
| 217 | agg | aag | R | K | AF034151 |
| 217 | agg | atg | R | M | AJ507165 |
| 217 | agg | agc | R | S | AM401863 |
| 218 | ctg | ccg | L | P | AB010249, AM402117, AM402332, D16435, D89872 |
| 218 | ctg | cgg | L | R | AM401915 |
| 218 | ctg | tcg | L | S | DQ492182 |
| 219 | gcc | gac | A | D | AJ000009, AJ132996, AM493493, AY044867, EF407499, |
| | | | | | EU155374, EU660388 |
| 219 | gcc | gat | A | D | AF356827 |
| 219 | gcc | gaa | A | E | AJ507172 |
| 219 | gcc | ggc | A | G | AY587844, EU155264, EU482879 |
| 219 | gcc | ccc | A | P | AY240968 |
| 219 | gcc | acc | A | T | AM401907 |
| 219 | gcc | gtc | A | V | AM401902, FN435993 |
| 220 | agg | ggg | R | G | DQ492017 |
| 222 | tct | tgt | S | C | AB049089, AM401905 |
| 222 | tct | cca | S | P | AM402295 |
| 222 | tct | ccc | S | P | DQ492060, FJ958931 |
| 222 | tct | cct | S | P | D63857, DQ491976, DQ491990, DQ492064, DQ492095 |
| 222 | tct | acc | S | T | EU155219, EU155333, EU482885 |
| 223 | ccc | gct | P | A | EU155219, EU155333, EU482885 |
| 223 | ccc | ggg | P | G | AY808017 |
| 223 | ccc | tcc | P | S | AF176573, EF407465 |
| 224 | ccc | ggg | P | G | AY808017 |
| 224 | ccc | tcc | P | S | DQ492154 |
| 225 | tcc | ttc | S | F | AB285075 |
| 226 | ttg | gca | L | A | EU155305 |
| 226 | ttg | atg | L | M | AB049091, AJ133096 |
| 226 | ttg | ccg | L | P | AM402269 |
| 226 | ttg | cgg | L | R | DQ492171 |
| 226 | ttg | tcg | L | S | AM402086, AM402329, DQ492100, DQ492149 |
| 226 | ttg | gtg | L | V | FJ390396 |
| 226 | ttg | tgg | L | W | AM401918 |
| 227 | gcc | ccc | A | P | DQ492142 |
| 227 | gcc | gtc | A | V | DQ492080 |
| 228 | agc | ggc | S | G | AM401850, AM402291, AM402308, DQ492021, HM042059 |
| 228 | agc | ggt | S | G | D89815, FJ958903 |
| 228 | agc | aac | S | N | DQ492162 |
| 228 | agc | agg | S | R | DQ492100 |
| 228 | agc | cgc | S | R | AB109543, AB191333, AB285050 |
| 229 | tca | cca | S | P | AM401902 |
| 230 | tca | gca | S | A | FJ958905 |
| 230 | tca | tta | S | L | AM401864 |
| 230 | tca | cca | S | P | DQ491991, DQ492103 |
| 231 | gct | acc | A | T | AM401962 |
| 231 | gct | act | A | T | AM402266, AM402299, AM402361 |
| 231 | gct | gtt | A | V | AM402337 |
| 232 | agc | tgt | S | C | DQ492186 |
| 232 | agc | acc | S | T | AB285063 |
| 232 | agc | act | S | T | AB049092 |
| 233 | cag | cgg | Q | R | DQ492042, DQ492161 |
| 234 | ctg | ttc | L | F | DQ492042 |
| 234 | ctg | ccg | L | P | DQ492172 |
| 234 | ctg | tcg | L | S | AM402320, DQ492019, DQ492032 |
| 235 | tct | ttt | S | F | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401862, |
| | | | | | AM401863, AM401864, AM401865, AM401866, AM401867, |
| | | | | | AM401868, AM401869, AM401870, AM401871, AM401872, |
| | | | | | AM401873, AM401874, AM401875, AM401877, AM401879, |
| | | | | | AM402303 |
| 236 | gcg | acg | A | T | DQ492034 |
| 236 | gcg | gtg | A | V | AM493485 |
| 237 | cct | gct | P | A | EU482849, EU529682, EU862835 |
| 237 | cct | ttt | P | F | AJ507169 |
| 237 | cct | att | P | I | GQ913873 |
| 237 | cct | ctt | P | L | AB010250, AB285041, AY008261, AY587016, D30613, D45172, |
| | | | | | D89815, EF407461, EF407468, EU857431, M84754 |
| 237 | cct | tct | P | S | AM402315 |
| 237 | cct | act | P | T | DQ492048 |
| 237 | cct | gtc | P | V | AB285040 |
| 237 | cct | gtt | P | V | AY240968, EF407467, EU155306, HM042056 |
| 238 | tcc | gcc | S | A | DQ492070 |
| 238 | tcc | ccc | S | P | AM402087 |
| 238 | tcc | cct | S | P | AM401900, AM401903 |
| 239 | ttg | atg | L | M | EU482874, HM042054 |
| 239 | ttg | ccg | L | P | DQ492025, DQ492033 |
| 239 | ttg | tcg | L | S | AM401862, AM402312, AY808031, D50483 |
| 240 | aag | gag | K | E | AM402274, AM402299, DQ492181, DQ492191 |
| 240 | aag | agg | K | R | AB285054, AB285055, AJ507183, AM402103, AY808013, |
| | | | | | AY808025, EU256081, EU482879, FJ024279, FJ958890, FJ958896, |
| | | | | | GQ913868, GQ913871, HM042050, HM042062 |
| 240 | aag | tgg | K | W | AY240967 |
| 241 | gca | ccg | A | P | EF407461 |
| 241 | gca | gtg | A | V | AY240969, FJ958895 |
| 242 | aca | gca | T | A | AB049091, AF165047, AF165048, AJ507169, AM401884, |
| | | | | | AM402327, DQ492034, DQ492136, EF407467, EF407468, |
| | | | | | EU155306, EU255961, EU529682, GQ913871, GQ913873, |
| | | | | | HM042056 |
| 242 | aca | gcg | T | A | AB285040, AY240968, DQ492165, M84754 |
| 242 | aca | cac | T | H | HM042062 |
| 242 | aca | aag | T | K | AY240969 |
| 243 | tgc | cgc | C | R | AM401939, AM402346, DQ491972, DQ491973 |
| 243 | tgc | tac | C | Y | AM401936, D50483, DQ492046, DQ492142 |
| 244 | act | gcc | T | A | AB285060 |
| 244 | act | gct | T | A | DQ244141, DQ492016, DQ492047, HM042062 |
| 244 | act | ggt | T | G | HM042058 |
| 244 | act | att | T | I | AY808031, D50483, DQ492158 |
| 244 | act | aat | T | N | DQ244140 |
| 244 | act | ccc | T | P | AB285076 |
| 244 | act | cct | T | P | AB154195, AB154196, AF165062, DQ492160 |
| 245 | acc | gcc | T | A | AB285040, AB429050, AF265154, AF265155, AF265156, AJ507175, |
| | | | | | AM402308, AY008261, AY808025, AY808029, AY808043, |
| | | | | | DQ492063, DQ492064, DQ492065, DQ492067, DQ492068, |
| | | | | | DQ492069, DQ492070, DQ492071, DQ492072, DQ492073, |
| | | | | | DQ492074, DQ492075, DQ492076, DQ492077, DQ492078, |
| | | | | | DQ492079, DQ492080, DQ492081, DQ492082, DQ492083, |
| | | | | | DQ492084, DQ492085, DQ492086, DQ492087, DQ492088, |
| | | | | | DQ492089, DQ492090, DQ492091, DQ492092, EU155260, |
| | | | | | EU155306, EU155360, EU155361, EU482879, EU482880, |
| | | | | | EU482888, EU529682, HM042059 |
| 245 | acc | atc | T | I | AB285070, AY240969, D14484, DQ492131 |
| 245 | acc | aac | T | N | EU155332, EU862835 |
| 245 | acc | tac | T | Y | AB154195, AB154196 |
| 246 | cgt | tgc | R | C | AB049087, AB049092, AF207768, D50484, EU155360 |
| 246 | cgt | tgt | R | C | AB285054, AB285055, AB285076, AJ507189, AM401908, |
| | | | | | AY240969, AY808013, AY808043, DQ492113, DQ492114, |
| | | | | | DQ492117, DQ492118, DQ492119, EF407494, EU482875, |
| | | | | | HM042059 |
| 246 | cgt | cac | R | H | AB010249, AB049099, AB080299, AB285035, AB285039, |
| | | | | | AB285048, AB285057, AB285060, AB285061, AB285072, |
| | | | | | AF207753, AF207754, AF207758, AF207769, AF207770, AF333324, |
| | | | | | AF483269, AJ507159, AJ507163, AJ507179, AJ507182, AJ507183, |
| | | | | | AM493484, AY808025, AY808032, AY808039, AY808040, D11168, |
| | | | | | D16435, D50482, D85516, D89872, DQ491943, DQ491944, |
| | | | | | DQ491945, DQ491946, DQ491947, DQ491948, DQ491949, |
| | | | | | DQ491950, DQ491951, DQ491952, DQ491953, DQ491954, |
| | | | | | DQ491955, DQ491956, DQ491957, DQ492080, EF407480, |
| | | | | | EF407498, EF407500, EF407504, EU155254, EU155302, |
| | | | | | EU155316, EU155336, EU155361, EU155375, EU155382, |
| | | | | | EU255961, EU256099, EU482874, EU482885, EU482886, |
| | | | | | GU451218, GU451219, GU451220, M58335 |
| 246 | cgt | cat | R | H | AB010250, AB036519, AB036520, AB036521, AB049088, |
| | | | | | AB049091, AB049094, AB049096, AB049098, AB049100, |
| | | | | | AB049101, AB109543, AB191333, AB249644, AB285036, |
| | | | | | AB285037, AB285038, AB285043, AB285044, AB285053, |
| | | | | | AB285058, AB285059, AB285062, AB285063, AB285064, |
| | | | | | AB285069, AB285070, AB285073, AB285077, AB285078, |
| | | | | | AB426117, AB442219, AB442220, AB442221, AB442222, |
| | | | | | AF165047, AF165048, AF165049, AF165050, AF165055, AF165056, |
| | | | | | AF165057, AF165058, AF165059, AF165060, AF165061, AF207752, |
| | | | | | AF207756, AF207762, AF207763, AF207764, AF207765, AF207772, |
| | | | | | AF207774, AF208024, AF265148, AF265158, AF265159, AJ133096, |
| | | | | | AJ507155, AJ507156, AJ507162, AJ507165, AJ507172, AJ507175, |
| | | | | | AJ507177, AJ507180, AJ507181, AJ507184, AJ507192, AJ507197, |
| | | | | | AJ507198, AM402329, AM402331, AM402334, AM402336, |
| | | | | | AM402339, AM402340, AM402341, AM402342, AM402343, |
| | | | | | AM402344, AM493497, AM493501, AM493504, AY045702, |
| | | | | | AY240967, AY240968, AY460204, AY587016, AY808010, |
| | | | | | AY808012, AY808017, AY808023, AY808024, AY808027, |
| | | | | | AY808034, AY808042, D10934, D14484, D30613, D45172, D50480, |
| | | | | | D50481, D63857, D89815, D90208, DQ244140, DQ491971, |
| | | | | | DQ491972, DQ491973, DQ491974, DQ491975, DQ491976, |
| | | | | | DQ491977, DQ491978, DQ492008, DQ492009, DQ492010, |
| | | | | | DQ492011, DQ492012, DQ492013, DQ492014, DQ492015, |
| | | | | | DQ492016, DQ492017, DQ492018, DQ492019, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ492040, |
| | | | | | DQ492041, DQ492042, DQ492043, DQ492044, DQ492045, |
| | | | | | DQ492046, DQ492049, DQ492051, DQ492052, DQ492053, |
| | | | | | DQ492056, DQ492057, DQ492058, DQ492059, DQ492060, |
| | | | | | DQ492061, DQ492062, DQ492065, DQ492066, DQ492067, |
| | | | | | DQ492068, DQ492070, DQ492073, DQ492074, DQ492078, |
| | | | | | DQ492079, DQ492082, DQ492083, DQ492084, DQ492085, |
| | | | | | DQ492086, DQ492087, DQ492088, DQ492089, DQ492090, |
| | | | | | DQ492091, DQ492092, DQ492127, DQ492129, DQ492130, |
| | | | | | DQ492131, DQ492140, DQ492157, EF032892, EF032893, |
| | | | | | EF032894, EF407462, EF407466, EF407477, EF407479, EF407485, |
| | | | | | EF407487, EF407491, EF407497, EF407502, EF638081, EU155219, |
| | | | | | EU155226, EU155227, EU155253, EU155257, EU155259, |
| | | | | | EU155262, EU155279, EU155328, EU155335, EU155357, |
| | | | | | EU155358, EU155359, EU155362, EU155367, EU155368, |
| | | | | | EU155369, EU155370, EU155371, EU155372, EU155377, |
| | | | | | EU155381, EU239714, EU255960, EU255962, EU256059, |
| | | | | | EU256077, EU256079, EU256082, EU256083, EU256088, |
| | | | | | EU256090, EU256101, EU256102, EU482833, EU482839, |
| | | | | | EU482859, EU482860, EU482877, EU482883, EU781832, |
| | | | | | EU857431, FJ024277, FJ390396, FJ478453, GU451221, GU451222, |
| | | | | | GU451223, GU451224, L02836, M96362, U01214, U16362 |
| 246 | cgt | aaa | R | K | EU155317, EU256080, EU256091 |
| 246 | cgt | ctt | R | L | AB154195, AB154196, AB285051, AY808026 |
| 246 | cgt | aac | R | N | EU155325 |
| 246 | cgt | aat | R | N | AB285047, AF165051, AF165052, AF165062, AY808009, |
| | | | | | DQ859970, EU155306, EU256084 |
| 246 | cgt | caa | R | Q | AM401916, AM401917, AM401918, AM401919, AM401920, |
| | | | | | AM401921, AM401922, AM401923, AM401924, AM401925, |
| | | | | | AM401926, AM401927, AM401928, AM401929, AM401931, |
| | | | | | AM401932, AM401933, AM401934, AM401935, AM401936, |
| | | | | | AM401937, AM401938, AM401939, AM401940, AM401941, |
| | | | | | AM401942, AM401943, AM401944, AM401945, AM401946, |
| | | | | | AM401947, AM401948, AM401949, AM401950, AM401951, |
| | | | | | AM401952, AM401953, AM401954, AM401955, AM401956, |
| | | | | | AM401957, AM401958, AM401959, AM401960, AM401961, |
| | | | | | AM401962, AM401963, AM401964, AM401965, AM401966, |
| | | | | | AM401967, AM401968, AM401969, AM401970, AM401971, |
| | | | | | AM401972, AM401973, AM401974, AM401975, AM401976, |
| | | | | | AM401977, AM401978, AM401979, AM401980, AM402330, |
| | | | | | AM402332, AM402333, AM402335, AM402337, AM402338, |
| | | | | | AM402345, AM402346, AM402347, AM402348, AM402349, |
| | | | | | AM402350, AM402351, AM402352, AM402353, AM402354, |
| | | | | | AM402355, AM402356, AM402357, AM402358, AM402359, |
| | | | | | AM402360, AM402361, AM402362, AM402363, AM402364, |
| | | | | | AM402365, AM402367, AM402369, AM402370, AM402371, |
| | | | | | AM402372, AM402373, AM402374, AM402375, AM402376, |
| | | | | | AM402377, AM402378, AM402379, AM402380, AM402381, |
| | | | | | AM402382, AM402383, AM402384, AY808031, D50483, EU155376 |
| 246 | cgt | cag | R | Q | AB154205, AB154206, AM401930, EU482880 |
| 246 | cgt | tac | R | Y | AB049089 |
| 246 | cgt | tat | R | Y | EF407481 |
| 247 | cat | ttt | H | F | HM042058 |
| 247 | cat | aaa | H | K | AB285059 |
| 247 | cat | ctt | H | L | AJ507189 |
| 247 | cat | aac | H | N | AY240969, HM042062 |
| 247 | cat | cct | H | P | AY808031 |
| 247 | cat | caa | H | Q | AB049090, AM402077, EU155377 |
| 247 | cat | cgt | H | R | AB285038, AB285044, AB285060, AF207762, EU155302 |
| 247 | cat | tat | H | Y | D50483 |
| 248 | gac | gaa | D | E | AF265156, EU256078 |
| 248 | gac | ggc | D | G | AB010250, AB049089, AF207769, AJ507189, AM401938, |
| | | | | | AY587016, D30613, D45172, D89815, EF407481, EU857431, |
| | | | | | HM042062 |
| 248 | gac | cac | D | H | AB049091 |
| 248 | gac | aac | D | N | AB049092, DQ244141, DQ492054, DQ492055, DQ492085 |
| 248 | gac | aat | D | N | EU256084 |
| 248 | gac | cgc | D | R | AB285043, DQ244140 |
| 248 | gac | acc | D | T | M84754 |
| 248 | gac | gtc | D | V | AF333324, AY808032, EU482875, M58335 |
| 249 | tcc | gcc | S | A | AB010250, AB049092, AY587016, D30613, D45172, D89815, |
| | | | | | EU155326, EU857431 |
| 249 | tcc | tgc | S | C | AJ507189 |
| 249 | tcc | ttc | S | F | AY808031, DQ492096 |
| 249 | tcc | ccc | S | P | AB285076, EF407467, EF407481, M84754 |
| 249 | tcc | cct | S | P | AJ507169, HM042062 |
| 250 | ccg | gca | P | A | AB285078 |
| 250 | ccg | ctg | P | L | DQ492191 |
| 250 | ccg | caa | P | Q | EU255962 |
| 250 | ccg | acg | P | T | AY460204, EF638081 |
| 251 | gac | gcc | D | A | AB285067 |
| 251 | gac | gag | D | E | AB049100 |
| 251 | gac | ggc | D | G | AF165062, AM402382, DQ859970 |
| 251 | gac | aac | D | N | AJ507189, DQ491979 |
| 252 | gct | ttt | A | F | D50483 |
| 252 | gct | att | A | I | AB285056 |
| 252 | gct | ctc | A | L | DQ491983 |
| 252 | gct | ctt | A | L | AB049091, AB285043, DQ244140, DQ244141, DQ491980, |
| | | | | | DQ491981, DQ491984, DQ491989, DQ491994, DQ492047, |
| | | | | | DQ492048, DQ492050, DQ492054, DQ492055, EF407480 |
| 252 | gct | cct | A | P | AB049100 |
| 252 | gct | aca | A | T | DQ492090 |
| 252 | gct | acc | A | T | AF207769, DQ492021, DQ492129, DQ492130, DQ492140, |
| | | | | | DQ492150 |
| 252 | gct | act | A | T | AB010250, AY587016, D30613, D45172, D89815, EU482849, |
| | | | | | EU857431 |
| 252 | gct | gta | A | V | EU529682 |
| 252 | gct | gtc | A | V | AB285044, AB429050, AM493443, AY008261, EF407467, |
| | | | | | EU155332, EU155366, HM042059, HM042062 |
| 252 | gct | gtt | A | V | AB285040, AB285076, AB285078, AF165061, AF165062, |
| | | | | | AF207757, AF207762, AJ507191, AY045702, AY240967, AY240969, |
| | | | | | AY808039, DQ491967, EF407461, EF407473, EF407488, |
| | | | | | EU155220, EU155221, EU155222, EU155264, EU155280, |
| | | | | | EU155306, EU155324, EU234061, EU255962, EU482839, |
| | | | | | FJ390397, FJ478453, GQ913872, GQ913873, HM042051 |
| 253 | gac | gcc | D | A | DQ491980, DQ491981, DQ491983, DQ491984, DQ491989, |
| | | | | | DQ491994 |
| 253 | gac | gaa | D | E | AB442220, EF407468 |
| 253 | gac | gag | D | E | AJ507169, AY240968 |
| 253 | gac | ggc | D | G | AF356827 |
| 254 | ctc | ttc | L | F | AM401890, AM402370, DQ492127 |
| 254 | ctc | ccc | L | P | DQ492106 |
| 255 | atc | ctc | I | L | AY808039, EU155306 |
| 255 | atc | agc | I | S | DQ492017 |
| 255 | atc | acc | I | T | EF407480, HM042062 |
| 255 | atc | act | I | T | AM401882 |
| 255 | atc | gtc | I | V | AB285040, AJ507169, AJ507196, AM402335, AM493471, |
| | | | | | AM493472, AM493473, AM493474, AM493475, AM493476, |
| | | | | | AM493477, AM493478, AM493479, AM493480, AM493481, |
| | | | | | AM493482, AM493483, AM493484, AM493485, AM493486, |
| | | | | | AM493487, AM493488, AY240969, DQ492098, EF407459, |
| | | | | | EF407478, EF407481 |
| 255 | atc | gtt | I | V | AF207769, AY008261, HM042058, HM042059 |
| 256 | gag | gac | E | D | AY808016 |
| 256 | gag | gat | E | D | AM401849, AY008261 |
| 257 | gcc | acc | A | T | AM402076, DQ492129, DQ492130, DQ492140 |
| 257 | gcc | gtc | A | V | AM493477, DQ491955 |
| 258 | aac | gac | N | D | AM401856, DQ491955 |
| 258 | aac | gat | N | D | AM402296, AM402315 |
| 258 | aac | cac | N | H | U45476 |
| 258 | aac | atc | N | I | DQ492172 |
| 258 | aac | agc | N | S | EF407473, EU155367 |
| 258 | aac | agt | N | S | AB154203, AB154204 |
| 259 | ctc | ttc | L | F | DQ492182 |
| 259 | ctc | ccc | L | P | AM402072, DQ492054, DQ492055 |
| 260 | ctg | atg | L | M | AF207769, AY808028, DQ491980, DQ491981, DQ491983, |
| | | | | | DQ491984, DQ491989, DQ491994, DQ492040, DQ492047, |
| | | | | | DQ492048, DQ492050, DQ492054, DQ492055, EF407481, |
| | | | | | HM042059 |
| 260 | ctg | ccg | L | P | DQ491967, DQ492087, DQ492099 |
| 261 | tgg | cgg | W | R | AM402313, DQ492175 |
| 262 | cgg | cac | R | H | AF165061, AF165062 |
| 262 | cgg | aag | R | K | AJ507155 |
| 262 | cgg | cag | R | Q | AB285040, DQ491965, DQ492114, DQ492169, EU155306, |
| | | | | | EU529682 |
| 262 | cgg | tgg | R | W | AB049088, AB285043, AB285044, AB285064, AY808026, |
| | | | | | AY808028, DQ244140, DQ244141, DQ491980, DQ491981, |
| | | | | | DQ491983, DQ491984, DQ491989, DQ491994, DQ492015, |
| | | | | | DQ492040, DQ492047, DQ492048, DQ492050, DQ492054, |
| | | | | | DQ492055, DQ492125, DQ492172, EF407480, EF407481, |
| | | | | | HM042059, HM042062 |
| 263 | cag | cat | Q | H | AB285076, AY808026 |
| 263 | cag | cgg | Q | R | AM401962, AM402287, AM402384, HM042059 |
| 264 | gag | gca | E | A | AJ507173 |
| 264 | gag | gcg | E | A | EU155232, EU155281, EU155359 |
| 264 | gag | aag | E | K | AM493487, AM493488 |
| 264 | gag | cag | E | Q | AB285044 |
| 264 | gag | acg | E | T | EF407494 |
| 264 | gag | gtg | E | V | AF207769, EU529682 |
| 265 | atg | ata | M | I | AM402102, DQ492072, DQ492151 |
| 265 | atg | aaa | M | K | DQ491980, DQ491981, DQ491983, DQ491984, DQ491989, |
| | | | | | DQ491994 |
| 265 | atg | aag | M | K | AB285043, AB285064, DQ244140, DQ244141, DQ492040, |
| | | | | | DQ492047, DQ492048, DQ492050, DQ492054, DQ492055 |
| 265 | atg | ctg | M | L | AY808014, AY808026, DQ492093, DQ492163, DQ492166, |
| | | | | | DQ492167, DQ492168, DQ492169, DQ492170, DQ492171, |
| | | | | | DQ492172, DQ492173, DQ492174, DQ492175, DQ492176, |
| | | | | | DQ492177, DQ492179, DQ492181, DQ492182, DQ492183, |
| | | | | | DQ492184, DQ492185, DQ492187, DQ492190, DQ492191, |
| | | | | | DQ492192, DQ492193, DQ492196 |
| 265 | atg | acg | M | T | DQ492073 |
| 265 | atg | gtg | M | V | DQ492075, DQ492104, EF407480, EU482880 |
| 266 | ggc | gcc | G | A | EF407480 |
| 266 | ggc | tgc | G | C | AY587844 |
| 266 | ggc | gac | G | D | DQ492019 |
| 266 | ggc | gat | G | D | EU256088 |
| 266 | ggc | aac | G | N | EU155281 |
| 266 | ggc | aat | G | N | EF407489 |
| 267 | ggg | gcg | G | A | EF407503 |
| 267 | ggg | gat | G | D | AB154203, AB154204 |
| 267 | ggg | gag | G | E | DQ491978, DQ492151 |
| 267 | ggg | aat | G | N | EU256075 |
| 267 | ggg | tca | G | S | AY808014, DQ492093, DQ492163, DQ492164, DQ492165, |
| | | | | | DQ492166, DQ492167, DQ492168, DQ492169, DQ492170, |
| | | | | | DQ492171, DQ492172, DQ492173, DQ492174, DQ492175, |
| | | | | | DQ492176, DQ492177, DQ492178, DQ492179, DQ492180, |
| | | | | | DQ492181, DQ492182, DQ492183, DQ492184, DQ492185, |
| | | | | | DQ492186, DQ492187, DQ492188, DQ492189, DQ492190, |
| | | | | | DQ492191, DQ492192, DQ492193, DQ492194, DQ492195, |
| | | | | | DQ492196, DQ492197 |
| 268 | aac | gac | N | D | AB285076, AM401964, AM402124, AM402275, AY808031, |
| | | | | | EF407481 |
| 268 | aac | gat | N | D | EF407466 |
| 268 | aac | gaa | N | E | AY008261 |
| 268 | aac | atc | N | I | AJ507163 |
| 268 | aac | aaa | N | K | AY808026 |
| 268 | aac | agc | N | S | AF207769, AJ507169, AY808037, DQ492167, EF407467, |
| | | | | | HM042062 |
| 268 | aac | acc | N | T | AB285040, GQ913873 |
| 269 | atc | cac | I | H | AY240969 |
| 269 | atc | acc | I | T | AM401853, AM401890, AM401976, AM402379 |
| 269 | atc | gtc | I | V | AM402083, AM402259, AY808026, DQ492020, DQ492026, |
| | | | | | DQ492144 |
| 270 | acc | gcc | T | A | AM401867, AM401878, AM402353, DQ492180 |
| 270 | acc | atc | T | I | DQ491997 |
| 270 | acc | ttg | T | L | EU155325 |
| 270 | acc | tcc | T | S | AB049088, AB285044, AY808037 |
| 270 | acc | gtc | T | V | EU256084 |
| 270 | acc | tac | T | Y | EF407480 |
| 271 | cgc | cac | R | H | AM401966, AM402321 |
| 271 | cgc | ccg | R | P | DQ492011 |
| 272 | gtg | gcg | V | A | DQ492113, DQ492117, DQ492118, DQ492119, DQ492191 |
| 272 | gtg | gag | V | E | AM401858 |
| 273 | gag | gat | E | D | AB285073 |
| 273 | gag | ggg | E | G | AM402356, DQ492002, DQ492087, FJ958895 |
| 273 | gag | aag | E | K | DQ492092 |
| 273 | gag | gtg | E | V | DQ492132, DQ492135 |
| 274 | tca | cca | S | P | AM402315 |
| 275 | gaa | gac | E | D | AY240967 |
| 275 | gaa | ggg | E | G | AM401973, DQ491957, DQ492034, DQ492188 |
| 276 | aat | gcc | N | A | EU256091 |
| 276 | aat | gct | N | A | AY587844 |
| 276 | aat | gac | N | D | AM401974 |
| 276 | aat | aaa | N | K | HM042056 |
| 276 | aat | aag | N | K | EU155306 |
| 276 | aat | agc | N | S | AM401857, DQ492114, DQ492167, EF407471, EU155325, |
| | | | | | EU862837 |
| 276 | aat | agt | N | S | DQ492053, U01214 |
| 277 | aag | gaa | K | E | DQ492176, DQ492183 |
| 277 | aag | gag | K | E | DQ492085 |
| 278 | gta | gca | V | A | AM402315, AM402328, AM402383 |
| 278 | gta | ata | V | I | AB010250, AF165053, AF165054, AY587016, D30613, D45172, |
| | | | | | D89815, DQ492028, EU155260, EU155362, EU857431 |
| 278 | gta | aca | V | T | AY808017 |
| 279 | gta | gcg | V | A | AM402113 |
| 279 | gta | ata | V | I | AB049088, AB049101, AB285043, AB285064, AB285071, |
| | | | | | AB442222, AJ507169, AM401877, AY808014, AY808028, |
| | | | | | DQ244140, DQ244141, DQ491980, DQ491981, DQ491983, |
| | | | | | DQ491984, DQ491989, DQ492040, DQ492047, DQ492048, |
| | | | | | DQ492050, DQ492054, DQ492055, DQ492093, DQ492163, |
| | | | | | DQ492164, DQ492165, DQ492166, DQ492167, DQ492168, |
| | | | | | DQ492169, DQ492170, DQ492171, DQ492172, DQ492173, |
| | | | | | DQ492174, DQ492175, DQ492176, DQ492177, DQ492178, |
| | | | | | DQ492179, DQ492180, DQ492181, DQ492182, DQ492183, |
| | | | | | DQ492184, DQ492185, DQ492186, DQ492187, DQ492188, |
| | | | | | DQ492189, DQ492190, DQ492191, DQ492192, DQ492193, |
| | | | | | DQ492194, DQ492195, DQ492196, DQ492197, EF407503, |
| | | | | | EU155333, EU482880, GU451219, GU451220 |
| 279 | gta | att | V | I | D50480 |
| 280 | att | acc | I | T | D50483 |
| 280 | att | gtc | I | V | AF207757, AF333324, AJ507199, AY808030, AY808042, AY808043, |
| | | | | | DQ491971, DQ491972, DQ491973, DQ491974, DQ491975, |
| | | | | | DQ491976, DQ491977, DQ491978, EF407459, EF407465, |
| | | | | | EF407471, EF407494, EU155217, EU155221, EU155264, |
| | | | | | EU155329, EU155367, EU234062, EU256085, EU256103, |
| | | | | | EU781832, FJ478453, M58335 |
| 280 | att | gtt | I | V | AB285046, AB285056, AB285081, AF265145, AF265146, |
| | | | | | AF265147, AJ507173, AY808011, DQ492124, DQ492125, |
| | | | | | DQ492126, DQ492128, DQ492129, DQ492132, DQ492133, |
| | | | | | DQ492134, DQ492135, DQ492136, DQ492137, DQ492138, |
| | | | | | DQ492139, DQ492141, DQ492142, DQ492143, DQ492144, |
| | | | | | DQ492145, DQ492146, DQ492147, DQ492148, DQ492149, |
| | | | | | DQ492150, DQ492151, DQ492152, DQ492153, DQ492154, |
| | | | | | DQ492155, DQ492156, DQ492157, DQ492158, DQ492159, |
| | | | | | DQ492160, DQ492161, DQ492162, EF407473, EF407484, |
| | | | | | EF407486, EU155223, EU155225, EU155226, EU155229, |
| | | | | | EU155230, EU155263, EU155324, EU155330, EU155332, |
| | | | | | EU155334, EU155375, EU155376, EU256061, EU256084, |
| | | | | | EU256090, EU256091, EU482839, EU482877, EU660386, |
| | | | | | FJ958890, FJ958891, FJ958892, FJ958893, FJ958894, FJ958895, |
| | | | | | FJ958896, FJ958897, FJ958898, FJ958899, FJ958900, FJ958901, |
| | | | | | FJ958902, FJ958903, FJ958904, FJ958905, FJ958906, FJ958907, |
| | | | | | FJ958909, FJ958910, FJ958911, FJ958912, FJ958913, FJ958914, |
| | | | | | FJ958915, FJ958916, FJ958917, FJ958918, FJ958919, FJ958920, |
| | | | | | FJ958921, FJ958922, FJ958923, FJ958924, FJ958925, FJ958926, |
| | | | | | FJ958927, FJ958928, FJ958929, FJ958930, FJ958931, FJ958932, |
| | | | | | FJ958933, FJ958934, FJ958935 |
| 281 | ttg | atg | L | M | AB285043, DQ244140, HM042051 |
| 281 | ttg | cca | L | P | HM042059 |
| 281 | ttg | ccg | L | P | AM401947, DQ492017, EF407481 |
| 282 | gac | ggc | D | G | AM402299, DQ492094, EF638081 |
| 283 | tct | gcc | S | A | DQ491977 |
| 283 | tct | ccc | S | P | DQ491963 |
| 283 | tct | cct | S | P | AM401953, DQ492054, DQ492055 |
| 284 | ttc | tta | F | L | DQ492188 |
| 284 | ttc | ttg | F | L | AM402106 |
| 284 | ttc | tcc | F | S | DQ492004, DQ492116, DQ492145 |
| 284 | ttc | gtc | F | V | DQ492044, DQ492045, DQ492051, DQ492052 |
| 285 | gag | tgc | E | C | AY808034, DQ492008, DQ492009, DQ492010, DQ492011, |
| | | | | | DQ492012, DQ492013, DQ492014, DQ492015, DQ492016, |
| | | | | | DQ492017, DQ492018, DQ492019, DQ492020, DQ492021, |
| | | | | | DQ492022, DQ492023, DQ492024, DQ492025, DQ492026, |
| | | | | | DQ492027, DQ492028, DQ492029, DQ492030, DQ492031, |
| | | | | | DQ492032, DQ492033, DQ492034, DQ492035, DQ492036, |
| | | | | | DQ492037, DQ492038, DQ492039 |
| 285 | gag | gac | E | D | AB010249, AB036519, AB036520, AB036521, AB049087, |
| | | | | | AB049088, AB049089, AB049094, AB049096, AB049099, |
| | | | | | AB049100, AB080299, AB109543, AB191333, AB285035, |
| | | | | | AB285036, AB285037, AB285038, AB285039, AB285044, |
| | | | | | AB285048, AB285050, AB285051, AB285052, AB285053, |
| | | | | | AB285054, AB285055, AB285056, AB285057, AB285061, |
| | | | | | AB285064, AB285069, AB285070, AB285073, AB285075, |
| | | | | | AB285077, AB285078, AB285079, AB442220, AF034151, |
| | | | | | AF165045, AF165046, AF165047, AF165048, AF165049, AF165050, |
| | | | | | AF165051, AF165052, AF165053, AF165054, AF165055, AF165056, |
| | | | | | AF165057, AF165058, AF165059, AF165060, AF165062, AF165063, |
| | | | | | AF165064, AF176573, AF207755, AF207756, AF207758, AF207763, |
| | | | | | AF207764, AF207765, AF207768, AF207770, AF207771, AF207772, |
| | | | | | AF207774, AF208024, AF265142, AF265143, AF265144, AF265154, |
| | | | | | AF265155, AF265156, AF265158, AF265159, AF265160, AF265161, |
| | | | | | AF265162, AF333324, AF483269, AJ000009, AJ133096, AJ507156, |
| | | | | | AJ507159, AJ507162, AJ507163, AJ507164, AJ507165, AJ507166, |
| | | | | | AJ507167, AJ507168, AJ507169, AJ507170, AJ507174, AJ507175, |
| | | | | | AJ507177, AJ507178, AJ507179, AJ507180, AJ507181, AJ507182, |
| | | | | | AJ507183, AJ507184, AJ507188, AJ507189, AJ507190, AJ507191, |
| | | | | | AJ507192, AJ507194, AJ507197, AJ507198, AJ507199, AM401853, |
| | | | | | AM401864, AM401865, AM401866, AM401867, AM401868, |
| | | | | | AM401869, AM401870, AM401871, AM401872, AM401873, |
| | | | | | AM401874, AM401875, AM401876, AM401877, AM401878, |
| | | | | | AM401879, AM401880, AM401881, AM401882, AM401883, |
| | | | | | AM401884, AM401885, AM401886, AM401887, AM401888, |
| | | | | | AM401889, AM401890, AM401891, AM401892, AM401893, |
| | | | | | AM401894, AM401895, AM401896, AM401897, AM401898, |
| | | | | | AM401899, AM401900, AM401901, AM401902, AM401903, |
| | | | | | AM401904, AM401905, AM401906, AM401907, AM401908, |
| | | | | | AM401909, AM401910, AM401911, AM401912, AM401913, |
| | | | | | AM401914, AM401915, AM401943, AM401959, AM402055, |
| | | | | | AM402056, AM402057, AM402058, AM402059, AM402060, |
| | | | | | AM402061, AM402062, AM402063, AM402064, AM402065, |
| | | | | | AM402066, AM402067, AM402068, AM402069, AM402070, |
| | | | | | AM402071, AM402072, AM402073, AM402074, AM402075, |
| | | | | | AM402076, AM402077, AM402078, AM402079, AM402080, |
| | | | | | AM402081, AM402082, AM402083, AM402084, AM402085, |
| | | | | | AM402086, AM402087, AM402088, AM402089, AM402090, |
| | | | | | AM402091, AM402092, AM402093, AM402094, AM402095, |
| | | | | | AM402096, AM402097, AM402098, AM402099, AM402100, |
| | | | | | AM402101, AM402102, AM402103, AM402104, AM402105, |
| | | | | | AM402106, AM402107, AM402108, AM402109, AM402110, |
| | | | | | AM402111, AM402112, AM402113, AM402114, AM402115, |
| | | | | | AM402116, AM402117, AM402118, AM402119, AM402120, |
| | | | | | AM402121, AM402122, AM402123, AM402124, AM402125, |
| | | | | | AM402126, AM402127, AM402128, AM402129, AM402329, |
| | | | | | AM402330, AM402331, AM402332, AM402333, AM402334, |
| | | | | | AM402335, AM402336, AM402337, AM402338, AM402339, |
| | | | | | AM402340, AM402341, AM402342, AM402343, AM402344, |
| | | | | | AM402345, AM402346, AM402347, AM402348, AM402349, |
| | | | | | AM402350, AM402351, AM402352, AM402353, AM402354, |
| | | | | | AM402355, AM402356, AM402357, AM402358, AM402359, |
| | | | | | AM402360, AM402361, AM402362, AM402363, AM402364, |
| | | | | | AM402365, AM402367, AM402369, AM402370, AM402371, |
| | | | | | AM402372, AM402373, AM402374, AM402375, AM402376, |
| | | | | | AM402377, AM402379, AM402380, AM402381, AM402382, |
| | | | | | AM402383, AM402384, AM493489, AM493490, AM493491, |
| | | | | | AM493492, AM493493, AM493494, AM493495, AM493496, |
| | | | | | AM493497, AM493498, AM493499, AM493500, AM493501, |
| | | | | | AM493502, AM493503, AM493504, AM493505, AM493506, |
| | | | | | AM493507, AM494937, AY240967, AY240968, AY240969, |
| | | | | | AY460204, AY808009, AY808010, AY808012, AY808013, |
| | | | | | AY808014, AY808015, AY808016, AY808017, AY808018, |
| | | | | | AY808019, AY808021, AY808023, AY808024, AY808027, |
| | | | | | AY808029, AY808030, AY808031, AY808035, AY808037, |
| | | | | | AY808039, AY808040, AY808041, AY808043, D11168, D16435, |
| | | | | | D50480, D50482, D50483, D50484, D63857, D85516, D89872, |
| | | | | | DQ491943, DQ491944, DQ491945, DQ491946, DQ491947, |
| | | | | | DQ491948, DQ491949, DQ491950, DQ491951, DQ491952, |
| | | | | | DQ491953, DQ491954, DQ491955, DQ491956, DQ491957, |
| | | | | | DQ491958, DQ491959, DQ491960, DQ491961, DQ491962, |
| | | | | | DQ491963, DQ491964, DQ491965, DQ491966, DQ491967, |
| | | | | | DQ491968, DQ491969, DQ491970, DQ492040, DQ492041, |
| | | | | | DQ492042, DQ492043, DQ492044, DQ492045, DQ492046, |
| | | | | | DQ492047, DQ492048, DQ492049, DQ492050, DQ492051, |
| | | | | | DQ492052, DQ492053, DQ492054, DQ492055, DQ492056, |
| | | | | | DQ492057, DQ492058, DQ492059, DQ492060, DQ492061, |
| | | | | | DQ492062, DQ492063, DQ492064, DQ492065, DQ492066, |
| | | | | | DQ492067, DQ492068, DQ492069, DQ492070, DQ492071, |
| | | | | | DQ492072, DQ492073, DQ492074, DQ492075, DQ492076, |
| | | | | | DQ492077, DQ492078, DQ492079, DQ492080, DQ492081, |
| | | | | | DQ492082, DQ492083, DQ492084, DQ492085, DQ492086, |
| | | | | | DQ492087, DQ492088, DQ492089, DQ492090, DQ492091, |
| | | | | | DQ492092, DQ492093, DQ492094, DQ492095, DQ492096, |
| | | | | | DQ492097, DQ492098, DQ492099, DQ492100, DQ492101, |
| | | | | | DQ492102, DQ492103, DQ492104, DQ492105, DQ492106, |
| | | | | | DQ492107, DQ492108, DQ492109, DQ492110, DQ492111, |
| | | | | | DQ492112, DQ492113, DQ492114, DQ492115, DQ492116, |
| | | | | | DQ492117, DQ492118, DQ492119, DQ492120, DQ492121, |
| | | | | | DQ492122, DQ492123, DQ492163, DQ492164, DQ492165, |
| | | | | | DQ492166, DQ492167, DQ492168, DQ492169, DQ492170, |
| | | | | | DQ492172, DQ492173, DQ492174, DQ492175, DQ492176, |
| | | | | | DQ492177, DQ492178, DQ492179, DQ492180, DQ492181, |
| | | | | | DQ492182, DQ492183, DQ492184, DQ492185, DQ492186, |
| | | | | | DQ492187, DQ492188, DQ492189, DQ492190, DQ492191, |
| | | | | | DQ492192, DQ492193, DQ492194, DQ492195, DQ492196, |
| | | | | | DQ492197, DQ859970, EF032892, EF032893, EF032894, |
| | | | | | EF407460, EF407462, EF407464, EF407466, EF407474, EF407475, |
| | | | | | EF407476, EF407477, EF407478, EF407479, EF407482, EF407483, |
| | | | | | EF407488, EF407497, EF407498, EF407501, EF407502, EF407504, |
| | | | | | EF638081, EU155218, EU155219, EU155220, EU155222, |
| | | | | | EU155227, EU155228, EU155231, EU155235, EU155253, |
| | | | | | EU155254, EU155257, EU155259, EU155279, EU155302, |
| | | | | | EU155303, EU155307, EU155315, EU155316, EU155317, |
| | | | | | EU155325, EU155327, EU155331, EU155333, EU155335, |
| | | | | | EU155336, EU155337, EU155356, EU155357, EU155358, |
| | | | | | EU155360, EU155362, EU155363, EU155364, EU155365, |
| | | | | | EU155366, EU155367, EU155368, EU155369, EU155370, |
| | | | | | EU155371, EU155372, EU155373, EU155375, EU155377, |
| | | | | | EU155381, EU155382, EU255961, EU256000, EU256045, |
| | | | | | EU256059, EU256062, EU256065, EU256066, EU256075, |
| | | | | | EU256076, EU256077, EU256078, EU256079, EU256081, |
| | | | | | EU256082, EU256085, EU256089, EU256090, EU256092, |
| | | | | | EU256101, EU256103, EU482833, EU482859, EU482860, |
| | | | | | EU482874, EU482875, EU482879, EU482883, EU482885, |
| | | | | | EU482886, EU482888, EU781825, EU862837, FJ024277, FJ390396, |
| | | | | | FJ390397, FJ390398, FN435993, GQ913869, GU451218, |
| | | | | | GU451219, GU451220, GU451221, GU451222, GU451223, |
| | | | | | GU451224, HM042050, HM042051, HM042052, HM042054, |
| | | | | | HM042055, HM042056, HM042057, HM042061, HM042063, L02836, |
| | | | | | M58335, M84754, M96362, U01214, U16362, U45476 |
| 285 | gag | gat | E | D | AB049091, AB049095, AB049098, AB249644, AB285047, |
| | | | | | AB285060, AB285062, AB285066, AB285068, AB285072, |
| | | | | | AB426117, AB442219, AB442221, AB442222, AF033373, |
| | | | | | AF165061, AF207752, AF207753, AF207754, AJ132996, AJ132997, |
| | | | | | AJ507155, AJ507157, AJ507158, AJ507160, AJ507161, AJ507171, |
| | | | | | AJ507172, AJ507186, AJ507193, AM401848, AM401849, |
| | | | | | AM401850, AM401851, AM401852, AM401854, AM401855, |
| | | | | | AM401856, AM401857, AM401858, AM401859, AM401860, |
| | | | | | AM401861, AM401862, AM401863, AM401916, AM401917, |
| | | | | | AM401918, AM401919, AM401920, AM401921, AM401922, |
| | | | | | AM401923, AM401924, AM401925, AM401926, AM401927, |
| | | | | | AM401928, AM401929, AM401930, AM401931, AM401932, |
| | | | | | AM401933, AM401934, AM401935, AM401936, AM401937, |
| | | | | | AM401938, AM401939, AM401940, AM401941, AM401942, |
| | | | | | AM401944, AM401945, AM401946, AM401947, AM401948, |
| | | | | | AM401949, AM401950, AM401951, AM401952, AM401953, |
| | | | | | AM401954, AM401955, AM401956, AM401957, AM401958, |
| | | | | | AM401960, AM401961, AM401962, AM401963, AM401964, |
| | | | | | AM401965, AM401966, AM401967, AM401968, AM401969, |
| | | | | | AM401970, AM401971, AM401972, AM401973, AM401974, |
| | | | | | AM401975, AM401976, AM401977, AM401978, AM401979, |
| | | | | | AM401980, AM402258, AM402259, AM402260, AM402261, |
| | | | | | AM402262, AM402263, AM402264, AM402265, AM402266, |
| | | | | | AM402267, AM402268, AM402269, AM402270, AM402271, |
| | | | | | AM402272, AM402273, AM402274, AM402275, AM402276, |
| | | | | | AM402277, AM402278, AM402279, AM402280, AM402281, |
| | | | | | AM402282, AM402283, AM402284, AM402285, AM402286, |
| | | | | | AM402287, AM402288, AM402289, AM402290, AM402291, |
| | | | | | AM402292, AM402293, AM402294, AM402295, AM402296, |
| | | | | | AM402297, AM402298, AM402299, AM402300, AM402301, |
| | | | | | AM402302, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402326, |
| | | | | | AM402327, AM402328, AM493436, AM493437, AM493438, |
| | | | | | AM493439, AM493440, AM493441, AM493442, AM493443, |
| | | | | | AM493444, AM493445, AM493446, AM493447, AM493448, |
| | | | | | AM493449, AM493450, AM493451, AM493471, AM493472, |
| | | | | | AM493473, AM493474, AM493475, AM493476, AM493477, |
| | | | | | AM493478, AM493479, AM493480, AM493481, AM493482, |
| | | | | | AM493483, AM493484, AM493485, AM493486, AM493487, |
| | | | | | AM493488, AY045702, AY808025, AY808032, AY808033, |
| | | | | | AY808036, AY808042, D90208, DQ244140, DQ244141, DQ491971, |
| | | | | | DQ491972, DQ491973, DQ491974, DQ491975, DQ491976, |
| | | | | | DQ491977, DQ491978, EF407468, EF407485, EF407487, |
| | | | | | EF407489, EF407491, EU155232, EU155256, EU155262, |
| | | | | | EU155318, EU155359, EU155361, EU255960, EU256064, |
| | | | | | EU256080, EU256083, EU256084, EU256088, EU256099, |
| | | | | | EU256102, EU482880, EU781827, EU781830, GQ913873 |
| 285 | gag | aac | E | N | DQ492171 |
| 285 | gag | acc | E | T | AB285059 |
| 285 | gag | gta | E | V | HM042059 |
| 285 | gag | gtc | E | V | AF207769, AM402378, EF407461, EU529682, EU862835, |
| | | | | | HM042058 |
| 285 | gag | gtg | E | V | EF407481 |
| 286 | ccg | aag | P | K | AB285054, AB285055, AB285073 |
| 286 | ccg | ctg | P | L | AM402076, DQ491989, DQ492053 |
| 286 | ccg | cag | P | Q | AB049096, AM494937 |
| 286 | ccg | tcg | P | S | AF165049, AF165050, EU256065, EU482886 |
| 287 | ctc | tgt | L | C | AB049101 |
| 287 | ctc | ttt | L | F | AM402277, HM042062 |
| 287 | ctc | att | L | I | AB285037, AF165053, AF165054, AF207772, D14484, D90208 |
| 287 | ctc | cct | L | P | AM401933 |
| 288 | caa | cac | Q | H | AF207756 |
| 288 | caa | cat | Q | H | AB049098, AF034151, AY808026, D85516, EF407490, EU155302, |
| | | | | | EU482874, HM042053 |
| 288 | caa | aat | Q | N | EU155258 |
| 288 | caa | aga | Q | R | AB154189, AB154190, AJ507166, AJ507169, AM402064, EU155331 |
| 288 | caa | cga | Q | R | AB010249, AB010250, AB036519, AB036520, AB036521, |
| | | | | | AB049089, AB049092, AB049094, AB049095, AB049099, |
| | | | | | AB049101, AB080299, AB109543, AB154177, AB154178, |
| | | | | | AB154179, AB154180, AB154181, AB154182, AB154183, |
| | | | | | AB154184, AB154185, AB154186, AB154187, AB154188, |
| | | | | | AB154191, AB154194, AB154195, AB154196, AB154197, |
| | | | | | AB154198, AB154199, AB154200, AB154201, AB154202, |
| | | | | | AB154203, AB154204, AB154205, AB154206, AB191333, |
| | | | | | AB249644, AB285036, AB285037, AB285038, AB285039, |
| | | | | | AB285040, AB285047, AB285048, AB285050, AB285051, |
| | | | | | AB285052, AB285053, AB285056, AB285057, AB285058, |
| | | | | | AB285059, AB285061, AB285063, AB285064, AB285065, |
| | | | | | AB285067, AB285068, AB285069, AB285070, AB285071, |
| | | | | | AB285072, AB285073, AB285074, AB285075, AB285078, |
| | | | | | AB285079, AB285081, AB426117, AB442219, AB442220, |
| | | | | | AB442221, AB442222, AF033373, AF165045, AF165046, AF165047, |
| | | | | | AF165048, AF165049, AF165050, AF165051, AF165052, AF165053, |
| | | | | | AF165054, AF165055, AF165056, AF165057, AF165058, AF165059, |
| | | | | | AF165060, AF165061, AF165062, AF165063, AF165064, AF176573, |
| | | | | | AF207752, AF207754, AF207755, AF207757, AF207758, AF207762, |
| | | | | | AF207763, AF207764, AF207765, AF207766, AF207768, AF207769, |
| | | | | | AF207770, AF207771, AF207772, AF207774, AF208024, AF265142, |
| | | | | | AF265143, AF265144, AF265145, AF265146, AF265147, AF265152, |
| | | | | | AF265153, AF265154, AF265155, AF265156, AF265158, AF265159, |
| | | | | | AF265160, AF265161, AF265162, AF265163, AF313916, AF333324, |
| | | | | | AF356827, AF483269, AJ000009, AJ132996, AJ132997, AJ238800, |
| | | | | | AJ507156, AJ507157, AJ507158, AJ507159, AJ507162, AJ507163, |
| | | | | | AJ507164, AJ507165, AJ507167, AJ507168, AJ507170, AJ507171, |
| | | | | | AJ507173, AJ507175, AJ507178, AJ507179, AJ507180, AJ507181, |
| | | | | | AJ507182, AJ507185, AJ507186, AJ507189, AJ507190, AJ507191, |
| | | | | | AJ507192, AJ507193, AJ507194, AJ507195, AJ507196, AJ507197, |
| | | | | | AJ507198, AJ507199, AM401848, AM401849, AM401850, |
| | | | | | AM401851, AM401852, AM401853, AM401854, AM401855, |
| | | | | | AM401856, AM401857, AM401858, AM401859, AM401860, |
| | | | | | AM401861, AM401862, AM401863, AM401864, AM401865, |
| | | | | | AM401866, AM401867, AM401868, AM401869, AM401870, |
| | | | | | AM401871, AM401872, AM401873, AM401874, AM401875, |
| | | | | | AM401876, AM401877, AM401878, AM401879, AM401880, |
| | | | | | AM401881, AM401882, AM401883, AM401884, AM401885, |
| | | | | | AM401886, AM401887, AM401888, AM401889, AM401890, |
| | | | | | AM401891, AM401892, AM401893, AM401894, AM401895, |
| | | | | | AM401896, AM401897, AM401898, AM401899, AM401900, |
| | | | | | AM401901, AM401902, AM401903, AM401904, AM401905, |
| | | | | | AM401906, AM401907, AM401908, AM401909, AM401910, |
| | | | | | AM401911, AM401912, AM401913, AM401914, AM401915, |
| | | | | | AM401916, AM401917, AM401918, AM401919, AM401920, |
| | | | | | AM401921, AM401922, AM401923, AM401924, AM401925, |
| | | | | | AM401926, AM401927, AM401928, AM401929, AM401930, |
| | | | | | AM401931, AM401932, AM401933, AM401934, AM401935, |
| | | | | | AM401936, AM401937, AM401938, AM401939, AM401940, |
| | | | | | AM401941, AM401942, AM401943, AM401944, AM401945, |
| | | | | | AM401946, AM401947, AM401948, AM401949, AM401950, |
| | | | | | AM401951, AM401952, AM401953, AM401954, AM401956, |
| | | | | | AM401957, AM401958, AM401959, AM401960, AM401961, |
| | | | | | AM401962, AM401963, AM401964, AM401965, AM401966, |
| | | | | | AM401967, AM401968, AM401969, AM401970, AM401971, |
| | | | | | AM401972, AM401973, AM401974, AM401975, AM401976, |
| | | | | | AM401977, AM401978, AM401979, AM401980, AM402055, |
| | | | | | AM402056, AM402057, AM402058, AM402059, AM402060, |
| | | | | | AM402061, AM402062, AM402063, AM402065, AM402066, |
| | | | | | AM402067, AM402068, AM402069, AM402070, AM402071, |
| | | | | | AM402072, AM402073, AM402074, AM402075, AM402076, |
| | | | | | AM402077, AM402078, AM402079, AM402080, AM402081, |
| | | | | | AM402082, AM402083, AM402084, AM402085, AM402086, |
| | | | | | AM402087, AM402088, AM402089, AM402090, AM402091, |
| | | | | | AM402092, AM402093, AM402094, AM402095, AM402096, |
| | | | | | AM402097, AM402098, AM402099, AM402100, AM402101, |
| | | | | | AM402102, AM402103, AM402104, AM402105, AM402106, |
| | | | | | AM402107, AM402108, AM402109, AM402110, AM402111, |
| | | | | | AM402112, AM402113, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AM402329, AM402330, |
| | | | | | AM402331, AM402332, AM402333, AM402334, AM402335, |
| | | | | | AM402336, AM402337, AM402338, AM402339, AM402340, |
| | | | | | AM402341, AM402342, AM402343, AM402344, AM402345, |
| | | | | | AM402346, AM402347, AM402348, AM402349, AM402350, |
| | | | | | AM402351, AM402352, AM402353, AM402354, AM402355, |
| | | | | | AM402356, AM402357, AM402358, AM402359, AM402360, |
| | | | | | AM402361, AM402362, AM402363, AM402364, AM402365, |
| | | | | | AM402367, AM402369, AM402370, AM402371, AM402372, |
| | | | | | AM402373, AM402374, AM402375, AM402376, AM402377, |
| | | | | | AM402378, AM402379, AM402380, AM402381, AM402382, |
| | | | | | AM402383, AM402384, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, |
| | | | | | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507, AM494937, |
| | | | | | AY008261, AY044867, AY045702, AY240967, AY240968, |
| | | | | | AY240969, AY460204, AY587016, AY587844, AY808009, |
| | | | | | AY808010, AY808011, AY808012, AY808014, AY808017, |
| | | | | | AY808018, AY808019, AY808021, AY808023, AY808024, |
| | | | | | AY808025, AY808027, AY808028, AY808029, AY808030, |
| | | | | | AY808032, AY808033, AY808035, AY808036, AY808037, |
| | | | | | AY808038, AY808039, AY808040, AY808041, AY808042, |
| | | | | | AY808043, D10934, D11168, D14484, D16435, D30613, D45172, |
| | | | | | D50480, D50481, D50482, D50483, D50484, D50485, D63857, |
| | | | | | D89815, D89872, DQ244140, DQ244141, DQ491943, DQ491944, |
| | | | | | DQ491945, DQ491946, DQ491947, DQ491948, DQ491949, |
| | | | | | DQ491950, DQ491951, DQ491952, DQ491953, DQ491954, |
| | | | | | DQ491955, DQ491956, DQ491957, DQ491958, DQ491959, |
| | | | | | DQ491960, DQ491961, DQ491962, DQ491963, DQ491964, |
| | | | | | DQ491965, DQ491966, DQ491967, DQ491968, DQ491969, |
| | | | | | DQ491970, DQ491971, DQ491972, DQ491973, DQ491974, |
| | | | | | DQ491975, DQ491976, DQ491977, DQ491978, DQ491979, |
| | | | | | DQ491980, DQ491981, DQ491982, DQ491983, DQ491984, |
| | | | | | DQ491985, DQ491986, DQ491987, DQ491988, DQ491989, |
| | | | | | DQ491990, DQ491991, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, DQ492040, DQ492041, DQ492042, |
| | | | | | DQ492043, DQ492044, DQ492045, DQ492046, DQ492047, |
| | | | | | DQ492048, DQ492049, DQ492050, DQ492051, DQ492052, |
| | | | | | DQ492053, DQ492054, DQ492055, DQ492056, DQ492057, |
| | | | | | DQ492058, DQ492059, DQ492060, DQ492061, DQ492062, |
| | | | | | DQ492063, DQ492064, DQ492065, DQ492066, DQ492067, |
| | | | | | DQ492068, DQ492069, DQ492070, DQ492071, DQ492072, |
| | | | | | DQ492073, DQ492074, DQ492075, DQ492076, DQ492077, |
| | | | | | DQ492078, DQ492079, DQ492080, DQ492081, DQ492082, |
| | | | | | DQ492083, DQ492084, DQ492085, DQ492086, DQ492087, |
| | | | | | DQ492088, DQ492089, DQ492090, DQ492091, DQ492092, |
| | | | | | DQ492093, DQ492094, DQ492095, DQ492096, DQ492097, |
| | | | | | DQ492098, DQ492099, DQ492100, DQ492101, DQ492102, |
| | | | | | DQ492103, DQ492104, DQ492105, DQ492106, DQ492107, |
| | | | | | DQ492108, DQ492109, DQ492110, DQ492111, DQ492112, |
| | | | | | DQ492113, DQ492114, DQ492115, DQ492116, DQ492117, |
| | | | | | DQ492118, DQ492119, DQ492120, DQ492121, DQ492122, |
| | | | | | DQ492123, DQ492124, DQ492125, DQ492126, DQ492127, |
| | | | | | DQ492128, DQ492129, DQ492130, DQ492131, DQ492132, |
| | | | | | DQ492133, DQ492134, DQ492135, DQ492136, DQ492137, |
| | | | | | DQ492138, DQ492139, DQ492140, DQ492141, DQ492142, |
| | | | | | DQ492143, DQ492144, DQ492145, DQ492146, DQ492147, |
| | | | | | DQ492148, DQ492149, DQ492150, DQ492151, DQ492152, |
| | | | | | DQ492153, DQ492154, DQ492155, DQ492156, DQ492157, |
| | | | | | DQ492158, DQ492159, DQ492160, DQ492161, DQ492162, |
| | | | | | DQ492163, DQ492164, DQ492165, DQ492166, DQ492167, |
| | | | | | DQ492168, DQ492169, DQ492170, DQ492171, DQ492173, |
| | | | | | DQ492174, DQ492175, DQ492176, DQ492177, DQ492179, |
| | | | | | DQ492180, DQ492181, DQ492182, DQ492183, DQ492184, |
| | | | | | DQ492185, DQ492186, DQ492187, DQ492188, DQ492189, |
| | | | | | DQ492190, DQ492191, DQ492192, DQ492193, DQ492194, |
| | | | | | DQ492195, DQ492196, DQ492197, DQ859970, EF032892, |
| | | | | | EF032893, EF032894, EF407459, EF407460, EF407461, EF407462, |
| | | | | | EF407463, EF407464, EF407465, EF407466, EF407468, EF407469, |
| | | | | | EF407470, EF407471, EF407472, EF407473, EF407475, EF407476, |
| | | | | | EF407477, EF407478, EF407480, EF407481, EF407482, EF407484, |
| | | | | | EF407485, EF407488, EF407489, EF407491, EF407492, EF407495, |
| | | | | | EF407497, EF407498, EF407499, EF407500, EF407501, EF407502, |
| | | | | | EF638081, EU155217, EU155218, EU155219, EU155221, |
| | | | | | EU155222, EU155223, EU155224, EU155226, EU155227, |
| | | | | | EU155228, EU155229, EU155230, EU155231, EU155232, |
| | | | | | EU155235, EU155253, EU155254, EU155256, EU155257, |
| | | | | | EU155259, EU155261, EU155262, EU155263, EU155264, |
| | | | | | EU155279, EU155280, EU155281, EU155303, EU155304, |
| | | | | | EU155306, EU155307, EU155315, EU155317, EU155318, |
| | | | | | EU155325, EU155326, EU155327, EU155328, EU155329, |
| | | | | | EU155330, EU155332, EU155334, EU155335, EU155336, |
| | | | | | EU155337, EU155356, EU155357, EU155358, EU155360, |
| | | | | | EU155361, EU155362, EU155364, EU155365, EU155366, |
| | | | | | EU155367, EU155368, EU155369, EU155370, EU155371, |
| | | | | | EU155372, EU155373, EU155375, EU155376, EU155377, |
| | | | | | EU155381, EU155382, EU234061, EU234062, EU255960, |
| | | | | | EU255961, EU255962, EU256000, EU256001, EU256045, |
| | | | | | EU256059, EU256061, EU256062, EU256064, EU256065, |
| | | | | | EU256075, EU256076, EU256079, EU256080, EU256081, |
| | | | | | EU256082, EU256083, EU256084, EU256085, EU256088, |
| | | | | | EU256089, EU256090, EU256101, EU256102, EU256103, |
| | | | | | EU482833, EU482839, EU482849, EU482859, EU482860, |
| | | | | | EU482875, EU482877, EU482883, EU482885, EU482886, |
| | | | | | EU482888, EU529682, EU660386, EU781825, EU781827, |
| | | | | | EU781830, EU781832, EU857431, EU862835, FJ024277, FJ390396, |
| | | | | | FJ390397, FJ390398, FJ478453, FJ958890, FJ958891, FJ958892, |
| | | | | | FJ958893, FJ958894, FJ958895, FJ958896, FJ958897, FJ958898, |
| | | | | | FJ958899, FJ958900, FJ958901, FJ958902, FJ958903, FJ958904, |
| | | | | | FJ958905, FJ958906, FJ958909, FJ958910, FJ958911, FJ958912, |
| | | | | | FJ958913, FJ958914, FJ958915, FJ958916, FJ958917, FJ958918, |
| | | | | | FJ958919, FJ958920, FJ958921, FJ958922, FJ958923, FJ958924, |
| | | | | | FJ958925, FJ958926, FJ958927, FJ958928, FJ958929, FJ958930, |
| | | | | | FJ958931, FJ958932, FJ958933, FJ958934, FJ958935, GQ913868, |
| | | | | | GQ913869, GQ913870, GQ913871, GQ913873, GU451218, |
| | | | | | GU451219, GU451220, GU451221, GU451222, GU451223, |
| | | | | | GU451224, HM042050, HM042051, HM042052, HM042054, |
| | | | | | HM042055, HM042056, HM042057, HM042058, HM042059, |
| | | | | | HM042061, HM042062, HM042063, M58335, M84754, M96362, |
| | | | | | U01214, U16362, U45476 |
| 288 | caa | cgc | Q | R | AB285066, AB285077, AJ507174, AJ507177, AJ507183, AM402258, |
| | | | | | AM402259, AM402260, AM402261, AM402262, AM402263, |
| | | | | | AM402264, AM402265, AM402266, AM402267, AM402268, |
| | | | | | AM402269, AM402270, AM402271, AM402272, AM402273, |
| | | | | | AM402274, AM402275, AM402276, AM402277, AM402278, |
| | | | | | AM402279, AM402280, AM402281, AM402282, AM402283, |
| | | | | | AM402284, AM402285, AM402286, AM402287, AM402288, |
| | | | | | AM402289, AM402290, AM402291, AM402292, AM402293, |
| | | | | | AM402294, AM402295, AM402296, AM402297, AM402298, |
| | | | | | AM402299, AM402300, AM402301, AM402302, AM402303, |
| | | | | | AM402304, AM402305, AM402306, AM402307, AM402308, |
| | | | | | AM402309, AM402310, AM402311, AM402312, AM402313, |
| | | | | | AM402314, AM402315, AM402316, AM402317, AM402318, |
| | | | | | AM402319, AM402320, AM402321, AM402322, AM402323, |
| | | | | | AM402324, AM402325, AM402326, AM402327, AM402328, |
| | | | | | AM493436, AM493437, AM493438, AM493439, AM493440, |
| | | | | | AM493441, AM493442, AM493443, AM493444, AM493445, |
| | | | | | AM493446, AM493447, AM493448, AM493449, AM493450, |
| | | | | | AM493451, EF407483, EU155220, EU155260 |
| 288 | caa | cgg | Q | R | AB049087, AB049088, AB049090, AB049091, AB049096, |
| | | | | | AB285043, AB285046, AB285060, AB285062, AB285076, |
| | | | | | AB429050, AF207753, AF207760, AF265148, AF265149, AF265150, |
| | | | | | AF265164, AF265165, AJ133096, AJ507160, AJ507161, AJ507172, |
| | | | | | AJ507188, AY808016, AY808031, D90208, DQ491992, DQ492172, |
| | | | | | EF407467, EF407474, EF407486, EF407496, EU155324, |
| | | | | | EU155359, EU155363, EU256091, EU256092, EU256099, |
| | | | | | EU482880, EU660388, EU862837, FJ958907, L02836, X61596 |
| 288 | caa | cgt | Q | R | AB049100, AM401955, DQ492178, EF407479, EU155225, |
| | | | | | EU155316, EU155333, EU256077, GQ913872 |
| 288 | caa | gta | Q | V | AB285044 |
| 288 | caa | gtg | Q | V | AJ507155 |
| 288 | caa | tgg | Q | W | EU155305 |
| 288 | caa | tat | Q | Y | AJ507184 |
| 289 | gcg | ccg | A | P | AB285043, AM402086 |
| 289 | gcg | aca | A | T | AF207769 |
| 289 | gcg | acg | A | T | HM042062 |
| 289 | gcg | gta | A | V | HM042059 |
| 289 | gcg | gtg | A | V | AF165062, AY008261, DQ492017, EU155281, HM042056 |
| 290 | gag | gca | E | A | AJ507187 |
| 290 | gag | gac | E | D | EU256064 |
| 290 | gag | ggg | E | G | AM402303, AY808023, DQ492002, DQ492105 |
| 290 | gag | aag | E | K | AM401951 |
| 290 | gag | gtg | E | V | D90208 |
| 291 | gag | gca | E | A | EU155376 |
| 291 | gag | gcg | E | A | EU155218 |
| 291 | gag | gat | E | D | EU482879, M96362, U16362 |
| 291 | gag | ggg | E | G | AM401861, AM401870, AM401969, DQ492039, DQ492105 |
| 291 | gag | aaa | E | K | AJ507158 |
| 291 | gag | aag | E | K | AY808040, DQ491943, DQ491944, DQ491945, DQ491946, |
| | | | | | DQ491947, DQ491948, DQ491949, DQ491950, DQ491951, |
| | | | | | DQ491952, DQ491953, DQ491954, DQ491955, DQ491956, |
| | | | | | DQ491957 |
| 291 | gag | gtg | E | V | EF407494 |
| 292 | gat | tgt | D | C | EU155302 |
| 292 | gat | gaa | D | E | EF407496 |
| 292 | gat | gag | D | E | AF207757, EU256061 |
| 292 | gat | ggt | D | G | AF265158, AF265159, DQ492120, EF638081, HM042062, U01214 |
| 292 | gat | aat | D | N | AB285040, AF207755, EU155304, EU256092 |
| 292 | gat | agt | D | S | AB285079, AJ507180, HM042056 |
| 293 | gag | gac | E | D | AB285051, DQ244141, EF407498, EU155218, EU155258 |
| 293 | gag | gat | E | D | AB285072, AY460204, AY808038, AY808043, DQ492148, |
| | | | | | EF407482, EF407483, EF407486, EF638081, EU155220, |
| | | | | | EU781830, EU862835, GU451221, GU451222, GU451223, |
| | | | | | GU451224 |
| 293 | gag | gga | E | G | AY808041, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, EU155228 |
| 293 | gag | ggg | E | G | AB049092, AJ507159, AM402327, AM402337, AM402352, |
| | | | | | AY808031 |
| 293 | gag | cag | E | Q | AY808042, DQ491971, DQ491972, DQ491973, DQ491974, |
| | | | | | DQ491975, DQ491976, DQ491977 |
| 293 | gag | cgg | E | R | DQ491978, EU256079 |
| 294 | agg | gga | R | G | EU256065 |
| 294 | agg | ggc | R | G | AJ507185 |
| 294 | agg | ggg | R | G | AB285076, AB442221, AF207760, AF265148, AF265150, AF265164, |
| | | | | | AF265165, AJ507168, AY240969, AY808041, DQ491958, |
| | | | | | DQ491959, DQ491960, DQ491961, DQ491962, DQ491963, |
| | | | | | DQ491964, DQ491965, DQ491966, DQ491967, DQ491968, |
| | | | | | DQ491969, DQ491970, DQ491988, DQ492072, DQ492114, |
| | | | | | EF407460, EF407467, EU155262, EU155336, EU155375, |
| | | | | | EU155381, EU256082, EU256084, EU256101, EU660386, |
| | | | | | HM042062, M96362, U01214, U16362 |
| 294 | agg | aag | R | K | AB285043, AF034151, DQ492085, DQ859970, EU155372, |
| | | | | | EU482877 |
| 294 | agg | atg | R | M | HM042056 |
| 294 | agg | cag | R | Q | AM494937, EU256092 |
| 294 | agg | acg | R | T | HM042051 |
| 295 | gaa | gac | E | D | AM402110 |
| 295 | gaa | gga | E | G | AM493496, DQ492136, DQ492149 |
| 295 | gaa | ggg | E | G | FJ958916 |
| 296 | gta | gag | V | E | AY808028 |
| 296 | gta | ggg | V | G | AM401848 |
| 296 | gta | ata | V | I | AB049096, AB154203, AB154204, AB285038, AB285040, |
| | | | | | AB285054, AB285055, AB285059, AB285073, AB285075, |
| | | | | | AB442221, AF207752, AF207753, AF207760, AF207761, AF265148, |
| | | | | | AF265149, AF265150, AF265154, AF265155, AF265156, AF265158, |
| | | | | | AF265159, AF265163, AF265164, AF265165, AJ132996, AJ132997, |
| | | | | | AJ507166, AJ507168, AJ507169, AJ507179, AJ507193, AJ507196, |
| | | | | | AM493471, AM493472, AM493473, AM493474, AM493475, |
| | | | | | AM493476, AM493477, AM493478, AM493479, AM493480, |
| | | | | | AM493481, AM493482, AM493483, AM493484, AM493485, |
| | | | | | AM493486, AM493487, AM493488, AY240968, AY587844, |
| | | | | | AY808009, AY808014, AY808020, AY808021, AY808029, |
| | | | | | AY808035, AY808036, AY808040, D90208, DQ244140, DQ491943, |
| | | | | | DQ491944, DQ491945, DQ491946, DQ491947, DQ491948, |
| | | | | | DQ491949, DQ491950, DQ491951, DQ491952, DQ491953, |
| | | | | | DQ491954, DQ491955, DQ491956, DQ491957, DQ492040, |
| | | | | | DQ492041, DQ492042, DQ492043, DQ492044, DQ492045, |
| | | | | | DQ492046, DQ492047, DQ492048, DQ492049, DQ492050, |
| | | | | | DQ492051, DQ492052, DQ492053, DQ492054, DQ492055, |
| | | | | | DQ492056, DQ492057, DQ492058, DQ492059, DQ492060, |
| | | | | | DQ492061, DQ492062, DQ492063, DQ492064, DQ492065, |
| | | | | | DQ492066, DQ492067, DQ492068, DQ492069, DQ492070, |
| | | | | | DQ492071, DQ492072, DQ492073, DQ492074, DQ492075, |
| | | | | | DQ492076, DQ492077, DQ492078, DQ492079, DQ492080, |
| | | | | | DQ492081, DQ492083, DQ492084, DQ492085, DQ492087, |
| | | | | | DQ492088, DQ492089, DQ492090, DQ492091, DQ492092, |
| | | | | | DQ492093, DQ492163, DQ492164, DQ492165, DQ492166, |
| | | | | | DQ492167, DQ492168, DQ492169, DQ492170, DQ492171, |
| | | | | | DQ492172, DQ492173, DQ492174, DQ492175, DQ492176, |
| | | | | | DQ492177, DQ492178, DQ492179, DQ492180, DQ492181, |
| | | | | | DQ492182, DQ492183, DQ492184, DQ492185, DQ492186, |
| | | | | | DQ492187, DQ492188, DQ492189, DQ492190, DQ492191, |
| | | | | | DQ492192, DQ492193, DQ492194, DQ492195, DQ492196, |
| | | | | | DQ492197, EF407460, EF407462, EF407464, EF407473, |
| | | | | | EF407494, EF407500, EU155218, EU155254, EU155256, |
| | | | | | EU155280, EU155326, EU155327, EU155329, EU155330, |
| | | | | | EU155331, EU155364, EU155365, EU155373, EU155375, |
| | | | | | EU155376, EU256075, EU256078, EU256079, EU256082, |
| | | | | | EU256084, EU256103, EU482833, EU482877, EU482879, |
| | | | | | EU482883, EU529682, EU660386, GU451220, HM042050, M96362, |
| | | | | | U16362 |
| 296 | gta | atc | V | I | AJ507155, HM042055, HM042058 |
| 296 | gta | ttg | V | L | DQ859970, EU155279 |
| 296 | gta | atg | V | M | AB285065, AJ507170, AJ507178, AJ507186, AJ507199, AM402258, |
| | | | | | AM402259, AM402260, AM402261, AM402262, AM402263, |
| | | | | | AM402264, AM402265, AM402266, AM402267, AM402268, |
| | | | | | AM402269, AM402270, AM402271, AM402272, AM402273, |
| | | | | | AM402274, AM402275, AM402276, AM402277, AM402278, |
| | | | | | AM402279, AM402280, AM402281, AM402282, AM402283, |
| | | | | | AM402284, AM402285, AM402286, AM402287, AM402288, |
| | | | | | AM402289, AM402290, AM402291, AM402292, AM402293, |
| | | | | | AM402294, AM402295, AM402296, AM402297, AM402298, |
| | | | | | AM402299, AM402300, AM402301, AM402302, AM402303, |
| | | | | | AM402304, AM402305, AM402306, AM402307, AM402308, |
| | | | | | AM402309, AM402310, AM402311, AM402312, AM402313, |
| | | | | | AM402314, AM402315, AM402316, AM402317, AM402318, |
| | | | | | AM402319, AM402320, AM402321, AM402322, AM402323, |
| | | | | | AM402324, AM402325, AM402326, AM402327, AM402328, |
| | | | | | AM493436, AM493437, AM493438, AM493439, AM493440, |
| | | | | | AM493441, AM493442, AM493443, AM493444, AM493445, |
| | | | | | AM493446, AM493447, AM493448, AM493449, AM493450, |
| | | | | | AM493451, AY240967, AY808033, DQ491989, EU155228, |
| | | | | | EU155229, EU155306, GQ913869, GQ913873, HM042051, |
| | | | | | HM042052, HM042054, HM042056, HM042061, HM042062, |
| | | | | | HM042063 |
| 296 | gta | cca | V | P | AF165049, AF165050, AF165051, AF165052, AF165057, AF165058, |
| | | | | | AF165061, AF165062, AF207762, AF207765, AF207770, AY045702 |
| 296 | gta | ccg | V | P | EU256059 |
| 296 | gta | aca | V | T | DQ492086 |
| 297 | tcc | ttc | S | F | DQ492025, DQ492033 |
| 298 | gtt | gcc | V | A | DQ491948 |
| 298 | gtt | gct | V | A | D89815, DQ491965 |
| 298 | gtt | ata | V | I | EU155318 |
| 298 | gtt | atc | V | I | AM402068, AY808016, AY808028, DQ491979, DQ491980, |
| | | | | | DQ491981, DQ491982, DQ491983, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, EF407482, EU155224, EU155335, |
| | | | | | EU256081, EU256088, EU256101 |
| 298 | gtt | att | V | I | AB285061, AF165063, AF165064, AJ507158, AJ507191, AM402055, |
| | | | | | AM402056, AM402057, AM402058, AM402059, AM402060, |
| | | | | | AM402061, AM402062, AM402063, AM402064, AM402065, |
| | | | | | AM402066, AM402067, AM402069, AM402070, AM402071, |
| | | | | | AM402072, AM402073, AM402074, AM402075, AM402076, |
| | | | | | AM402077, AM402078, AM402079, AM402080, AM402081, |
| | | | | | AM402082, AM402083, AM402084, AM402085, AM402086, |
| | | | | | AM402087, AM402088, AM402089, AM402090, AM402091, |
| | | | | | AM402092, AM402093, AM402094, AM402095, AM402096, |
| | | | | | AM402097, AM402098, AM402099, AM402100, AM402101, |
| | | | | | AM402102, AM402103, AM402104, AM402105, AM402106, |
| | | | | | AM402107, AM402108, AM402109, AM402110, AM402111, |
| | | | | | AM402112, AM402113, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AY808030, AY808033, |
| | | | | | AY808043, D50480, D50483, EF407474, EF407475, EF407478, |
| | | | | | EF407488, EU155219, EU155221, EU155303, EU155307, |
| | | | | | EU155315, EU155361, EU155367, EU256045, EU256085, |
| | | | | | EU482859, EU482880, EU862835, FJ390396 |
| 298 | gtt | ctc | V | L | D50485 |
| 298 | gtt | ctt | V | L | D50481 |
| 298 | gtt | atg | V | M | EF407464 |
| 298 | gtt | act | V | T | EU155365, EU529682 |
| 299 | ccg | gca | P | A | AB010249, AB285043, AB285053, AF207761, AM401911, |
| | | | | | AY008261, AY240967, AY460204, D11168, D16435, D89872, |
| | | | | | EF638081, EU155279, EU256090, EU256102, GU451221, |
| | | | | | GU451222, GU451223, GU451224 |
| 299 | ccg | gcg | P | A | AB010250, AB049087, AB049090, AB049094, AB049095, |
| | | | | | AB049098, AB049100, AB049101, AB080299, AB249644, |
| | | | | | AB285035, AB285037, AB285040, AB285042, AB285044, |
| | | | | | AB285046, AB285048, AB285054, AB285055, AB285056, |
| | | | | | AB285057, AB285058, AB285060, AB285061, AB285063, |
| | | | | | AB285068, AB285069, AB285075, AB285077, AB285078, |
| | | | | | AB285079, AB426117, AB429050, AB442219, AB442220, |
| | | | | | AB442221, AF165045, AF165046, AF165047, AF165048, AF165053, |
| | | | | | AF165054, AF165055, AF165056, AF165059, AF165060, AF165061, |
| | | | | | AF165062, AF165063, AF165064, AF207752, AF207755, AF207756, |
| | | | | | AF207757, AF207758, AF207765, AF207766, AF207768, AF207771, |
| | | | | | AF207774, AJ000009, AJ507169, AJ507171, AJ507174, AJ507182, |
| | | | | | AJ507196, AM401848, AM401849, AM401850, AM401851, |
| | | | | | AM401852, AM401853, AM401854, AM401855, AM401856, |
| | | | | | AM401857, AM401858, AM401859, AM401860, AM401861, |
| | | | | | AM401862, AM401863, AM401864, AM401865, AM401866, |
| | | | | | AM401867, AM401868, AM401869, AM401870, AM401871, |
| | | | | | AM401872, AM401873, AM401874, AM401875, AM401876, |
| | | | | | AM401877, AM401878, AM401879, AM401880, AM401881, |
| | | | | | AM401882, AM401883, AM401884, AM401885, AM401886, |
| | | | | | AM401887, AM401888, AM401889, AM401890, AM401891, |
| | | | | | AM401892, AM401893, AM401894, AM401895, AM401896, |
| | | | | | AM401897, AM401898, AM401899, AM401900, AM401901, |
| | | | | | AM401902, AM401903, AM401904, AM401905, AM401906, |
| | | | | | AM401907, AM401908, AM401909, AM401910, AM401912, |
| | | | | | AM401913, AM401914, AM401915, AY045702, AY587016, |
| | | | | | AY808009, AY808031, AY808039, D10934, D30613, D45172, |
| | | | | | D50482, D50484, D63857, D85516, D89815, DQ244140, DQ244141, |
| | | | | | EU155258, EU155302, EU155332, EU155374, EU239714, |
| | | | | | EU482833, EU482874, EU857431, GU451218, GU451219, |
| | | | | | GU451220, L02836, U01214, X61596 |
| 299 | ccg | gct | P | A | AB049089, AB285047 |
| 299 | ccg | gag | P | E | AB049096, AB285052, AB285059, AB285064, AF207763, |
| | | | | | AF207764, AM493471, AM493472, AM493473, AM493474, |
| | | | | | AM493475, AM493476, AM493477, AM493478, AM493479, |
| | | | | | AM493480, AM493481, AM493482, AM493483, AM493484, |
| | | | | | AM493485, AM493486, AM493487, AM493488, AY240968, |
| | | | | | DQ859970 |
| 299 | ccg | ctg | P | L | DQ491967, DQ492127 |
| 299 | ccg | tcg | P | S | AM494937, DQ492159, DQ492167, EU155330 |
| 300 | gcg | gag | A | E | D63857 |
| 300 | gcg | tcg | A | S | EU862835 |
| 300 | gcg | gtg | A | V | AM493488, FJ958905 |
| 301 | gag | ggg | E | G | AM402304, DQ492090, DQ492105, DQ492184, FJ958907 |
| 301 | gag | aag | E | K | DQ492186 |
| 301 | gag | gtg | E | V | DQ491976 |
| 302 | atc | ttc | I | F | AB285073 |
| 302 | atc | acc | I | T | AM402071, DQ244141, DQ492040, DQ492047, DQ492120 |
| 302 | atc | gtc | I | V | AF207766, AY808031, DQ491982, DQ492074, EU482833, |
| | | | | | EU660388, EU862837, FJ958934 |
| 303 | ctg | ccg | L | P | AM401923, DQ492170 |
| 303 | ctg | cag | L | Q | DQ491971 |
| 304 | cgg | cta | R | L | DQ492054, DQ492055, GQ913869 |
| 304 | cgg | ctg | R | L | AJ507164, AJ507170, AJ507178, AJ507187, AJ507199, AY808035, |
| | | | | | DQ492041, DQ492042, DQ492043, DQ492044, DQ492045, |
| | | | | | DQ492046, DQ492047, DQ492048, DQ492049, DQ492050, |
| | | | | | DQ492051, DQ492052, DQ492053, DQ492056, DQ492057, |
| | | | | | DQ492058, DQ492059, DQ492060, DQ492061, DQ492062, |
| | | | | | EF407481, EU155222, EU155228, EU155256, EU155333, |
| | | | | | EU155356, EU256076, FJ390397, GQ913873, HM042051, |
| | | | | | HM042052, HM042054, HM042056, HM042061, HM042063 |
| 304 | cgg | cag | R | Q | AM402089 |
| 304 | cgg | tcg | R | S | EU155373 |
| 304 | cgg | tgg | R | W | AB154205, AB154206, AM402354 |
| 305 | agg | gaa | R | E | DQ492169 |
| 305 | agg | aaa | R | K | AB010249, AB036519, AB036520, AB036521, AB049087, |
| | | | | | AB049088, AB049089, AB049090, AB049091, AB049094, |
| | | | | | AB049095, AB049096, AB049098, AB049099, AB049100, |
| | | | | | AB080299, AB109543, AB191333, AB249644, AB285035, |
| | | | | | AB285036, AB285037, AB285038, AB285039, AB285040, |
| | | | | | AB285042, AB285043, AB285044, AB285046, AB285047, |
| | | | | | AB285048, AB285050, AB285051, AB285052, AB285053, |
| | | | | | AB285054, AB285055, AB285056, AB285057, AB285058, |
| | | | | | AB285059, AB285060, AB285062, AB285063, AB285065, |
| | | | | | AB285066, AB285067, AB285068, AB285069, AB285070, |
| | | | | | AB285071, AB285072, AB285074, AB285075, AB285076, |
| | | | | | AB285077, AB285078, AB285081, AB426117, AB429050, |
| | | | | | AB442219, AB442221, AF165045, AF165046, AF165049, AF165050, |
| | | | | | AF165051, AF165052, AF165053, AF165054, AF165055, AF165056, |
| | | | | | AF165057, AF165058, AF165061, AF165062, AF165063, AF165064, |
| | | | | | AF176573, AF207752, AF207753, AF207755, AF207756, AF207757, |
| | | | | | AF207758, AF207760, AF207761, AF207762, AF207763, AF207764, |
| | | | | | AF207765, AF207766, AF207768, AF207770, AF207771, AF207772, |
| | | | | | AF207774, AF208024, AF265142, AF265143, AF265144, AF265145, |
| | | | | | AF265146, AF265147, AF265148, AF265149, AF265150, AF265152, |
| | | | | | AF265153, AF265154, AF265155, AF265156, AF265160, AF265161, |
| | | | | | AF265162, AF265163, AF265164, AF265165, AF333324, AF356827, |
| | | | | | AF483269, AJ000009, AJ132997, AJ133096, AJ507156, AJ507158, |
| | | | | | AJ507159, AJ507160, AJ507161, AJ507162, AJ507163, AJ507164, |
| | | | | | AJ507166, AJ507167, AJ507168, AJ507169, AJ507170, AJ507171, |
| | | | | | AJ507172, AJ507173, AJ507174, AJ507175, AJ507179, AJ507180, |
| | | | | | AJ507181, AJ507182, AJ507183, AJ507184, AJ507185, AJ507186, |
| | | | | | AJ507187, AJ507188, AJ507189, AJ507190, AJ507191, AJ507192, |
| | | | | | AJ507193, AJ507194, AJ507195, AJ507197, AJ507198, AJ507199, |
| | | | | | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401862, |
| | | | | | AM401863, AM401864, AM401865, AM401866, AM401867, |
| | | | | | AM401868, AM401869, AM401870, AM401871, AM401872, |
| | | | | | AM401873, AM401874, AM401875, AM401876, AM401877, |
| | | | | | AM401878, AM401879, AM401880, AM401881, AM401882, |
| | | | | | AM401883, AM401884, AM401885, AM401886, AM401887, |
| | | | | | AM401888, AM401889, AM401890, AM401891, AM401892, |
| | | | | | AM401893, AM401894, AM401895, AM401896, AM401897, |
| | | | | | AM401898, AM401899, AM401900, AM401901, AM401902, |
| | | | | | AM401903, AM401904, AM401905, AM401906, AM401907, |
| | | | | | AM401908, AM401909, AM401910, AM401911, AM401912, |
| | | | | | AM401913, AM401914, AM401915, AM401916, AM401919, |
| | | | | | AM401920, AM401921, AM401925, AM401929, AM401931, |
| | | | | | AM401934, AM401935, AM401937, AM401938, AM401942, |
| | | | | | AM401946, AM401948, AM401957, AM402058, AM402070, |
| | | | | | AM402098, AM402103, AM402258, AM402259, AM402260, |
| | | | | | AM402261, AM402262, AM402263, AM402264, AM402265, |
| | | | | | AM402266, AM402267, AM402268, AM402269, AM402270, |
| | | | | | AM402271, AM402272, AM402273, AM402274, AM402275, |
| | | | | | AM402276, AM402277, AM402278, AM402279, AM402280, |
| | | | | | AM402281, AM402282, AM402283, AM402284, AM402285, |
| | | | | | AM402286, AM402287, AM402288, AM402289, AM402290, |
| | | | | | AM402291, AM402292, AM402293, AM402294, AM402295, |
| | | | | | AM402296, AM402297, AM402298, AM402299, AM402300, |
| | | | | | AM402301, AM402302, AM402303, AM402304, AM402305, |
| | | | | | AM402306, AM402307, AM402308, AM402309, AM402310, |
| | | | | | AM402311, AM402312, AM402313, AM402314, AM402315, |
| | | | | | AM402316, AM402317, AM402318, AM402319, AM402320, |
| | | | | | AM402321, AM402322, AM402323, AM402324, AM402325, |
| | | | | | AM402326, AM402327, AM402328, AM402329, AM402330, |
| | | | | | AM402331, AM402332, AM402333, AM402334, AM402335, |
| | | | | | AM402336, AM402337, AM402338, AM402339, AM402340, |
| | | | | | AM402341, AM402342, AM402343, AM402344, AM402345, |
| | | | | | AM402346, AM402347, AM402348, AM402349, AM402350, |
| | | | | | AM402352, AM402353, AM402354, AM402355, AM402356, |
| | | | | | AM402357, AM402358, AM402359, AM402360, AM402361, |
| | | | | | AM402362, AM402363, AM402364, AM402365, AM402367, |
| | | | | | AM402369, AM402370, AM402371, AM402372, AM402373, |
| | | | | | AM402374, AM402375, AM402376, AM402377, AM402378, |
| | | | | | AM402379, AM402380, AM402381, AM402382, AM402383, |
| | | | | | AM402384, AM493436, AM493437, AM493438, AM493439, |
| | | | | | AM493440, AM493441, AM493442, AM493443, AM493444, |
| | | | | | AM493445, AM493446, AM493447, AM493448, AM493449, |
| | | | | | AM493450, AM493451, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, |
| | | | | | AM493489, AM493490, AM493491, AM493492, AM493493, |
| | | | | | AM493494, AM493495, AM493496, AM493497, AM493498, |
| | | | | | AM493499, AM493500, AM493502, AM493503, AM493504, |
| | | | | | AM493505, AM493506, AM493507, AM494937, AY008261, |
| | | | | | AY044867, AY045702, AY240969, AY460204, AY587844, |
| | | | | | AY808009, AY808010, AY808011, AY808012, AY808013, |
| | | | | | AY808014, AY808015, AY808017, AY808018, AY808019, |
| | | | | | AY808020, AY808023, AY808024, AY808025, AY808026, |
| | | | | | AY808027, AY808028, AY808030, AY808031, AY808032, |
| | | | | | AY808034, AY808035, AY808036, AY808038, AY808040, |
| | | | | | AY808041, AY808042, D10934, D11168, D16435, D50480, D50481, |
| | | | | | D50482, D50483, D50484, D50485, D85516, D89872, D90208, |
| | | | | | DQ071885, DQ244141, DQ491943, DQ491944, DQ491945, |
| | | | | | DQ491946, DQ491947, DQ491948, DQ491949, DQ491950, |
| | | | | | DQ491951, DQ491952, DQ491953, DQ491954, DQ491955, |
| | | | | | DQ491956, DQ491957, DQ491958, DQ491959, DQ491960, |
| | | | | | DQ491961, DQ491962, DQ491963, DQ491964, DQ491965, |
| | | | | | DQ491966, DQ491967, DQ491968, DQ491969, DQ491970, |
| | | | | | DQ491971, DQ491972, DQ491973, DQ491974, DQ491975, |
| | | | | | DQ491976, DQ491977, DQ491978, DQ491979, DQ491980, |
| | | | | | DQ491981, DQ491982, DQ491983, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, DQ492008, DQ492009, DQ492010, |
| | | | | | DQ492011, DQ492012, DQ492013, DQ492014, DQ492015, |
| | | | | | DQ492016, DQ492017, DQ492018, DQ492019, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ492040, |
| | | | | | DQ492041, DQ492042, DQ492043, DQ492044, DQ492045, |
| | | | | | DQ492046, DQ492047, DQ492048, DQ492049, DQ492050, |
| | | | | | DQ492051, DQ492052, DQ492053, DQ492054, DQ492055, |
| | | | | | DQ492056, DQ492057, DQ492058, DQ492059, DQ492060, |
| | | | | | DQ492061, DQ492062, DQ492063, DQ492065, DQ492066, |
| | | | | | DQ492067, DQ492068, DQ492070, DQ492073, DQ492074, |
| | | | | | DQ492078, DQ492079, DQ492080, DQ492082, DQ492083, |
| | | | | | DQ492084, DQ492085, DQ492086, DQ492087, DQ492088, |
| | | | | | DQ492089, DQ492090, DQ492091, DQ492092, DQ492093, |
| | | | | | DQ492094, DQ492095, DQ492096, DQ492097, DQ492098, |
| | | | | | DQ492099, DQ492100, DQ492101, DQ492102, DQ492103, |
| | | | | | DQ492104, DQ492105, DQ492106, DQ492107, DQ492108, |
| | | | | | DQ492109, DQ492110, DQ492111, DQ492112, DQ492113, |
| | | | | | DQ492114, DQ492115, DQ492116, DQ492117, DQ492118, |
| | | | | | DQ492119, DQ492120, DQ492121, DQ492122, DQ492123, |
| | | | | | DQ492124, DQ492125, DQ492126, DQ492127, DQ492128, |
| | | | | | DQ492129, DQ492130, DQ492131, DQ492132, DQ492133, |
| | | | | | DQ492134, DQ492135, DQ492136, DQ492137, DQ492138, |
| | | | | | DQ492139, DQ492140, DQ492141, DQ492142, DQ492143, |
| | | | | | DQ492144, DQ492145, DQ492146, DQ492147, DQ492148, |
| | | | | | DQ492149, DQ492150, DQ492152, DQ492153, DQ492154, |
| | | | | | DQ492155, DQ492156, DQ492157, DQ492158, DQ492159, |
| | | | | | DQ492160, DQ492161, DQ492162, DQ492163, DQ492164, |
| | | | | | DQ492165, DQ492166, DQ492167, DQ492168, DQ492170, |
| | | | | | DQ492171, DQ492172, DQ492173, DQ492174, DQ492175, |
| | | | | | DQ492176, DQ492177, DQ492178, DQ492179, DQ492180, |
| | | | | | DQ492181, DQ492182, DQ492183, DQ492184, DQ492185, |
| | | | | | DQ492186, DQ492187, DQ492188, DQ492189, DQ492190, |
| | | | | | DQ492191, DQ492192, DQ492193, DQ492194, DQ492195, |
| | | | | | DQ492196, DQ492197, DQ859970, EF032892, EF032893, |
| | | | | | EF032894, EF407459, EF407460, EF407462, EF407463, EF407464, |
| | | | | | EF407465, EF407466, EF407467, EF407468, EF407469, EF407471, |
| | | | | | EF407472, EF407474, EF407475, EF407476, EF407477, EF407478, |
| | | | | | EF407480, EF407481, EF407483, EF407484, EF407485, EF407486, |
| | | | | | EF407487, EF407488, EF407489, EF407490, EF407492, EF407494, |
| | | | | | EF407495, EF407496, EF407498, EF407499, EF407502, EF407503, |
| | | | | | EF407504, EF638081, EU155217, EU155218, EU155219, |
| | | | | | EU155220, EU155221, EU155223, EU155224, EU155225, |
| | | | | | EU155226, EU155227, EU155228, EU155230, EU155231, |
| | | | | | EU155232, EU155235, EU155253, EU155254, EU155255, |
| | | | | | EU155256, EU155257, EU155258, EU155259, EU155260, |
| | | | | | EU155261, EU155263, EU155264, EU155280, EU155281, |
| | | | | | EU155300, EU155301, EU155302, EU155305, EU155306, |
| | | | | | EU155307, EU155308, EU155315, EU155316, EU155317, |
| | | | | | EU155318, EU155324, EU155326, EU155327, EU155328, |
| | | | | | EU155331, EU155332, EU155333, EU155335, EU155336, |
| | | | | | EU155337, EU155356, EU155357, EU155358, EU155359, |
| | | | | | EU155361, EU155362, EU155363, EU155364, EU155366, |
| | | | | | EU155368, EU155369, EU155370, EU155372, EU155373, |
| | | | | | EU155374, EU155375, EU155377, EU155381, EU155382, |
| | | | | | EU234061, EU234062, EU239714, EU255960, EU255961, |
| | | | | | EU255962, EU256000, EU256001, EU256045, EU256059, |
| | | | | | EU256061, EU256062, EU256065, EU256066, EU256075, |
| | | | | | EU256076, EU256077, EU256078, EU256079, EU256080, |
| | | | | | EU256081, EU256082, EU256083, EU256084, EU256085, |
| | | | | | EU256090, EU256091, EU256092, EU256098, EU256099, |
| | | | | | EU256100, EU256101, EU482833, EU482839, EU482849, |
| | | | | | EU482860, EU482874, EU482877, EU482879, EU482880, |
| | | | | | EU482883, EU482886, EU482888, EU660386, EU660388, |
| | | | | | EU781825, EU781827, EU781830, EU781832, EU862835, |
| | | | | | EU862837, FJ024279, FJ390397, FJ390398, FJ478453, FJ958890, |
| | | | | | FJ958891, FJ958892, FJ958893, FJ958894, FJ958895, FJ958896, |
| | | | | | FJ958897, FJ958898, FJ958899, FJ958900, FJ958901, FJ958902, |
| | | | | | FJ958903, FJ958904, FJ958905, FJ958906, FJ958907, FJ958909, |
| | | | | | FJ958910, FJ958911, FJ958912, FJ958913, FJ958914, FJ958915, |
| | | | | | FJ958916, FJ958917, FJ958918, FJ958919, FJ958920, FJ958921, |
| | | | | | FJ958922, FJ958923, FJ958924, FJ958925, FJ958926, FJ958927, |
| | | | | | FJ958928, FJ958929, FJ958931, FJ958933, FJ958934, FJ958935, |
| | | | | | FN435993, GQ913869, GQ913871, GQ913872, GQ913873, |
| | | | | | GU451218, GU451219, GU451220, GU451221, GU451222, |
| | | | | | GU451223, GU451224, HM042051, HM042052, HM042054, |
| | | | | | HM042055, HM042057, HM042058, HM042059, HM042061, |
| | | | | | HM042062, HM042063, L02836, M58335, M84754, M96362, |
| | | | | | U01214, U16362, U45476, X61596 |
| 305 | agg | aag | R | K | AB010250, AB285061, AB285079, AB442220, AB442222, |
| | | | | | AF033373, AF034151, AF165047, AF165048, AF207754, AF207769, |
| | | | | | AF265158, AF265159, AJ507155, AM401917, AM401918, |
| | | | | | AM401922, AM401923, AM401924, AM401926, AM401927, |
| | | | | | AM401928, AM401930, AM401932, AM401933, AM401936, |
| | | | | | AM401939, AM401940, AM401941, AM401943, AM401944, |
| | | | | | AM401945, AM401947, AM401949, AM401950, AM401951, |
| | | | | | AM401952, AM401953, AM401954, AM401955, AM401956, |
| | | | | | AM401958, AM401959, AM401960, AM401961, AM401962, |
| | | | | | AM401963, AM401964, AM401965, AM401966, AM401967, |
| | | | | | AM401968, AM401969, AM401970, AM401971, AM401972, |
| | | | | | AM401973, AM401974, AM401975, AM401976, AM401977, |
| | | | | | AM401978, AM401979, AM401980, AM402351, AY240967, |
| | | | | | AY240968, AY587016, AY808043, D30613, D45172, D89815, |
| | | | | | EF407461, EF407479, EF407482, EF407497, EF407500, EU155279, |
| | | | | | EU155303, EU155329, EU256064, EU256088, EU256089, |
| | | | | | EU256102, EU256103, EU482875, EU857431, FJ024277, FJ390396, |
| | | | | | FJ958930, FJ958932, HM042050, HM042056 |
| 305 | agg | cca | R | P | EF407491, EU155222 |
| 305 | agg | caa | R | Q | DQ244140 |
| 305 | agg | aca | R | T | AB285064, AY808037, EU155262, EU482885 |
| 306 | tcc | gct | S | A | EU256088 |
| 306 | tcc | ttc | S | F | AM402107 |
| 306 | tcc | cac | S | H | EU155318 |
| 306 | tcc | aag | S | K | EU155228 |
| 306 | tcc | aat | S | N | AF207764 |
| 306 | tcc | ccc | S | P | AF207753, AJ507157, AJ507192, D90208, DQ491978, DQ492016, |
| | | | | | DQ492020, EU155376, EU862837, GQ913870, HM042053 |
| 306 | tcc | acc | S | T | AB010250, AB036519, AB036520, AB036521, AB049087, |
| | | | | | AB049089, AB049092, AB049094, AB049095, AB049096, |
| | | | | | AB109543, AB191333, AB285035, AB285040, AB285043, |
| | | | | | AB285047, AB285048, AB285050, AB285052, AB285053, |
| | | | | | AB285054, AB285055, AB285056, AB285057, AB285060, |
| | | | | | AB285061, AB285064, AB285068, AB285073, AB285075, |
| | | | | | AB285077, AB285078, AB285079, AB429050, AB442219, |
| | | | | | AB442220, AF165045, AF165046, AF165047, AF165048, AF165053, |
| | | | | | AF165054, AF165055, AF165056, AF165057, AF165058, AF165063, |
| | | | | | AF165064, AF176573, AF207755, AF207757, AF207769, AF207771, |
| | | | | | AF265158, AF265159, AJ507158, AJ507161, AJ507164, AJ507184, |
| | | | | | AJ507189, AM401864, AM401865, AM401866, AM401869, |
| | | | | | AM401870, AM401871, AM401872, AM401873, AM401874, |
| | | | | | AM401875, AM401877, AM401879, AM401883, AM401884, |
| | | | | | AM401885, AM401887, AM401888, AM401889, AM401890, |
| | | | | | AM401893, AM401894, AM401895, AM401896, AM401897, |
| | | | | | AM401898, AM401899, AM401900, AM401901, AM401902, |
| | | | | | AM401903, AM401904, AM401905, AM401906, AM401907, |
| | | | | | AM401908, AM401909, AM401910, AM401911, AM401912, |
| | | | | | AM401913, AM401914, AM401915, AM493489, AM493490, |
| | | | | | AM493491, AM493492, AM493493, AM493494, AM493495, |
| | | | | | AM493496, AM493497, AM493498, AM493499, AM493501, |
| | | | | | AM493502, AM493503, AM493504, AM493505, AM493506, |
| | | | | | AM493507, AY045702, AY460204, AY587016, AY808025, |
| | | | | | AY808041, D10934, D30613, D45172, D50482, D50484, D89815, |
| | | | | | DQ071885, DQ244141, DQ491958, DQ491959, DQ491960, |
| | | | | | DQ491961, DQ491962, DQ491963, DQ491964, DQ491965, |
| | | | | | DQ491966, DQ491967, DQ491968, DQ491969, DQ491970, |
| | | | | | EF407478, EF407479, EF407490, EF407498, EF638081, EU155222, |
| | | | | | EU155231, EU155253, EU155324, EU155361, EU155369, |
| | | | | | EU155382, EU256064, EU256065, EU256076, EU256089, |
| | | | | | EU256100, EU256101, EU482874, EU482875, EU482880, |
| | | | | | EU857431, FJ958911, FJ958924, GU451218, GU451219, |
| | | | | | GU451220, GU451221, GU451222, GU451223, GU451224, |
| | | | | | HM042056 |
| 306 | tcc | acg | S | T | EF407487 |
| 306 | tcc | act | S | T | AB285042, AM493500, AY808012, EF032892, EF032893, EF032894 |
| 307 | agg | ggg | R | G | EU155368 |
| 307 | agg | aaa | R | K | AB010249, AB285061, AB442221, EF407468, EF407487, |
| | | | | | EU155303, EU155361, EU155372, EU256066, EU482839, |
| | | | | | EU482881, EU529682, EU862835 |
| 307 | agg | aag | R | K | AB285044, AB285053, AB285055, AB285056, AB285064, |
| | | | | | AB285070, AF034151, AF165049, AF165050, AF165057, AF165058, |
| | | | | | AF207762, AF207765, AF333324, AJ507166, AJ507171, AJ507172, |
| | | | | | AJ507174, AJ507180, AJ507183, AJ507186, AM402055, AM402056, |
| | | | | | AM402057, AM402058, AM402059, AM402060, AM402061, |
| | | | | | AM402062, AM402063, AM402064, AM402065, AM402066, |
| | | | | | AM402067, AM402068, AM402069, AM402070, AM402071, |
| | | | | | AM402072, AM402073, AM402074, AM402075, AM402076, |
| | | | | | AM402077, AM402078, AM402079, AM402080, AM402081, |
| | | | | | AM402082, AM402083, AM402084, AM402085, AM402086, |
| | | | | | AM402087, AM402088, AM402089, AM402090, AM402091, |
| | | | | | AM402092, AM402093, AM402094, AM402095, AM402096, |
| | | | | | AM402097, AM402098, AM402099, AM402100, AM402101, |
| | | | | | AM402102, AM402103, AM402104, AM402105, AM402106, |
| | | | | | AM402107, AM402108, AM402109, AM402110, AM402111, |
| | | | | | AM402112, AM402113, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AM494937, AY044867, |
| | | | | | AY045702, AY240969, AY808016, AY808030, AY808033, |
| | | | | | AY808035, AY808036, D11168, D16435, D50480, D50483, D50484, |
| | | | | | D89872, DQ244141, DQ491971, DQ492040, DQ492041, DQ492042, |
| | | | | | DQ492043, DQ492044, DQ492045, DQ492046, DQ492047, |
| | | | | | DQ492048, DQ492049, DQ492050, DQ492051, DQ492052, |
| | | | | | DQ492053, DQ492054, DQ492055, DQ492056, DQ492057, |
| | | | | | DQ492058, DQ492059, DQ492060, DQ492061, DQ492062, |
| | | | | | EF407460, EF407462, EF407464, EF407466, EF407475, EF407479, |
| | | | | | EF407488, EF407503, EF638081, EU155221, EU155228, |
| | | | | | EU155232, EU155255, EU155257, EU155307, EU155315, |
| | | | | | EU155357, EU155365, EU155367, EU155375, EU255962, |
| | | | | | EU256064, EU256078, EU256081, EU256085, EU256090, |
| | | | | | EU256091, EU256103, EU482859, EU482875, EU482880, |
| | | | | | EU781825, FJ390397, FJ390398, GU451221, GU451222, |
| | | | | | GU451223, GU451224, HM042050, HM042056, M58335, U01214 |
| 308 | aaa | gca | K | A | EU862835 |
| 308 | aaa | gcc | K | A | AM402109 |
| 308 | aaa | gct | K | A | AM402055, AM402056, AM402057, AM402058, AM402059, |
| | | | | | AM402060, AM402061, AM402062, AM402063, AM402064, |
| | | | | | AM402065, AM402066, AM402067, AM402068, AM402069, |
| | | | | | AM402070, AM402071, AM402072, AM402073, AM402074, |
| | | | | | AM402075, AM402076, AM402077, AM402078, AM402079, |
| | | | | | AM402080, AM402081, AM402082, AM402083, AM402084, |
| | | | | | AM402085, AM402086, AM402087, AM402088, AM402089, |
| | | | | | AM402091, AM402092, AM402093, AM402094, AM402095, |
| | | | | | AM402096, AM402097, AM402099, AM402101, AM402102, |
| | | | | | AM402106, AM402107, AM402108, AM402110, AM402111, |
| | | | | | AM402112, AM402113, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AY808030, EU155221, |
| | | | | | EU155365, EU155367, EU529682 |
| 308 | aaa | gaa | K | E | AY808034, DQ491946, DQ492008, DQ492009, DQ492010, |
| | | | | | DQ492011, DQ492012, DQ492013, DQ492014, DQ492015, |
| | | | | | DQ492017, DQ492018, DQ492020, DQ492021, DQ492022, |
| | | | | | DQ492023, DQ492024, DQ492025, DQ492026, DQ492027, |
| | | | | | DQ492028, DQ492029, DQ492030, DQ492031, DQ492032, |
| | | | | | DQ492033, DQ492034, DQ492035, DQ492036, DQ492037, |
| | | | | | DQ492038, DQ492039, DQ492040, DQ492046, DQ492047, |
| | | | | | DQ492048, DQ492050, DQ492054, DQ492055, EU256079 |
| 308 | aaa | gag | K | E | AB049088 |
| 308 | aaa | gga | K | G | DQ492019 |
| 308 | aaa | ggc | K | G | EU256085 |
| 308 | aaa | ggg | K | G | AB049099 |
| 308 | aaa | ata | K | I | EU256066 |
| 308 | aaa | atg | K | M | EU256075 |
| 308 | aaa | aac | K | N | AB285039, EF407485, EF407500, EU482885 |
| 308 | aaa | aat | K | N | AB010249, AB049091, AF207752, DQ491980, DQ491981, |
| | | | | | DQ491983, DQ491984, DQ491989, DQ492065, DQ492067, |
| | | | | | DQ492068, DQ492070, DQ492074, DQ492080, DQ492082, |
| | | | | | DQ492085, DQ492086, DQ492089, DQ492090, DQ492091, |
| | | | | | DQ492092, EU155257 |
| 308 | aaa | aga | K | R | AB049089, AF207760, AF207768, AF483269, AJ000009, AJ507155, |
| | | | | | AJ507171, AJ507175, AM402266, AM402329, AM402330, |
| | | | | | AM402331, AM402332, AM402333, AM402334, AM402335, |
| | | | | | AM402336, AM402337, AM402338, AM402339, AM402340, |
| | | | | | AM402341, AM402342, AM402343, AM402344, AM402345, |
| | | | | | AM402346, AM402347, AM402348, AM402349, AM402350, |
| | | | | | AM402351, AM402352, AM402353, AM402354, AM402355, |
| | | | | | AM402356, AM402357, AM402358, AM402359, AM402360, |
| | | | | | AM402361, AM402362, AM402363, AM402364, AM402365, |
| | | | | | AM402367, AM402369, AM402370, AM402371, AM402372, |
| | | | | | AM402373, AM402374, AM402375, AM402376, AM402377, |
| | | | | | AM402378, AM402379, AM402380, AM402381, AM402382, |
| | | | | | AM402383, AM402384, AM493489, AM493490, AM493491, |
| | | | | | AM493492, AM493493, AM493494, AM493495, AM493496, |
| | | | | | AM493498, AM493499, AM493500, AM493502, AM493503, |
| | | | | | AM493505, AM493506, AM493507, AY044867, D10934, EF407460, |
| | | | | | EF407488, EU155220, EU155256, EU155318, EU155336, |
| | | | | | EU155372, EU256064, EU256081, EU482880, EU862837, |
| | | | | | HM042050 |
| 308 | aaa | agg | K | R | AB049101, AB285056, AB285076, AB442221, AF034151, |
| | | | | | AF207762, AF207765, AM401848, AM401849, AM401850, |
| | | | | | AM401851, AM401852, AM401853, AM401854, AM401855, |
| | | | | | AM401856, AM401858, AM401859, AM401860, AM401861, |
| | | | | | AM401862, AM401863, AM401867, AM401868, AM401876, |
| | | | | | AM401878, AM401880, AM401881, AM401882, AM401886, |
| | | | | | AM401891, AM401892, AM494937, AY045702, EU155306, |
| | | | | | EU155315, EU155376 |
| 308 | aaa | agc | K | S | AJ132997, EU256091 |
| 308 | aaa | agt | K | S | AF165063, AF165064, HM042056 |
| 308 | aaa | gta | K | V | FJ390397 |
| 308 | aaa | gtc | K | V | AM402100 |
| 308 | aaa | gtt | K | V | AM402090, AM402098, AM402103, AM402104, AM402105, |
| | | | | | AY808016 |
| 309 | ttc | ctc | F | L | DQ492085 |
| 309 | ttc | tcc | F | S | AM401977, DQ491961, DQ491964, DQ491966 |
| 309 | ttc | tac | F | Y | AB285048, AY460204, EF407462, EF638081, GU451221, |
| | | | | | GU451222, GU451223, GU451224 |
| 310 | cct | ctc | P | L | AM402092, DQ492180 |
| 310 | cct | tcc | P | S | EU155219 |
| 310 | cct | acc | P | T | AJ507155, AM402329, AM402330, AM402331, AM402332, |
| | | | | | AM402333, AM402334, AM402335, AM402336, AM402337, |
| | | | | | AM402338, AM402339, AM402340, AM402341, AM402342, |
| | | | | | AM402343, AM402344, AM402345, AM402346, AM402347, |
| | | | | | AM402348, AM402349, AM402350, AM402351, AM402352, |
| | | | | | AM402353, AM402354, AM402355, AM402356, AM402357, |
| | | | | | AM402358, AM402359, AM402360, AM402361, AM402362, |
| | | | | | AM402363, AM402364, AM402365, AM402367, AM402369, |
| | | | | | AM402370, AM402371, AM402372, AM402373, AM402374, |
| | | | | | AM402375, AM402376, AM402377, AM402378, AM402379, |
| | | | | | AM402380, AM402381, AM402382, AM402383, AM402384 |
| 311 | cga | gca | R | A | AB010250, AB249644, AB285037, AB285040, AB285044, |
| | | | | | AB285062, AB285063, AB426117, AF207762, AF207772, |
| | | | | | AF207774, AF333324, AJ507179, AY587016, AY808022, D14484, |
| | | | | | D30613, D45172, D89815, EU155318, EU155335, EU155376, |
| | | | | | EU256081, EU857431, M58335 |
| 311 | cga | gcg | R | A | AB049098, AB285070, AM401848, AM401849, AM401850, |
| | | | | | AM401851, AM401852, AM401853, AM401854, AM401855, |
| | | | | | AM401856, AM401857, AM401858, AM401859, AM401860, |
| | | | | | AM401861, AM401862, AM401863, AM401864, AM401865, |
| | | | | | AM401866, AM401869, AM401870, AM401871, AM401872, |
| | | | | | AM401873, AM401874, AM401875, AM401877, AM401879, |
| | | | | | AM401881, AM401882, AM401883, AM401884, AM401885, |
| | | | | | AM401886, AM401887, AM401888, AM401889, AM401890, |
| | | | | | AM401891, AM401892, AM401893, AM401894, AM401895, |
| | | | | | AM401896, AM401897, AM401898, AM401899, AM401900, |
| | | | | | AM401901, AM401902, AM401903, AM401904, AM401905, |
| | | | | | AM401906, AM401907, AM401908, AM401909, AM401910, |
| | | | | | AM401911, AM401912, AM401913, AM401914, AM401915, |
| | | | | | DQ244141, EF407459, EU155358, EU256099 |
| 311 | cga | gct | R | A | AB285061, EU155382 |
| 311 | cga | cac | R | H | EU256061 |
| 311 | cga | cat | R | H | AB285076 |
| 311 | cga | cta | R | L | AB049092, AB049096, AB285069, AF165050, AJ507170, EF407502, |
| | | | | | HM042062 |
| 311 | cga | ctc | R | L | EU155316 |
| 311 | cga | ctg | R | L | AB154188, AF165049, EF407462, HM042058 |
| 311 | cga | atg | R | M | AF165051, AF165052 |
| 311 | cga | cca | R | P | AB049087, AB049088, AB049089, AB049091, AB049094, |
| | | | | | AB049095, AB049099, AB154189, AB154190, AB285035, |
| | | | | | AB285042, AB285047, AB285051, AB285053, AB285054, |
| | | | | | AB285055, AB285056, AB285057, AB285058, AB285064, |
| | | | | | AB285068, AB285071, AB285073, AB285077, AB285078, |
| | | | | | AB285079, AB442219, AB442222, AF034151, AF165045, |
| | | | | | AF165046, AF165055, AF165056, AF165058, AF165059, AF165060, |
| | | | | | AF165061, AF165062, AF165064, AF207753, AF207755, AF207758, |
| | | | | | AF207764, AF207766, AF207768, AF207769, AF207770, AF265142, |
| | | | | | AF265143, AF265144, AF265158, AF265159, AF265160, AF265162, |
| | | | | | AJ507156, AJ507158, AJ507159, AJ507160, AJ507163, AJ507164, |
| | | | | | AJ507167, AJ507168, AJ507174, AJ507178, AJ507181, AJ507182, |
| | | | | | AJ507190, AJ507193, AJ507194, AM402329, AM402330, |
| | | | | | AM402331, AM402332, AM402333, AM402334, AM402335, |
| | | | | | AM402336, AM402337, AM402338, AM402339, AM402340, |
| | | | | | AM402341, AM402342, AM402343, AM402344, AM402345, |
| | | | | | AM402346, AM402347, AM402348, AM402349, AM402350, |
| | | | | | AM402351, AM402352, AM402353, AM402354, AM402355, |
| | | | | | AM402356, AM402357, AM402358, AM402359, AM402360, |
| | | | | | AM402361, AM402362, AM402363, AM402364, AM402365, |
| | | | | | AM402367, AM402369, AM402370, AM402371, AM402372, |
| | | | | | AM402373, AM402374, AM402375, AM402376, AM402377, |
| | | | | | AM402378, AM402379, AM402380, AM402381, AM402382, |
| | | | | | AM402383, AM402384, AM494937, AY008261, AY240967, |
| | | | | | AY240968, AY240969, AY460204, AY587844, AY808009, |
| | | | | | AY808017, AY808020, AY808021, AY808025, AY808026, |
| | | | | | AY808031, AY808034, AY808035, AY808038, AY808039, |
| | | | | | AY808040, D10934, D50480, D50482, D50484, D63857, D85516, |
| | | | | | D90208, DQ244140, DQ491943, DQ491944, DQ491945, DQ491946, |
| | | | | | DQ491947, DQ491948, DQ491949, DQ491950, DQ491951, |
| | | | | | DQ491952, DQ491953, DQ491954, DQ491955, DQ491956, |
| | | | | | DQ491957, DQ492008, DQ492009, DQ492010, DQ492011, |
| | | | | | DQ492012, DQ492013, DQ492014, DQ492015, DQ492016, |
| | | | | | DQ492017, DQ492019, DQ492020, DQ492021, DQ492022, |
| | | | | | DQ492023, DQ492024, DQ492025, DQ492026, DQ492027, |
| | | | | | DQ492028, DQ492029, DQ492030, DQ492031, DQ492032, |
| | | | | | DQ492033, DQ492034, DQ492035, DQ492036, DQ492037, |
| | | | | | DQ492038, DQ492039, DQ492041, DQ492042, DQ492043, |
| | | | | | DQ492044, DQ492045, DQ492046, DQ492047, DQ492048, |
| | | | | | DQ492049, DQ492050, DQ492051, DQ492052, DQ492053, |
| | | | | | DQ492054, DQ492055, DQ492056, DQ492057, DQ492058, |
| | | | | | DQ492059, DQ492060, DQ492061, DQ492062, DQ492064, |
| | | | | | DQ492069, DQ492071, DQ492072, DQ492075, DQ492076, |
| | | | | | DQ492077, DQ492081, EF407460, EF407461, EF407464, |
| | | | | | EF407468, EF407477, EF407478, EF407483, EF407488, EF407491, |
| | | | | | EF407501, EF407503, EF407504, EF638081, EU155219, |
| | | | | | EU155220, EU155222, EU155227, EU155228, EU155229, |
| | | | | | EU155231, EU155232, EU155235, EU155256, EU155257, |
| | | | | | EU155281, EU155302, EU155306, EU155325, EU155330, |
| | | | | | EU155334, EU155337, EU155356, EU155359, EU155360, |
| | | | | | EU155361, EU155363, EU155364, EU155366, EU155369, |
| | | | | | EU155373, EU155375, EU155377, EU234061, EU234062, |
| | | | | | EU255960, EU256045, EU256064, EU256075, EU256076, |
| | | | | | EU256083, EU256084, EU256088, EU256089, EU256090, |
| | | | | | EU256092, EU482833, EU482849, EU482859, EU482886, |
| | | | | | FJ024277, FJ390397, GQ913869, GQ913873, GU451218, |
| | | | | | GU451219, GU451220, GU451221, GU451222, GU451223, |
| | | | | | GU451224, HM042050, HM042051, HM042052, HM042054, |
| | | | | | HM042055, HM042056, HM042057, HM042061, HM042063, |
| | | | | | M84754, M96362, U16362, U45476, X61596 |
| 311 | cga | ccc | R | P | AB285043, AB285066, AF265161, AJ507157, AJ507183, AY808019, |
| | | | | | AY808042, D11168, D16435, D89872, DQ491971, DQ491972, |
| | | | | | DQ491973, DQ491974, DQ491975, DQ491976, DQ491977, |
| | | | | | DQ491978, DQ492094, DQ492095, DQ492096, DQ492097, |
| | | | | | DQ492098, DQ492099, DQ492100, DQ492101, DQ492102, |
| | | | | | DQ492103, DQ492104, DQ492105, DQ492106, DQ492107, |
| | | | | | DQ492108, DQ492109, DQ492110, DQ492111, DQ492112, |
| | | | | | DQ492113, DQ492114, DQ492115, DQ492116, DQ492117, |
| | | | | | DQ492118, DQ492119, DQ492120, DQ492121, DQ492122, |
| | | | | | DQ492123, EU155263, EU155327, EU155336, EU482874, |
| | | | | | FJ390398, FJ958934 |
| 311 | cga | ccg | R | P | AB010249, AB049090, AB049100, AB285048, AB285052, |
| | | | | | AB285065, AB429050, AB442221, AF165047, AF165048, |
| | | | | | AF165053, AF165054, AF165063, AF207757, AF207771, AF208024, |
| | | | | | AF265145, AF265146, AF265147, AJ507155, AJ507161, AJ507165, |
| | | | | | AJ507166, AJ507171, AJ507180, AJ507184, AJ507186, AJ507187, |
| | | | | | AJ507189, AJ507192, AJ507199, AM402055, AM402056, |
| | | | | | AM402057, AM402058, AM402059, AM402060, AM402061, |
| | | | | | AM402062, AM402063, AM402064, AM402065, AM402066, |
| | | | | | AM402067, AM402068, AM402069, AM402070, AM402071, |
| | | | | | AM402072, AM402073, AM402074, AM402075, AM402076, |
| | | | | | AM402077, AM402078, AM402079, AM402080, AM402081, |
| | | | | | AM402082, AM402083, AM402084, AM402085, AM402086, |
| | | | | | AM402087, AM402088, AM402089, AM402090, AM402091, |
| | | | | | AM402092, AM402093, AM402094, AM402095, AM402096, |
| | | | | | AM402097, AM402098, AM402099, AM402100, AM402101, |
| | | | | | AM402102, AM402103, AM402104, AM402105, AM402106, |
| | | | | | AM402107, AM402108, AM402109, AM402110, AM402111, |
| | | | | | AM402112, AM402113, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AM493489, AM493490, |
| | | | | | AM493491, AM493492, AM493493, AM493494, AM493495, |
| | | | | | AM493496, AM493497, AM493498, AM493499, AM493500, |
| | | | | | AM493501, AM493502, AM493503, AM493504, AM493505, |
| | | | | | AM493506, AM493507, AY045702, AY808016, AY808018, |
| | | | | | AY808029, AY808030, AY808033, D50485, DQ492018, DQ492063, |
| | | | | | DQ492065, DQ492066, DQ492067, DQ492068, DQ492070, |
| | | | | | DQ492073, DQ492074, DQ492078, DQ492079, DQ492080, |
| | | | | | DQ492082, DQ492083, DQ492084, DQ492085, DQ492086, |
| | | | | | DQ492087, DQ492088, DQ492089, DQ492090, DQ492091, |
| | | | | | DQ492092, EF407469, EF407475, EF407476, EF407485, |
| | | | | | EF407486, EF407490, EU155221, EU155223, EU155307, |
| | | | | | EU155315, EU155317, EU155332, EU155365, EU155367, |
| | | | | | EU155368, EU155370, EU155371, EU155372, EU155374, |
| | | | | | EU256080, EU256085, EU256091, EU256100, EU256103, |
| | | | | | EU482880, EU482885, EU482888, EU529682, EU781827, |
| | | | | | EU862835, EU862837, FJ958890, FJ958891, FJ958892, FJ958893, |
| | | | | | FJ958894, FJ958895, FJ958896, FJ958897, FJ958898, FJ958899, |
| | | | | | FJ958900, FJ958901, FJ958902, FJ958903, FJ958904, FJ958905, |
| | | | | | FJ958906, FJ958907, FJ958909, FJ958910, FJ958911, FJ958912, |
| | | | | | FJ958913, FJ958914, FJ958915, FJ958916, FJ958917, FJ958918, |
| | | | | | FJ958919, FJ958920, FJ958921, FJ958922, FJ958923, FJ958924, |
| | | | | | FJ958925, FJ958926, FJ958927, FJ958928, FJ958929, FJ958930, |
| | | | | | FJ958931, FJ958932, FJ958933, FJ958935, FN435993, U01214 |
| 311 | cga | cct | R | P | AB080299, AB442220, AF165057, AY808012, DQ492040 |
| 311 | cga | caa | R | Q | AJ133096, AJ507191, AY808028, DQ491979, DQ491980, |
| | | | | | DQ491981, DQ491982, DQ491983, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, EF407496, EU155254, EU155305, |
| | | | | | EU155324, EU155328, HM042059 |
| 311 | cga | cag | R | Q | AJ507177 |
| 311 | cga | tca | R | S | AB036519, AB036520, AB036521, AB109543, AB191333, |
| | | | | | AB285036, AB285038, AB285050, AB285059, AB285060, |
| | | | | | AB285067, AB285072, AB285074, AB285075, AB285081, |
| | | | | | AF033373, AF176573, AF207752, AF207754, AF207765, AF483269, |
| | | | | | AJ507162, AJ507172, AJ507175, AJ507197, AJ507198, AY044867, |
| | | | | | AY808036, AY808043, D50483, DQ859970, EF407487, EU155357, |
| | | | | | EU155362, EU256062, EU256066, EU256078, EU781825, |
| | | | | | EU781830, FJ478453, L02836 |
| 311 | cga | tcg | R | S | AB049101, AB285046, AJ507188, AM401867, AM401868, |
| | | | | | AM401876, AM401878, AM401880, AY808013, AY808024, |
| | | | | | EU256000 |
| 311 | cga | aca | R | T | AF207756, EF407479, EF407498, EU482839 |
| 312 | gcg | acg | A | T | DQ492070 |
| 313 | atg | ata | M | I | AB049087, AB049094, AB049095, AB049099, AB285052, |
| | | | | | AB285053, AB285056, AB285060, AB285068, AB429050, |
| | | | | | AF165045, AF165046, AF165047, AF165048, AF165057, AF165058, |
| | | | | | AF207755, AF207764, AM401926, DQ492128, EF407467, |
| | | | | | EU155365, EU256059, GQ913873, HM042051, HM042056, |
| | | | | | HM042058 |
| 313 | atg | att | M | I | AF165053, AF165054, AF165063, AF165064 |
| 313 | atg | cta | M | L | AF207757, AF207771, AY808032 |
| 313 | atg | ctc | M | L | D50482, D50484 |
| 313 | atg | ctg | M | L | AB049088, AB049089, AB049090, AB049092, AB049100, |
| | | | | | AB080299, AB285043, AB285047, AB285048, AB285051, |
| | | | | | AB285065, AB285075, AB285077, AB442221, AF165059, |
| | | | | | AF165060, AF207754, AF207756, AF207762, AF207763, AF207765, |
| | | | | | AF265150, AF265164, AJ507155, AJ507156, AJ507163, AJ507164, |
| | | | | | AJ507180, AJ507182, AJ507187, AJ507191, AJ507192, AJ507194, |
| | | | | | AY240967, AY240968, AY240969, AY460204, AY587844, |
| | | | | | AY808028, AY808033, AY808043, DQ071885, DQ491979, |
| | | | | | DQ491982, DQ491985, DQ491986, DQ491987, DQ491988, |
| | | | | | DQ491990, DQ491991, DQ491992, DQ491993, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, DQ492057, DQ492189, |
| | | | | | DQ859970, EF407462, EF407465, EF407470, EF407476, |
| | | | | | EF407490, EF407494, EU155219, EU155255, EU155256, |
| | | | | | EU155327, EU155328, EU155364, EU155374, EU155377, |
| | | | | | EU239714, EU256078, EU256079, EU256080, EU256085, |
| | | | | | EU482833, EU482859, EU482880, EU529682, EU862837, |
| | | | | | GQ913868, L02836, M96362, U16362 |
| 313 | atg | tta | M | L | AB285035, D50480, D50483, DQ492184, DQ492185 |
| 313 | atg | ttg | M | L | AB010249, AB036519, AB036520, AB036521, AB049101, |
| | | | | | AB109543, AB154190, AB191333, AB249644, AB285042, |
| | | | | | AB285050, AB285058, AB285066, AB285069, AB285073, |
| | | | | | AB285076, AB285079, AB426117, AB442222, AF034151, |
| | | | | | AF165055, AF165056, AF207753, AF207758, AF207766, AF207768, |
| | | | | | AF207770, AJ507157, AJ507173, AJ507179, AJ507190, AJ507196, |
| | | | | | AM402060, AM402066, AM402070, AM402074, AM402075, |
| | | | | | AM402081, AM402084, AM402088, AM402090, AM402096, |
| | | | | | AM402101, AM402102, AM402106, AM402108, AM402111, |
| | | | | | AM402112, AM402115, AM402127, AM402329, AM402330, |
| | | | | | AM402331, AM402332, AM402333, AM402334, AM402335, |
| | | | | | AM402336, AM402337, AM402338, AM402339, AM402340, |
| | | | | | AM402341, AM402342, AM402343, AM402344, AM402345, |
| | | | | | AM402346, AM402347, AM402348, AM402349, AM402350, |
| | | | | | AM402351, AM402352, AM402353, AM402354, AM402355, |
| | | | | | AM402356, AM402357, AM402358, AM402359, AM402360, |
| | | | | | AM402361, AM402362, AM402363, AM402364, AM402365, |
| | | | | | AM402367, AM402369, AM402370, AM402371, AM402372, |
| | | | | | AM402373, AM402374, AM402375, AM402376, AM402377, |
| | | | | | AM402378, AM402379, AM402380, AM402381, AM402382, |
| | | | | | AM402383, AM402384, AM493489, AM493490, AM493491, |
| | | | | | AM493492, AM493493, AM493494, AM493495, AM493496, |
| | | | | | AM493497, AM493498, AM493499, AM493500, AM493501, |
| | | | | | AM493502, AM493503, AM493504, AM493505, AM493506, |
| | | | | | AM493507, AM494937, AY044867, AY808014, D11168, D16435, |
| | | | | | D85516, D89872, D90208, DQ244140, DQ491980, DQ491981, |
| | | | | | DQ491983, DQ491984, DQ491989, DQ492093, DQ492164, |
| | | | | | DQ492165, DQ492166, DQ492167, DQ492168, DQ492169, |
| | | | | | DQ492170, DQ492171, DQ492172, DQ492173, DQ492174, |
| | | | | | DQ492175, DQ492176, DQ492177, DQ492178, DQ492179, |
| | | | | | DQ492180, DQ492181, DQ492182, DQ492183, DQ492186, |
| | | | | | DQ492187, DQ492188, DQ492190, DQ492191, DQ492192, |
| | | | | | DQ492193, DQ492194, DQ492195, DQ492196, DQ492197, |
| | | | | | EF407471, EF407477, EF407486, EF407491, EF407503, EF407504, |
| | | | | | EU155227, EU155281, EU155302, EU155316, EU155335, |
| | | | | | EU155360, EU155369, EU256083, EU256089, EU256091, |
| | | | | | FN435993, HM042062, M84754, U01214, X61596 |
| 313 | atg | tcg | M | S | DQ492163 |
| 313 | atg | acg | M | T | DQ491963 |
| 313 | atg | gtg | M | V | AM401853, AM402276, AM402282, AY808017, HM042059 |
| 313 | atg | tgg | M | W | AM402125 |
| 314 | ccc | ctc | P | L | DQ492109 |
| 314 | ccc | tcc | P | S | AM493438, AM493445, DQ492192 |
| 315 | ata | gaa | I | E | U01214 |
| 315 | ata | ttt | I | F | AB285067 |
| 315 | ata | aca | I | T | DQ492188 |
| 315 | ata | gta | I | V | AB010250, AB049090, AB049098, AB049100, AB080299, |
| | | | | | AB154179, AB154180, AB154188, AB285037, AB285065, |
| | | | | | AB285066, AB285069, AB285074, AF165051, AF165052, |
| | | | | | AF165055, AF165056, AF165059, AF165060, AF165063, AF165064, |
| | | | | | AF207752, AF207760, AF207762, AF207763, AF207769, AF207774, |
| | | | | | AJ507158, AJ507160, AJ507167, AJ507179, AJ507181, AJ507188, |
| | | | | | AJ507191, AJ507196, AJ507198, AM401848, AM401849, |
| | | | | | AM401850, AM401851, AM401852, AM401853, AM401854, |
| | | | | | AM401855, AM401856, AM401857, AM401858, AM401859, |
| | | | | | AM401860, AM401861, AM401862, AM401863, AM401864, |
| | | | | | AM401865, AM401866, AM401867, AM401868, AM401869, |
| | | | | | AM401870, AM401871, AM401872, AM401873, AM401874, |
| | | | | | AM401875, AM401876, AM401877, AM401878, AM401879, |
| | | | | | AM401880, AM401881, AM401882, AM401883, AM401884, |
| | | | | | AM401885, AM401886, AM401887, AM401888, AM401889, |
| | | | | | AM401890, AM401891, AM401892, AM401893, AM401894, |
| | | | | | AM401895, AM401896, AM401897, AM401898, AM401899, |
| | | | | | AM401900, AM401901, AM401902, AM401903, AM401904, |
| | | | | | AM401905, AM401906, AM401907, AM401908, AM401909, |
| | | | | | AM401910, AM401911, AM401912, AM401913, AM401914, |
| | | | | | AM401915, AM401932, AM401933, AM401939, AM401940, |
| | | | | | AM401945, AM402055, AM402056, AM402057, AM402058, |
| | | | | | AM402059, AM402060, AM402061, AM402062, AM402063, |
| | | | | | AM402064, AM402065, AM402066, AM402067, AM402068, |
| | | | | | AM402069, AM402070, AM402071, AM402072, AM402073, |
| | | | | | AM402074, AM402075, AM402076, AM402077, AM402078, |
| | | | | | AM402079, AM402080, AM402081, AM402082, AM402083, |
| | | | | | AM402084, AM402085, AM402086, AM402087, AM402088, |
| | | | | | AM402089, AM402090, AM402091, AM402092, AM402093, |
| | | | | | AM402094, AM402095, AM402096, AM402097, AM402098, |
| | | | | | AM402099, AM402100, AM402101, AM402102, AM402103, |
| | | | | | AM402104, AM402105, AM402106, AM402107, AM402108, |
| | | | | | AM402109, AM402110, AM402111, AM402112, AM402113, |
| | | | | | AM402114, AM402115, AM402116, AM402117, AM402118, |
| | | | | | AM402119, AM402120, AM402121, AM402122, AM402123, |
| | | | | | AM402124, AM402125, AM402126, AM402127, AM402128, |
| | | | | | AM402129, AM402258, AM402259, AM402260, AM402261, |
| | | | | | AM402262, AM402263, AM402264, AM402265, AM402266, |
| | | | | | AM402267, AM402268, AM402269, AM402270, AM402271, |
| | | | | | AM402272, AM402273, AM402274, AM402275, AM402276, |
| | | | | | AM402277, AM402278, AM402279, AM402280, AM402281, |
| | | | | | AM402282, AM402283, AM402284, AM402285, AM402286, |
| | | | | | AM402287, AM402288, AM402289, AM402290, AM402291, |
| | | | | | AM402292, AM402293, AM402294, AM402295, AM402296, |
| | | | | | AM402297, AM402298, AM402299, AM402300, AM402301, |
| | | | | | AM402302, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402326, |
| | | | | | AM402327, AM402328, AM402330, AM402331, AM402332, |
| | | | | | AM402333, AM402335, AM402337, AM402338, AM402340, |
| | | | | | AM402341, AM402346, AM402348, AM402349, AM402351, |
| | | | | | AM402352, AM402353, AM402354, AM402355, AM402356, |
| | | | | | AM402357, AM402358, AM402359, AM402361, AM402363, |
| | | | | | AM402364, AM402365, AM402367, AM402369, AM402370, |
| | | | | | AM402372, AM402373, AM402374, AM402375, AM402376, |
| | | | | | AM402377, AM402378, AM402381, AM402383, AM402384, |
| | | | | | AM493436, AM493437, AM493438, AM493439, AM493440, |
| | | | | | AM493441, AM493442, AM493443, AM493444, AM493445, |
| | | | | | AM493446, AM493447, AM493448, AM493449, AM493450, |
| | | | | | AM493451, AM493491, AM493492, AM493493, AM493496, |
| | | | | | AM493498, AM493499, AM493501, AM493502, AM493503, |
| | | | | | AM493504, AM493507, AY044867, AY460204, AY587016, |
| | | | | | AY808009, AY808011, AY808018, AY808028, AY808031, |
| | | | | | AY808032, AY808033, D10934, D30613, D45172, D89815, |
| | | | | | DQ244140, DQ244141, DQ491946, DQ491979, DQ491980, |
| | | | | | DQ491981, DQ491982, DQ491983, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, DQ492124, DQ492125, DQ492126, |
| | | | | | DQ492127, DQ492128, DQ492129, DQ492130, DQ492131, |
| | | | | | DQ492132, DQ492133, DQ492134, DQ492135, DQ492136, |
| | | | | | DQ492137, DQ492138, DQ492139, DQ492140, DQ492141, |
| | | | | | DQ492142, DQ492143, DQ492144, DQ492145, DQ492146, |
| | | | | | DQ492147, DQ492148, DQ492149, DQ492150, DQ492151, |
| | | | | | DQ492152, DQ492153, DQ492154, DQ492155, DQ492156, |
| | | | | | DQ492157, DQ492158, DQ492159, DQ492160, DQ492161, |
| | | | | | DQ492162, EF407469, EF407479, EF407483, EF407487, |
| | | | | | EF407491, EF407497, EF407499, EF407502, EF407504, EU155227, |
| | | | | | EU155258, EU155261, EU155317, EU155331, EU155335, |
| | | | | | EU155337, EU155356, EU155357, EU155359, EU155361, |
| | | | | | EU155366, EU155369, EU155371, EU155374, EU155375, |
| | | | | | EU155376, EU155377, EU155382, EU255961, EU256062, |
| | | | | | EU256077, EU256079, EU256083, EU256088, EU256089, |
| | | | | | EU256099, EU256101, EU482859, EU482888, EU529682, |
| | | | | | EU660388, EU857431, FJ024279, FJ958890, FJ958891, FJ958892, |
| | | | | | FJ958893, FJ958894, FJ958895, FJ958896, FJ958897, FJ958898, |
| | | | | | FJ958899, FJ958900, FJ958901, FJ958902, FJ958903, FJ958904, |
| | | | | | FJ958905, FJ958906, FJ958907, FJ958909, FJ958910, FJ958911, |
| | | | | | FJ958912, FJ958913, FJ958914, FJ958915, FJ958916, FJ958917, |
| | | | | | FJ958918, FJ958919, FJ958920, FJ958921, FJ958922, FJ958923, |
| | | | | | FJ958924, FJ958925, FJ958926, FJ958927, FJ958928, FJ958929, |
| | | | | | FJ958930, FJ958931, FJ958932, FJ958933, FJ958934, FJ958935, |
| | | | | | GU451221, GU451222, GU451223, GU451224, HM042057, M84754 |
| 315 | ata | gtc | I | V | AJ507178 |
| 315 | ata | gtg | I | V | AB285040, AF207761, AF207765, AF483269, AJ507168, AJ507184, |
| | | | | | AY808025, EU155232, EU155336, EU255960, EU862837, FJ390398 |
| 315 | ata | gtt | I | V | AJ507197, EF407498, EU155358, FN435993 |
| 316 | tgg | agg | W | R | DQ492038 |
| 316 | tgg | cgg | W | R | AM402314, DQ492024, DQ492029 |
| 317 | gca | aca | A | T | AM401913 |
| 317 | gca | gta | A | V | DQ492077 |
| 318 | cgc | tgc | R | C | DQ491947, DQ492050 |
| 318 | cgc | tgt | R | C | DQ492082 |
| 318 | cgc | cac | R | H | AB285050, AB285078, AY808033 |
| 318 | cgc | ctc | R | L | DQ491950 |
| 318 | cgc | ccg | R | P | M96362, U16362 |
| 318 | cgc | cag | R | Q | EU529682 |
| 318 | cgc | agc | R | S | EF407500 |
| 319 | ccg | gcg | P | A | AF207757, EU155328 |
| 319 | ccg | cta | P | L | AM493488 |
| 319 | ccg | ctg | P | L | AM401889, DQ491976 |
| 319 | ccg | tcg | P | S | AM401977, DQ492022 |
| 319 | ccg | aca | P | T | DQ491965 |
| 320 | gat | gag | D | E | AY808017, EF407500, HM042059 |
| 320 | gat | ggc | D | G | AM493473, DQ492132, DQ492135 |
| 320 | gat | ggt | D | G | DQ492178 |
| 320 | gat | aat | D | N | AB285078, AY808033 |
| 320 | gat | agc | D | S | X61596 |
| 321 | tac | tgc | Y | C | AM401892, AM401896, AM401974 |
| 321 | tac | gac | Y | D | DQ491959, DQ491960, DQ491965 |
| 321 | tac | aac | Y | N | DQ491996 |
| 322 | aac | gac | N | D | AM401885, DQ492027, DQ492130, DQ492139, DQ492140 |
| 322 | aac | cac | N | H | AM493497 |
| 322 | aac | agc | N | S | AM402118, AM402349 |
| 323 | cct | ctt | P | L | AM402306 |
| 324 | cca | cta | P | L | DQ491992 |
| 324 | cca | cgg | P | R | DQ492028 |
| 325 | ctg | cca | L | P | AM402305 |
| 325 | ctg | gtg | L | V | AF207754 |
| 326 | tta | ata | L | I | AB049090, AB154183, AB154184, AB154195, AB154196, |
| | | | | | AB285048, AB285062, AB285075, AB285081, AB442220, |
| | | | | | AF165045, AF165046, AF165053, AF165054, AF207755, AF207758, |
| | | | | | AF207760, AF207771, AF265142, AF265143, AF265144, AF265145, |
| | | | | | AF265146, AF265147, AF265158, AF265159, AF265161, AJ507164, |
| | | | | | AJ507166, AJ507167, AJ507170, AJ507178, AJ507181, AJ507182, |
| | | | | | AJ507186, AJ507189, AJ507199, AM402055, AM402056, |
| | | | | | AM402057, AM402058, AM402059, AM402060, AM402061, |
| | | | | | AM402062, AM402063, AM402064, AM402065, AM402066, |
| | | | | | AM402067, AM402068, AM402069, AM402070, AM402071, |
| | | | | | AM402072, AM402073, AM402074, AM402075, AM402076, |
| | | | | | AM402077, AM402078, AM402079, AM402080, AM402081, |
| | | | | | AM402082, AM402083, AM402084, AM402085, AM402086, |
| | | | | | AM402087, AM402088, AM402089, AM402090, AM402091, |
| | | | | | AM402092, AM402093, AM402094, AM402095, AM402096, |
| | | | | | AM402097, AM402098, AM402099, AM402100, AM402101, |
| | | | | | AM402102, AM402103, AM402104, AM402105, AM402106, |
| | | | | | AM402107, AM402108, AM402109, AM402110, AM402111, |
| | | | | | AM402112, AM402113, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AM493489, AM493490, |
| | | | | | AM493493, AM493494, AM493495, AM493496, AM493497, |
| | | | | | AM493499, AM493500, AM493502, AM493505, AM493506, |
| | | | | | AY044867, AY808016, AY808018, AY808027, AY808029, |
| | | | | | AY808035, D50481, D50485, DQ492040, DQ492041, DQ492042, |
| | | | | | DQ492043, DQ492044, DQ492045, DQ492046, DQ492047, |
| | | | | | DQ492048, DQ492049, DQ492050, DQ492051, DQ492052, |
| | | | | | DQ492053, DQ492054, DQ492055, DQ492056, DQ492057, |
| | | | | | DQ492058, DQ492059, DQ492060, DQ492061, DQ492062, |
| | | | | | DQ492063, DQ492064, DQ492065, DQ492066, DQ492067, |
| | | | | | DQ492068, DQ492069, DQ492070, DQ492071, DQ492072, |
| | | | | | DQ492073, DQ492074, DQ492075, DQ492076, DQ492077, |
| | | | | | DQ492078, DQ492079, DQ492080, DQ492081, DQ492082, |
| | | | | | DQ492083, DQ492084, DQ492085, DQ492086, DQ492087, |
| | | | | | DQ492088, DQ492089, DQ492090, DQ492091, DQ492092, |
| | | | | | DQ859970, EF407460, EF407461, EF407475, EF407479, |
| | | | | | EF407482, EF407483, EF407485, EF407488, EF407492, EF407497, |
| | | | | | EF407500, EF407501, EF407502, EF407503, EU155219, |
| | | | | | EU155220, EU155221, EU155222, EU155223, EU155224, |
| | | | | | EU155226, EU155228, EU155229, EU155232, EU155255, |
| | | | | | EU155256, EU155279, EU155280, EU155300, EU155301, |
| | | | | | EU155303, EU155317, EU155326, EU155327, EU155337, |
| | | | | | EU155356, EU155357, EU155364, EU255962, EU256001, |
| | | | | | EU256045, EU256062, EU256076, EU256078, EU256081, |
| | | | | | EU256083, EU256091, EU256100, EU482833, EU781825, |
| | | | | | EU781827, EU862835, FJ024279, FJ390396, FJ958890, FJ958891, |
| | | | | | FJ958892, FJ958893, FJ958894, FJ958895, FJ958896, FJ958897, |
| | | | | | FJ958898, FJ958899, FJ958900, FJ958901, FJ958902, FJ958903, |
| | | | | | FJ958904, FJ958905, FJ958906, FJ958907, FJ958909, FJ958910, |
| | | | | | FJ958911, FJ958912, FJ958913, FJ958914, FJ958915, FJ958916, |
| | | | | | FJ958917, FJ958918, FJ958919, FJ958920, FJ958921, FJ958922, |
| | | | | | FJ958923, FJ958924, FJ958925, FJ958926, FJ958927, FJ958928, |
| | | | | | FJ958929, FJ958930, FJ958931, FJ958932, FJ958933, FJ958935, |
| | | | | | GQ913869, GQ913870, GQ913873, HM042050, HM042051, |
| | | | | | HM042052, HM042054, HM042055, HM042056, HM042057, |
| | | | | | HM042058, HM042061 |
| 326 | tta | att | L | I | EU155218, EU256000, FJ024277 |
| 326 | tta | cca | L | P | AM401855, DQ492170 |
| 326 | tta | gta | L | V | AB049096, AB154178, AB154179, AB154180, AB154181, |
| | | | | | AB154182, AB154187, AB154188, AB154191, AB154193, |
| | | | | | AB154194, AB154198, AB154199, AB154200, AB154201, |
| | | | | | AB154202, AB154203, AB154204, AB154205, AB154206, |
| | | | | | AB285038, AB285052, AB285074, AF265160, AF265162, |
| | | | | | AF313916, AJ507155, AJ507174, AJ507185, AJ507195, AY808012, |
| | | | | | DQ491999, DQ492016, EF407464, EF407469, EF407478, |
| | | | | | EU155235, EU155315, EU155325, EU256064, EU781830, |
| | | | | | FJ958934, HM042063 |
| 326 | tta | gtc | L | V | AY808034, DQ492008, DQ492009, DQ492010, DQ492011, |
| | | | | | DQ492012, DQ492013, DQ492014, DQ492015, DQ492017, |
| | | | | | DQ492018, DQ492020, DQ492021, DQ492022, DQ492023, |
| | | | | | DQ492024, DQ492025, DQ492026, DQ492027, DQ492028, |
| | | | | | DQ492029, DQ492030, DQ492031, DQ492032, DQ492033, |
| | | | | | DQ492034, DQ492035, DQ492036, DQ492037, DQ492038, |
| | | | | | DQ492039 |
| 326 | tta | gtg | L | V | AB154185, AB154186, AB154189, AB154190, AB154192, |
| | | | | | AB154197, AB285078, DQ492019, EU482881 |
| 327 | gag | ggg | E | G | AM401849, FJ958917 |
| 327 | gag | caa | E | Q | AJ507163 |
| 327 | gag | cag | E | Q | AB285073, AM493490, AM493491, AM493492, AM493493, |
| | | | | | AM493494, AM493495, AM493496, AM493497, AM493498, |
| | | | | | AM493500, AM493501, AM493502, AM493503, AM493504, |
| | | | | | AM493505, AM493506, AM493507, DQ071885, EF407504, |
| | | | | | EU660386 |
| 327 | gag | cgg | E | R | AM493489 |
| 328 | tcc | gca | S | A | DQ492165 |
| 328 | tcc | gcc | S | A | AM494937, AY808009, AY808014, AY808032, DQ492093, |
| | | | | | DQ492163, DQ492164, DQ492167, DQ492168, DQ492169, |
| | | | | | DQ492170, DQ492171, DQ492172, DQ492173, DQ492174, |
| | | | | | DQ492175, DQ492176, DQ492177, DQ492178, DQ492179, |
| | | | | | DQ492180, DQ492181, DQ492182, DQ492183, DQ492184, |
| | | | | | DQ492185, DQ492186, DQ492187, DQ492188, DQ492189, |
| | | | | | DQ492190, DQ492192, DQ492193, DQ492194, DQ492195, |
| | | | | | DQ492196, DQ492197 |
| 328 | tcc | gct | S | A | DQ492166, DQ492191 |
| 328 | tcc | ctc | S | L | EF407471 |
| 328 | tcc | cca | S | P | AY587844 |
| 328 | tcc | ccc | S | P | AB080299, AB154189, AB154190, AB285038, AB285067, |
| | | | | | AB285073, AB285079, AF034151, AF165061, AF165062, AF207769, |
| | | | | | AF265162, AJ507157, AJ507169, AM401875, AM402094, |
| | | | | | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507, AY808033, |
| | | | | | AY808034, AY808042, D50482, D50483, DQ071885, DQ244140, |
| | | | | | DQ491971, DQ491972, DQ491973, DQ491974, DQ491975, |
| | | | | | DQ491976, DQ491977, DQ491978, DQ492008, DQ492009, |
| | | | | | DQ492010, DQ492011, DQ492012, DQ492013, DQ492014, |
| | | | | | DQ492015, DQ492016, DQ492017, DQ492018, DQ492019, |
| | | | | | DQ492020, DQ492021, DQ492022, DQ492023, DQ492024, |
| | | | | | DQ492025, DQ492026, DQ492027, DQ492028, DQ492029, |
| | | | | | DQ492030, DQ492031, DQ492032, DQ492033, DQ492034, |
| | | | | | DQ492035, DQ492036, DQ492037, DQ492038, DQ492039, |
| | | | | | DQ492040, DQ492046, DQ492047, DQ492048, DQ492049, |
| | | | | | DQ492050, DQ492054, DQ492055, DQ492120, DQ492121, |
| | | | | | DQ492122, DQ492123, EF407461, EF407477, EF407491, |
| | | | | | EF407500, EF407501, EU155235, EU155256, EU155315, |
| | | | | | EU155357, EU155364, EU256079, EU256083, EU256091, |
| | | | | | EU862835, FJ958890, FJ958891, FJ958892, FJ958893, FJ958894, |
| | | | | | FJ958895, FJ958896, FJ958897, FJ958898, FJ958899, FJ958900, |
| | | | | | FJ958901, FJ958902, FJ958903, FJ958904, FJ958905, FJ958906, |
| | | | | | FJ958907, FJ958909, FJ958910, FJ958911, FJ958912, FJ958913, |
| | | | | | FJ958914, FJ958915, FJ958916, FJ958917, FJ958918, FJ958919, |
| | | | | | FJ958920, FJ958921, FJ958922, FJ958923, FJ958925, FJ958926, |
| | | | | | FJ958927, FJ958928, FJ958929, FJ958930, FJ958931, FJ958932, |
| | | | | | FJ958934, FJ958935, FN435993, GQ913873, HM042050, |
| | | | | | HM042054, HM042055, HM042056, HM042058, M84754 |
| 328 | tcc | cct | S | P | AB049096, AB049100, AB285043, AB285048, AB285075, |
| | | | | | AB285078, AF165055, AF165056, AF207771, AJ507163, AY808023, |
| | | | | | DQ244141, EU155306, GU451218 |
| 328 | tcc | acc | S | T | AB285056, AF265160, AY808028, DQ491979, DQ491980, |
| | | | | | DQ491981, DQ491982, DQ491983, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007 |
| 328 | tcc | acg | S | T | AJ507155 |
| 328 | tcc | act | S | T | AB285047 |
| 329 | tgg | agg | W | R | AM402113 |
| 329 | tgg | cgg | W | R | AM401888, AM401913 |
| 330 | aag | gag | K | E | AF483269 |
| 330 | aag | aga | K | R | AB036519, AB036520, AB036521, AB109543, AB191333, |
| | | | | | AB285050, AJ507157, DQ491971, DQ491972, DQ491973, |
| | | | | | DQ491974, DQ491976 |
| 330 | aag | agg | K | R | AB049089, AB049094, AB049095, AB285040, AB285043, |
| | | | | | AB285052, AB285057, AB285068, AB442220, AF165045, |
| | | | | | AF165046, AF165053, AF165054, AF165055, AF165056, AF207755, |
| | | | | | AF207757, AF207771, AM402121, D50482, D50484, DQ244141, |
| | | | | | DQ491977, DQ492149, DQ492158, EF407467, EU155217, |
| | | | | | EU155281, FJ958891, FJ958892, FJ958894, FJ958907, FJ958912, |
| | | | | | FJ958913, FJ958928, GU451218, GU451219, GU451220 |
| 330 | aag | acg | K | T | DQ492084 |
| 331 | gac | gcc | D | A | AF165057, AJ507162, AY240967, AY240968, AY808040, D50484, |
| | | | | | DQ491943, DQ491944, DQ491945, DQ491946, DQ491947, |
| | | | | | DQ491948, DQ491949, DQ491950, DQ491951, DQ491952, |
| | | | | | DQ491953, DQ491954, DQ491955, DQ491956, DQ491957, |
| | | | | | EU256075, HM042062 |
| 331 | gac | gga | D | G | AB285074 |
| 331 | gac | ggc | D | G | AF207768, AM402121, AM402129, DQ492067 |
| 331 | gac | cac | D | H | AF265158, AF265159 |
| 331 | gac | aaa | D | K | AB049089, AF207769, EU155263 |
| 331 | gac | aag | D | K | AJ507155 |
| 331 | gac | aac | D | N | AB010250, AB049101, AB285040, AB285044, AB285046, |
| | | | | | AB285053, AB285060, AB285061, AB429050, AB442219, |
| | | | | | AF207770, AF265148, AF265149, AF265150, AF265163, AF265164, |
| | | | | | AF356827, AF483269, AJ133096, AY587016, AY808025, AY808030, |
| | | | | | AY808036, AY808042, D30613, D45172, D89815, DQ244141, |
| | | | | | DQ491971, DQ491972, DQ491973, DQ491974, DQ491975, |
| | | | | | DQ491976, DQ491977, DQ491978, DQ491983, EF407473, |
| | | | | | EU155261, EU155306, EU155315, EU155375, EU155376, |
| | | | | | EU857431, FJ958933 |
| 331 | gac | aat | D | N | AY008261 |
| 331 | gac | aga | D | R | AY808031 |
| 331 | gac | agc | D | S | AB049100, AB285051, AB285064 |
| 331 | gac | agt | D | S | AB010249, D11168, D16435, D89872 |
| 331 | gac | gtc | D | V | AB285058 |
| 333 | gac | gcc | D | A | AJ507178, AJ507199, AY808034, DQ492008, DQ492009, |
| | | | | | DQ492010, DQ492011, DQ492012, DQ492013, DQ492014, |
| | | | | | DQ492015, DQ492016, DQ492017, DQ492018, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ492191, |
| | | | | | EU155223, GQ913869, HM042051, HM042052, HM042054, |
| | | | | | HM042055, HM042056, HM042057, HM042058, HM042061, |
| | | | | | HM042063 |
| 333 | gac | gcg | D | A | DQ492019 |
| 333 | gac | gct | D | A | AJ507170, GQ913873 |
| 333 | gac | gaa | D | E | AB285074, AB285079, AF207756 |
| 333 | gac | gag | D | E | AF033373, AM401978 |
| 333 | gac | ggc | D | G | AF165062, AY808035, DQ491955, DQ492041, DQ492042, |
| | | | | | DQ492043, DQ492044, DQ492045, DQ492051, DQ492052, |
| | | | | | DQ492056, DQ492057, DQ492058, DQ492059, DQ492060, |
| | | | | | DQ492061, DQ492062, EF407481, EU155264, EU256079, |
| | | | | | HM042059 |
| 333 | gac | aac | D | N | AB049095, AB285042, AF207764, AJ507186, AM401882, |
| | | | | | AM402115, AM402125, DQ492176, DQ492183, EU155308, |
| | | | | | EU155328, EU256059, GQ913868, GU451218 |
| 333 | gac | aat | D | N | AB285056 |
| 334 | tac | tag | Y | * | DQ492113 |
| 334 | tac | tgc | Y | C | AM402317 |
| 334 | tac | cat | Y | H | AM493506 |
| 335 | gtc | gcc | V | A | AF265142, AF265143, AF265144, AF265161, AJ507156, AM401928, |
| | | | | | D50482, D50484, DQ491948, DQ492143, DQ859970, EU155222, |
| | | | | | HM042062 |
| 335 | gtc | gct | V | A | EU155255 |
| 335 | gtc | gaa | V | E | AJ507155 |
| 335 | gtc | atc | V | I | AF207766, AM401911, DQ492094, DQ492177, EU155217 |
| 335 | gtc | att | V | I | AJ507160, EF407469, FN435993 |
| 335 | gtc | aaa | V | K | EF407480 |
| 335 | gtc | ctc | V | L | DQ492113 |
| 335 | gtc | aac | V | N | AJ507186 |
| 335 | gtc | ccc | V | P | AF207768 |
| 335 | gtc | tcc | V | S | AJ507165 |
| 335 | gtc | acc | V | T | AB285065, AY008261, AY808018, EU155223, EU256091 |
| 335 | gtc | act | V | T | EF407501, EU155361, EU155370 |
| 336 | cct | ctt | P | L | AM402306, DQ492069, DQ492164 |
| 337 | cca | gcg | P | A | EU234061 |
| 337 | cca | cgg | P | R | DQ492114 |
| 337 | cca | acg | P | T | DQ492094 |
| 338 | gtg | gcg | V | A | AB010249, AJ507179, AM402073, AM402271, AM402306, |
| | | | | | AM402346, D11168, D16435, D89872 |
| 338 | gtg | aag | V | K | DQ492194 |
| 338 | gtg | ctg | V | L | AF207765, EU155327 |
| 338 | gtg | atg | V | M | DQ492104, EU256084, FJ478453 |
| 338 | gtg | aca | V | T | EU256080 |
| 339 | gta | gca | V | A | AM493500 |
| 339 | gta | ata | V | I | DQ492007 |
| 340 | cac | cgc | H | R | AM402084, DQ491995 |
| 340 | cac | tcc | H | S | AB285051 |
| 341 | ggg | gag | G | E | AM402092, DQ492011, DQ492012, DQ492013 |
| 341 | ggg | agg | G | R | AM402365 |
| 342 | tgt | ggc | C | G | DQ492188 |
| 342 | tgt | cgc | C | R | AM401856 |
| 342 | tgt | agc | C | S | DQ491961, DQ491964, DQ491966 |
| 342 | tgt | tac | C | Y | DQ492024, DQ492050 |
| 343 | cca | gca | P | A | AY808038, HM042059 |
| 343 | cca | cta | P | L | DQ492177 |
| 343 | cca | tca | P | S | DQ492030, DQ492091 |
| 343 | cca | aca | P | T | AM401921 |
| 344 | ttg | tgc | L | C | DQ492105 |
| 345 | ccg | cac | P | H | DQ492105 |
| 345 | ccg | tca | P | S | AM493481 |
| 346 | cct | gcc | P | A | AF265158, AF265159 |
| 346 | cct | ctt | P | L | DQ492105 |
| 346 | cct | tca | P | S | DQ492079 |
| 346 | cct | tcc | P | S | AB285042, AB285048, AM493489, AM493490, AM493491, |
| | | | | | AM493492, AM493493, AM493494, AM493495, AM493496, |
| | | | | | AM493498, AM493499, AM493502, AM493503, AM493505, |
| | | | | | AM493506, AY240969, GQ913872 |
| 346 | cct | tct | P | S | AB049089, AB249644, AB426117, AJ507164, AJ507190, AY808029, |
| | | | | | D90208, DQ492063, DQ492064, DQ492065, DQ492066, DQ492067, |
| | | | | | DQ492068, DQ492070, DQ492071, DQ492073, DQ492074, |
| | | | | | DQ492075, DQ492076, DQ492078, DQ492080, DQ492082, |
| | | | | | DQ492083, DQ492084, DQ492085, DQ492086, DQ492087, |
| | | | | | DQ492088, DQ492089, DQ492090, DQ492091, DQ492092, |
| | | | | | EU482833 |
| 346 | cct | act | P | T | FJ958921 |
| 347 | gcc | gac | A | D | EU155256 |
| 347 | gcc | atc | A | I | AB285055, AF265154, AF333324, AF356827, AJ507180, AJ507181, |
| | | | | | AM493489, AM493490, AM493491, AM493492, AM493493, |
| | | | | | AM493494, AM493495, AM493496, AM493498, AM493499, |
| | | | | | AM493501, AM493502, AM493503, AM493505, AM493506, |
| | | | | | AM493507, EU155361, EU155372, EU155375, EU781825, M58335 |
| 347 | gcc | att | A | I | AB154181, AB154182, AF165061, AF165062, AM402374, |
| | | | | | EU155227, EU256103 |
| 347 | gcc | atg | A | M | AB285079 |
| 347 | gcc | aac | A | N | AF034151, EU155337, EU155381, EU781827 |
| 347 | gcc | aat | A | N | AB249644, AB426117 |
| 347 | gcc | cca | A | P | AJ507155 |
| 347 | gcc | ccc | A | P | AB285065, AB285075, AY808035, DQ492040, DQ492041, |
| | | | | | DQ492042, DQ492043, DQ492044, DQ492045, DQ492046, |
| | | | | | DQ492047, DQ492048, DQ492049, DQ492050, DQ492051, |
| | | | | | DQ492052, DQ492053, DQ492054, DQ492055, DQ492056, |
| | | | | | DQ492057, DQ492058, DQ492059, DQ492060, DQ492061, |
| | | | | | DQ492062 |
| 347 | gcc | agc | A | S | AF265155, AJ507161 |
| 347 | gcc | aca | A | T | AB285056, AJ507197, DQ492149, DQ492151, EF407464, |
| | | | | | EU155258, EU155363, GQ913870, HM042053, HM042059, |
| | | | | | HM042060 |
| 347 | gcc | acc | A | T | AB010249, AB010250, AB036519, AB036520, AB036521, |
| | | | | | AB049087, AB049088, AB049089, AB049091, AB049092, |
| | | | | | AB049096, AB049098, AB049100, AB049101, AB080299, |
| | | | | | AB109543, AB154191, AB191333, AB285035, AB285036, |
| | | | | | AB285037, AB285038, AB285040, AB285042, AB285043, |
| | | | | | AB285044, AB285046, AB285048, AB285050, AB285051, |
| | | | | | AB285054, AB285057, AB285060, AB285061, AB285062, |
| | | | | | AB285063, AB285064, AB285069, AB285070, AB285071, |
| | | | | | AB285072, AB285074, AB285076, AB285077, AB285078, |
| | | | | | AB285081, AB429050, AB442219, AB442222, AF165045, |
| | | | | | AF165046, AF165047, AF165048, AF165049, AF165050, AF165051, |
| | | | | | AF165052, AF165055, AF165056, AF165057, AF165058, AF165059, |
| | | | | | AF165060, AF165063, AF165064, AF176573, AF207752, AF207753, |
| | | | | | AF207754, AF207755, AF207756, AF207757, AF207758, AF207760, |
| | | | | | AF207761, AF207762, AF207763, AF207764, AF207766, AF207768, |
| | | | | | AF207769, AF207770, AF207771, AF207772, AF207774, AF208024, |
| | | | | | AF265142, AF265143, AF265144, AF265145, AF265146, AF265147, |
| | | | | | AF265156, AF265158, AF265159, AF265160, AF265161, AF265162, |
| | | | | | AF265165, AJ000009, AJ132997, AJ507156, AJ507158, AJ507160, |
| | | | | | AJ507162, AJ507164, AJ507167, AJ507169, AJ507170, AJ507171, |
| | | | | | AJ507172, AJ507173, AJ507179, AJ507183, AJ507184, AJ507185, |
| | | | | | AJ507186, AJ507187, AJ507188, AJ507190, AJ507191, AJ507195, |
| | | | | | AJ507196, AJ507198, AM401848, AM401849, AM401850, |
| | | | | | AM401851, AM401852, AM401853, AM401854, AM401855, |
| | | | | | AM401856, AM401857, AM401858, AM401859, AM401860, |
| | | | | | AM401861, AM401862, AM401863, AM401864, AM401865, |
| | | | | | AM401866, AM401867, AM401868, AM401869, AM401870, |
| | | | | | AM401871, AM401872, AM401873, AM401874, AM401875, |
| | | | | | AM401876, AM401877, AM401878, AM401879, AM401880, |
| | | | | | AM401881, AM401882, AM401883, AM401884, AM401885, |
| | | | | | AM401886, AM401887, AM401888, AM401889, AM401890, |
| | | | | | AM401891, AM401892, AM401893, AM401894, AM401895, |
| | | | | | AM401896, AM401897, AM401898, AM401899, AM401900, |
| | | | | | AM401901, AM401902, AM401903, AM401904, AM401905, |
| | | | | | AM401906, AM401907, AM401908, AM401909, AM401910, |
| | | | | | AM401911, AM401912, AM401913, AM401914, AM401915, |
| | | | | | AM401916, AM401917, AM401918, AM401919, AM401920, |
| | | | | | AM401921, AM401922, AM401923, AM401924, AM401925, |
| | | | | | AM401926, AM401927, AM401928, AM401929, AM401930, |
| | | | | | AM401931, AM401932, AM401933, AM401934, AM401935, |
| | | | | | AM401936, AM401937, AM401938, AM401939, AM401940, |
| | | | | | AM401941, AM401942, AM401943, AM401944, AM401945, |
| | | | | | AM401946, AM401947, AM401948, AM401949, AM401950, |
| | | | | | AM401951, AM401952, AM401953, AM401954, AM401955, |
| | | | | | AM401956, AM401957, AM401958, AM401959, AM401960, |
| | | | | | AM401961, AM401962, AM401963, AM401964, AM401965, |
| | | | | | AM401966, AM401967, AM401968, AM401969, AM401970, |
| | | | | | AM401971, AM401972, AM401973, AM401974, AM401975, |
| | | | | | AM401976, AM401977, AM401978, AM401979, AM401980, |
| | | | | | AM402059, AM402062, AM402063, AM402065, AM402079, |
| | | | | | AM402083, AM402091, AM402123, AM402124, AM402258, |
| | | | | | AM402259, AM402260, AM402261, AM402262, AM402263, |
| | | | | | AM402264, AM402265, AM402266, AM402267, AM402268, |
| | | | | | AM402269, AM402270, AM402271, AM402272, AM402273, |
| | | | | | AM402274, AM402275, AM402276, AM402277, AM402278, |
| | | | | | AM402279, AM402280, AM402281, AM402282, AM402283, |
| | | | | | AM402284, AM402285, AM402286, AM402287, AM402288, |
| | | | | | AM402289, AM402290, AM402291, AM402292, AM402293, |
| | | | | | AM402294, AM402295, AM402296, AM402297, AM402298, |
| | | | | | AM402299, AM402300, AM402301, AM402302, AM402303, |
| | | | | | AM402304, AM402305, AM402306, AM402307, AM402308, |
| | | | | | AM402309, AM402310, AM402311, AM402312, AM402313, |
| | | | | | AM402314, AM402315, AM402316, AM402317, AM402318, |
| | | | | | AM402319, AM402320, AM402321, AM402322, AM402323, |
| | | | | | AM402324, AM402325, AM402326, AM402327, AM402328, |
| | | | | | AM493437, AM493438, AM493439, AM493441, AM493443, |
| | | | | | AM493444, AM493445, AM493446, AM493447, AM493448, |
| | | | | | AM493449, AM493450, AM493451, AM493471, AM493472, |
| | | | | | AM493473, AM493474, AM493475, AM493476, AM493477, |
| | | | | | AM493478, AM493479, AM493480, AM493481, AM493482, |
| | | | | | AM493483, AM493484, AM493485, AM493486, AM493487, |
| | | | | | AM493488, AM493497, AM493500, AM494937, AY044867, |
| | | | | | AY045702, AY240967, AY240968, AY240969, AY460204, |
| | | | | | AY587016, AY587844, AY808014, AY808017, AY808020, |
| | | | | | AY808021, AY808022, AY808025, AY808026, AY808027, |
| | | | | | AY808029, AY808031, AY808032, AY808034, AY808038, |
| | | | | | AY808039, AY808041, AY808042, AY808043, D11168, D14484, |
| | | | | | D16435, D30613, D45172, D50481, D50485, D85516, D89815, |
| | | | | | D89872, D90208, DQ244141, DQ491958, DQ491959, DQ491960, |
| | | | | | DQ491961, DQ491962, DQ491963, DQ491964, DQ491965, |
| | | | | | DQ491966, DQ491967, DQ491968, DQ491969, DQ491970, |
| | | | | | DQ491971, DQ491972, DQ491973, DQ491974, DQ491975, |
| | | | | | DQ491976, DQ491977, DQ491978, DQ492008, DQ492009, |
| | | | | | DQ492010, DQ492011, DQ492012, DQ492013, DQ492014, |
| | | | | | DQ492015, DQ492017, DQ492018, DQ492019, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ492063, |
| | | | | | DQ492064, DQ492065, DQ492066, DQ492067, DQ492068, |
| | | | | | DQ492069, DQ492070, DQ492071, DQ492072, DQ492073, |
| | | | | | DQ492074, DQ492075, DQ492076, DQ492077, DQ492078, |
| | | | | | DQ492079, DQ492080, DQ492081, DQ492082, DQ492083, |
| | | | | | DQ492084, DQ492085, DQ492086, DQ492087, DQ492088, |
| | | | | | DQ492089, DQ492090, DQ492091, DQ492092, DQ492093, |
| | | | | | DQ492127, DQ492130, DQ492131, DQ492139, DQ492140, |
| | | | | | DQ492163, DQ492164, DQ492165, DQ492166, DQ492167, |
| | | | | | DQ492168, DQ492169, DQ492170, DQ492171, DQ492173, |
| | | | | | DQ492174, DQ492175, DQ492176, DQ492177, DQ492178, |
| | | | | | DQ492179, DQ492180, DQ492181, DQ492182, DQ492183, |
| | | | | | DQ492184, DQ492185, DQ492186, DQ492187, DQ492188, |
| | | | | | DQ492189, DQ492190, DQ492191, DQ492192, DQ492193, |
| | | | | | DQ492194, DQ492195, DQ492196, DQ492197, DQ859970, |
| | | | | | EF032892, EF032893, EF032894, EF407459, EF407460, EF407462, |
| | | | | | EF407463, EF407466, EF407468, EF407469, EF407470, EF407471, |
| | | | | | EF407473, EF407474, EF407475, EF407476, EF407478, EF407479, |
| | | | | | EF407480, EF407482, EF407483, EF407484, EF407485, EF407486, |
| | | | | | EF407487, EF407488, EF407490, EF407491, EF407492, EF407495, |
| | | | | | EF407497, EF407498, EF407500, EF407502, EF407503, EF407504, |
| | | | | | EF638081, EU155217, EU155218, EU155220, EU155222, |
| | | | | | EU155223, EU155224, EU155226, EU155229, EU155230, |
| | | | | | EU155232, EU155255, EU155257, EU155259, EU155260, |
| | | | | | EU155263, EU155279, EU155281, EU155300, EU155301, |
| | | | | | EU155302, EU155304, EU155308, EU155316, EU155318, |
| | | | | | EU155324, EU155325, EU155326, EU155327, EU155328, |
| | | | | | EU155329, EU155330, EU155331, EU155332, EU155333, |
| | | | | | EU155335, EU155336, EU155356, EU155358, EU155364, |
| | | | | | EU155366, EU155368, EU155369, EU155373, EU155374, |
| | | | | | EU155377, EU234061, EU234062, EU239714, EU255961, |
| | | | | | EU255962, EU256000, EU256001, EU256059, EU256061, |
| | | | | | EU256062, EU256064, EU256066, EU256075, EU256077, |
| | | | | | EU256079, EU256080, EU256081, EU256082, EU256083, |
| | | | | | EU256084, EU256085, EU256089, EU256092, EU256098, |
| | | | | | EU256099, EU256100, EU256101, EU482849, EU482859, |
| | | | | | EU482860, EU482877, EU482883, EU482885, EU482888, |
| | | | | | EU781830, EU857431, EU862837, FJ024277, FJ024279, FJ390398, |
| | | | | | FJ958890, FJ958891, FJ958892, FJ958893, FJ958894, FJ958895, |
| | | | | | FJ958896, FJ958897, FJ958898, FJ958899, FJ958900, FJ958901, |
| | | | | | FJ958902, FJ958903, FJ958904, FJ958905, FJ958906, FJ958907, |
| | | | | | FJ958909, FJ958910, FJ958911, FJ958912, FJ958913, FJ958914, |
| | | | | | FJ958915, FJ958916, FJ958917, FJ958918, FJ958919, FJ958920, |
| | | | | | FJ958921, FJ958922, FJ958923, FJ958924, FJ958925, FJ958926, |
| | | | | | FJ958927, FJ958928, FJ958929, FJ958930, FJ958931, FJ958932, |
| | | | | | FJ958933, FJ958934, GQ913868, GQ913871, GU451218, |
| | | | | | GU451219, GU451220, GU451221, GU451222, GU451223, |
| | | | | | GU451224, HM042050, HM042058, HM042062, HM042063, L02836, |
| | | | | | M96362, U16362, U45476, X61596 |
| 347 | gcc | acg | A | T | AB285066 |
| 347 | gcc | act | A | T | AB049090, AB049099, AB154177, AB154178, AB154179, |
| | | | | | AB154180, AB154183, AB154184, AB154185, AB154186, |
| | | | | | AB154187, AB154188, AB154189, AB154190, AB154192, |
| | | | | | AB154193, AB154194, AB154195, AB154196, AB154197, |
| | | | | | AB154198, AB154199, AB154200, AB154201, AB154202, |
| | | | | | AB154203, AB154204, AB154205, AB154206, AB285047, |
| | | | | | AB285059, AB285067, AB442221, AF265148, AF265149, |
| | | | | | AF265150, AF265152, AF265153, AF265163, AF265164, AF313916, |
| | | | | | AF483269, AJ507157, AJ507159, AJ507163, AJ507166, AJ507178, |
| | | | | | AJ507182, AJ507192, AJ507199, AM402056, AM402057, |
| | | | | | AM402058, AM402060, AM402061, AM402064, AM402066, |
| | | | | | AM402067, AM402068, AM402069, AM402070, AM402071, |
| | | | | | AM402072, AM402073, AM402074, AM402075, AM402076, |
| | | | | | AM402077, AM402078, AM402080, AM402081, AM402082, |
| | | | | | AM402084, AM402085, AM402086, AM402087, AM402088, |
| | | | | | AM402089, AM402090, AM402092, AM402093, AM402094, |
| | | | | | AM402095, AM402096, AM402097, AM402098, AM402099, |
| | | | | | AM402100, AM402101, AM402102, AM402103, AM402104, |
| | | | | | AM402105, AM402106, AM402107, AM402108, AM402109, |
| | | | | | AM402110, AM402111, AM402112, AM402113, AM402114, |
| | | | | | AM402115, AM402116, AM402117, AM402118, AM402119, |
| | | | | | AM402120, AM402121, AM402122, AM402125, AM402126, |
| | | | | | AM402127, AM402128, AM402129, AM402329, AM402330, |
| | | | | | AM402331, AM402332, AM402333, AM402334, AM402335, |
| | | | | | AM402336, AM402337, AM402338, AM402339, AM402340, |
| | | | | | AM402341, AM402342, AM402343, AM402344, AM402345, |
| | | | | | AM402346, AM402347, AM402348, AM402349, AM402350, |
| | | | | | AM402351, AM402352, AM402353, AM402354, AM402355, |
| | | | | | AM402356, AM402357, AM402358, AM402359, AM402360, |
| | | | | | AM402361, AM402362, AM402363, AM402364, AM402365, |
| | | | | | AM402367, AM402369, AM402370, AM402371, AM402372, |
| | | | | | AM402373, AM402375, AM402376, AM402377, AM402378, |
| | | | | | AM402379, AM402380, AM402381, AM402382, AM402383, |
| | | | | | AM402384, AY808011, AY808012, AY808016, AY808024, |
| | | | | | AY808028, AY808030, AY808033, AY808036, AY808040, D10934, |
| | | | | | DQ491943, DQ491944, DQ491945, DQ491946, DQ491947, |
| | | | | | DQ491948, DQ491949, DQ491950, DQ491951, DQ491952, |
| | | | | | DQ491953, DQ491954, DQ491955, DQ491956, DQ491957, |
| | | | | | DQ491979, DQ491980, DQ491981, DQ491982, DQ491983, |
| | | | | | DQ491984, DQ491985, DQ491986, DQ491987, DQ491988, |
| | | | | | DQ491989, DQ491990, DQ491991, DQ491992, DQ491993, |
| | | | | | DQ491994, DQ491995, DQ491996, DQ491997, DQ491998, |
| | | | | | DQ491999, DQ492000, DQ492001, DQ492002, DQ492003, |
| | | | | | DQ492004, DQ492005, DQ492006, DQ492007, DQ492124, |
| | | | | | DQ492125, DQ492126, DQ492128, DQ492129, DQ492132, |
| | | | | | DQ492133, DQ492134, DQ492135, DQ492136, DQ492137, |
| | | | | | DQ492138, DQ492141, DQ492142, DQ492143, DQ492144, |
| | | | | | DQ492145, DQ492146, DQ492147, DQ492150, DQ492152, |
| | | | | | DQ492153, DQ492154, DQ492155, DQ492156, DQ492157, |
| | | | | | DQ492158, DQ492159, DQ492160, DQ492161, DQ492162, |
| | | | | | DQ492172, EF407481, EF407489, EF407496, EU155219, |
| | | | | | EU155221, EU155225, EU155228, EU155231, EU155235, |
| | | | | | EU155253, EU155254, EU155261, EU155262, EU155303, |
| | | | | | EU155305, EU155307, EU155317, EU155359, EU155360, |
| | | | | | EU155362, EU155367, EU255960, EU256045, EU256076, |
| | | | | | EU256078, EU256090, EU482833, EU482839, EU482875, |
| | | | | | EU482879, EU482880, EU529682, EU660386, EU660388, |
| | | | | | EU862835, FJ390396, FJ390397, GQ913869, GQ913872, |
| | | | | | HM042051, HM042052, HM042054, HM042055, HM042056, |
| | | | | | HM042057, HM042061 |
| 347 | gcc | gtc | A | V | AB285058, AF207765, AJ507168, AJ507194, EF407501, EU256065, |
| | | | | | EU482874, M84754 |
| 347 | gcc | gtt | A | V | AJ507175, EF407467 |
| 348 | aag | gga | K | G | FJ958904 |
| 348 | aag | ggg | K | G | AJ507189, AY808040, DQ491943, DQ491944, DQ491945, |
| | | | | | DQ491946, DQ491947, DQ491948, DQ491949, DQ491950, |
| | | | | | DQ491951, DQ491952, DQ491953, DQ491954, DQ491955, |
| | | | | | DQ491956, DQ491957, EU660388 |
| 348 | aag | atg | K | M | DQ492189, X61596 |
| 348 | aag | ccg | K | P | DQ492040, DQ492073 |
| 348 | aag | cag | K | Q | AB285063 |
| 348 | aag | aga | K | R | AM402328, FJ958890, FJ958893, FJ958896, FJ958900, FJ958901, |
| | | | | | FJ958902, FJ958903, FJ958906, FJ958914, FJ958915, FJ958916, |
| | | | | | FJ958917, FJ958919, FJ958920, FJ958921, FJ958927, FJ958928, |
| | | | | | FJ958931, FJ958935 |
| 348 | aag | agg | K | R | AB049099, AB429050, AF165052, AJ132997, AJ507157, AJ507172, |
| | | | | | AM401904, AM402258, AM402259, AM402260, AM402261, |
| | | | | | AM402262, AM402263, AM402264, AM402265, AM402266, |
| | | | | | AM402267, AM402268, AM402269, AM402270, AM402271, |
| | | | | | AM402272, AM402273, AM402274, AM402275, AM402276, |
| | | | | | AM402277, AM402278, AM402279, AM402280, AM402281, |
| | | | | | AM402282, AM402283, AM402284, AM402285, AM402286, |
| | | | | | AM402287, AM402288, AM402289, AM402290, AM402291, |
| | | | | | AM402292, AM402293, AM402294, AM402295, AM402296, |
| | | | | | AM402297, AM402298, AM402299, AM402300, AM402301, |
| | | | | | AM402302, AM402303, AM402304, AM402305, AM402306, |
| | | | | | AM402307, AM402308, AM402309, AM402310, AM402311, |
| | | | | | AM402312, AM402313, AM402314, AM402315, AM402316, |
| | | | | | AM402317, AM402318, AM402319, AM402320, AM402321, |
| | | | | | AM402322, AM402323, AM402324, AM402325, AM402326, |
| | | | | | AM402327, AM402329, AM402330, AM402331, AM402332, |
| | | | | | AM402333, AM402334, AM402335, AM402336, AM402337, |
| | | | | | AM402338, AM402339, AM402340, AM402341, AM402342, |
| | | | | | AM402343, AM402344, AM402345, AM402346, AM402347, |
| | | | | | AM402348, AM402350, AM402351, AM402352, AM402353, |
| | | | | | AM402354, AM402355, AM402357, AM402358, AM402359, |
| | | | | | AM402360, AM402361, AM402362, AM402363, AM402364, |
| | | | | | AM402365, AM402367, AM402369, AM402370, AM402371, |
| | | | | | AM402372, AM402373, AM402374, AM402375, AM402376, |
| | | | | | AM402377, AM402378, AM402379, AM402380, AM402381, |
| | | | | | AM402382, AM402383, AM402384, AM493436, AM493437, |
| | | | | | AM493438, AM493439, AM493440, AM493441, AM493442, |
| | | | | | AM493443, AM493444, AM493445, AM493446, AM493447, |
| | | | | | AM493448, AM493449, AM493450, AM493451, AM493504, |
| | | | | | AY808034, AY808043, DQ491967, DQ492008, DQ492009, |
| | | | | | DQ492010, DQ492011, DQ492012, DQ492013, DQ492014, |
| | | | | | DQ492017, DQ492020, DQ492021, DQ492022, DQ492023, |
| | | | | | DQ492024, DQ492025, DQ492026, DQ492027, DQ492028, |
| | | | | | DQ492029, DQ492030, DQ492031, DQ492032, DQ492033, |
| | | | | | DQ492034, DQ492035, DQ492036, DQ492037, DQ492038, |
| | | | | | DQ492039, DQ492124, DQ492125, DQ492129, DQ492138, |
| | | | | | EF407466, EF407470, EF407495, EF407497, EU155361, |
| | | | | | EU155370, HM042051 |
| 348 | aag | cgg | K | R | AJ507155, AY808035, DQ492041, DQ492042, DQ492043, |
| | | | | | DQ492044, DQ492045, DQ492046, DQ492047, DQ492048, |
| | | | | | DQ492049, DQ492050, DQ492051, DQ492052, DQ492053, |
| | | | | | DQ492054, DQ492055, DQ492056, DQ492057, DQ492058, |
| | | | | | DQ492059, DQ492060, DQ492061, DQ492062 |
| 348 | aag | acg | K | T | AB010249, AB154188, AY808029, D11168, D16435, D89872, |
| | | | | | DQ492063, DQ492064, DQ492065, DQ492066, DQ492067, |
| | | | | | DQ492068, DQ492069, DQ492070, DQ492071, DQ492072, |
| | | | | | DQ492074, DQ492075, DQ492076, DQ492077, DQ492078, |
| | | | | | DQ492079, DQ492080, DQ492081, DQ492082, DQ492083, |
| | | | | | DQ492084, DQ492085, DQ492086, DQ492087, DQ492088, |
| | | | | | DQ492089, DQ492090, DQ492091, DQ492092, EU256079, |
| | | | | | FJ390398, GQ913871, L02836 |
| 349 | gcc | gac | A | D | D50482, D50484 |
| 349 | gcc | gag | A | E | AY808022 |
| 349 | gcc | ggc | A | G | AB010249, AB049088, AB049089, AB285036, AB285038, |
| | | | | | AF207752, AF356827, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, D11168, |
| | | | | | D16435, D89872, EU155260, EU256001, EU256066, FN435993 |
| 349 | gcc | ggg | A | G | AF207753 |
| 349 | gcc | atc | A | I | AY808018, FJ958891, FJ958923 |
| 349 | gcc | aat | A | N | DQ492041, DQ492043, DQ492044, DQ492045, DQ492051, |
| | | | | | DQ492052 |
| 349 | gcc | ccc | A | P | AF165048, EF407485, EU155259 |
| 349 | gcc | agc | A | S | AY808010 |
| 349 | gcc | acc | A | T | AB285052, AB285054, AB285056, AB285059, AB285068, |
| | | | | | AB442220, AF165045, AF165046, AF165053, AF165054, AF207771, |
| | | | | | AJ507163, AJ507189, AM401931, AM402090, AM402299, |
| | | | | | AY808027, AY808043, EF407463, EF407494, EF407499, |
| | | | | | EU155226, EU155300, EU155332, EU155359, EU155377, |
| | | | | | EU255960, FJ958892, FJ958894, FJ958895, FJ958897, FJ958898, |
| | | | | | FJ958899, FJ958905, FJ958907, FJ958909, FJ958910, FJ958911, |
| | | | | | FJ958912, FJ958913, FJ958918, FJ958922, FJ958924, FJ958925, |
| | | | | | FJ958926, FJ958929, FJ958930, FJ958932, FJ958934 |
| 349 | gcc | acg | A | T | AY808028, DQ491979, DQ491980, DQ491981, DQ491982, |
| | | | | | DQ491983, DQ491984, DQ491985, DQ491986, DQ491987, |
| | | | | | DQ491988, DQ491989, DQ491990, DQ491991, DQ491992, |
| | | | | | DQ491993, DQ491994, DQ491995, DQ491996, DQ491997, |
| | | | | | DQ491998, DQ491999, DQ492000, DQ492001, DQ492002, |
| | | | | | DQ492003, DQ492004, DQ492005, DQ492006, DQ492007 |
| 349 | gcc | act | A | T | AB285063, AB285078, AF207757, AY808035, DQ244140, |
| | | | | | DQ492040, DQ492042, DQ492046, DQ492047, DQ492048, |
| | | | | | DQ492049, DQ492050, DQ492053, DQ492054, DQ492055, |
| | | | | | DQ492056, DQ492057, DQ492058, DQ492059, DQ492060, |
| | | | | | DQ492061, DQ492062 |
| 349 | gcc | gta | A | V | AB285042, FJ390396 |
| 349 | gcc | gtc | A | V | AB285075, AF176573, AY008261, AY808030, DQ492156, |
| | | | | | EU155370, EU155371, FJ958917, GU451218, GU451219, |
| | | | | | GU451220 |
| 349 | gcc | gtg | A | V | AJ507161 |
| 350 | cct | gcc | P | A | AY808010, EU256102 |
| 350 | cct | gct | P | A | D14484 |
| 350 | cct | cat | P | H | AJ507192 |
| 350 | cct | ctt | P | L | AF207762 |
| 350 | cct | tcc | P | S | DQ492002 |
| 351 | ccg | tca | P | S | AM402297, DQ244141 |
| 352 | ata | tta | I | L | AF207753, AF207772 |
| 352 | ata | atg | I | M | AM402066, AM402319 |
| 352 | ata | aca | I | T | AY808025, DQ492019, DQ492153 |
| 352 | ata | acg | I | T | EU482883 |
| 352 | ata | gta | I | V | AB285038, AB285048, AB285063, AB285071, AB285077, |
| | | | | | AF165053, AF165054, AF265154, AF265155, AF265156, AJ132996, |
| | | | | | AJ507162, AJ507166, AM493489, AM493490, AM493491, |
| | | | | | AM493492, AM493493, AM493494, AM493495, AM493496, |
| | | | | | AM493497, AM493498, AM493499, AM493500, AM493501, |
| | | | | | AM493502, AM493503, AM493504, AM493505, AM493506, |
| | | | | | AM493507, AY008261, AY808023, AY808024, AY808043, |
| | | | | | DQ492090, DQ492172, EF407487, EU155305, EU155360, |
| | | | | | EU256080, EU256102, EU482881, L02836 |
| 352 | ata | gtg | I | V | AB049101, AF165061, AF165062, AF207760, AF207762, AF207769, |
| | | | | | AJ507155, AM401848, AM401849, AM401850, AM401851, |
| | | | | | AM401852, AM401853, AM401854, AM401855, AM401856, |
| | | | | | AM401857, AM401858, AM401859, AM401860, AM401861, |
| | | | | | AM401862, AM401863, AM401864, AM401865, AM401866, |
| | | | | | AM401867, AM401868, AM401869, AM401870, AM401871, |
| | | | | | AM401872, AM401873, AM401874, AM401875, AM401876, |
| | | | | | AM401877, AM401878, AM401879, AM401880, AM401881, |
| | | | | | AM401882, AM401883, AM401884, AM401885, AM401886, |
| | | | | | AM401887, AM401888, AM401889, AM401890, AM401891, |
| | | | | | AM401892, AM401893, AM401894, AM401895, AM401896, |
| | | | | | AM401897, AM401898, AM401899, AM401900, AM401901, |
| | | | | | AM401902, AM401903, AM401904, AM401905, AM401906, |
| | | | | | AM401907, AM401908, AM401909, AM401910, AM401911, |
| | | | | | AM401912, AM401913, AM401914, AM401915, DQ244141, |
| | | | | | EF407489, EU155219, EU155315, EU155317, EU155376, |
| | | | | | EU482859, GQ913871 |
| 353 | cca | tca | P | S | AM402071 |
| 354 | cct | ctt | P | L | AM402125 |
| 355 | cca | cta | P | L | DQ492004 |
| 355 | cca | caa | P | Q | AF207772 |
| 355 | cca | tca | P | S | AM402374, DQ492164 |
| 356 | cgg | ctg | R | L | DQ492172, DQ492197 |
| 356 | cgg | cag | R | Q | DQ492114 |
| 356 | cgg | tgg | R | W | DQ492133 |
| 357 | agg | ggg | R | G | DQ492008 |
| 357 | agg | aaa | R | K | AB010249, AB049094, AB285039, AB285078, AF207774, AJ000009, |
| | | | | | AJ132997, AM493471, AM493472, AM493473, AM493474, |
| | | | | | AM493475, AM493476, AM493477, AM493478, AM493479, |
| | | | | | AM493480, AM493481, AM493482, AM493483, AM493484, |
| | | | | | AM493485, AM493486, AM493487, AM493488, AM493489, |
| | | | | | AM493490, AM493491, AM493492, AM493493, AM493494, |
| | | | | | AM493495, AM493496, AM493497, AM493498, AM493499, |
| | | | | | AM493500, AM493502, AM493503, AM493504, AM493505, |
| | | | | | AM493506, AM493507, AY587844, AY808020, AY808022, D11168, |
| | | | | | D16435, D89872, DQ492182, EF407471, EF407489, EF407496, |
| | | | | | EF407497, EU155331, EU155373, EU256079, EU256083, FJ024279 |
| 357 | agg | aag | R | K | AB049095, AM493501, AY808011, AY808025, DQ492124, |
| | | | | | DQ492125, DQ492126, DQ492127, DQ492128, DQ492129, |
| | | | | | DQ492130, DQ492131, DQ492132, DQ492133, DQ492134, |
| | | | | | DQ492135, DQ492136, DQ492137, DQ492138, DQ492139, |
| | | | | | DQ492140, DQ492141, DQ492142, DQ492143, DQ492144, |
| | | | | | DQ492145, DQ492146, DQ492147, DQ492148, DQ492149, |
| | | | | | DQ492150, DQ492151, DQ492152, DQ492153, DQ492154, |
| | | | | | DQ492155, DQ492156, DQ492157, DQ492158, DQ492159, |
| | | | | | DQ492160, DQ492161, DQ492162, EU155364, EU256061, |
| | | | | | EU256077 |
| 357 | agg | tgg | R | W | DQ492053 |
| 358 | aag | gaa | K | E | DQ492090 |
| 358 | aag | gag | K | E | AM493501 |
| 358 | aag | aga | K | R | EF407459 |
| 358 | aag | agg | K | R | DQ492083, EF407498, EU862835, FJ958921, FJ958933 |
| 359 | agg | gga | R | G | DQ492072 |
| 359 | agg | ggg | R | G | AM401932 |
| 359 | agg | aag | R | K | AB049094, AB249644, AB285052, AB285069, AB426117, |
| | | | | | AF165045, AF165046, AF207755, AF207757, AF207771, AF265148, |
| | | | | | AF265149, AF265150, AF265163, AF265164, AF265165, AJ507161, |
| | | | | | AJ507179, AJ507189, AM402055, AM402056, AM402057, |
| | | | | | AM402058, AM402059, AM402060, AM402061, AM402062, |
| | | | | | AM402063, AM402064, AM402065, AM402066, AM402067, |
| | | | | | AM402068, AM402069, AM402070, AM402071, AM402072, |
| | | | | | AM402073, AM402074, AM402075, AM402076, AM402077, |
| | | | | | AM402078, AM402079, AM402080, AM402081, AM402082, |
| | | | | | AM402083, AM402084, AM402085, AM402086, AM402087, |
| | | | | | AM402088, AM402089, AM402090, AM402091, AM402092, |
| | | | | | AM402093, AM402094, AM402095, AM402096, AM402097, |
| | | | | | AM402098, AM402099, AM402100, AM402101, AM402102, |
| | | | | | AM402103, AM402104, AM402105, AM402106, AM402107, |
| | | | | | AM402108, AM402109, AM402110, AM402111, AM402112, |
| | | | | | AM402113, AM402114, AM402115, AM402116, AM402117, |
| | | | | | AM402118, AM402119, AM402120, AM402121, AM402122, |
| | | | | | AM402123, AM402124, AM402125, AM402126, AM402127, |
| | | | | | AM402128, AM402129, AY808028, AY808030, DQ491979, |
| | | | | | DQ491980, DQ491981, DQ491982, DQ491983, DQ491984, |
| | | | | | DQ491985, DQ491986, DQ491987, DQ491988, DQ491989, |
| | | | | | DQ491990, DQ491991, DQ491992, DQ491993, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, DQ492190, EF407459, |
| | | | | | EF407468, EF407480, EF407484, EU155221, EU155231, |
| | | | | | EU155257, EU155258, EU155304, EU155307, EU155308, |
| | | | | | EU155316, EU155318, EU155365, EU155367, EU155377, |
| | | | | | EU256001, EU256045, EU256066, EU256085, EU256088, |
| | | | | | EU256098, EU482886, EU529682, EU862835, FJ390397, FJ390398, |
| | | | | | GU451218, GU451219, GU451220 |
| 360 | acg | gca | T | A | AM401855 |
| 360 | acg | gcc | T | A | AJ507191 |
| 360 | acg | gcg | T | A | EF407495, EU155304, EU256085, GQ913873 |
| 360 | acg | atg | T | M | DQ491998, DQ492003, DQ492004, DQ492005 |
| 361 | gtt | gct | V | A | AM401957, AM402059, AM402280, DQ492179 |
| 361 | gtt | ttc | V | F | AF207760 |
| 361 | gtt | ata | V | I | AJ507156, EU256061 |
| 361 | gtt | atc | V | I | AB049099, AM493491, AM493492, DQ492131, DQ492155, |
| | | | | | EU155262, EU256059, FJ958909, FJ958927 |
| 361 | gtt | att | V | I | AB285036, AB285060, AB442221, AF207764, AM401901, |
| | | | | | AM493489, AM493490, AM493493, AM493494, AM493495, |
| | | | | | AM493496, AM493497, AM493498, AM493499, AM493500, |
| | | | | | AM493501, AM493502, AM493503, AM493504, AM493505, |
| | | | | | AM493506, AM493507, AY044867, AY808011, D14484, DQ492124, |
| | | | | | DQ492125, DQ492126, DQ492127, DQ492128, DQ492129, |
| | | | | | DQ492130, DQ492132, DQ492133, DQ492134, DQ492135, |
| | | | | | DQ492136, DQ492137, DQ492138, DQ492139, DQ492140, |
| | | | | | DQ492141, DQ492142, DQ492143, DQ492144, DQ492145, |
| | | | | | DQ492146, DQ492147, DQ492148, DQ492149, DQ492150, |
| | | | | | DQ492151, DQ492152, DQ492153, DQ492154, DQ492156, |
| | | | | | DQ492157, DQ492159, DQ492160, DQ492161, DQ492162, |
| | | | | | EF407476, EU155261, EU155304, EU155305, EU155336, |
| | | | | | EU155376, EU256084, EU256090, EU256091, EU482880, |
| | | | | | FJ958890, FJ958891, FJ958892, FJ958893, FJ958894, FJ958895, |
| | | | | | FJ958896, FJ958897, FJ958898, FJ958899, FJ958900, FJ958901, |
| | | | | | FJ958902, FJ958903, FJ958904, FJ958905, FJ958906, FJ958907, |
| | | | | | FJ958910, FJ958911, FJ958912, FJ958913, FJ958914, FJ958915, |
| | | | | | FJ958916, FJ958917, FJ958918, FJ958919, FJ958920, FJ958921, |
| | | | | | FJ958922, FJ958923, FJ958924, FJ958925, FJ958926, FJ958928, |
| | | | | | FJ958929, FJ958930, FJ958931, FJ958932, FJ958933, FJ958934, |
| | | | | | FJ958935, HM042050 |
| 362 | gtc | gcc | V | A | AB080299, AM402337, AM493489, AM493490, AM493491, |
| | | | | | AM493492, AM493493, AM493494, AM493495, AM493496, |
| | | | | | AM493498, AM493499, AM493501, AM493502, AM493503, |
| | | | | | AM493504, AM493505, AM493506, AM493507, DQ491947, |
| | | | | | EU155376 |
| 362 | gtc | gac | V | D | AM402096 |
| 362 | gtc | atc | V | I | AB036519, AB036520, AB036521, AB049087, AB049089, |
| | | | | | AB049096, AB109543, AB191333, AB285035, AB285042, |
| | | | | | AB285048, AB285050, AB285053, AB285060, AB285068, |
| | | | | | AB285073, AB429050, AF165055, AF165056, AF165063, AF165064, |
| | | | | | AM402258, AM402259, AM402260, AM402261, AM402262, |
| | | | | | AM402263, AM402264, AM402265, AM402266, AM402267, |
| | | | | | AM402268, AM402269, AM402270, AM402271, AM402272, |
| | | | | | AM402273, AM402274, AM402275, AM402276, AM402277, |
| | | | | | AM402278, AM402279, AM402280, AM402281, AM402282, |
| | | | | | AM402283, AM402284, AM402285, AM402286, AM402287, |
| | | | | | AM402288, AM402289, AM402290, AM402291, AM402292, |
| | | | | | AM402293, AM402294, AM402295, AM402296, AM402298, |
| | | | | | AM402299, AM402300, AM402301, AM402302, AM402303, |
| | | | | | AM402304, AM402305, AM402306, AM402307, AM402308, |
| | | | | | AM402309, AM402310, AM402311, AM402312, AM402313, |
| | | | | | AM402314, AM402315, AM402316, AM402317, AM402318, |
| | | | | | AM402319, AM402320, AM402321, AM402322, AM402323, |
| | | | | | AM402324, AM402325, AM402326, AM402327, AM402328, |
| | | | | | AM493436, AM493437, AM493438, AM493439, AM493440, |
| | | | | | AM493441, AM493442, AM493443, AM493444, AM493445, |
| | | | | | AM493446, AM493447, AM493448, AM493449, AM493450, |
| | | | | | AM493451, DQ492094, DQ492102, DQ859970, EU256000, |
| | | | | | EU256059, GU451218 |
| 362 | gtc | att | V | I | AB285043, AB285054, AB285055, AB285079, AY240967 |
| 362 | gtc | act | V | T | AM493497, AM493500 |
| 363 | ctg | ccg | L | P | AM401979, AM402316, DQ492112 |
| 363 | ctg | cag | L | Q | AM401951, DQ492115 |
| 364 | tca | gca | S | A | AM401965, AM402069, AM402094, DQ491979, DQ492180 |
| 364 | tca | aca | S | T | AB010249, AB010250, AB036519, AB036520, AB036521, |
| | | | | | AB049087, AB049089, AB049090, AB049091, AB049092, |
| | | | | | AB049095, AB049096, AB049098, AB049099, AB049101, |
| | | | | | AB080299, AB109543, AB154177, AB154178, AB154179, |
| | | | | | AB154180, AB154181, AB154182, AB154183, AB154184, |
| | | | | | AB154185, AB154186, AB154187, AB154188, AB154189, |
| | | | | | AB154190, AB154191, AB154192, AB154193, AB154194, |
| | | | | | AB154195, AB154196, AB154197, AB154198, AB154199, |
| | | | | | AB154200, AB154201, AB154202, AB154203, AB154204, |
| | | | | | AB154205, AB154206, AB191333, AB285035, AB285036, |
| | | | | | AB285037, AB285038, AB285039, AB285040, AB285042, |
| | | | | | AB285043, AB285044, AB285046, AB285047, AB285048, |
| | | | | | AB285050, AB285052, AB285053, AB285054, AB285055, |
| | | | | | AB285056, AB285058, AB285059, AB285060, AB285061, |
| | | | | | AB285062, AB285063, AB285064, AB285065, AB285066, |
| | | | | | AB285067, AB285068, AB285069, AB285070, AB285071, |
| | | | | | AB285072, AB285073, AB285075, AB285076, AB285077, |
| | | | | | AB285078, AB285079, AB285081, AB429050, AB442219, |
| | | | | | AB442220, AB442221, AB442222, AF033373, AF034151, |
| | | | | | AF165045, AF165046, AF165047, AF165048, AF165049, AF165050, |
| | | | | | AF165051, AF165052, AF165053, AF165054, AF165055, AF165056, |
| | | | | | AF165057, AF165058, AF165059, AF165060, AF165061, AF165062, |
| | | | | | AF165063, AF165064, AF207752, AF207753, AF207754, AF207755, |
| | | | | | AF207756, AF207758, AF207760, AF207762, AF207763, AF207764, |
| | | | | | AF207765, AF207766, AF207768, AF207769, AF207770, AF207771, |
| | | | | | AF207774, AF208024, AF265145, AF265146, AF265147, AF265152, |
| | | | | | AF265153, AF265158, AF265159, AF313916, AF333324, AF356827, |
| | | | | | AF483269, AJ000009, AJ132996, AJ132997, AJ507155, AJ507156, |
| | | | | | AJ507157, AJ507158, AJ507159, AJ507160, AJ507161, AJ507162, |
| | | | | | AJ507165, AJ507166, AJ507168, AJ507169, AJ507172, AJ507173, |
| | | | | | AJ507177, AJ507179, AJ507180, AJ507181, AJ507182, AJ507183, |
| | | | | | AJ507184, AJ507185, AJ507186, AJ507187, AJ507188, AJ507189, |
| | | | | | AJ507191, AJ507192, AJ507193, AJ507195, AJ507196, AJ507198, |
| | | | | | AJ507199, AM401848, AM401849, AM401850, AM401851, |
| | | | | | AM401852, AM401853, AM401854, AM401855, AM401856, |
| | | | | | AM401857, AM401858, AM401859, AM401860, AM401861, |
| | | | | | AM401862, AM401863, AM401864, AM401865, AM401866, |
| | | | | | AM401867, AM401868, AM401869, AM401870, AM401871, |
| | | | | | AM401872, AM401873, AM401874, AM401875, AM401876, |
| | | | | | AM401877, AM401878, AM401879, AM401880, AM401881, |
| | | | | | AM401882, AM401883, AM401884, AM401885, AM401886, |
| | | | | | AM401887, AM401888, AM401889, AM401890, AM401891, |
| | | | | | AM401892, AM401893, AM401894, AM401895, AM401896, |
| | | | | | AM401897, AM401898, AM401899, AM401900, AM401901, |
| | | | | | AM401902, AM401903, AM401904, AM401905, AM401906, |
| | | | | | AM401907, AM401908, AM401909, AM401910, AM401911, |
| | | | | | AM401912, AM401913, AM401914, AM401915, AM401916, |
| | | | | | AM401917, AM401918, AM401919, AM401920, AM401921, |
| | | | | | AM401922, AM401923, AM401924, AM401925, AM401926, |
| | | | | | AM401927, AM401928, AM401929, AM401930, AM401932, |
| | | | | | AM401933, AM401934, AM401935, AM401936, AM401937, |
| | | | | | AM401938, AM401939, AM401940, AM401941, AM401942, |
| | | | | | AM401943, AM401944, AM401945, AM401946, AM401947, |
| | | | | | AM401948, AM401949, AM401950, AM401951, AM401952, |
| | | | | | AM401953, AM401954, AM401955, AM401956, AM401957, |
| | | | | | AM401958, AM401959, AM401960, AM401961, AM401962, |
| | | | | | AM401963, AM401964, AM401966, AM401967, AM401968, |
| | | | | | AM401969, AM401970, AM401971, AM401972, AM401973, |
| | | | | | AM401974, AM401975, AM401976, AM401977, AM401978, |
| | | | | | AM401979, AM401980, AM402055, AM402056, AM402057, |
| | | | | | AM402058, AM402059, AM402060, AM402061, AM402062, |
| | | | | | AM402063, AM402064, AM402065, AM402066, AM402067, |
| | | | | | AM402068, AM402070, AM402071, AM402072, AM402073, |
| | | | | | AM402074, AM402075, AM402076, AM402077, AM402078, |
| | | | | | AM402079, AM402080, AM402081, AM402082, AM402083, |
| | | | | | AM402084, AM402085, AM402086, AM402087, AM402088, |
| | | | | | AM402089, AM402090, AM402092, AM402093, AM402095, |
| | | | | | AM402097, AM402098, AM402099, AM402100, AM402101, |
| | | | | | AM402102, AM402103, AM402104, AM402105, AM402106, |
| | | | | | AM402107, AM402108, AM402109, AM402110, AM402111, |
| | | | | | AM402112, AM402113, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AM402329, AM402330, |
| | | | | | AM402331, AM402332, AM402333, AM402335, AM402336, |
| | | | | | AM402337, AM402338, AM402339, AM402340, AM402341, |
| | | | | | AM402342, AM402343, AM402344, AM402345, AM402346, |
| | | | | | AM402347, AM402348, AM402349, AM402350, AM402351, |
| | | | | | AM402352, AM402353, AM402354, AM402355, AM402356, |
| | | | | | AM402357, AM402358, AM402359, AM402360, AM402361, |
| | | | | | AM402362, AM402363, AM402364, AM402365, AM402367, |
| | | | | | AM402369, AM402370, AM402371, AM402372, AM402373, |
| | | | | | AM402374, AM402375, AM402376, AM402377, AM402378, |
| | | | | | AM402379, AM402380, AM402381, AM402382, AM402383, |
| | | | | | AM402384, AM493471, AM493472, AM493473, AM493474, |
| | | | | | AM493475, AM493476, AM493477, AM493478, AM493479, |
| | | | | | AM493480, AM493481, AM493482, AM493483, AM493484, |
| | | | | | AM493485, AM493486, AM493487, AM493488, AM494937, |
| | | | | | AY008261, AY045702, AY240967, AY240968, AY240969, |
| | | | | | AY460204, AY587016, AY587844, AY808009, AY808010, |
| | | | | | AY808011, AY808012, AY808013, AY808014, AY808015, |
| | | | | | AY808016, AY808017, AY808018, AY808019, AY808020, |
| | | | | | AY808021, AY808022, AY808023, AY808024, AY808025, |
| | | | | | AY808026, AY808028, AY808030, AY808031, AY808032, |
| | | | | | AY808033, AY808034, AY808036, AY808037, AY808038, |
| | | | | | AY808039, AY808040, AY808041, AY808042, AY808043, D10934, |
| | | | | | D11168, D14484, D16435, D30613, D45172, D50480, D50481, |
| | | | | | D50482, D50483, D50484, D50485, D63857, D85516, D89815, |
| | | | | | D89872, D90208, DQ244140, DQ244141, DQ491943, DQ491944, |
| | | | | | DQ491945, DQ491946, DQ491947, DQ491948, DQ491949, |
| | | | | | DQ491950, DQ491951, DQ491952, DQ491953, DQ491954, |
| | | | | | DQ491955, DQ491956, DQ491957, DQ491958, DQ491959, |
| | | | | | DQ491960, DQ491961, DQ491962, DQ491963, DQ491964, |
| | | | | | DQ491965, DQ491966, DQ491967, DQ491968, DQ491969, |
| | | | | | DQ491970, DQ491971, DQ491972, DQ491973, DQ491974, |
| | | | | | DQ491975, DQ491976, DQ491977, DQ491978, DQ491980, |
| | | | | | DQ491981, DQ491982, DQ491983, DQ491984, DQ491985, |
| | | | | | DQ491988, DQ491989, DQ491991, DQ491992, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, DQ492008, DQ492009, |
| | | | | | DQ492010, DQ492011, DQ492012, DQ492013, DQ492014, |
| | | | | | DQ492015, DQ492016, DQ492017, DQ492018, DQ492019, |
| | | | | | DQ492020, DQ492021, DQ492022, DQ492023, DQ492024, |
| | | | | | DQ492025, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ492093, |
| | | | | | DQ492094, DQ492095, DQ492096, DQ492097, DQ492098, |
| | | | | | DQ492099, DQ492100, DQ492101, DQ492102, DQ492103, |
| | | | | | DQ492104, DQ492105, DQ492106, DQ492107, DQ492108, |
| | | | | | DQ492109, DQ492110, DQ492111, DQ492112, DQ492113, |
| | | | | | DQ492114, DQ492115, DQ492116, DQ492117, DQ492118, |
| | | | | | DQ492119, DQ492120, DQ492121, DQ492122, DQ492123, |
| | | | | | DQ492124, DQ492125, DQ492126, DQ492127, DQ492128, |
| | | | | | DQ492129, DQ492130, DQ492131, DQ492132, DQ492133, |
| | | | | | DQ492134, DQ492135, DQ492136, DQ492137, DQ492138, |
| | | | | | DQ492139, DQ492140, DQ492141, DQ492142, DQ492143, |
| | | | | | DQ492144, DQ492145, DQ492146, DQ492147, DQ492148, |
| | | | | | DQ492150, DQ492151, DQ492152, DQ492153, DQ492154, |
| | | | | | DQ492155, DQ492156, DQ492157, DQ492158, DQ492159, |
| | | | | | DQ492160, DQ492161, DQ492162, DQ492163, DQ492164, |
| | | | | | DQ492165, DQ492166, DQ492167, DQ492168, DQ492169, |
| | | | | | DQ492170, DQ492171, DQ492172, DQ492173, DQ492174, |
| | | | | | DQ492175, DQ492176, DQ492177, DQ492178, DQ492179, |
| | | | | | DQ492181, DQ492182, DQ492183, DQ492184, DQ492185, |
| | | | | | DQ492186, DQ492187, DQ492188, DQ492189, DQ492190, |
| | | | | | DQ492191, DQ492192, DQ492193, DQ492194, DQ492195, |
| | | | | | DQ492197, DQ859970, EF032892, EF032893, EF032894, |
| | | | | | EF407459, EF407461, EF407463, EF407464, EF407465, EF407466, |
| | | | | | EF407467, EF407468, EF407469, EF407470, EF407471, EF407472, |
| | | | | | EF407474, EF407475, EF407476, EF407477, EF407478, EF407480, |
| | | | | | EF407482, EF407484, EF407485, EF407486, EF407487, EF407489, |
| | | | | | EF407490, EF407491, EF407494, EF407495, EF407496, EF407497, |
| | | | | | EF407498, EF407499, EF407500, EF407502, EF407503, EF407504, |
| | | | | | EU155218, EU155219, EU155221, EU155223, EU155224, |
| | | | | | EU155225, EU155226, EU155227, EU155229, EU155230, |
| | | | | | EU155231, EU155232, EU155253, EU155255, EU155256, |
| | | | | | EU155257, EU155260, EU155261, EU155262, EU155263, |
| | | | | | EU155264, EU155279, EU155280, EU155281, EU155300, |
| | | | | | EU155301, EU155302, EU155303, EU155304, EU155305, |
| | | | | | EU155306, EU155308, EU155316, EU155318, EU155324, |
| | | | | | EU155326, EU155327, EU155329, EU155330, EU155331, |
| | | | | | EU155332, EU155333, EU155334, EU155335, EU155336, |
| | | | | | EU155337, EU155358, EU155359, EU155360, EU155361, |
| | | | | | EU155362, EU155363, EU155364, EU155365, EU155366, |
| | | | | | EU155367, EU155368, EU155369, EU155370, EU155371, |
| | | | | | EU155372, EU155373, EU155374, EU155375, EU155376, |
| | | | | | EU155377, EU155382, EU234061, EU234062, EU255961, |
| | | | | | EU255962, EU256000, EU256001, EU256045, EU256059, |
| | | | | | EU256061, EU256064, EU256066, EU256075, EU256077, |
| | | | | | EU256078, EU256079, EU256080, EU256081, EU256083, |
| | | | | | EU256084, EU256085, EU256088, EU256089, EU256090, |
| | | | | | EU256092, EU256098, EU256099, EU256100, EU256101, |
| | | | | | EU256102, EU256103, EU482833, EU482839, EU482849, |
| | | | | | EU482859, EU482860, EU482875, EU482880, EU482881, |
| | | | | | EU482883, EU482885, EU482886, EU482888, EU529682, |
| | | | | | EU660386, EU781825, EU781832, EU857431, EU862835, |
| | | | | | EU862837, FJ024277, FJ024279, FJ390397, FJ390398, FJ478453, |
| | | | | | FJ958890, FJ958891, FJ958893, FJ958895, FJ958896, FJ958897, |
| | | | | | FJ958898, FJ958899, FJ958900, FJ958901, FJ958902, FJ958903, |
| | | | | | FJ958904, FJ958905, FJ958906, FJ958907, FJ958909, FJ958910, |
| | | | | | FJ958911, FJ958912, FJ958913, FJ958914, FJ958915, FJ958916, |
| | | | | | FJ958917, FJ958918, FJ958919, FJ958920, FJ958921, FJ958922, |
| | | | | | FJ958923, FJ958924, FJ958925, FJ958926, FJ958927, FJ958928, |
| | | | | | FJ958929, FJ958930, FJ958931, FJ958932, FJ958933, FJ958934, |
| | | | | | FJ958935, FN435993, GQ913870, GU451218, GU451219, |
| | | | | | GU451220, GU451221, GU451223, GU451224, HM042050, |
| | | | | | HM042053, HM042057, HM042059, HM042060, L02836, M58335, |
| | | | | | M84754, M96362, U01214, U16362, U45476, X61596 |
| 364 | tca | acc | S | T | AF265156, AJ507167, AJ507175, EU255960 |
| 364 | tca | acg | S | T | AB049088, AB049100, AB285051, AB285057, AB285074, |
| | | | | | AF176573, AF207757, AF207761, AF265154, AF265155, AJ133096, |
| | | | | | AJ507171, AJ507197, AM401931, AM402091, AM402096, |
| | | | | | AM402334, AY808027, AY808035, DQ491986, DQ491987, |
| | | | | | DQ491993, DQ492026, DQ492040, DQ492041, DQ492042, |
| | | | | | DQ492043, DQ492044, DQ492045, DQ492046, DQ492047, |
| | | | | | DQ492048, DQ492049, DQ492050, DQ492051, DQ492052, |
| | | | | | DQ492053, DQ492054, DQ492055, DQ492056, DQ492057, |
| | | | | | DQ492058, DQ492059, DQ492060, DQ492061, DQ492062, |
| | | | | | DQ492149, DQ492196, EF407492, EF638081, EU155217, |
| | | | | | EU155258, EU155357, EU256065, EU482877, EU482879, |
| | | | | | FJ390396, FJ958892, FJ958894 |
| 364 | tca | act | S | T | AB249644, AB426117, AJ507163, AJ507190, AJ507194, AM402258, |
| | | | | | AM402259, AM402260, AM402261, AM402262, AM402263, |
| | | | | | AM402264, AM402265, AM402266, AM402267, AM402268, |
| | | | | | AM402269, AM402270, AM402271, AM402272, AM402273, |
| | | | | | AM402274, AM402275, AM402276, AM402277, AM402278, |
| | | | | | AM402279, AM402280, AM402281, AM402282, AM402283, |
| | | | | | AM402284, AM402285, AM402286, AM402287, AM402288, |
| | | | | | AM402289, AM402290, AM402291, AM402292, AM402293, |
| | | | | | AM402294, AM402295, AM402296, AM402297, AM402298, |
| | | | | | AM402299, AM402300, AM402301, AM402302, AM402303, |
| | | | | | AM402304, AM402305, AM402306, AM402307, AM402308, |
| | | | | | AM402309, AM402310, AM402311, AM402312, AM402313, |
| | | | | | AM402314, AM402315, AM402316, AM402317, AM402318, |
| | | | | | AM402319, AM402320, AM402321, AM402322, AM402323, |
| | | | | | AM402324, AM402325, AM402326, AM402327, AM402328, |
| | | | | | AM493436, AM493437, AM493438, AM493439, AM493440, |
| | | | | | AM493441, AM493442, AM493443, AM493444, AM493445, |
| | | | | | AM493446, AM493447, AM493448, AM493449, AM493450, |
| | | | | | AM493451, AY808029, DQ491990, DQ492063, DQ492064, |
| | | | | | DQ492065, DQ492066, DQ492067, DQ492068, DQ492069, |
| | | | | | DQ492070, DQ492071, DQ492072, DQ492073, DQ492074, |
| | | | | | DQ492075, DQ492076, DQ492077, DQ492078, DQ492079, |
| | | | | | DQ492080, DQ492081, DQ492082, DQ492083, DQ492084, |
| | | | | | DQ492085, DQ492086, DQ492087, DQ492088, DQ492089, |
| | | | | | DQ492090, DQ492091, DQ492092, EU155307, EU155328, |
| | | | | | EU256082, GU451222 |
| 365 | gaa | gac | E | D | AB049100, AB285079, AB429050, AF165047, AF165048, AJ507169, |
| | | | | | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401862, |
| | | | | | AM401863, AM401864, AM401865, AM401866, AM401867, |
| | | | | | AM401868, AM401869, AM401870, AM401871, AM401872, |
| | | | | | AM401873, AM401874, AM401875, AM401876, AM401877, |
| | | | | | AM401878, AM401879, AM401880, AM401881, AM401882, |
| | | | | | AM401883, AM401884, AM401885, AM401886, AM401887, |
| | | | | | AM401888, AM401889, AM401890, AM401891, AM401892, |
| | | | | | AM401893, AM401894, AM401895, AM401896, AM401897, |
| | | | | | AM401898, AM401899, AM401900, AM401901, AM401902, |
| | | | | | AM401903, AM401904, AM401905, AM401906, AM401907, |
| | | | | | AM401908, AM401909, AM401910, AM401911, AM401912, |
| | | | | | AM401913, AM401914, AM401915, EU155260, EU155363, |
| | | | | | HM042059 |
| 365 | gaa | gat | E | D | AB285055, AB285060, DQ859970, EU155372, HM042056 |
| 365 | gaa | gga | E | G | AJ507174, EU256059 |
| 365 | gaa | ggg | E | G | AB285076, DQ492164 |
| 366 | tct | ttc | S | F | DQ492181 |
| 366 | tct | ttt | S | F | DQ492150 |
| 366 | tct | ccc | S | P | DQ491956 |
| 367 | acc | gct | T | A | DQ492182 |
| 367 | acc | atc | T | I | EU155223, EU256084 |
| 367 | acc | att | T | I | EU256089 |
| 367 | acc | aac | T | N | AB285060, AJ507186, AY808038, EU155363 |
| 367 | acc | aat | T | N | EU482833 |
| 367 | acc | agt | T | S | AB285036, AF165060, AF207760 |
| 367 | acc | tcc | T | S | AF333324, EF407474, EF407494, EU256076, L02836, M58335 |
| 367 | acc | tct | T | S | AJ507177, AJ507184, AM402258, AM402259, AM402260, |
| | | | | | AM402261, AM402262, AM402263, AM402264, AM402265, |
| | | | | | AM402266, AM402267, AM402268, AM402269, AM402270, |
| | | | | | AM402271, AM402272, AM402273, AM402274, AM402275, |
| | | | | | AM402276, AM402277, AM402278, AM402279, AM402280, |
| | | | | | AM402281, AM402282, AM402283, AM402284, AM402285, |
| | | | | | AM402286, AM402287, AM402288, AM402289, AM402290, |
| | | | | | AM402291, AM402292, AM402293, AM402294, AM402295, |
| | | | | | AM402296, AM402297, AM402298, AM402299, AM402300, |
| | | | | | AM402301, AM402302, AM402303, AM402304, AM402305, |
| | | | | | AM402306, AM402307, AM402308, AM402309, AM402310, |
| | | | | | AM402311, AM402312, AM402313, AM402314, AM402315, |
| | | | | | AM402316, AM402317, AM402318, AM402319, AM402320, |
| | | | | | AM402321, AM402322, AM402323, AM402324, AM402325, |
| | | | | | AM402326, AM402327, AM402328, AM493436, AM493437, |
| | | | | | AM493438, AM493439, AM493440, AM493441, AM493442, |
| | | | | | AM493443, AM493444, AM493445, AM493446, AM493447, |
| | | | | | AM493448, AM493449, AM493450, AM493451, EU155217, |
| | | | | | EU155336, EU155360, EU482874 |
| 368 | gtg | gcg | V | A | DQ492082, EU256084 |
| 368 | gtg | ata | V | I | GQ913873, HM042056 |
| 368 | gtg | ctg | V | L | AB285081 |
| 368 | gtg | ttg | V | L | AB285078 |
| 368 | gtg | atg | V | M | AM494937, AY240967, DQ491968, DQ492030, EU155232 |
| 369 | tct | gct | S | A | AM493471, AM493472, AM493473, AM493474, AM493475, |
| | | | | | AM493476, AM493477, AM493478, AM493479, AM493480, |
| | | | | | AM493481, AM493482, AM493483, AM493484, AM493485, |
| | | | | | AM493486, AM493487, AM493488, AY808028, DQ491979, |
| | | | | | DQ491980, DQ491981, DQ491982, DQ491984, DQ491985, |
| | | | | | DQ491986, DQ491987, DQ491988, DQ491989, DQ491990, |
| | | | | | DQ491991, DQ491992, DQ491993, DQ491994, DQ491995, |
| | | | | | DQ491996, DQ491997, DQ491998, DQ491999, DQ492000, |
| | | | | | DQ492001, DQ492002, DQ492003, DQ492004, DQ492005, |
| | | | | | DQ492006, DQ492007, EF638081, EU239714 |
| 369 | tct | ttt | S | F | AM402352, DQ492143 |
| 369 | tct | cct | S | P | AY240967 |
| 369 | tct | acc | S | T | EU155257, EU155337 |
| 369 | tct | gtt | S | V | DQ491983 |
| 370 | tct | gct | S | A | AF207754, EU239714 |
| 370 | tct | ctt | S | L | EU155281 |
| 370 | tct | cct | S | P | DQ492024 |
| 370 | tct | act | S | T | AB285060, AB285067, AB285076, EU155333, HM042056 |
| 371 | gcc | ggc | A | G | AM402263, AM402269 |
| 371 | gcc | acc | A | T | AM402310, AM402311, AM402312, AM402313, AM402314, |
| | | | | | AM402315, AM402316, AM402317, AM402318, AM402319, |
| | | | | | AM402320, AM402321, AM402322, AM402323, AM402324, |
| | | | | | AM402325, AM402326, AM402327, AM402328, DQ492092 |
| 371 | gcc | gtc | A | V | AB049094, AB285044, AY240967, DQ492165, EU155381, |
| | | | | | EU256001, EU482880 |
| 371 | gcc | gtt | A | V | AB285076, AJ507191, DQ244140 |
| 372 | ttg | cag | L | Q | AM402326 |
| 372 | ttg | tgg | L | W | DQ492149 |
| 373 | gcg | ggg | A | G | EU482880 |
| 373 | gcg | tcg | A | S | DQ491995 |
| 373 | gcg | gta | A | V | EU155263 |
| 373 | gcg | gtg | A | V | DQ491967 |
| 374 | gag | gac | E | D | FN435993 |
| 374 | gag | gat | E | D | AY808010, EU155328 |
| 374 | gag | ggg | E | G | AJ507175, AM402282, AM402315, EU155372, EU256075 |
| 374 | gag | aag | E | K | AM401884, AM401889, AM401893, DQ491999 |
| 375 | ctc | ttc | L | F | AF207762 |
| 375 | ctc | ttt | L | F | AF207756, AM493439 |
| 375 | ctc | cct | L | P | DQ492058, DQ492125 |
| 376 | gcc | acc | A | T | AM402066, DQ492182, EU239714 |
| 376 | gcc | gtt | A | V | AM401924, DQ491951 |
| 377 | aca | gca | T | A | AJ507192, AM494937, AY808024, AY808026, AY808033, |
| | | | | | EF407469, EF407472, EU155225, EU155227, EU155328, |
| | | | | | EU155376, EU234062, EU529682 |
| 377 | aca | gcg | T | A | AB285048, AB285055, AJ507160, AY240969, EU256085 |
| 377 | aca | gct | T | A | AF207769 |
| 377 | aca | gag | T | E | EU256075 |
| 377 | aca | ata | T | I | AF265148, AF265149, AF265150, AF265163, AF265164, AF265165, |
| | | | | | AJ507161, AJ507175, AM493491, AM493492, AM493500, |
| | | | | | AM493501, AM493503, AM493504, AM493505, GQ913872, |
| | | | | | GU451222 |
| 377 | aca | aaa | T | K | AY808042, DQ491975, DQ491978, DQ492108, EU155219 |
| 377 | aca | aag | T | K | HM042062 |
| 377 | aca | agg | T | R | EU256084 |
| 377 | aca | tcg | T | S | EU256081 |
| 377 | aca | gta | T | V | AF165045, AF165046, AF207755, AJ507179, EF407461, EU155372, |
| | | | | | EU482886 |
| 377 | aca | gtt | T | V | AF165058, U01214 |
| 378 | aag | gag | K | E | AJ507157, DQ492020, DQ492057, FJ958927 |
| 378 | aag | cag | K | Q | AY808040, DQ491943, DQ491944, DQ491945, DQ491946, |
| | | | | | DQ491947, DQ491948, DQ491949, DQ491950, DQ491951, |
| | | | | | DQ491952, DQ491953, DQ491954, DQ491955, DQ491956, |
| | | | | | DQ491957 |
| 378 | aag | agg | K | R | AJ507189, AM493499, DQ491982, DQ491991, DQ492125, |
| | | | | | DQ492188, EU155280 |
| 378 | aag | tcg | K | S | AJ507184 |
| 378 | aag | aca | K | T | AJ507161 |
| 378 | aag | acc | K | T | AB285048 |
| 379 | acc | gcc | T | A | AB049092, AB285040, AM402055, AM402060, AM402066, |
| | | | | | AM402070, AM402071, AM402074, AM402075, AM402081, |
| | | | | | AM402088, AM402090, AM402096, AM402098, AM402101, |
| | | | | | AM402102, AM402106, AM402108, AM402111, AM402112, |
| | | | | | AM402115, AM402125, AM402127, AY460204, EF407470, |
| | | | | | EF407477, EU155218, EU155381, EU781832, EU862837, |
| | | | | | HM042053 |
| 379 | acc | gct | T | A | AF165045, AF165046, AF207755 |
| 379 | acc | agc | T | S | AF165057, AF165058 |
| 379 | acc | agt | T | S | GQ913869 |
| 379 | acc | tcc | T | S | AB442220, EF407481, EU256083 |
| 379 | acc | tct | T | S | EU155375 |
| 380 | ttc | ctc | F | L | AM402296, AM493443, DQ491949, DQ492017 |
| 380 | ttc | ctt | F | L | DQ492049 |
| 380 | ttc | tcc | F | S | DQ492027, DQ492151, DQ492165 |
| 381 | ggc | gcc | G | A | AB285073 |
| 381 | ggc | gct | G | A | EF407489, EU155373 |
| 381 | ggc | gac | G | D | AF208024, AM402347, AY808026 |
| 381 | ggc | aat | G | N | DQ492080 |
| 381 | ggc | agc | G | S | AB049094, AB049098, AB049099, AB285037, AJ507160, AJ507186, |
| | | | | | AJ507192, AY240969, AY808024, AY808033, EF407461, EF407470, |
| | | | | | EF407476, EF407482, EF638081, EU155219, EU155227, |
| | | | | | EU155261, EU155326, EU155333, EU155334, EU155363, |
| | | | | | EU256084, EU482885, EU862837, FJ958898 |
| 381 | ggc | agt | G | S | DQ492063, DQ492065, DQ492067, DQ492068, DQ492070, |
| | | | | | DQ492073, DQ492074, DQ492075, DQ492078, DQ492079, |
| | | | | | DQ492081, DQ492082, DQ492083, DQ492084, DQ492085, |
| | | | | | DQ492086, DQ492087, DQ492088, DQ492089, DQ492090, |
| | | | | | DQ492091, DQ492092, EF407490, EU256001 |
| 381 | ggc | act | G | T | EU155281 |
| 382 | agc | gac | S | D | AB285044, AB285055, EU155228, EU155370, EU155371, |
| | | | | | GU451222 |
| 382 | agc | gga | S | G | AF207755 |
| 382 | agc | ggc | S | G | AB049098, AB285062, AB442221, AF207774, AJ507160, AJ507182, |
| | | | | | AJ507188, AJ507189, AM402115, AY240969, AY808017, |
| | | | | | AY808031, AY808032, AY808033, D14484, EF407470, EU155279, |
| | | | | | EU155281, EU155305, EU155306, EU155307, EU155333, |
| | | | | | EU256001, EU256075, EU256084, EU256091, FJ958897, |
| | | | | | GQ913872 |
| 382 | agc | ggt | S | G | AF207769, D89815, EU155369 |
| 382 | agc | aac | S | N | AB285054, AF207771, AJ507192, AM402061, AM493493, |
| | | | | | GU451218, GU451219, GU451220 |
| 382 | agc | aga | S | R | DQ491976 |
| 383 | tcc | ttc | S | F | HM042058 |
| 383 | tcc | ttt | S | F | AM402367 |
| 383 | tcc | ccc | S | P | AF165061, AF207769, AM401885, AM493449, DQ244140, |
| | | | | | EF407461, EF407470, FJ958916 |
| 383 | tcc | cct | S | P | EF407482, EU862837 |
| 384 | gaa | gca | E | A | EU155260, EU155364, EU256080, EU256084 |
| 384 | gaa | gcg | E | A | EU155232 |
| 384 | gaa | gac | E | D | AM402329, AM402330, AM402331, AM402332, AM402333, |
| | | | | | AM402334, AM402335, AM402336, AM402337, AM402338, |
| | | | | | AM402339, AM402340, AM402341, AM402342, AM402343, |
| | | | | | AM402344, AM402345, AM402346, AM402347, AM402348, |
| | | | | | AM402349, AM402350, AM402351, AM402352, AM402353, |
| | | | | | AM402354, AM402355, AM402356, AM402357, AM402358, |
| | | | | | AM402359, AM402360, AM402361, AM402362, AM402363, |
| | | | | | AM402364, AM402365, AM402367, AM402369, AM402370, |
| | | | | | AM402371, AM402372, AM402373, AM402374, AM402375, |
| | | | | | AM402376, AM402377, AM402378, AM402379, AM402380, |
| | | | | | AM402381, AM402382, AM402383, AM402384, AY808027, |
| | | | | | HM042059 |
| 384 | gaa | gat | E | D | AB285081 |
| 384 | gaa | gga | E | G | AB010249, AB010250, AB036519, AB036520, AB036521, |
| | | | | | AB049087, AB049088, AB049089, AB049090, AB049092, |
| | | | | | AB049095, AB049098, AB049099, AB049100, AB080299, |
| | | | | | AB109543, AB154183, AB154184, AB191333, AB285035, |
| | | | | | AB285037, AB285038, AB285040, AB285042, AB285043, |
| | | | | | AB285046, AB285048, AB285050, AB285052, AB285054, |
| | | | | | AB285055, AB285056, AB285061, AB285068, AB285070, |
| | | | | | AB285071, AB285073, AB285078, AB285079, AB429050, |
| | | | | | AB442219, AB442220, AB442221, AB442222, AF034151, |
| | | | | | AF165045, AF165046, AF165049, AF165050, AF165051, AF165052, |
| | | | | | AF165053, AF165054, AF165055, AF165056, AF165057, AF165058, |
| | | | | | AF165059, AF165060, AF165061, AF165062, AF165063, AF165064, |
| | | | | | AF207752, AF207755, AF207757, AF207758, AF207762, AF207764, |
| | | | | | AF207765, AF207768, AF207771, AF207772, AF208024, AF265142, |
| | | | | | AF265144, AF265160, AF265162, AF356827, AF483269, AJ507157, |
| | | | | | AJ507159, AJ507160, AJ507161, AJ507165, AJ507167, AJ507171, |
| | | | | | AJ507172, AJ507174, AJ507175, AJ507189, AJ507190, AJ507192, |
| | | | | | AJ507193, AM401848, AM401849, AM401850, AM401851, |
| | | | | | AM401852, AM401853, AM401854, AM401855, AM401856, |
| | | | | | AM401857, AM401858, AM401859, AM401860, AM401861, |
| | | | | | AM401862, AM401863, AM401864, AM401865, AM401866, |
| | | | | | AM401867, AM401868, AM401869, AM401870, AM401871, |
| | | | | | AM401872, AM401873, AM401874, AM401875, AM401876, |
| | | | | | AM401877, AM401878, AM401879, AM401880, AM401881, |
| | | | | | AM401882, AM401883, AM401884, AM401885, AM401886, |
| | | | | | AM401887, AM401888, AM401889, AM401890, AM401891, |
| | | | | | AM401892, AM401893, AM401894, AM401895, AM401896, |
| | | | | | AM401897, AM401898, AM401899, AM401900, AM401901, |
| | | | | | AM401902, AM401903, AM401904, AM401905, AM401906, |
| | | | | | AM401907, AM401908, AM401909, AM401910, AM401911, |
| | | | | | AM401912, AM401913, AM401914, AM401915, AM402310, |
| | | | | | AM402311, AM402312, AM402313, AM402314, AM402315, |
| | | | | | AM402316, AM402317, AM402318, AM402319, AM402320, |
| | | | | | AM402321, AM402322, AM402323, AM402324, AM402325, |
| | | | | | AM402326, AM402327, AM402328, AY008261, AY587016, |
| | | | | | AY808009, AY808015, AY808017, AY808020, AY808021, |
| | | | | | AY808029, AY808033, AY808036, AY808039, AY808041, D10934, |
| | | | | | D11168, D14484, D16435, D30613, D45172, D50480, D63857, |
| | | | | | D85516, D89815, D89872, DQ244141, DQ491958, DQ491959, |
| | | | | | DQ491960, DQ491961, DQ491962, DQ491963, DQ491964, |
| | | | | | DQ491965, DQ491966, DQ491967, DQ491968, DQ491969, |
| | | | | | DQ491970, DQ492063, DQ492065, DQ492066, DQ492067, |
| | | | | | DQ492068, DQ492069, DQ492070, DQ492072, DQ492073, |
| | | | | | DQ492074, DQ492075, DQ492077, DQ492078, DQ492079, |
| | | | | | DQ492080, DQ492081, DQ492082, DQ492083, DQ492084, |
| | | | | | DQ492085, DQ492086, DQ492087, DQ492088, DQ492089, |
| | | | | | DQ492090, DQ492091, DQ492110, DQ859970, EF407460, |
| | | | | | EF407462, EF407468, EF407477, EF407479, EF407480, EF407491, |
| | | | | | EF407501, EU155218, EU155222, EU155228, EU155257, |
| | | | | | EU155302, EU155306, EU155315, EU155317, EU155336, |
| | | | | | EU155356, EU155360, EU155363, EU155368, EU155370, |
| | | | | | EU155371, EU155372, EU155373, EU155382, EU255962, |
| | | | | | EU256000, EU256062, EU256064, EU256075, EU256076, |
| | | | | | EU256078, EU256079, EU256082, EU256089, EU256090, |
| | | | | | EU256091, EU256099, EU256101, EU482859, EU482874, |
| | | | | | EU482875, EU660386, EU660388, EU781827, EU857431, |
| | | | | | EU862837, GQ913869, GQ913873, GU451218, GU451219, |
| | | | | | GU451220, GU451222, HM042051, HM042052, HM042054, |
| | | | | | HM042056, HM042057, HM042058, HM042061, M96362, U16362, |
| | | | | | X61596 |
| 384 | gaa | ggc | E | G | AJ507188, AY808040, DQ491943, DQ491944, DQ491945, |
| | | | | | DQ491946, DQ491947, DQ491948, DQ491949, DQ491950, |
| | | | | | DQ491951, DQ491952, DQ491953, DQ491954, DQ491955, |
| | | | | | DQ491956, DQ491957 |
| 384 | gaa | ggg | E | G | AB049096, AB285041, AB285057, AB285064, AF165047, |
| | | | | | AF165048, AF207753, AF207769, AJ132997, AJ507155, AJ507164, |
| | | | | | AJ507178, AM401916, AM401917, AM401918, AM401922, |
| | | | | | AM401923, AM401924, AM401926, AM401927, AM401928, |
| | | | | | AM401930, AM401931, AM401932, AM401933, AM401936, |
| | | | | | AM401939, AM401940, AM401941, AM401943, AM401944, |
| | | | | | AM401945, AM401947, AM401949, AM401950, AM401951, |
| | | | | | AM401952, AM401953, AM401954, AM401955, AM401956, |
| | | | | | AM401958, AM401959, AM401960, AM401961, AM401962, |
| | | | | | AM401963, AM401964, AM401965, AM401966, AM401967, |
| | | | | | AM401968, AM401969, AM401970, AM401971, AM401972, |
| | | | | | AM401973, AM401974, AM401975, AM401976, AM401977, |
| | | | | | AM401978, AM401979, AM401980, AM493471, AM493472, |
| | | | | | AM493473, AM493474, AM493475, AM493476, AM493477, |
| | | | | | AM493478, AM493479, AM493480, AM493481, AM493482, |
| | | | | | AM493483, AM493484, AM493485, AM493486, AM493487, |
| | | | | | AM493488, AY045702, D50482, D50484, D90208, DQ492092, |
| | | | | | DQ492177, EU155231, EU155235, EU155325, EU155361, |
| | | | | | HM042055 |
| 384 | gaa | ggt | E | G | AB285062, EU155357 |
| 384 | gaa | aaa | E | K | AM402120, AY460204, AY808026, EF407467, EU155308, |
| | | | | | EU529682, HM042053 |
| 384 | gaa | aag | E | K | AY808010 |
| 384 | gaa | atg | E | M | AY808031 |
| 384 | gaa | aga | E | R | AY808042, DQ491971, DQ491972, DQ491973, DQ491974, |
| | | | | | DQ491975, DQ491976, DQ491977, DQ491978, EF407470 |
| 384 | gaa | gta | E | V | AB049094 |
| 385 | tcg | gcg | S | A | DQ492094 |
| 385 | tcg | gag | S | E | AB285073 |
| 385 | tcg | ttg | S | L | EU155359 |
| 385 | tcg | cca | S | P | AB285076, HM042058 |
| 385 | tcg | ccg | S | P | AB285040, AJ507169, EF407470, EU155264, EU155369, |
| | | | | | EU255960, EU256083, HM042059, HM042062 |
| 385 | tcg | acg | S | T | AY044867, AY808032, DQ492184 |
| 385 | tcg | act | S | T | AJ507161 |
| 386 | tcg | gcg | S | A | AY008261 |
| 386 | tcg | gag | S | E | AF207769 |
| 386 | tcg | ctg | S | L | AY240969, AY808042, DQ491971, DQ491972, DQ491973, |
| | | | | | DQ491974, DQ491975, DQ491976, DQ491977, DQ491978 |
| 386 | tcg | cca | S | P | AB285076, EU155376, EU482833 |
| 386 | tcg | ccg | S | P | AB154196, AJ507169, AM402381, AY808024, EF407467, |
| | | | | | EF407470, EF407481, EU155359, EU256102, EU529682, |
| | | | | | GQ913873, HM042058, HM042059 |
| 386 | tcg | cgc | S | R | AJ507161 |
| 386 | tcg | acg | S | T | AY808033, EF407465, EU155375 |
| 387 | gcc | tgc | A | C | AJ507161 |
| 387 | gcc | ggc | A | G | AF165063, AF165064, AF265162, AJ507184, EU155300, EU155368, |
| | | | | | EU256083, EU482885, EU482888, EU862837 |
| 387 | gcc | ccc | A | P | EU155306 |
| 387 | gcc | tcc | A | S | AY240969, EU482833 |
| 387 | gcc | tct | A | S | EU256102 |
| 387 | gcc | acc | A | T | AB154194, AB285063, AF207769, AM402061, AM402066, |
| | | | | | AY808024, EU255960, GU451218, GU451219, GU451220, |
| | | | | | HM042062 |
| 387 | gcc | gtc | A | V | AM401958, AM402259, AM402323, AY808034, DQ492008, |
| | | | | | DQ492009, DQ492010, DQ492011, DQ492012, DQ492013, |
| | | | | | DQ492014, DQ492016, DQ492017, DQ492018, DQ492020, |
| | | | | | DQ492021, DQ492022, DQ492023, DQ492024, DQ492025, |
| | | | | | DQ492026, DQ492027, DQ492028, DQ492029, DQ492030, |
| | | | | | DQ492031, DQ492032, DQ492033, DQ492034, DQ492035, |
| | | | | | DQ492036, DQ492037, DQ492038, DQ492039, DQ859970 |
| 387 | gcc | gtt | A | V | DQ492015, DQ492019, EF407468 |
| 388 | gtc | gca | V | A | AB285042 |
| 388 | gtc | gcc | V | A | AB049089, AB049091, AB049100, AB080299, AB154188, |
| | | | | | AB285036, AB285039, AB285044, AB285051, AB285054, |
| | | | | | AB285055, AB285062, AB285068, AB285071, AB285076, |
| | | | | | AB285077, AB285078, AB442221, AF165045, AF165046, |
| | | | | | AF207753, AF207754, AF207755, AF207768, AF207769, AF207770, |
| | | | | | AF265149, AJ238800, AJ507163, AJ507172, AJ507188, AM402096, |
| | | | | | AY240969, AY808026, AY808028, AY808038, AY808043, D14484, |
| | | | | | D50481, D50485, D85516, DQ491979, DQ491980, DQ491981, |
| | | | | | DQ491985, DQ491986, DQ491987, DQ491988, DQ491990, |
| | | | | | DQ491991, DQ491993, DQ491994, DQ491995, DQ491996, |
| | | | | | DQ491997, DQ491999, DQ492000, DQ492001, DQ492002, |
| | | | | | DQ492003, DQ492004, DQ492005, DQ492006, DQ492007, |
| | | | | | DQ492046, EF407463, EF407470, EF407496, EU155256, |
| | | | | | EU155263, EU155316, EU155318, EU155324, EU155335, |
| | | | | | EU155365, EU155369, EU155375, EU155376, EU255960, |
| | | | | | EU256075, EU256082, EU256091, EU256103, EU482879, |
| | | | | | GQ913871, GU451218, GU451219, GU451220, GU451222, |
| | | | | | HM042050, HM042053, HM042055, HM042059, HM042060, |
| | | | | | HM042061 |
| 388 | gtc | gcg | V | A | AY808010, EF407495 |
| 388 | gtc | gct | V | A | AB285040, AM493489, AM493490, AM493491, AM493492, |
| | | | | | AM493493, AM493494, AM493495, AM493496, AM493497, |
| | | | | | AM493498, AM493499, AM493500, AM493501, AM493502, |
| | | | | | AM493503, AM493504, AM493505, AM493506, AM493507, |
| | | | | | AY808033, D50480, D50483, DQ244140, DQ244141, EF407465, |
| | | | | | EF407472, EF407477, EF407482, EF407486, EF407494, EU155264, |
| | | | | | EU155280, EU155306, EU155370, EU155371, EU256099, |
| | | | | | EU482881, FJ390396, M84754 |
| 388 | gtc | gac | V | D | AF207771 |
| 388 | gtc | ttc | V | F | EU862835 |
| 388 | gtc | ggc | V | G | AJ507161, AY008261, EF407467, EF407468, EU155359 |
| 388 | gtc | atc | V | I | AB049098, AB285037, AY808013, EF407492, EF407499, |
| | | | | | EF638081, EU256084, GU451221, GU451223, GU451224, M96362, |
| | | | | | U16362 |
| 388 | gtc | att | V | I | AY808020, EU256080 |
| 388 | gtc | ctt | V | L | AB249644, AB426117 |
| 388 | gtc | ccc | V | P | AF165061, AF165062 |
| 388 | gtc | cgc | V | R | EF407501 |
| 388 | gtc | tcc | V | S | AF207757 |
| 388 | gtc | acc | V | T | AY460204, DQ491998 |
| 389 | gac | gct | D | A | AJ507169 |
| 389 | gac | ggc | D | G | AY808026 |
| 389 | gac | ggt | D | G | M84754 |
| 389 | gac | aac | D | N | AY808030, EU256080 |
| 389 | gac | agc | D | S | EU529682 |
| 390 | agc | tgc | S | C | DQ492170 |
| 390 | agc | gga | S | G | AF207757, EU155376 |
| 390 | agc | ggc | S | G | AB285040, AB285041, AM402081, AY460204, DQ492091, |
| | | | | | DQ492149, EU862835 |
| 390 | agc | aag | S | K | EU155306 |
| 390 | agc | aac | S | N | AB154196, AJ507169, DQ492031 |
| 390 | agc | aga | S | R | D50480, D50483 |
| 390 | agc | acc | S | T | EU155359 |
| 391 | ggc | gac | G | D | AM402066 |
| 391 | ggc | aac | G | N | EU155359 |
| 391 | ggc | cgc | G | R | AF207757 |
| 391 | ggc | agc | G | S | AB154195, AB154196, AJ507169, EU155306, EU155376 |
| 391 | ggc | agt | G | S | DQ492158 |
| 392 | acg | gca | T | A | AF207757, EU256080 |
| 392 | acg | gcg | T | A | EF407467, EU155306, HM042056 |
| 392 | acg | ata | T | I | AF207771 |
| 392 | acg | atg | T | M | AB285044, AB285078, AF265147, AJ507161, AJ507169, AJ507182, |
| | | | | | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401863, |
| | | | | | AM401864, AM401865, AM401866, AM401867, AM401868, |
| | | | | | AM401869, AM401870, AM401871, AM401872, AM401873, |
| | | | | | AM401874, AM401875, AM401876, AM401877, AM401878, |
| | | | | | AM401879, AM401880, AM401881, AM401882, AM401883, |
| | | | | | AM401884, AM401885, AM401886, AM401887, AM401888, |
| | | | | | AM401889, AM401890, AM401891, AM401892, AM401893, |
| | | | | | AM401894, AM401895, AM401896, AM401897, AM401898, |
| | | | | | AM401899, AM401900, AM401901, AM401902, AM401903, |
| | | | | | AM401904, AM401905, AM401906, AM401907, AM401908, |
| | | | | | AM401909, AM401910, AM401911, AM401912, AM401913, |
| | | | | | AM401914, AM401915, AY808042, DQ071885, DQ244140, |
| | | | | | DQ491971, DQ491972, DQ491973, DQ491974, DQ491975, |
| | | | | | DQ491976, DQ491977, DQ491978, EF407471, EU155359, |
| | | | | | EU155364 |
| 392 | acg | tcg | T | S | DQ492054, DQ492055 |
| 392 | acg | gtg | T | V | AB285041, AF207766, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, |
| | | | | | AY808024, EU155369, EU482880 |
| 393 | gca | gaa | A | E | HM042062 |
| 393 | gca | gag | A | E | AJ507189, EF407501, EF407504 |
| 393 | gca | ggg | A | G | DQ492006, EU256080, EU256083 |
| 393 | gca | tcg | A | S | AF165053, AF165054, EU155255 |
| 393 | gca | acg | A | T | AB285040, AY008261, EU155306, GQ913873 |
| 393 | gca | gtg | A | V | AJ507169, AM401942, EF407467, EU482880 |
| 394 | acg | gca | T | A | HM042062 |
| 394 | acg | gcc | T | A | AB285040, AF176573, AM402337, DQ492076, EU155359, |
| | | | | | EU482880, HM042058 |
| 394 | acg | gcg | T | A | EF407481 |
| 394 | acg | gct | T | A | DQ492157, DQ492162, DQ492167 |
| 394 | acg | atc | T | I | AB154195, AY808031, EU155369 |
| 394 | acg | att | T | I | AB154196 |
| 394 | acg | agc | T | S | AB154187, AB154188, AB442219, AF034151, AF356827, |
| | | | | | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507, AY808023, |
| | | | | | AY808032, EU155264, EU155332 |
| 394 | acg | tcc | T | S | AB049101, AB285046, AJ507161, AJ507194, AY808017, |
| | | | | | DQ491951, EF407487, EU155281, EU155375, EU256084, |
| | | | | | EU256102, EU781825, HM042054 |
| 394 | acg | tct | T | S | AB285081, AJ507160 |
| 395 | gcc | ggc | A | G | AB010250, AB154183, AB154184, AB285063, AB285064, |
| | | | | | AF265152, AJ507162, AJ507182, AY587016, AY808042, D30613, |
| | | | | | D45172, D89815, D90208, DQ859970, EU256061, EU256076, |
| | | | | | EU857431, HM042059 |
| 395 | gcc | ggt | A | G | AF265153, AJ507189 |
| 395 | gcc | atc | A | I | AY808009, EF407478, EF407498, EU155218 |
| 395 | gcc | tcc | A | S | DQ492109 |
| 395 | gcc | acc | A | T | AM402355 |
| 395 | gcc | act | A | T | AF207757, AM401874, AM401904 |
| 395 | gcc | gtc | A | V | EU155376 |
| 396 | tct | ctc | S | L | AB285077, AB429050, EU529682 |
| 396 | tct | ctt | S | L | AB285065, AF333324, D63857, DQ492074, EF407470, EF407474, |
| | | | | | EU781825, M58335 |
| 396 | tct | cca | S | P | AB249644, AB285040, AB285069, AB426117, EF407489 |
| 396 | tct | ccc | S | P | AB049098, AB154194, AB154195, AB154196, AB154198, |
| | | | | | AB154200, AB285038, AB285042, AB285074, AB285076, |
| | | | | | AB442219, AF165049, AF165050, AF165059, AF165060, AF356827, |
| | | | | | AJ507167, AJ507171, AJ507189, AM402087, AM402105, |
| | | | | | AY808013, AY808014, AY808017, AY808021, AY808022, |
| | | | | | AY808033, AY808036, AY808040, AY808041, D50480, D50483, |
| | | | | | DQ491943, DQ491944, DQ491945, DQ491946, DQ491947, |
| | | | | | DQ491948, DQ491949, DQ491950, DQ491951, DQ491952, |
| | | | | | DQ491953, DQ491954, DQ491955, DQ491956, DQ491957, |
| | | | | | DQ491958, DQ491959, DQ491960, DQ491961, DQ491962, |
| | | | | | DQ491963, DQ491964, DQ491965, DQ491966, DQ491967, |
| | | | | | DQ491968, DQ491969, DQ491970, DQ492021, DQ492093, |
| | | | | | DQ492163, DQ492164, DQ492165, DQ492166, DQ492167, |
| | | | | | DQ492168, DQ492169, DQ492170, DQ492171, DQ492172, |
| | | | | | DQ492173, DQ492174, DQ492175, DQ492176, DQ492177, |
| | | | | | DQ492178, DQ492179, DQ492180, DQ492181, DQ492182, |
| | | | | | DQ492183, DQ492184, DQ492185, DQ492186, DQ492187, |
| | | | | | DQ492188, DQ492189, DQ492190, DQ492191, DQ492192, |
| | | | | | DQ492193, DQ492194, DQ492195, DQ492196, DQ492197, |
| | | | | | EF407472, EF407496, EU256045, EU256085, EU482883, |
| | | | | | FJ390397, HM042056 |
| 396 | tct | ccg | S | P | EU155221, EU256084 |
| 396 | tct | cct | S | P | AB010249, AB010250, AB036519, AB036520, AB036521, |
| | | | | | AB049087, AB049088, AB049089, AB049090, AB049091, |
| | | | | | AB049092, AB049094, AB049095, AB049096, AB049099, |
| | | | | | AB049100, AB080299, AB109543, AB154177, AB154178, |
| | | | | | AB154179, AB154180, AB154181, AB154182, AB154183, |
| | | | | | AB154184, AB154185, AB154186, AB154188, AB154189, |
| | | | | | AB154190, AB154193, AB154197, AB154199, AB154201, |
| | | | | | AB154202, AB154203, AB154204, AB154205, AB154206, |
| | | | | | AB191333, AB285035, AB285036, AB285037, AB285039, |
| | | | | | AB285041, AB285043, AB285044, AB285046, AB285047, |
| | | | | | AB285048, AB285050, AB285051, AB285052, AB285053, |
| | | | | | AB285054, AB285055, AB285056, AB285057, AB285058, |
| | | | | | AB285059, AB285060, AB285061, AB285062, AB285063, |
| | | | | | AB285064, AB285066, AB285067, AB285068, AB285070, |
| | | | | | AB285071, AB285072, AB285073, AB285075, AB285078, |
| | | | | | AB285079, AB285081, AB442220, AB442221, AB442222, |
| | | | | | AF033373, AF034151, AF165045, AF165046, AF165047, AF165048, |
| | | | | | AF165051, AF165052, AF165053, AF165054, AF165055, AF165056, |
| | | | | | AF165057, AF165058, AF165061, AF165062, AF165063, AF165064, |
| | | | | | AF207752, AF207753, AF207754, AF207755, AF207756, AF207757, |
| | | | | | AF207758, AF207760, AF207761, AF207762, AF207763, AF207764, |
| | | | | | AF207765, AF207766, AF207768, AF207769, AF207770, AF207771, |
| | | | | | AF207772, AF207774, AF208024, AF265142, AF265143, AF265144, |
| | | | | | AF265145, AF265146, AF265147, AF265148, AF265149, AF265150, |
| | | | | | AF265152, AF265153, AF265154, AF265155, AF265156, AF265158, |
| | | | | | AF265159, AF265160, AF265161, AF265162, AF265163, AF265164, |
| | | | | | AF265165, AF313916, AF483269, AJ000009, AJ132996, AJ132997, |
| | | | | | AJ133096, AJ238800, AJ507155, AJ507156, AJ507158, AJ507159, |
| | | | | | AJ507160, AJ507161, AJ507162, AJ507163, AJ507164, AJ507165, |
| | | | | | AJ507166, AJ507168, AJ507169, AJ507170, AJ507172, AJ507174, |
| | | | | | AJ507175, AJ507177, AJ507178, AJ507179, AJ507180, AJ507181, |
| | | | | | AJ507182, AJ507183, AJ507184, AJ507185, AJ507186, AJ507187, |
| | | | | | AJ507188, AJ507190, AJ507191, AJ507192, AJ507193, AJ507194, |
| | | | | | AJ507195, AJ507196, AJ507197, AJ507198, AJ507199, AM401848, |
| | | | | | AM401849, AM401850, AM401851, AM401852, AM401853, |
| | | | | | AM401854, AM401855, AM401856, AM401857, AM401858, |
| | | | | | AM401859, AM401860, AM401861, AM401862, AM401863, |
| | | | | | AM401864, AM401865, AM401866, AM401867, AM401868, |
| | | | | | AM401869, AM401870, AM401871, AM401872, AM401873, |
| | | | | | AM401874, AM401875, AM401876, AM401877, AM401878, |
| | | | | | AM401879, AM401880, AM401881, AM401882, AM401883, |
| | | | | | AM401884, AM401885, AM401886, AM401887, AM401888, |
| | | | | | AM401889, AM401890, AM401891, AM401892, AM401893, |
| | | | | | AM401894, AM401895, AM401896, AM401897, AM401898, |
| | | | | | AM401899, AM401900, AM401901, AM401902, AM401903, |
| | | | | | AM401904, AM401905, AM401906, AM401907, AM401908, |
| | | | | | AM401909, AM401910, AM401911, AM401912, AM401913, |
| | | | | | AM401914, AM401915, AM401916, AM401917, AM401918, |
| | | | | | AM401919, AM401920, AM401921, AM401922, AM401923, |
| | | | | | AM401924, AM401925, AM401926, AM401927, AM401928, |
| | | | | | AM401929, AM401930, AM401931, AM401932, AM401933, |
| | | | | | AM401934, AM401935, AM401936, AM401937, AM401938, |
| | | | | | AM401939, AM401940, AM401941, AM401942, AM401943, |
| | | | | | AM401944, AM401945, AM401946, AM401947, AM401948, |
| | | | | | AM401949, AM401950, AM401951, AM401952, AM401953, |
| | | | | | AM401954, AM401955, AM401956, AM401957, AM401958, |
| | | | | | AM401959, AM401960, AM401961, AM401962, AM401963, |
| | | | | | AM401964, AM401965, AM401966, AM401967, AM401968, |
| | | | | | AM401969, AM401970, AM401971, AM401972, AM401973, |
| | | | | | AM401974, AM401975, AM401976, AM401977, AM401978, |
| | | | | | AM401979, AM401980, AM402055, AM402056, AM402057, |
| | | | | | AM402058, AM402059, AM402060, AM402061, AM402062, |
| | | | | | AM402063, AM402064, AM402065, AM402066, AM402067, |
| | | | | | AM402068, AM402069, AM402070, AM402071, AM402072, |
| | | | | | AM402073, AM402074, AM402075, AM402076, AM402077, |
| | | | | | AM402078, AM402079, AM402080, AM402081, AM402082, |
| | | | | | AM402083, AM402084, AM402085, AM402086, AM402088, |
| | | | | | AM402089, AM402090, AM402091, AM402092, AM402093, |
| | | | | | AM402094, AM402095, AM402096, AM402097, AM402098, |
| | | | | | AM402099, AM402100, AM402101, AM402102, AM402103, |
| | | | | | AM402104, AM402106, AM402107, AM402108, AM402109, |
| | | | | | AM402110, AM402111, AM402112, AM402114, AM402115, |
| | | | | | AM402117, AM402118, AM402119, AM402120, AM402121, |
| | | | | | AM402122, AM402123, AM402124, AM402125, AM402126, |
| | | | | | AM402127, AM402129, AM402258, AM402259, AM402260, |
| | | | | | AM402261, AM402262, AM402263, AM402264, AM402265, |
| | | | | | AM402266, AM402267, AM402268, AM402269, AM402270, |
| | | | | | AM402271, AM402272, AM402273, AM402274, AM402275, |
| | | | | | AM402276, AM402277, AM402278, AM402279, AM402280, |
| | | | | | AM402281, AM402282, AM402283, AM402284, AM402285, |
| | | | | | AM402286, AM402287, AM402288, AM402289, AM402290, |
| | | | | | AM402291, AM402292, AM402293, AM402294, AM402295, |
| | | | | | AM402296, AM402297, AM402298, AM402299, AM402300, |
| | | | | | AM402301, AM402302, AM402303, AM402304, AM402305, |
| | | | | | AM402306, AM402307, AM402308, AM402309, AM402310, |
| | | | | | AM402311, AM402312, AM402313, AM402314, AM402315, |
| | | | | | AM402316, AM402317, AM402318, AM402319, AM402320, |
| | | | | | AM402321, AM402322, AM402323, AM402324, AM402325, |
| | | | | | AM402326, AM402327, AM402328, AM402329, AM402330, |
| | | | | | AM402331, AM402332, AM402333, AM402334, AM402335, |
| | | | | | AM402336, AM402337, AM402338, AM402339, AM402340, |
| | | | | | AM402341, AM402342, AM402343, AM402344, AM402345, |
| | | | | | AM402346, AM402347, AM402348, AM402349, AM402350, |
| | | | | | AM402351, AM402352, AM402353, AM402354, AM402355, |
| | | | | | AM402356, AM402357, AM402358, AM402359, AM402360, |
| | | | | | AM402361, AM402362, AM402363, AM402365, AM402367, |
| | | | | | AM402369, AM402370, AM402371, AM402372, AM402373, |
| | | | | | AM402374, AM402375, AM402376, AM402377, AM402378, |
| | | | | | AM402379, AM402380, AM402381, AM402382, AM402383, |
| | | | | | AM402384, AM493436, AM493437, AM493438, AM493439, |
| | | | | | AM493440, AM493441, AM493442, AM493443, AM493444, |
| | | | | | AM493445, AM493446, AM493447, AM493448, AM493449, |
| | | | | | AM493450, AM493451, AM493471, AM493472, AM493473, |
| | | | | | AM493474, AM493475, AM493476, AM493477, AM493478, |
| | | | | | AM493479, AM493480, AM493481, AM493482, AM493483, |
| | | | | | AM493484, AM493485, AM493486, AM493487, AM493488, |
| | | | | | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507, AM494937, |
| | | | | | AY008261, AY044867, AY045702, AY240967, AY240968, |
| | | | | | AY240969, AY460204, AY587016, AY587844, AY808009, |
| | | | | | AY808010, AY808011, AY808012, AY808015, AY808016, |
| | | | | | AY808018, AY808019, AY808020, AY808023, AY808024, |
| | | | | | AY808025, AY808026, AY808027, AY808028, AY808029, |
| | | | | | AY808030, AY808032, AY808034, AY808035, AY808037, |
| | | | | | AY808038, AY808039, AY808042, AY808043, D10934, D11168, |
| | | | | | D14484, D16435, D30613, D45172, D50481, D50482, D50484, |
| | | | | | D50485, D85516, D89815, D89872, D90208, DQ071885, DQ244140, |
| | | | | | DQ244141, DQ491979, DQ491980, DQ491981, DQ491982, |
| | | | | | DQ491983, DQ491984, DQ491985, DQ491986, DQ491987, |
| | | | | | DQ491988, DQ491989, DQ491990, DQ491991, DQ491992, |
| | | | | | DQ491993, DQ491994, DQ491995, DQ491996, DQ491997, |
| | | | | | DQ491998, DQ491999, DQ492000, DQ492001, DQ492002, |
| | | | | | DQ492003, DQ492004, DQ492005, DQ492006, DQ492007, |
| | | | | | DQ492008, DQ492009, DQ492010, DQ492011, DQ492012, |
| | | | | | DQ492013, DQ492014, DQ492015, DQ492016, DQ492017, |
| | | | | | DQ492018, DQ492019, DQ492020, DQ492022, DQ492023, |
| | | | | | DQ492024, DQ492025, DQ492026, DQ492027, DQ492028, |
| | | | | | DQ492029, DQ492030, DQ492031, DQ492032, DQ492033, |
| | | | | | DQ492034, DQ492035, DQ492036, DQ492037, DQ492038, |
| | | | | | DQ492039, DQ492040, DQ492041, DQ492042, DQ492043, |
| | | | | | DQ492044, DQ492045, DQ492046, DQ492047, DQ492048, |
| | | | | | DQ492049, DQ492050, DQ492051, DQ492052, DQ492053, |
| | | | | | DQ492054, DQ492055, DQ492056, DQ492057, DQ492058, |
| | | | | | DQ492059, DQ492060, DQ492061, DQ492062, DQ492063, |
| | | | | | DQ492064, DQ492065, DQ492066, DQ492067, DQ492068, |
| | | | | | DQ492069, DQ492070, DQ492071, DQ492072, DQ492073, |
| | | | | | DQ492075, DQ492076, DQ492077, DQ492078, DQ492079, |
| | | | | | DQ492080, DQ492081, DQ492082, DQ492083, DQ492084, |
| | | | | | DQ492085, DQ492086, DQ492087, DQ492088, DQ492089, |
| | | | | | DQ492090, DQ492091, DQ492092, DQ492094, DQ492095, |
| | | | | | DQ492096, DQ492097, DQ492098, DQ492099, DQ492100, |
| | | | | | DQ492101, DQ492102, DQ492103, DQ492104, DQ492105, |
| | | | | | DQ492106, DQ492107, DQ492108, DQ492109, DQ492110, |
| | | | | | DQ492111, DQ492112, DQ492113, DQ492114, DQ492115, |
| | | | | | DQ492116, DQ492117, DQ492118, DQ492119, DQ492120, |
| | | | | | DQ492121, DQ492122, DQ492123, DQ492124, DQ492125, |
| | | | | | DQ492126, DQ492127, DQ492128, DQ492129, DQ492130, |
| | | | | | DQ492131, DQ492132, DQ492133, DQ492134, DQ492135, |
| | | | | | DQ492136, DQ492137, DQ492138, DQ492139, DQ492140, |
| | | | | | DQ492141, DQ492142, DQ492143, DQ492144, DQ492145, |
| | | | | | DQ492146, DQ492147, DQ492148, DQ492149, DQ492150, |
| | | | | | DQ492151, DQ492152, DQ492153, DQ492154, DQ492155, |
| | | | | | DQ492156, DQ492157, DQ492158, DQ492159, DQ492160, |
| | | | | | DQ492161, DQ492162, DQ859970, EF032892, EF032893, |
| | | | | | EF032894, EF407459, EF407460, EF407461, EF407462, EF407463, |
| | | | | | EF407464, EF407465, EF407466, EF407467, EF407468, EF407469, |
| | | | | | EF407471, EF407473, EF407475, EF407476, EF407477, EF407478, |
| | | | | | EF407479, EF407480, EF407481, EF407482, EF407483, EF407484, |
| | | | | | EF407485, EF407486, EF407487, EF407488, EF407490, EF407491, |
| | | | | | EF407492, EF407494, EF407495, EF407497, EF407498, EF407499, |
| | | | | | EF407500, EF407501, EF407502, EF407503, EF407504, EF638081, |
| | | | | | EU155217, EU155218, EU155219, EU155220, EU155222, |
| | | | | | EU155223, EU155224, EU155225, EU155226, EU155227, |
| | | | | | EU155228, EU155229, EU155230, EU155231, EU155232, |
| | | | | | EU155235, EU155253, EU155254, EU155255, EU155256, |
| | | | | | EU155257, EU155258, EU155259, EU155260, EU155261, |
| | | | | | EU155262, EU155263, EU155264, EU155279, EU155281, |
| | | | | | EU155300, EU155301, EU155302, EU155303, EU155304, |
| | | | | | EU155305, EU155306, EU155307, EU155316, EU155317, |
| | | | | | EU155318, EU155324, EU155325, EU155326, EU155327, |
| | | | | | EU155328, EU155329, EU155330, EU155331, EU155332, |
| | | | | | EU155333, EU155334, EU155335, EU155336, EU155337, |
| | | | | | EU155356, EU155357, EU155358, EU155359, EU155360, |
| | | | | | EU155361, EU155362, EU155363, EU155365, EU155366, |
| | | | | | EU155367, EU155368, EU155369, EU155370, EU155371, |
| | | | | | EU155372, EU155373, EU155374, EU155375, EU155376, |
| | | | | | EU155377, EU155381, EU155382, EU234061, EU234062, |
| | | | | | EU239714, EU255960, EU255961, EU255962, EU256000, |
| | | | | | EU256001, EU256059, EU256061, EU256062, EU256064, |
| | | | | | EU256065, EU256066, EU256075, EU256076, EU256077, |
| | | | | | EU256078, EU256079, EU256080, EU256081, EU256082, |
| | | | | | EU256083, EU256088, EU256089, EU256090, EU256091, |
| | | | | | EU256092, EU256098, EU256099, EU256100, EU256101, |
| | | | | | EU256102, EU256103, EU482833, EU482839, EU482849, |
| | | | | | EU482859, EU482860, EU482874, EU482875, EU482877, |
| | | | | | EU482879, EU482880, EU482885, EU482886, EU482888, |
| | | | | | EU660386, EU660388, EU781827, EU781830, EU781832, |
| | | | | | EU857431, EU862835, EU862837, FJ024277, FJ024279, FJ390396, |
| | | | | | FJ390398, FJ478453, FJ958890, FJ958891, FJ958892, FJ958893, |
| | | | | | FJ958894, FJ958895, FJ958896, FJ958897, FJ958898, FJ958899, |
| | | | | | FJ958900, FJ958901, FJ958902, FJ958903, FJ958904, FJ958905, |
| | | | | | FJ958906, FJ958907, FJ958909, FJ958910, FJ958911, FJ958912, |
| | | | | | FJ958913, FJ958914, FJ958915, FJ958916, FJ958917, FJ958918, |
| | | | | | FJ958919, FJ958920, FJ958921, FJ958922, FJ958923, FJ958924, |
| | | | | | FJ958925, FJ958926, FJ958927, FJ958928, FJ958929, FJ958930, |
| | | | | | FJ958931, FJ958932, FJ958933, FJ958934, FJ958935, FN435993, |
| | | | | | GQ913868, GQ913869, GQ913870, GQ913871, GQ913872, |
| | | | | | GQ913873, GU451218, GU451219, GU451220, GU451221, |
| | | | | | GU451222, GU451223, GU451224, HM042050, HM042052, |
| | | | | | HM042053, HM042054, HM042055, HM042057, HM042058, |
| | | | | | HM042059, HM042060, HM042061, HM042062, HM042063, L02836, |
| | | | | | M84754, M96362, U01214, U16362, U45476, X61596 |
| 397 | cct | cat | P | H | AB154192, AB154199, AB285054, AB285055, AB285068, |
| | | | | | AF165055, AF165056, DQ492082 |
| 397 | cct | cta | P | L | AB154197 |
| 397 | cct | ctc | P | L | EU155232 |
| 397 | cct | ctt | P | L | AB154198, AB285063, AB285069, AF265158, AF265159, |
| | | | | | AM402112, AM402127, AM402277, AY808032, AY808039, |
| | | | | | DQ492157, EU256077, EU256083, GU451224 |
| 397 | cct | tcc | P | S | AJ507169 |
| 397 | cct | tct | P | S | EU529682 |
| 398 | gac | gcc | D | A | EU155306 |
| 398 | gac | gag | D | E | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401862, |
| | | | | | AM401863, AM401864, AM401865, AM401866, AM401867, |
| | | | | | AM401868, AM401869, AM401870, AM401871, AM401872, |
| | | | | | AM401873, AM401874, AM401875, AM401876, AM401877, |
| | | | | | AM401878, AM401879, AM401880, AM401881, AM401882, |
| | | | | | AM401883, AM401884, AM401885, AM401886, AM401887, |
| | | | | | AM401888, AM401889, AM401890, AM401891, AM401892, |
| | | | | | AM401893, AM401894, AM401895, AM401896, AM401897, |
| | | | | | AM401898, AM401899, AM401900, AM401901, AM401902, |
| | | | | | AM401903, AM401904, AM401905, AM401906, AM401907, |
| | | | | | AM401908, AM401909, AM401910, AM401911, AM401912, |
| | | | | | AM401913, AM401914, AM401915 |
| 398 | gac | ggc | D | G | AB049100, AB285039, AB285041, AB285046, AB285077, |
| | | | | | AF265148, AF265149, AF265150, AF265163, AF265164, AF265165, |
| | | | | | AJ132996, AJ507159, AJ507171, AJ507174, AJ507188, AJ507198, |
| | | | | | AY808012, AY808013, AY808014, AY808020, AY808037, |
| | | | | | AY808041, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, DQ492093, |
| | | | | | DQ492123, DQ492163, DQ492164, DQ492165, DQ492166, |
| | | | | | DQ492167, DQ492168, DQ492169, DQ492170, DQ492171, |
| | | | | | DQ492172, DQ492173, DQ492174, DQ492175, DQ492176, |
| | | | | | DQ492177, DQ492178, DQ492179, DQ492180, DQ492181, |
| | | | | | DQ492182, DQ492183, DQ492184, DQ492185, DQ492186, |
| | | | | | DQ492187, DQ492188, DQ492189, DQ492190, DQ492191, |
| | | | | | DQ492192, DQ492193, DQ492194, DQ492195, DQ492196, |
| | | | | | DQ492197, EF407467, EF407489, EF407496, EU155377, |
| | | | | | EU155382, EU256103, GU451218, GU451219, GU451220, |
| | | | | | GU451222, HM042063, U45476 |
| 398 | gac | ggt | D | G | AF165045, AF165046, AF207755, AF207757, AY808026, |
| | | | | | DQ491977, DQ491978, EU155316 |
| 398 | gac | cac | D | H | EU256077 |
| 398 | gac | aac | D | N | AB285056, AM493471, AM493472, AM493473, AM493474, |
| | | | | | AM493475, AM493476, AM493477, AM493478, AM493479, |
| | | | | | AM493480, AM493481, AM493482, AM493483, AM493484, |
| | | | | | AM493485, AM493486, AM493487, AM493488, AM494937 |
| 398 | gac | aat | D | N | AB285035, AB285053, AF207764, AJ507189 |
| 398 | gac | agc | D | S | EU155376 |
| 398 | gac | agt | D | S | AB285073 |
| 398 | gac | acc | D | T | AB285038, AF165047, AF165048 |
| 398 | gac | act | D | T | AB285054, AB285055 |
| 398 | gac | gtt | D | V | AB285079 |
| 398 | gac | tac | D | Y | AB285075 |
| 399 | cag | tgc | Q | C | GU451221, GU451223, GU451224 |
| 399 | cag | gag | Q | E | AB285042, AB285053, AB442221, EU255961, EU256059, L02836 |
| 399 | cag | ggg | Q | G | AB036519, AB036520, AB036521, AB109543, AB191333, AB285050 |
| 399 | cag | cac | Q | H | AB285068, AY460204, EF407489, EF638081 |
| 399 | cag | cat | Q | H | AY808037 |
| 399 | cag | cta | Q | L | EU155316 |
| 399 | cag | ctg | Q | L | AB249644, AB285035, AB285039, AB285075, AB426117, |
| | | | | | AF165055, AF165056, AF265142, AF265143, AF265161, AY808026, |
| | | | | | AY808043, EF407485, EF407496 |
| 399 | cag | ccg | Q | P | AB285066, EU256090 |
| 399 | cag | cga | Q | R | EU155359 |
| 399 | cag | cgg | Q | R | AB285073, AB285076, AJ507161, AM401877, AM402057, |
| | | | | | AY008261, DQ492095 |
| 400 | ccc | gcc | P | A | AB010250, AB049091, AB049094, AB049099, AB049100, |
| | | | | | AB080299, AB154185, AB154186, AB154193, AB249644, |
| | | | | | AB285035, AB285037, AB285041, AB285044, AB285046, |
| | | | | | AB285047, AB285053, AB285058, AB285059, AB285061, |
| | | | | | AB285062, AB285077, AB285078, AB426117, AB442221, |
| | | | | | AB442222, AF165049, AF165050, AF165051, AF165059, AF165060, |
| | | | | | AF165061, AF165062, AF207752, AF207754, AF207756, AF207758, |
| | | | | | AF207761, AF207762, AF207763, AF207765, AF207769, AF207770, |
| | | | | | AF207774, AF265154, AF265155, AF265156, AF333324, AJ507166, |
| | | | | | AJ507169, AJ507177, AJ507178, AJ507179, AJ507181, AJ507182, |
| | | | | | AJ507190, AJ507191, AJ507197, AM402258, AM402259, |
| | | | | | AM402260, AM402261, AM402262, AM402263, AM402264, |
| | | | | | AM402265, AM402266, AM402267, AM402268, AM402269, |
| | | | | | AM402270, AM402271, AM402272, AM402273, AM402274, |
| | | | | | AM402275, AM402276, AM402277, AM402278, AM402279, |
| | | | | | AM402280, AM402281, AM402282, AM402283, AM402284, |
| | | | | | AM402285, AM402286, AM402287, AM402288, AM402289, |
| | | | | | AM402290, AM402291, AM402292, AM402293, AM402294, |
| | | | | | AM402295, AM402296, AM402297, AM402298, AM402299, |
| | | | | | AM402300, AM402301, AM402302, AM402303, AM402304, |
| | | | | | AM402305, AM402306, AM402307, AM402308, AM402309, |
| | | | | | AM402310, AM402311, AM402312, AM402313, AM402314, |
| | | | | | AM402315, AM402316, AM402317, AM402318, AM402319, |
| | | | | | AM402320, AM402321, AM402322, AM402323, AM402324, |
| | | | | | AM402325, AM402326, AM402327, AM402328, AM493436, |
| | | | | | AM493437, AM493438, AM493439, AM493440, AM493441, |
| | | | | | AM493442, AM493443, AM493444, AM493445, AM493446, |
| | | | | | AM493447, AM493448, AM493449, AM493450, AM493451, |
| | | | | | AY240967, AY240968, AY460204, AY587016, AY587844, |
| | | | | | AY808010, AY808018, AY808019, AY808036, AY808040, D30613, |
| | | | | | D45172, D50480, D85516, D89815, D90208, DQ244140, DQ244141, |
| | | | | | DQ491943, DQ491944, DQ491945, DQ491946, DQ491947, |
| | | | | | DQ491948, DQ491949, DQ491950, DQ491951, DQ491952, |
| | | | | | DQ491953, DQ491954, DQ491955, DQ491956, DQ491957, |
| | | | | | DQ492094, DQ492095, DQ492096, DQ492097, DQ492098, |
| | | | | | DQ492099, DQ492100, DQ492101, DQ492102, DQ492103, |
| | | | | | DQ492104, DQ492105, DQ492106, DQ492107, DQ492109, |
| | | | | | DQ492111, DQ492112, DQ492115, DQ492116, DQ492120, |
| | | | | | DQ492121, DQ492122, DQ492123, EF407459, EF407462, |
| | | | | | EF407465, EF407468, EF407474, EF407475, EF407478, EF407479, |
| | | | | | EF407482, EF407486, EF407494, EF407495, EF407498, EF638081, |
| | | | | | EU155232, EU155253, EU155256, EU155263, EU155302, |
| | | | | | EU155305, EU155324, EU155328, EU155336, EU155337, |
| | | | | | EU155358, EU155368, EU155373, EU155374, EU234061, |
| | | | | | EU239714, EU255961, EU256079, EU256101, EU482839, |
| | | | | | EU482874, EU482875, EU482879, EU482888, EU781827, |
| | | | | | EU857431, EU862835, GQ913869, GU451221, GU451223, |
| | | | | | GU451224, L02836, M58335, M96362, U16362 |
| 400 | ccc | gcg | P | A | AB285063 |
| 400 | ccc | gct | P | A | AB285051, AF165052, AF165057, AF165058, AF207772, AY045702, |
| | | | | | AY808028, DQ491979, DQ491980, DQ491981, DQ491982, |
| | | | | | DQ491983, DQ491984, DQ491985, DQ491986, DQ491987, |
| | | | | | DQ491988, DQ491989, DQ491990, DQ491991, DQ491992, |
| | | | | | DQ491993, DQ491994, DQ491995, DQ491996, DQ491997, |
| | | | | | DQ491998, DQ491999, DQ492000, DQ492001, DQ492002, |
| | | | | | DQ492003, DQ492004, DQ492005, DQ492006, DQ492007, |
| | | | | | DQ492108, DQ492110, DQ492113, DQ492114, DQ492117, |
| | | | | | DQ492118, DQ492119, DQ859970, EF407477 |
| 400 | ccc | ttc | P | F | AM493506 |
| 400 | ccc | atc | P | I | AJ507168 |
| 400 | ccc | ctc | P | L | AB154177, AB154178, AB154189, AB154190, AB154195, |
| | | | | | AB154196, AB285056, AB285075, AF207764, AJ507156, |
| | | | | | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401862, |
| | | | | | AM401863, AM401864, AM401867, AM401868, AM401869, |
| | | | | | AM401871, AM401872, AM401873, AM401874, AM401875, |
| | | | | | AM401876, AM401877, AM401878, AM401879, AM401880, |
| | | | | | AM401881, AM401882, AM401883, AM401884, AM401885, |
| | | | | | AM401886, AM401887, AM401888, AM401889, AM401890, |
| | | | | | AM401891, AM401892, AM401893, AM401894, AM401895, |
| | | | | | AM401896, AM401897, AM401898, AM401899, AM401900, |
| | | | | | AM401901, AM401902, AM401903, AM401904, AM401905, |
| | | | | | AM401906, AM401907, AM401908, AM401909, AM401910, |
| | | | | | AM401911, AM401912, AM401913, AM401914, AM401915, |
| | | | | | AY808031, AY808034, DQ492008, DQ492009, DQ492010, |
| | | | | | DQ492011, DQ492012, DQ492013, DQ492014, DQ492015, |
| | | | | | DQ492016, DQ492017, DQ492018, DQ492019, DQ492020, |
| | | | | | DQ492022, DQ492023, DQ492024, DQ492025, DQ492026, |
| | | | | | DQ492027, DQ492028, DQ492029, DQ492030, DQ492031, |
| | | | | | DQ492032, DQ492033, DQ492034, DQ492035, DQ492036, |
| | | | | | DQ492037, DQ492038, DQ492039, EF407492, EF407499, |
| | | | | | EU155369, EU155381, EU256062, EU256075, EU256077, |
| | | | | | EU482849 |
| 400 | ccc | ctt | P | L | AY808015 |
| 400 | ccc | cgc | P | R | AM401865, AM401866, AM401870, EU155259 |
| 400 | ccc | tcc | P | S | AB049096, AB285038, AB285048, AB285065, AB285068, |
| | | | | | AF034151, AF165047, AF165048, AF265148, AF265152, AF265164, |
| | | | | | AF483269, AJ507155, AJ507159, AJ507171, AJ507192, AJ507198, |
| | | | | | AM402121, AM402129, AM493489, AM493490, AM493491, |
| | | | | | AM493492, AM493493, AM493494, AM493495, AM493496, |
| | | | | | AM493497, AM493498, AM493499, AM493500, AM493501, |
| | | | | | AM493502, AM493503, AM493504, AM493505, AM493507, |
| | | | | | AM494937, AY808013, AY808020, AY808021, AY808027, |
| | | | | | AY808033, AY808035, DQ492041, DQ492042, DQ492043, |
| | | | | | DQ492044, DQ492045, DQ492050, DQ492051, DQ492052, |
| | | | | | DQ492056, DQ492057, DQ492058, DQ492059, DQ492060, |
| | | | | | DQ492061, DQ492062, EF032892, EF032893, EF032894, |
| | | | | | EF407460, EF407461, EF407472, EF407473, EF407476, EU155235, |
| | | | | | EU155366, EU155382, EU255960, EU256045, EU256080, |
| | | | | | EU256081, EU256083, EU256088, EU256103, EU482833, |
| | | | | | EU529682, EU781825, FJ958934, GU451218, GU451219, |
| | | | | | GU451220, GU451222, HM042058, U45476 |
| 400 | ccc | tct | P | S | AB285072, AF265145, AF265146, AF265147, EF407500, EF407503, |
| | | | | | HM042063 |
| 400 | ccc | acc | P | T | AB010249, AB049088, AB049089, AB285036, AB285040, |
| | | | | | AB285070, AF207753, AY808017, AY808022, D10934, D11168, |
| | | | | | D14484, D16435, D50482, D50483, D63857, D89872, EU155224, |
| | | | | | EU256090, EU256099, FJ390397 |
| 400 | ccc | act | P | T | AB049098 |
| 400 | ccc | gtc | P | V | AB285069, AF207757, EU155306, U01214 |
| 401 | tcc | gcc | S | A | EU155359 |
| 401 | tcc | gct | S | A | AJ507172 |
| 401 | tcc | ttc | S | F | AF165054, AF207766, AF207769, AF207771, AY808025 |
| 401 | tcc | ttt | S | F | AF165053 |
| 401 | tcc | ctc | S | L | AB049092, AB442220, AF165057, AF165058, AF207768, AJ507161, |
| | | | | | AJ507189, AJ507196, AY808035, AY808038, DQ492040, |
| | | | | | DQ492041, DQ492042, DQ492043, DQ492044, DQ492045, |
| | | | | | DQ492046, DQ492047, DQ492048, DQ492049, DQ492050, |
| | | | | | DQ492051, DQ492052, DQ492053, DQ492054, DQ492055, |
| | | | | | DQ492056, DQ492057, DQ492058, DQ492059, DQ492061, |
| | | | | | DQ492062, EF407478, EF407498, EU155260, EU155301, |
| | | | | | EU155358, EU155361 |
| 401 | tcc | ctt | S | L | AB285065 |
| 401 | tcc | ccc | S | P | AB285042, AF265158, AF483269, AJ000009, AJ507156, AJ507169, |
| | | | | | AJ507182, AM493489, AM493490, AM493491, AM493492, |
| | | | | | AM493493, AM493494, AM493495, AM493496, AM493497, |
| | | | | | AM493498, AM493499, AM493500, AM493501, AM493502, |
| | | | | | AM493503, AM493504, AM493505, AM493506, AM493507, |
| | | | | | AY808015, AY808026, AY808030, AY808031, AY808043, |
| | | | | | DQ492060, DQ492152, EU155281, EU256062 |
| 401 | tcc | cct | S | P | AF165061, AF165062, AY008261, EU256080 |
| 401 | tcc | acc | S | T | AF034151, AM493478, EU256084 |
| 402 | gac | gcc | D | A | AB010250, AY587016, D30613, D45172, D89815, DQ244141, |
| | | | | | EU857431 |
| 402 | gac | gaa | D | E | AM402059, AM402062, AM402065, AM402079, AM402083, |
| | | | | | AM402086, AM402091, AM402123, AM402124, AM402350, |
| | | | | | AM402353, AM402358 |
| 402 | gac | gag | D | E | AJ507190 |
| 402 | gac | ggc | D | G | AB285038, AB285042, AB285072, AF207761, AJ507155, AJ507165, |
| | | | | | AJ507196, AY808015, AY808022, AY808034, DQ244140, |
| | | | | | DQ492008, DQ492009, DQ492010, DQ492011, DQ492012, |
| | | | | | DQ492013, DQ492014, DQ492015, DQ492016, DQ492017, |
| | | | | | DQ492018, DQ492019, DQ492020, DQ492021, DQ492022, |
| | | | | | DQ492023, DQ492024, DQ492025, DQ492026, DQ492027, |
| | | | | | DQ492028, DQ492029, DQ492030, DQ492031, DQ492032, |
| | | | | | DQ492033, DQ492034, DQ492035, DQ492036, DQ492037, |
| | | | | | DQ492038, DQ492039, DQ492100, DQ492108, DQ492109, |
| | | | | | DQ492110, DQ492113, DQ492114, DQ492116, DQ492117, |
| | | | | | DQ492118, DQ492119, DQ859970, EF407486, EU155328, |
| | | | | | EU155337, EU155368, EU256075, EU781827, HM042063, L02836 |
| 402 | gac | ggt | D | G | AY808027, DQ491948, EF407472, EU155256, M96362, U16362 |
| 402 | gac | atc | D | I | EU256081 |
| 402 | gac | aaa | D | K | AB154195, AB154196 |
| 402 | gac | aac | D | N | AB285081, AF207762, AF265154, AF265155, AF265156, AJ507189, |
| | | | | | AM402258, AM402259, AM402260, AM402261, AM402262, |
| | | | | | AM402263, AM402264, AM402265, AM402266, AM402267, |
| | | | | | AM402268, AM402269, AM402270, AM402271, AM402272, |
| | | | | | AM402273, AM402274, AM402275, AM402276, AM402277, |
| | | | | | AM402278, AM402279, AM402280, AM402281, AM402282, |
| | | | | | AM402283, AM402284, AM402285, AM402286, AM402287, |
| | | | | | AM402288, AM402289, AM402290, AM402291, AM402292, |
| | | | | | AM402293, AM402294, AM402295, AM402296, AM402297, |
| | | | | | AM402298, AM402299, AM402300, AM402301, AM402302, |
| | | | | | AM402303, AM402304, AM402305, AM402306, AM402307, |
| | | | | | AM402308, AM402309, AM402310, AM402311, AM402312, |
| | | | | | AM402313, AM402314, AM402315, AM402316, AM402317, |
| | | | | | AM402318, AM402319, AM402320, AM402321, AM402322, |
| | | | | | AM402323, AM402324, AM402325, AM402326, AM402327, |
| | | | | | AM402328, AM402361, AM493436, AM493437, AM493438, |
| | | | | | AM493439, AM493440, AM493441, AM493442, AM493443, |
| | | | | | AM493444, AM493445, AM493446, AM493447, AM493448, |
| | | | | | AM493449, AM493450, AM493451, AY240969, AY808017, D10934, |
| | | | | | D50485, DQ491988, DQ492112, EF032892, EF032893, EF032894, |
| | | | | | EF407495, EU155259, EU155375, EU255961, EU482860, |
| | | | | | EU482879, FJ390397, FJ958903, GQ913873 |
| 402 | gac | aat | D | N | AB049101, AB249644, AB426117, AF207769, AJ507160, AY808028, |
| | | | | | D50480, D50482, D50483, D50484, DQ491979, DQ491980, |
| | | | | | DQ491981, DQ491982, DQ491983, DQ491984, DQ491985, |
| | | | | | DQ491986, DQ491987, DQ491990, DQ491991, DQ491992, |
| | | | | | DQ491993, DQ491994, DQ491995, DQ491996, DQ491997, |
| | | | | | DQ491998, DQ491999, DQ492000, DQ492001, DQ492002, |
| | | | | | DQ492003, DQ492004, DQ492005, DQ492006, DQ492007, |
| | | | | | EU256102 |
| 402 | gac | agc | D | S | AB285048, AY808032, AY808041, D50481, DQ491958, DQ491959, |
| | | | | | DQ491960, DQ491961, DQ491962, DQ491963, DQ491964, |
| | | | | | DQ491965, DQ491966, DQ491967, DQ491968, DQ491969, |
| | | | | | DQ491970, EF407477, EU155257, EU155308, EU155370 |
| 402 | gac | agt | D | S | EF407480, EU155376 |
| 402 | gac | acc | D | T | AM494937 |
| 402 | gac | gtc | D | V | EU155219 |
| 403 | gac | gcc | D | A | AF207766, AF265158, AF265159, AJ507157, AJ507183, AY808009, |
| | | | | | AY808023, EF407471, EF407475, EU155318, EU256081, EU482875 |
| 403 | gac | tgc | D | C | AF207755, AF207757 |
| 403 | gac | tgt | D | C | AF165045, AF165046 |
| 403 | gac | gaa | D | E | AB010250, AB049095, AB285056, AF207764, AY587016, D30613, |
| | | | | | D45172, D89815, EU857431 |
| 403 | gac | gag | D | E | AJ507187 |
| 403 | gac | ttt | D | F | AB285052 |
| 403 | gac | ggc | D | G | AB285045, AB285051, AJ132997, AJ507161, AJ507179, AM401884, |
| | | | | | AM493437, AM493438, AM493439, AM493441, AM493443, |
| | | | | | AM493445, AM493446, AM493449, AM493450, AM493451, |
| | | | | | AY808011, AY808043, DQ492124, DQ492125, DQ492126, |
| | | | | | DQ492127, DQ492128, DQ492129, DQ492131, DQ492132, |
| | | | | | DQ492133, DQ492134, DQ492135, DQ492136, DQ492137, |
| | | | | | DQ492138, DQ492141, DQ492142, DQ492143, DQ492144, |
| | | | | | DQ492145, DQ492146, DQ492147, DQ492148, DQ492149, |
| | | | | | DQ492150, DQ492151, DQ492152, DQ492153, DQ492154, |
| | | | | | DQ492155, DQ492156, DQ492157, DQ492158, DQ492159, |
| | | | | | DQ492160, DQ492161, DQ492162, EF407459, EF407462, |
| | | | | | EF407468, EF407476, EF407479, EF407487, EF407502, EU155225, |
| | | | | | EU155368, EU255960, EU256045, EU482888, L02836 |
| 403 | gac | ggt | D | G | AJ132996 |
| 403 | gac | cac | D | H | DQ491982, DQ491983, DQ491984, DQ491989, DQ491992 |
| 403 | gac | aaa | D | K | AB285048 |
| 403 | gac | aac | D | N | AB049094, AB285078, AF034151, AF176573, AF207771, AF208024, |
| | | | | | AF265142, AF265143, AF265144, AF265160, AF265161, AF265162, |
| | | | | | AF356827, AJ000009, AJ507158, AJ507164, AJ507166, AJ507167, |
| | | | | | AJ507168, AJ507170, AJ507174, AJ507177, AJ507178, AJ507192, |
| | | | | | AJ507194, AJ507198, AJ507199, AM402337, AM493489, |
| | | | | | AM493490, AM493491, AM493492, AM493493, AM493494, |
| | | | | | AM493495, AM493496, AM493497, AM493498, AM493499, |
| | | | | | AM493500, AM493501, AM493502, AM493503, AM493504, |
| | | | | | AM493505, AM493506, AM493507, AY808021, AY808027, |
| | | | | | AY808035, AY808037, AY808039, DQ492040, DQ492041, |
| | | | | | DQ492042, DQ492043, DQ492044, DQ492045, DQ492046, |
| | | | | | DQ492047, DQ492048, DQ492049, DQ492050, DQ492051, |
| | | | | | DQ492052, DQ492053, DQ492056, DQ492057, DQ492058, |
| | | | | | DQ492059, DQ492060, DQ492061, DQ492062, EF032892, |
| | | | | | EF032893, EF032894, EF407460, EF407461, EF407465, EF407482, |
| | | | | | EF407483, EF407488, EF407494, EF407497, EF407500, EF407501, |
| | | | | | EF407503, EF407504, EU155220, EU155222, EU155228, |
| | | | | | EU155235, EU155263, EU155303, EU155317, EU155324, |
| | | | | | EU155325, EU155327, EU155331, EU155356, EU155360, |
| | | | | | EU155366, EU155373, EU155375, EU155376, EU234061, |
| | | | | | EU256064, EU256075, EU256076, EU256083, EU256088, |
| | | | | | EU256091, EU256092, EU256102, EU256103, EU482833, |
| | | | | | EU482874, EU482880, EU482881, EU781825, FJ390396, FJ390398, |
| | | | | | FJ958917, GQ913869, HM042051, HM042052, HM042054, |
| | | | | | HM042055, HM042056, HM042057, HM042061, HM042062, |
| | | | | | HM042063, U01214 |
| 403 | gac | aat | D | N | AJ507156, EU482859, FJ024277 |
| 403 | gac | agc | D | S | AB442220, AF165053, AF165054, AJ507175, AJ507186, AJ507189, |
| | | | | | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401862, |
| | | | | | AM401863, AM401864, AM401865, AM401866, AM401867, |
| | | | | | AM401868, AM401869, AM401870, AM401871, AM401872, |
| | | | | | AM401873, AM401874, AM401875, AM401876, AM401877, |
| | | | | | AM401878, AM401879, AM401880, AM401881, AM401882, |
| | | | | | AM401883, AM401885, AM401886, AM401887, AM401888, |
| | | | | | AM401889, AM401890, AM401891, AM401892, AM401893, |
| | | | | | AM401894, AM401895, AM401896, AM401897, AM401898, |
| | | | | | AM401899, AM401900, AM401901, AM401902, AM401903, |
| | | | | | AM401904, AM401905, AM401906, AM401907, AM401908, |
| | | | | | AM401909, AM401910, AM401911, AM401912, AM401913, |
| | | | | | AM401914, AM401915, AY808031, AY808038, DQ492054, |
| | | | | | DQ492055, DQ492139, EF407470, EF407473, EF407481, |
| | | | | | EU155254, EU155255, EU155316, EU155332, EU155334, |
| | | | | | EU155335, EU155382, EU256062, EU482885, EU781830, |
| | | | | | FJ024279, GQ913868, GQ913871, GQ913872, GQ913873 |
| 403 | gac | acc | D | T | AB285068, EU256000 |
| 403 | gac | tac | D | Y | AF207758 |
| 404 | ggc | tgc | G | C | AB285069, EU155302 |
| 404 | ggc | gac | G | D | AF207772, AJ000009, AJ507161, AJ507165, DQ492067, EF407487, |
| | | | | | EF407502, EU155364, EU155369, EU255960, EU256078, |
| | | | | | EU256081, EU482874 |
| 404 | ggc | gat | G | D | AF207768, AF207774, AJ507159, AY808028, DQ491979, |
| | | | | | DQ491980, DQ491981, DQ491982, DQ491983, DQ491984, |
| | | | | | DQ491985, DQ491986, DQ491987, DQ491988, DQ491989, |
| | | | | | DQ491990, DQ491991, DQ491992, DQ491993, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, DQ492101, EF407462, |
| | | | | | EF407479, EU155227, EU155228, FJ390397, HM042063 |
| 404 | ggc | gag | G | E | AJ507189 |
| 404 | ggc | aat | G | N | EU155362 |
| 404 | ggc | agc | G | S | EF407491, EU155370 |
| 404 | ggc | agt | G | S | AB285038, AF208024, AM401902, AM401907, EU155368 |
| 404 | ggc | gtc | G | V | U45476 |
| 405 | gac | gcc | D | A | AY808026, DQ492172 |
| 405 | gac | gag | D | E | AY808041, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970 |
| 405 | gac | ggc | D | G | AB154201, AB154202, AB285036, AB285074, AF207761, |
| | | | | | AF207768, AF265145, AF265146, AF265147, AJ133096, AJ507165, |
| | | | | | AJ507169, AJ507185, AJ507187, AJ507191, AY808013, AY808018, |
| | | | | | AY808019, AY808028, AY808031, AY808034, AY808040, |
| | | | | | AY808042, DQ491943, DQ491944, DQ491945, DQ491946, |
| | | | | | DQ491947, DQ491949, DQ491950, DQ491951, DQ491952, |
| | | | | | DQ491953, DQ491954, DQ491955, DQ491956, DQ491957, |
| | | | | | DQ491977, DQ491978, DQ491979, DQ491980, DQ491981, |
| | | | | | DQ491982, DQ491983, DQ491984, DQ491985, DQ491986, |
| | | | | | DQ491987, DQ491988, DQ491989, DQ491990, DQ491991, |
| | | | | | DQ491992, DQ491993, DQ491994, DQ491995, DQ491996, |
| | | | | | DQ491997, DQ491998, DQ491999, DQ492000, DQ492001, |
| | | | | | DQ492002, DQ492003, DQ492004, DQ492005, DQ492006, |
| | | | | | DQ492007, DQ492008, DQ492009, DQ492010, DQ492011, |
| | | | | | DQ492012, DQ492013, DQ492014, DQ492015, DQ492016, |
| | | | | | DQ492017, DQ492018, DQ492019, DQ492020, DQ492021, |
| | | | | | DQ492022, DQ492023, DQ492024, DQ492025, DQ492026, |
| | | | | | DQ492027, DQ492028, DQ492030, DQ492031, DQ492032, |
| | | | | | DQ492033, DQ492034, DQ492035, DQ492036, DQ492037, |
| | | | | | DQ492038, DQ492039, DQ492094, DQ492095, DQ492096, |
| | | | | | DQ492097, DQ492098, DQ492099, DQ492101, DQ492102, |
| | | | | | DQ492103, DQ492104, DQ492105, DQ492106, DQ492107, |
| | | | | | DQ492116, DQ492120, DQ492121, DQ492122, DQ492123, |
| | | | | | DQ492159, EF407459, EF407467, EF407492, EF407499, |
| | | | | | EU155227, EU155230, EU155253, EU155260, EU155305, |
| | | | | | EU155357, EU155364, EU155372, EU255961, EU256001, |
| | | | | | EU256059, EU256078, EU256089, GQ913869, GQ913870, |
| | | | | | HM042059 |
| 405 | gac | ggg | D | G | DQ492029 |
| 405 | gac | ggt | D | G | AB049092, AB285075, AF165047, AF165048, AJ507182, AY808029, |
| | | | | | DQ492063, DQ492064, DQ492065, DQ492066, DQ492067, |
| | | | | | DQ492068, DQ492069, DQ492070, DQ492071, DQ492072, |
| | | | | | DQ492073, DQ492074, DQ492075, DQ492077, DQ492078, |
| | | | | | DQ492079, DQ492080, DQ492081, DQ492082, DQ492083, |
| | | | | | DQ492084, DQ492085, DQ492086, DQ492087, DQ492088, |
| | | | | | DQ492089, DQ492090, DQ492091, DQ492092, EU155279, |
| | | | | | EU155369, EU256084 |
| 405 | gac | aac | D | N | AB285063, AJ507197 |
| 405 | gac | aat | D | N | AF207757 |
| 405 | gac | agc | D | S | DQ491971, DQ491972, DQ491973, DQ491974, DQ491975, |
| | | | | | DQ491976, DQ492188 |
| 405 | gac | agt | D | S | DQ492076 |
| 406 | gcg | gaa | A | E | DQ859970 |
| 406 | gcg | gga | A | G | AJ507182 |
| 406 | gcg | ata | A | I | EU155259, EU256083, EU482879 |
| 406 | gcg | aaa | A | K | AB010249, AB049088, AB049091, AB049099, AB249644, |
| | | | | | AB285038, AB285041, AB285058, AB285059, AB285061, |
| | | | | | AB285062, AB285063, AB285069, AB285070, AB285077, |
| | | | | | AB426117, AB442221, AB442222, AF165049, AF165050, |
| | | | | | AF165051, AF165052, AF165057, AF165058, AF165059, AF165060, |
| | | | | | AF165061, AF165062, AF207752, AF207753, AF207754, AF207758, |
| | | | | | AF207762, AF207770, AF207772, AF207774, AF333324, AJ507159, |
| | | | | | AJ507197, AY460204, AY587844, AY808010, D11168, D14484, |
| | | | | | D16435, D63857, D89872, D90208, DQ244140, EF407474, |
| | | | | | EF407478, EF407498, EF638081, EU155231, EU155302, |
| | | | | | EU155336, EU155362, EU155381, EU239714, EU256090, |
| | | | | | EU256099, EU256101, GU451221, GU451223, GU451224, L02836, |
| | | | | | M58335, U01214 |
| 406 | gcg | aag | A | K | AB049100, AB080299, AF207756, AF207763, AJ507181, D85516 |
| 406 | gcg | ttg | A | L | AJ507175 |
| 406 | gcg | aac | A | N | AF207769 |
| 406 | gcg | cca | A | P | DQ492008, DQ492127, DQ492130, DQ492131, DQ492139, |
| | | | | | DQ492140, GU451218, GU451219, GU451220, GU451222 |
| 406 | gcg | ccg | A | P | AB285051 |
| 406 | gcg | caa | A | Q | AB285037, AY045702 |
| 406 | gcg | aga | A | R | AB049098, AB285036, AB285044, AF207765, D10934, M96362, |
| | | | | | U16362, X61596 |
| 406 | gcg | agg | A | R | AB285046 |
| 406 | gcg | tca | A | S | AY808011, AY808034, DQ492009, DQ492010, DQ492011, |
| | | | | | DQ492012, DQ492013, DQ492014, DQ492015, DQ492016, |
| | | | | | DQ492017, DQ492018, DQ492019, DQ492020, DQ492021, |
| | | | | | DQ492022, DQ492023, DQ492025, DQ492026, DQ492027, |
| | | | | | DQ492028, DQ492029, DQ492030, DQ492031, DQ492032, |
| | | | | | DQ492033, DQ492034, DQ492035, DQ492036, DQ492037, |
| | | | | | DQ492038, DQ492039, DQ492124, DQ492125, DQ492126, |
| | | | | | DQ492128, DQ492129, DQ492132, DQ492133, DQ492134, |
| | | | | | DQ492135, DQ492136, DQ492137, DQ492138, DQ492141, |
| | | | | | DQ492142, DQ492143, DQ492144, DQ492145, DQ492146, |
| | | | | | DQ492148, DQ492149, DQ492150, DQ492151, DQ492152, |
| | | | | | DQ492153, DQ492154, DQ492155, DQ492156, DQ492157, |
| | | | | | DQ492158, DQ492159, DQ492160, DQ492161, DQ492162 |
| 406 | gcg | tcg | A | S | AJ133096, AY808015, DQ492024, DQ492147 |
| 406 | gcg | aca | A | T | AB049087, AB049092, AB049094, AB049095, AB049096, |
| | | | | | AB154186, AB285042, AB285048, AB285050, AB285052, |
| | | | | | AB285054, AB285055, AB285057, AB285060, AB285064, |
| | | | | | AB285073, AB442220, AF034151, AF165045, AF165046, AF165053, |
| | | | | | AF165054, AF176573, AF207755, AF207757, AF207764, AF207768, |
| | | | | | AF207771, AF265158, AF265159, AF265160, AF265162, AJ000009, |
| | | | | | AJ132996, AJ132997, AJ507156, AJ507157, AJ507158, AJ507160, |
| | | | | | AJ507165, AJ507171, AJ507172, AJ507183, AJ507185, AJ507187, |
| | | | | | AJ507192, AM401970, AM402069, AM402258, AM402260, |
| | | | | | AM402261, AM402263, AM402264, AM402265, AM402266, |
| | | | | | AM402267, AM402268, AM402269, AM402271, AM402272, |
| | | | | | AM402273, AM402274, AM402275, AM402276, AM402278, |
| | | | | | AM402279, AM402280, AM402281, AM402282, AM402283, |
| | | | | | AM402284, AM402285, AM402286, AM402287, AM402288, |
| | | | | | AM402289, AM402290, AM402291, AM402292, AM402293, |
| | | | | | AM402294, AM402295, AM402296, AM402297, AM402298, |
| | | | | | AM402299, AM402300, AM402301, AM402302, AM402303, |
| | | | | | AM402304, AM402305, AM402306, AM402307, AM402308, |
| | | | | | AM402309, AM402310, AM402311, AM402312, AM402313, |
| | | | | | AM402314, AM402315, AM402316, AM402317, AM402318, |
| | | | | | AM402319, AM402320, AM402321, AM402322, AM402323, |
| | | | | | AM402324, AM402325, AM402326, AM402327, AM402328, |
| | | | | | AM493436, AM493437, AM493438, AM493439, AM493440, |
| | | | | | AM493441, AM493442, AM493443, AM493444, AM493445, |
| | | | | | AM493446, AM493447, AM493448, AM493449, AM493450, |
| | | | | | AM493451, AM493472, AM493473, AM493474, AM493475, |
| | | | | | AM493476, AM493477, AM493478, AM493479, AM493480, |
| | | | | | AM493481, AM493482, AM493483, AM493484, AM493485, |
| | | | | | AM493486, AM493487, AM493488, AY240968, AY808009, |
| | | | | | AY808012, AY808018, AY808021, AY808022, AY808023, |
| | | | | | AY808024, AY808026, AY808027, AY808028, AY808035, |
| | | | | | AY808037, AY808039, AY808043, D50482, D50484, DQ491979, |
| | | | | | DQ491980, DQ491981, DQ491982, DQ491983, DQ491984, |
| | | | | | DQ491985, DQ491986, DQ491987, DQ491988, DQ491989, |
| | | | | | DQ491990, DQ491991, DQ491992, DQ491993, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, DQ492041, DQ492042, |
| | | | | | DQ492043, DQ492044, DQ492045, DQ492050, DQ492051, |
| | | | | | DQ492052, DQ492056, DQ492057, DQ492058, DQ492059, |
| | | | | | DQ492060, DQ492061, DQ492062, EF032892, EF032893, |
| | | | | | EF032894, EF407459, EF407466, EF407467, EF407471, EF407475, |
| | | | | | EF407477, EF407483, EF407488, EF407497, EF407500, EF407501, |
| | | | | | EF407503, EF407504, EU155220, EU155256, EU155260, |
| | | | | | EU155303, EU155305, EU155318, EU155327, EU155331, |
| | | | | | EU155357, EU155358, EU155360, EU155361, EU155363, |
| | | | | | EU155373, EU155375, EU155377, EU256000, EU256075, |
| | | | | | EU256079, EU256084, EU256089, EU256091, EU256092, |
| | | | | | EU256102, EU256103, EU482860, EU482880, EU482881, |
| | | | | | EU482888, EU781825, EU781827, EU862837, FJ390396, FJ390398, |
| | | | | | FJ958916, GQ913873, HM042050, HM042055, HM042061, |
| | | | | | HM042063, U45476 |
| 406 | gcg | acc | A | T | EU155317 |
| 406 | gcg | acg | A | T | AB429050, AJ507163, AJ507164, AJ507166, AJ507167, AJ507170, |
| | | | | | AJ507174, AJ507178, AJ507179, AJ507193, AJ507196, AJ507198, |
| | | | | | AJ507199, AM402277, AM493471, AM494937, AY808014, |
| | | | | | AY808017, AY808025, AY808038, DQ492040, DQ492047, |
| | | | | | DQ492048, DQ492049, DQ492054, DQ492055, DQ492093, |
| | | | | | DQ492163, DQ492164, DQ492165, DQ492166, DQ492167, |
| | | | | | DQ492168, DQ492169, DQ492170, DQ492171, DQ492173, |
| | | | | | DQ492174, DQ492175, DQ492176, DQ492177, DQ492178, |
| | | | | | DQ492179, DQ492180, DQ492182, DQ492183, DQ492184, |
| | | | | | DQ492185, DQ492186, DQ492187, DQ492188, DQ492189, |
| | | | | | DQ492190, DQ492191, DQ492192, DQ492193, DQ492194, |
| | | | | | DQ492195, DQ492196, DQ492197, EF407461, EU155222, |
| | | | | | EU155228, EU155235, EU155325, EU155337, EU155356, |
| | | | | | EU155372, EU155374, EU256001, EU256064, EU256076, |
| | | | | | EU482859, FJ024277, HM042051, HM042052, HM042054, |
| | | | | | HM042056, HM042057, HM042058 |
| 406 | gcg | act | A | T | AB285075, EU155359 |
| 406 | gcg | gta | A | V | AY808041, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970, EF407465, |
| | | | | | FJ478453 |
| 406 | gcg | gtg | A | V | DQ492172 |
| 407 | gga | gca | G | A | AF207772 |
| 407 | gga | tgc | G | C | AJ507182 |
| 407 | gga | gaa | G | E | AB010249, AB049091, AB049099, AB049100, AB249644, |
| | | | | | AB285036, AB285038, AB285046, AB285058, AB285063, |
| | | | | | AB426117, AB442222, AF165057, AF165058, AF165059, AF165060, |
| | | | | | AF165061, AF165062, AF207754, AF207762, AF207763, AF207765, |
| | | | | | AF207770, AJ507196, AM402374, AY587844, AY808013, D10934, |
| | | | | | D11168, D14484, D16435, D89872, DQ491959, DQ491960, |
| | | | | | EF407489, EU155231, EU155232, EU155336, EU155376, |
| | | | | | EU155381, EU239714, EU256090, EU256099, EU256101 |
| 407 | gga | gag | G | E | AB285070, AJ507175, DQ244140, M96362, U16362 |
| 407 | gga | aaa | G | K | EF407491 |
| 407 | gga | aga | G | R | AB285078, AM493500, DQ492067, DQ492073 |
| 407 | gga | agg | G | R | HM042050 |
| 407 | gga | cga | G | R | AM401916, AM401917, AM401918, AM401919, AM401920, |
| | | | | | AM401921, AM401922, AM401923, AM401924, AM401925, |
| | | | | | AM401926, AM401927, AM401928, AM401929, AM401930, |
| | | | | | AM401931, AM401932, AM401933, AM401934, AM401935, |
| | | | | | AM401936, AM401937, AM401938, AM401939, AM401940, |
| | | | | | AM401941, AM401942, AM401943, AM401944, AM401945, |
| | | | | | AM401946, AM401947, AM401948, AM401949, AM401950, |
| | | | | | AM401951, AM401952, AM401953, AM401954, AM401955, |
| | | | | | AM401956, AM401957, AM401958, AM401959, AM401960, |
| | | | | | AM401961, AM401962, AM401963, AM401964, AM401965, |
| | | | | | AM401966, AM401967, AM401968, AM401969, AM401970, |
| | | | | | AM401971, AM401972, AM401973, AM401974, AM401975, |
| | | | | | AM401976, AM401977, AM401978, AM401979, AM401980 |
| 408 | tcc | ttc | S | F | AM401906 |
| 408 | tcc | ccc | S | P | AM401890, AM402364, AM402374 |
| 408 | tcc | acc | S | T | AJ507171, AJ507174, AJ507190, EU155369, EU256078 |
| 408 | tcc | tac | S | Y | DQ491984 |
| 409 | gac | gaa | D | E | EF407461 |
| 409 | gac | ggc | D | G | AM401884, AM402072 |
| 409 | gac | aac | D | N | DQ492152, EU482874 |
| 409 | gac | tac | D | Y | DQ492020 |
| 410 | gtt | gca | V | A | EU155362 |
| 410 | gtt | gcc | V | A | AB285041, AB285060, AM401932, AM401933, AM401939, |
| | | | | | AM401945, AM401956, AM402113, AY808026, AY808031, |
| | | | | | DQ244141, EF407467, EU155361, EU155381, EU256103, |
| | | | | | EU482881, GU451220 |
| 410 | gtt | gcg | V | A | AB285036 |
| 410 | gtt | gct | V | A | AB010250, AB036519, AB036520, AB036521, AB049096, |
| | | | | | AB109543, AB154187, AB154188, AB191333, AB285037, |
| | | | | | AB285042, AB285047, AB285052, AB285059, AB285066, |
| | | | | | AB285068, AF165045, AF165046, AF165053, AF165054, AF207755, |
| | | | | | AF207757, AF207771, AF265152, AF265153, AF356827, AF483269, |
| | | | | | AJ132996, AJ507156, AJ507159, AJ507162, AJ507168, AJ507169, |
| | | | | | AJ507171, AJ507172, AJ507175, AJ507188, AJ507189, AJ507194, |
| | | | | | AJ507196, AJ507197, AJ507198, AM401859, AM401916, |
| | | | | | AM401917, AM401918, AM401919, AM401920, AM401921, |
| | | | | | AM401922, AM401923, AM401924, AM401925, AM401926, |
| | | | | | AM401927, AM401928, AM401929, AM401930, AM401931, |
| | | | | | AM401934, AM401935, AM401936, AM401937, AM401938, |
| | | | | | AM401940, AM401941, AM401942, AM401943, AM401944, |
| | | | | | AM401946, AM401947, AM401948, AM401949, AM401950, |
| | | | | | AM401951, AM401952, AM401953, AM401954, AM401955, |
| | | | | | AM401957, AM401958, AM401959, AM401960, AM401961, |
| | | | | | AM401962, AM401963, AM401964, AM401965, AM401966, |
| | | | | | AM401967, AM401968, AM401969, AM401970, AM401971, |
| | | | | | AM401972, AM401973, AM401974, AM401975, AM401976, |
| | | | | | AM401977, AM401978, AM401979, AM401980, AM402055, |
| | | | | | AM402056, AM402057, AM402058, AM402059, AM402060, |
| | | | | | AM402061, AM402062, AM402063, AM402064, AM402065, |
| | | | | | AM402066, AM402067, AM402068, AM402069, AM402070, |
| | | | | | AM402071, AM402072, AM402073, AM402074, AM402075, |
| | | | | | AM402076, AM402077, AM402078, AM402079, AM402080, |
| | | | | | AM402081, AM402082, AM402083, AM402084, AM402085, |
| | | | | | AM402086, AM402087, AM402088, AM402089, AM402090, |
| | | | | | AM402091, AM402092, AM402093, AM402094, AM402095, |
| | | | | | AM402096, AM402097, AM402098, AM402099, AM402100, |
| | | | | | AM402101, AM402102, AM402103, AM402104, AM402105, |
| | | | | | AM402106, AM402107, AM402108, AM402109, AM402110, |
| | | | | | AM402111, AM402112, AM402114, AM402115, AM402116, |
| | | | | | AM402117, AM402118, AM402119, AM402120, AM402121, |
| | | | | | AM402122, AM402123, AM402124, AM402125, AM402126, |
| | | | | | AM402127, AM402128, AM402129, AM402348, AY587016, |
| | | | | | AY808009, AY808016, AY808019, AY808023, AY808030, |
| | | | | | AY808036, AY808039, D10934, D30613, D45172, D89815, |
| | | | | | DQ491986, DQ491987, DQ491993, DQ492023, DQ492024, |
| | | | | | DQ492025, DQ492026, DQ492027, DQ492028, DQ492029, |
| | | | | | DQ492030, DQ492031, DQ492032, DQ492033, DQ492034, |
| | | | | | DQ492035, DQ492036, DQ492037, DQ492038, DQ492039, |
| | | | | | DQ492077, DQ492094, DQ492095, DQ492096, DQ492097, |
| | | | | | DQ492098, DQ492099, DQ492100, DQ492101, DQ492102, |
| | | | | | DQ492103, DQ492104, DQ492105, DQ492106, DQ492107, |
| | | | | | DQ492108, DQ492109, DQ492110, DQ492111, DQ492112, |
| | | | | | DQ492113, DQ492114, DQ492115, DQ492116, DQ492117, |
| | | | | | DQ492118, DQ492119, DQ492120, DQ492121, DQ492122, |
| | | | | | DQ492123, DQ492165, DQ492197, DQ859970, EF407461, |
| | | | | | EF407482, EF407500, EF407503, EU155218, EU155221, |
| | | | | | EU155259, EU155279, EU155302, EU155306, EU155307, |
| | | | | | EU155316, EU155324, EU155365, EU155367, EU155373, |
| | | | | | EU155374, EU239714, EU256045, EU256061, EU256066, |
| | | | | | EU256076, EU256077, EU256079, EU256085, EU256088, |
| | | | | | EU256101, EU482833, EU482874, EU482875, EU529682, |
| | | | | | EU660388, EU781830, EU857431, EU862835, FJ024277, FJ390396, |
| | | | | | FJ390397, GQ913870, HM042050, U01214 |
| 410 | gtt | gat | V | D | DQ491988, EF407497, EU155231, X61596 |
| 410 | gtt | ggt | V | G | EF407462 |
| 410 | gtt | att | V | I | AB010249, AJ507170, D16435, D89872 |
| 410 | gtt | act | V | T | AJ507182 |
| 411 | gag | gcg | E | A | EU256079 |
| 411 | gag | gac | E | D | DQ492142, EU155324 |
| 411 | gag | gat | E | D | AM493503 |
| 411 | gag | gga | E | G | AM402055, AM402056, AM402057, AM402058, AM402059, |
| | | | | | AM402060, AM402061, AM402062, AM402063, AM402064, |
| | | | | | AM402065, AM402066, AM402067, AM402068, AM402069, |
| | | | | | AM402070, AM402071, AM402072, AM402073, AM402074, |
| | | | | | AM402076, AM402077, AM402078, AM402079, AM402080, |
| | | | | | AM402081, AM402082, AM402083, AM402084, AM402085, |
| | | | | | AM402086, AM402087, AM402088, AM402089, AM402090, |
| | | | | | AM402091, AM402092, AM402093, AM402094, AM402095, |
| | | | | | AM402096, AM402097, AM402098, AM402099, AM402100, |
| | | | | | AM402101, AM402102, AM402103, AM402104, AM402105, |
| | | | | | AM402106, AM402107, AM402108, AM402109, AM402110, |
| | | | | | AM402111, AM402112, AM402113, AM402114, AM402115, |
| | | | | | AM402116, AM402117, AM402118, AM402119, AM402120, |
| | | | | | AM402121, AM402122, AM402123, AM402124, AM402125, |
| | | | | | AM402126, AM402127, AM402128, AM402129, AY808009, |
| | | | | | EU155381 |
| 411 | gag | ggg | E | G | AB049087, AB049094, AB049095, AB049096, AB154198, |
| | | | | | AB285040, AB285050, AB285060, AB285066, AF165053, |
| | | | | | AF165054, AF165055, AF165056, AF207764, AF207771, AF207772, |
| | | | | | AF483269, AJ238800, AJ507159, AJ507160, AJ507162, AJ507163, |
| | | | | | AJ507166, AJ507168, AJ507172, AJ507175, AJ507188, AJ507196, |
| | | | | | AM493471, AM493472, AM493473, AM493474, AM493475, |
| | | | | | AM493476, AM493477, AM493478, AM493479, AM493480, |
| | | | | | AM493481, AM493482, AM493483, AM493484, AM493485, |
| | | | | | AM493486, AM493487, AM493488, AM493497, AY008261, |
| | | | | | AY808016, AY808024, AY808028, AY808030, AY808032, |
| | | | | | AY808036, AY808039, D50480, D50482, D50484, DQ491979, |
| | | | | | DQ491980, DQ491981, DQ491985, DQ491986, DQ491987, |
| | | | | | DQ491988, DQ491990, DQ491991, DQ491993, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, DQ492111, DQ492148, |
| | | | | | DQ492170, DQ492192, EF407461, EF407468, EF407470, |
| | | | | | EF407487, EF407494, EF407497, EU155218, EU155221, |
| | | | | | EU155306, EU155307, EU155358, EU155361, EU155362, |
| | | | | | EU155363, EU155365, EU155367, EU155373, EU155374, |
| | | | | | EU155377, EU255960, EU256045, EU256061, EU256085, |
| | | | | | EU256088, EU256098, EU256103, EU529682, EU660388, |
| | | | | | EU862835, EU862837, FJ390396, HM042053 |
| 411 | gag | gtg | E | V | DQ492131 |
| 412 | tcg | gcg | S | A | AJ507174 |
| 412 | tcg | gag | S | E | AJ507182 |
| 412 | tcg | ttg | S | L | AM402384 |
| 412 | tcg | ccg | S | P | AM401886 |
| 413 | tac | gcc | Y | A | AJ507182 |
| 413 | tac | tgc | Y | C | AB154195, AB154196, AB285076, AB429050, AJ000009, AJ507175, |
| | | | | | AM401894, AM402267, AM402294, DQ491988, DQ491989, |
| | | | | | EF407467, EF407478, EF407489, EU155305, EU256080, |
| | | | | | EU256092, EU482875, EU529682, GQ913873, M84754 |
| 413 | tac | tgt | Y | C | AB036521, AJ132996 |
| 413 | tac | ttc | Y | F | EF407487, EF407502, EU155381, M96362, U16362 |
| 413 | tac | ttt | Y | F | FJ390397 |
| 413 | tac | cac | Y | H | AJ507178, AM402090, AY808025, AY808026, HM042056 |
| 414 | tcc | ttc | S | F | DQ491979 |
| 414 | tcc | acc | S | T | EU155307 |
| 416 | atg | ata | M | I | AM401874, AM493443 |
| 416 | atg | ttg | M | L | AM401879 |
| 416 | atg | acg | M | T | AM493478 |
| 416 | atg | gtg | M | V | AM401888, DQ491947 |
| 417 | ccc | ctc | P | L | AM401951 |
| 417 | ccc | tcc | P | S | AM401967, AM493507, DQ492084 |
| 418 | ccc | gcc | P | A | EU256077 |
| 418 | ccc | tcc | P | S | AM402311 |
| 419 | ctt | ttt | L | F | DQ492104 |
| 419 | ctt | cac | L | H | DQ492066 |
| 419 | ctt | cat | L | H | DQ492106 |
| 419 | ctt | cct | L | P | AM402326, DQ492156 |
| 420 | gag | ggg | E | G | AM401866, DQ492018, DQ492107 |
| 420 | gag | aag | E | K | AM401956 |
| 420 | gag | agg | E | R | AB285081 |
| 422 | gag | ggg | E | G | AM402092, AM402329, AM493445, AM493446, DQ492072, |
| | | | | | DQ492180 |
| 423 | ccg | ctg | P | L | AB285078, AM402077 |
| 423 | ccg | cag | P | Q | AY808037 |
| 423 | ccg | tcg | P | S | AM401851 |
| 424 | ggg | agg | G | R | AM401964 |
| 425 | gat | gaa | D | E | AB285075 |
| 425 | gat | ggc | D | G | DQ492080, FJ958897 |
| 425 | gat | aac | D | N | DQ491945 |
| 426 | ccc | ctc | P | L | AM402344 |
| 427 | gat | ggt | D | G | DQ492060 |
| 427 | gat | aat | D | N | AM401907, AM402334 |
| 428 | ctc | ttc | L | F | AF165045, AF165046, AF207755, AM401911, AY808040, |
| | | | | | AY808041, DQ491943, DQ491944, DQ491945, DQ491946, |
| | | | | | DQ491947, DQ491948, DQ491949, DQ491950, DQ491951, |
| | | | | | DQ491952, DQ491953, DQ491954, DQ491955, DQ491956, |
| | | | | | DQ491957, DQ491958, DQ491959, DQ491960, DQ491961, |
| | | | | | DQ491962, DQ491963, DQ491964, DQ491965, DQ491966, |
| | | | | | DQ491967, DQ491968, DQ491969, DQ491970 |
| 429 | agc | ggc | S | G | AM401851, AM401898, AM401905, DQ491978 |
| 429 | agc | aac | S | N | AM402262, D30613, D45172 |
| 429 | agc | acc | S | T | DQ492181 |
| 430 | gac | gaa | D | E | AB285078 |
| 430 | gac | ggc | D | G | FJ958904 |
| 430 | gac | aac | D | N | EU155305 |
| 430 | gac | aat | D | N | DQ492193 |
| 431 | ggg | gag | G | E | DQ492029 |
| 432 | tct | ttt | S | F | DQ492042 |
| 432 | tct | ccc | S | P | DQ492037 |
| 433 | tgg | ggg | W | G | AY808033, AY808038, DQ492071, DQ492193 |
| 433 | tgg | cgg | W | R | DQ492180 |
| 434 | tct | ttc | S | F | AM402064, AM402094 |
| 434 | tct | ttt | S | F | DQ491970 |
| 435 | acc | gcc | T | A | DQ492084 |
| 436 | gta | gca | V | A | DQ491983 |
| 436 | gta | gcg | V | A | AM401923 |
| 436 | gta | atg | V | M | AB049100, AB285037, AB285052, AB285057, AB285058, |
| | | | | | AB285064, AB442222, AF165057, AF165058, AJ507156, AJ507185, |
| | | | | | AM402293, AM494937, AY808014, AY808015, AY808022, |
| | | | | | AY808026, DQ492064, DQ492066, DQ492069, DQ492072, |
| | | | | | DQ492076, DQ492077, DQ492081, DQ492093, DQ492127, |
| | | | | | DQ492130, DQ492131, DQ492132, DQ492135, DQ492139, |
| | | | | | DQ492140, DQ492148, DQ492163, DQ492164, DQ492165, |
| | | | | | DQ492166, DQ492167, DQ492168, DQ492169, DQ492170, |
| | | | | | DQ492171, DQ492172, DQ492173, DQ492174, DQ492175, |
| | | | | | DQ492176, DQ492177, DQ492178, DQ492179, DQ492180, |
| | | | | | DQ492181, DQ492182, DQ492183, DQ492184, DQ492185, |
| | | | | | DQ492186, DQ492187, DQ492188, DQ492189, DQ492190, |
| | | | | | DQ492191, DQ492192, DQ492193, DQ492194, DQ492195, |
| | | | | | DQ492196, DQ492197, EF407471, EU155235, EU155257, |
| | | | | | EU155264, EU482859, HM042061 |
| 437 | agc | tgt | S | C | DQ492141 |
| 437 | agc | gac | S | D | EF407464 |
| 437 | agc | ggc | S | G | AJ507189, DQ491978 |
| 437 | agc | ggt | S | G | DQ492095 |
| 437 | agc | aac | S | N | AF207765, DQ491970, DQ492061 |
| 437 | agc | acc | S | T | DQ492181 |
| 438 | gag | gac | E | D | D63857, EU155223, EU155253 |
| 438 | gag | gat | E | D | EU256084, HM042059 |
| 438 | gag | gga | E | G | EF407470 |
| 438 | gag | ggg | E | G | AB010249, AB285054, AB285055, AF207757, AM401931, |
| | | | | | AY240969, D11168, D16435, D89872, D90208, DQ492179, |
| | | | | | EU155359 |
| 438 | gag | aaa | E | K | AM402290, AM402293, AM402294, AM402296, AM402297, |
| | | | | | AM402299, AM402301, AM402302, AM402303, AM402304, |
| | | | | | AM402305, AM402306, AM402308 |
| 438 | gag | aag | E | K | DQ492181 |
| 438 | gag | gtg | E | V | AM402347 |
| 439 | gag | gac | E | D | AB285046, EU529682 |
| 439 | gag | gat | E | D | FJ390398 |
| 439 | gag | ggg | E | G | AM402291, AM402364, AY808026 |
| 439 | gag | cag | E | Q | AF165053 |
| 439 | gag | gtg | E | V | DQ492087 |
| 440 | gct | gac | A | D | AJ507169, EU155359 |
| 440 | gct | gat | A | D | AF165053, AY808026, M84754 |
| 440 | gct | ggc | A | G | EF407470 |
| 440 | gct | ggt | A | G | AB049092, AF165054, EU256090 |
| 440 | gct | cat | A | H | EU256077 |
| 440 | gct | ccc | A | P | AY808029, DQ492064, DQ492066, DQ492069, DQ492072, |
| | | | | | DQ492076, DQ492077, DQ492081 |
| 440 | gct | cct | A | P | AB442222, AF165057, AF165058 |
| 440 | gct | acc | A | T | AB285061, AM493497, DQ492071, DQ492073, DQ492078, |
| | | | | | DQ492079, DQ492083, DQ492088, DQ492092, EU256088 |
| 440 | gct | act | A | T | AB285054, AB285055, AM493489, AM493490, AM493491, |
| | | | | | AM493492, AM493493, AM493494, AM493495, AM493496, |
| | | | | | AM493498, AM493499, AM493500, AM493501, AM493502, |
| | | | | | AM493503, AM493504, AM493505, AM493506, AM493507, |
| | | | | | DQ492063, DQ492065, DQ492067, DQ492068, DQ492070, |
| | | | | | DQ492074, DQ492075, DQ492080, DQ492082, DQ492084, |
| | | | | | DQ492085, DQ492086, DQ492087, DQ492089, DQ492090, |
| | | | | | DQ492091, FJ390398 |
| 440 | gct | gtc | A | V | AF265164, AM402339, AY808014, EF407460 |
| 440 | gct | gtt | A | V | AB049089, AF207760, AF265148, AF265149, AF265150, AF265152, |
| | | | | | AF265153, AF265163, AF265165, DQ244141, DQ492056, |
| | | | | | EU155280, GQ913868 |
| 441 | agt | gcc | S | A | AF207770 |
| 441 | agt | gct | S | A | EU155306 |
| 441 | agt | tgt | S | C | DQ492017 |
| 441 | agt | gac | S | D | AB442221, AF207760, AM401978 |
| 441 | agt | gat | S | D | AM401916, AM401917, AM401918, AM401919, AM401920, |
| | | | | | AM401921, AM401922, AM401923, AM401924, AM401925, |
| | | | | | AM401926, AM401927, AM401928, AM401929, AM401930, |
| | | | | | AM401931, AM401932, AM401933, AM401934, AM401935, |
| | | | | | AM401936, AM401937, AM401938, AM401939, AM401940, |
| | | | | | AM401941, AM401942, AM401943, AM401944, AM401945, |
| | | | | | AM401946, AM401947, AM401948, AM401949, AM401950, |
| | | | | | AM401951, AM401952, AM401953, AM401954, AM401955, |
| | | | | | AM401956, AM401957, AM401958, AM401959, AM401960, |
| | | | | | AM401961, AM401962, AM401963, AM401964, AM401965, |
| | | | | | AM401966, AM401967, AM401968, AM401969, AM401970, |
| | | | | | AM401971, AM401972, AM401973, AM401974, AM401975, |
| | | | | | AM401976, AM401977, AM401979, AM401980, GQ913873 |
| 441 | agt | gga | S | G | EF407467 |
| 441 | agt | ggc | S | G | AB010249, AB285038, AB285039, AB442220, AF165061, AJ507157, |
| | | | | | AJ507167, AJ507173, D16435, D50480, D50483, D89872, |
| | | | | | EF407496, EU482877, EU482885, FJ390398 |
| 441 | agt | ggt | S | G | AB049087, AB049088, AB049089, AB049092, AB049094, |
| | | | | | AB049098, AB049100, AB154180, AB154189, AB154190, |
| | | | | | AB154196, AB249644, AB285036, AB285041, AB285042, |
| | | | | | AB285044, AB285047, AB285051, AB285052, AB285054, |
| | | | | | AB285055, AB285057, AB285058, AB285060, AB285061, |
| | | | | | AB285064, AB285065, AB285067, AB285071, AB285076, |
| | | | | | AB426117, AB442222, AF034151, AF165053, AF165054, AF165057, |
| | | | | | AF165058, AF165062, AF207753, AF207756, AF207764, AF207765, |
| | | | | | AF207768, AF207771, AF207772, AJ238800, AJ507163, AJ507168, |
| | | | | | AJ507169, AJ507171, AJ507181, AJ507184, AJ507186, AJ507189, |
| | | | | | AJ507194, AJ507195, AM493489, AM493490, AM493491, |
| | | | | | AM493492, AM493493, AM493494, AM493495, AM493496, |
| | | | | | AM493497, AM493498, AM493499, AM493500, AM493501, |
| | | | | | AM493502, AM493503, AM493504, AM493505, AM493506, |
| | | | | | AM493507, AY008261, AY044867, AY045702, AY240967, |
| | | | | | AY240968, AY240969, AY808026, AY808028, AY808029, |
| | | | | | AY808033, AY808034, AY808036, AY808039, D50481, D50482, |
| | | | | | D50484, D50485, D90208, DQ244140, DQ244141, DQ491979, |
| | | | | | DQ491980, DQ491981, DQ491982, DQ491983, DQ491984, |
| | | | | | DQ491985, DQ491986, DQ491987, DQ491988, DQ491989, |
| | | | | | DQ491990, DQ491991, DQ491992, DQ491993, DQ491994, |
| | | | | | DQ491995, DQ491996, DQ491997, DQ491998, DQ491999, |
| | | | | | DQ492000, DQ492001, DQ492002, DQ492003, DQ492004, |
| | | | | | DQ492005, DQ492006, DQ492007, DQ492047, DQ492048, |
| | | | | | DQ492054, DQ492055, DQ492063, DQ492064, DQ492065, |
| | | | | | DQ492066, DQ492067, DQ492068, DQ492069, DQ492070, |
| | | | | | DQ492071, DQ492072, DQ492073, DQ492074, DQ492075, |
| | | | | | DQ492076, DQ492077, DQ492078, DQ492079, DQ492080, |
| | | | | | DQ492081, DQ492082, DQ492083, DQ492084, DQ492085, |
| | | | | | DQ492086, DQ492087, DQ492088, DQ492089, DQ492090, |
| | | | | | DQ492091, DQ492092, DQ492125, EF407470, EF407472, |
| | | | | | EF407475, EF407477, EF407480, EF407481, EF407485, EF407490, |
| | | | | | EF407491, EF407495, EF407500, EF407501, EF407503, EF407504, |
| | | | | | EU155232, EU155257, EU155261, EU155305, EU155315, |
| | | | | | EU155318, EU155335, EU155359, EU155363, EU155364, |
| | | | | | EU155368, EU155370, EU256080, EU256081, EU256082, |
| | | | | | EU256083, EU256090, EU482849, EU781827, EU862835, |
| | | | | | FN435993, GQ913869, GQ913872, HM042050, HM042052, |
| | | | | | HM042054, HM042056, HM042057, HM042059, HM042061, M84754 |
| 441 | agt | aac | S | N | AM493450, U45476 |
| 441 | agt | aat | S | N | AB285043, HM042051, HM042058 |
| 441 | agt | cgc | S | R | AF207769 |
| 441 | agt | act | S | T | AB049091, HM042062 |
| 442 | gag | gac | E | D | AB010249, D11168, D16435, D89872 |
| 442 | gag | gat | E | D | AM493489, AM493490, AM493491, AM493492, AM493493, |
| | | | | | AM493494, AM493495, AM493496, AM493497, AM493498, |
| | | | | | AM493499, AM493500, AM493501, AM493502, AM493503, |
| | | | | | AM493504, AM493505, AM493506, AM493507, EF407481 |
| 442 | gag | ggg | E | G | AM402112, AM402352, D10934, EU155358, EU256080 |
| 442 | gag | aag | E | K | FJ958919 |
| 442 | gag | cag | E | Q | AB285061, AY240967, AY240968, AY240969 |
| 443 | gac | gcc | D | A | EF407468 |
| 443 | gac | gaa | D | E | EU155358 |
| 443 | gac | ggc | D | G | AB285040, AJ507169, AM402318, AM494937, M84754 |
| 443 | gac | cac | D | H | AM493450 |
| 443 | gac | aac | D | N | AB285041, AJ507161, EF407461, EF407481, EU529682, |
| | | | | | EU862835, GQ913873, HM042058, HM042059 |
| 443 | gac | agc | D | S | AB285076, EF407467, EU155306, HM042056 |
| 444 | gtc | gct | V | A | AJ507164 |
| 444 | gtc | atc | V | I | AB049098, AB285041, AJ507169, D11168, D16435, D89872, |
| | | | | | EF407461, GQ913873, HM042059 |
| 444 | gtc | att | V | I | AB285040, EF407481, EU529682 |
| 444 | gtc | ctt | V | L | AF165056 |
| 445 | gtc | gcc | V | A | AM401848, AM401849, AM401850, AM401851, AM401852, |
| | | | | | AM401853, AM401854, AM401855, AM401856, AM401857, |
| | | | | | AM401858, AM401859, AM401860, AM401861, AM401862, |
| | | | | | AM401863, AM401864, AM401865, AM401866, AM401867, |
| | | | | | AM401868, AM401869, AM401870, AM401871, AM401873, |
| | | | | | AM401874, AM401875, AM401876, AM401877, AM401878, |
| | | | | | AM401879, AM401880, AM401881, AM401882, AM401883, |
| | | | | | AM401884, AM401885, AM401886, AM401887, AM401888, |
| | | | | | AM401889, AM401890, AM401891, AM401892, AM401893, |
| | | | | | AM401894, AM401895, AM401896, AM401897, AM401898, |
| | | | | | AM401899, AM401900, AM401901, AM401902, AM401903, |
| | | | | | AM401904, AM401905, AM401906, AM401907, AM401908, |
| | | | | | AM401909, AM401910, AM401911, AM401912, AM401913, |
| | | | | | AM401914, AM401915, AM402111, AM493436, AM493437, |
| | | | | | AM493438, AM493439, AM493440, AM493441, AM493442, |
| | | | | | AM493443, AM493444, AM493445, AM493446, AM493447, |
| | | | | | AM493448, AM493449, AM493450, AM493451 |
| 445 | gtc | gct | V | A | X61596 |
| 445 | gtc | atc | V | I | AB285045, AB285053, AF165054, AJ507189, AM494937, |
| | | | | | AY045702, EU256083, M84754 |
| 445 | gtc | acc | V | T | AM401872 |
| 446 | tgc | cgc | C | R | AM401864, AM401944, AM402115, AM402264 |
| 447 | tgc | tac | C | Y | AM401856, AM401889 |

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method for reducing the mutation rate of a DNA or an RNA virus in a cell infected with the virus, comprising contacting the cell with an effective amount of an iron chelator or an antioxidant, thereby reducing the mutation rate of the virus in the cell.

2. The method of claim 1, wherein the iron chelator is one or more selected from the group diethylene triamine pentaacetic acid (DETAPAC), dipyridyl, pyridoxal isonicotinoyl hydrazone (PIH), desferrioxamine (DFO), deferiprone (DFP) or deferasirox (DFS).

3. The method of claim 1, wherein the antioxidant is one or more selected from the group glutathione, N-acetylcysteine, ascorbic acid, vitamin E, beta-carotene, flavonoid, a CYP2E1 inhibitor, an NAD(P)H oxidase inhibitor or a nitric oxide synthase inhibitor.

4. The method of claim 1, wherein the virus is an RNA virus.

5. The method of claim 4, wherein the RNA virus is a positive sense single strand RNA virus.

6. The method of claim 5, wherein the positive sense single strand RNA virus is a Flaviviridae family virus.

7. The method of claim 6, wherein the Flaviviridae family virus is selected from the group of a Yellow fever virus, a West Nile virus, a Dengue Fever virus or a hepatitis C virus (HCV).

8. The method of claim 1, wherein the virus is a hepatitis C virus (HCV).

9. The method of claim 1, wherein the virus is a DNA virus.

10. The method of claim 9, wherein the DNA virus is selected from Epstein-Barr viruses (EBV), Herpes simplex virus-1 (HSV-1) or hepatitis B viruses (HBV).

11. The method of claim 1, wherein the cell is a liver cell.

12. A method for enhancing the efficacy of an agent or a therapy to reduce or prevent infection of a cell by a DNA or a RNA virus by reducing the ability of the virus to persist in the cell, comprising contacting the cell with (i) an effective amount of an iron chelator or an antioxidant and (ii) the agent or the therapy, thereby enhancing the efficacy of the agent or the therapy to reduce or prevent infection of the cell by the virus.

13. The method of claim 12, wherein the contacting of (i) the iron chelator or antioxidant and (ii) the agent or the therapy to the cell is sequential or concurrent.

14. The method of claim 12, wherein the virus is a HCV.

15. The method of claim 12, wherein the agent is one or more selected from the group interferon, plerixafor, ribavirin, pegylated interferon-alpha-2a or pegylated interferon-alpha-2b.

16. The method of claim 12 wherein the iron chelator is one or more selected from diethylene triamine pentaacetic acid (DETAPAC), dipyridyl, pyridoxal isonicotinoyl hydrazone (PIH), desferrioxamine (DFO), deferiprone (DFP) or deferasirox (DFS).

17. The method of claim 12, wherein the antioxidant is one or more selected from glutathione, N-acetylcysteine, ascorbic acid, vitamin E, beta-carotene, flavonoid, a CYP2E1 inhibitor, an NAD(P)H oxidase inhibitor or a nitric oxide synthase inhibitor.

18. The method of claim 12, wherein the virus is a DNA virus.

19. The method of claim 18, wherein the DNA virus is selected from HBV, EBV or HSV-1.

20. A method for reducing the mutation rate of a DNA or a RNA virus in a subject, comprising administering an effective amount of an iron chelator or an antioxidant to the subject, thereby reducing the mutation rate of the virus in the subject.

21. The method of claim 20, further comprising administering to the subject an effective amount of an agent or a therapy directed at the virus.

22. The method of claim 21, wherein the agent is one or more selected from the group interferon, plerixafor, ribavirin, pegylated interferon-alpha-2a or pegylated interferon-alpha-2b.

23. The method of claim 20, wherein the iron chelator is one or more selected from the group diethylene triamine pentaacetic acid (DETAPAC), dipyridyl, pyridoxal isonicotinoyl hydrazone (PIH), desferrioxamine (DFO), deferiprone (DFP) or deferasirox (DFS).

24. The method of claim 20, wherein the antioxidant is one or more selected from the group glutathione, N-acetylcysteine, ascorbic acid, vitamin E, beta-carotene, flavonoid, a CYP2E1 inhibitor, an NAD(P)H oxidase inhibitor or a nitric oxide synthase inhibitor.

25. A method for screening an agent useful for reducing the mutation rate of an RNA or DNA virus in a cell, comprising contacting the cell with an agent, the cell being infected with a virus or in contact with a virus, wherein a reduction of the mutation rate of the virus in the cell as compared to a cell not being contacted with the agent indicates that the agent is useful for reducing the mutation rate of the virus.

26. A method for treating a disease incident to DNA or RNA viral infection in a subject, comprising administering to the subject an effective amount of an iron chelator or an antioxidant that reduces the mutation rate of an RNA or DNA virus in the subject.

27. The method of claim 26, wherein the disease is a liver disease.


SEQUENCE LISTING (4 sequences)

SEQ ID NO: 1 - DNA, 37 bp, Artificial Sequence
  Description of Artificial Sequence Synthetic primer
atcaatcgattgtctagagctgaagaggcttcaccag

SEQ ID NO: 2 - DNA, 36 bp, Artificial Sequence
  Description of Artificial Sequence Synthetic primer
gcaatcttgtacaagcttcgcagcgcatggcgtgat

SEQ ID NO: 3 - DNA, 27 bp, Artificial Sequence
  Description of Artificial Sequence Synthetic primer
gcacgcccggattgcaaccctccactg

SEQ ID NO: 4 - DNA, 27 bp, Artificial Sequence
  Description of Artificial Sequence Synthetic primer
cagtggagggttgcaatccgggcgtgc